(19) United States
(12) Patent Application Publication
(10) Pub. No.: US 2026/0355190 A1
(43) Pub. Date: Nov. 12, 2026

(54) ANTHELMINTIC COMBINATIONS

(71) Applicant: Boehringer Ingelheim Vetmedica GmbH, Ingelheim am Rhein (DE)

(72) Inventors: Hannes Fiepko Koolman, Mittelbiberach (DE); Bart Herlé, Mittelbiberach (DE); Daniel Kulke, Leverkusen (DE); Sandra Noack, Münster-Sarmsheim (DE); Ricarda Suessenberger, Engelstadt (DE); Navini Wijeratne, Mainz (DE); Elke Timmerman, Duluth, GA (US)

(21) Appl. No.: 19/632,868

(22) Filed: Mar. 30, 2026

Related U.S. Application Data
(60) Provisional application No. 63/781,752, filed on Apr. 1, 2025.

(30) Foreign Application Priority Data
May 8, 2025 (EP) 25175127.7

(51) Int. Cl. A61K 31/5025; A61K 31/352; A61K 31/42; A61K 31/4745; A61P 33/10; A61P 33/14

(52) U.S. Cl. CPC A61K 31/5025; A61K 31/352; A61K 31/42; A61K 31/4745; A61P 33/10; A61P 33/14

(57) ABSTRACT

The invention relates to novel compounds of formula (I), in which Aa, Ab, Ac, Ad, R¹, R², R³, Q and n have the meanings indicated in the description, to the use thereof as acaricides and/or insecticides for controlling animal pests, and to methods and intermediate products for the production thereof.

### FIELD OF INVENTION

This patent application relates to antiparasitic combinations comprising agonists of the calcium-activated and voltage-gated potassium nematode channels, SLO-1, and one or more additional antiparasitic active agents, compositions comprising the active agent combinations, and methods of using the active agent combinations to prevent, treat and/or control parasites that harm animals and humans.

### CROSS REFERENCE TO RELATED APPLICATIONS

This application claims the benefit of priority to U.S. Provisional Patent Application No. 63/781,752 filed on Apr. 1, 2025, and European Patent Application No. 25175127.7 filed on May 8, 2025, both which are incorporated herein by reference in their entirety.

### BACKGROUND OF INVENTION

Animals, such as mammals and birds, are often susceptible to parasite infestations. These parasites may be ectoparasites, such as fleas and ticks. Animals and humans also suffer from endoparasitic infections including, for example, helminthiasis which is most frequently caused by a group of parasitic worms described as nematodes or roundworms. These parasites cause severe economic losses in pigs, sheep, horses, and cattle as well as affecting companion animals (e.g. cats and dogs) and poultry. Endoparasites include those which occur in the gastrointestinal tract of animals and humans, such as those from the genera Ancylostoma, Necator, Ascaris, Strongyloides, Trichinella, Capillaria, Toxocara, Toxascaris, Trichuris and Enterobius. Other parasites which are found in the blood or other tissues and organs include filarial worms and the extra-intestinal stages of Strongyloides and Trichinella.

One type of endoparasite that seriously harms mammals is Dirofilaria immitis, also known as heartworm. Other filarial endoparasites include Dirofilaria repens and Dirofilaria honkongensis, which can also infect humans. The most common hosts are dogs and cats but other mammals such as ferrets and raccoons may also be infected. Heartworms go through several life stages before they become adults, when they infect the pulmonary artery of the host mammal. These worms require the mosquito as an intermediate host to complete their life cycle.

Thus, there is an ongoing need to develop new anthelmintic agents with improved safety and efficacy against endoparasites, including Dirofilaria immitis and nematodes that infect the gastrointestinal tract of animals, including hookworms and roundworms.

WO 2023/073641, which is incorporated herein by reference in its entirety, describes anthelmintic pyrrolopyridazine compounds, including compound A472 described herein, that are active against endoparasites such as Dirofilaria immitis and parasitic nematodes. The WO publication describes compounds and composition for the control, treatment and/or prevention of parasitic infections in animals.

WO 2017/178416, which is incorporated herein by reference in its entirety, provides pyrazolopyrimidine derivatives for the control, treatment and/or prevention of helminths. This WO publication describes anthelmintic compounds having a different 5-6 fused nitrogen-containing heteroaryl core.

WO 2018/197401 provides bicyclic pyrazole derivatives for the control, treatment and/or prevention of helminths.

WO 2018/087036 provides quinolone-3-carboxamide derivatives for the control, treatment and/or prevention of helminths.

WO 2019/025341 provides quinoline compounds for the treatment, control and/or prevention of helminth infections and WO 2019/002132 describes azaquinoline derivatives for the control, treatment and/or prevention of helminths. WO 2019/215182 also describes quinoline derivates for the treatment and control of helminth infections.

WO 2020/083971 describes additional carboxamide compounds substituted with bicyclic ring systems for the treatment and control of parasitic infections.

WO 2021/204930 describes substituted condensed azines containing a bicyclic nitrogen-containing bicyclic ring system that have anthelmintic properties.

WO 2021/018839 describes isoquinoline derivatives for use in the treatment of parasite infections; and WO 2019/115768 describes a process for preparing anthelmintic 4-amino-quinoline-3-carboxyamide derivatives.

WO 2021/242581 describes imidazopyridazine compounds that are useful for the control, treatment and/or prevention of endoparasitic infections and/or ectoparasitic infestations in or on animals.

WO 2020/131629, WO 2020/131631 and WO 2020/247747 describe bicyclic derivatives which are useful in the control of endoparasites, including heartworm, in warm-blooded animals.

WO 2021/122906 describes anthelmintic compounds comprising azaindole compounds and WO 2021/122911 describes quinoline compounds that are useful for the treatment of disease caused by helminths such as Dirofilaria immitis.

WO 2020/014068 describes anthelmintic heterocyclic compounds having a core having a core bicyclic pyrazolo[1,5-a]pyridine ring system that were found to be active against Dirofilaria immitis. The pyrazolo[1,5-a]pyridine ring system of the compounds described in this WO publication is a 5-6-fused ring system having two nitrogen atoms, one at the 5-6 ring junction and the other in the 5-membered ring.

WO 2020/191091 describes aza-benzothiophene and aza-benzofuran compounds that are useful for the control or prevention of parasitic infections, including against infections by Dirofilaria immitis.

WO 2022/106469 describes N-(2,3-Dihydro-1,4-benzoxazin-4-yl)-3-isopropyl-7-(2,3,5-trifluorophenyl)benzo-thiophene-2-carboxamide derivatives and similar compounds for the treatment of heartworm infections.

WO 2022/117783 describes bicyclic compounds disclosed for the treatment and/or control of endoparasite infections in warm-blooded animals.

WO 2022/122987 describes anthelmintic compounds comprising a pyridine structure for the treatment of heartworm disease caused by Dirofilaria immitis.

WO 2022/122988 describes anthelmintic compounds comprising a thienopyridine structure for the treatment of heartworm disease caused by Dirofilaria immitis.

WO 2022/152918 describes azaquinazoline derivatives for use in treating or preventing Dirofilaria immitis infections in a mammal.

WO 2024/213752 describes long-term prevention and/or treatment of disease by SLO-1 inhibitors, particularly diseases caused by endoparasites that infect animals.

### SUMMARY OF THE INVENTION

The present invention provides for novel and inventive combinations of antiparasitic active agents that include agonists of the calcium-activated and voltage-gated potassium nematode channels, SLO-1, and one or more additional antiparasitic active agents. In one embodiment, the present invention provides a combination that is for use in treating and/or preventing parasitic infections and/or infestations. In another embodiment, the invention provides a method for the treatment and/or prevention of a parasitic infection and/or infestation in an animal comprising administering an effective amount of the antiparasitic combination of the present invention to an animal. The one or more additional antiparasitic active agents combined with the SLO-1 agonists may be endoparasiticide active agents from different compound classes that act against different targets (e.g. macrocyclic lactones or other active agents) or ectoparasiticides that act against arthropod parasites such as fleas and ticks.

In one embodiment, the present invention includes antiparasitic combinations of antiparasitic active agents comprising an anthelmintic compound of Formula (IA), Formula (IB) and/or Formula (IC), or combinations thereof:

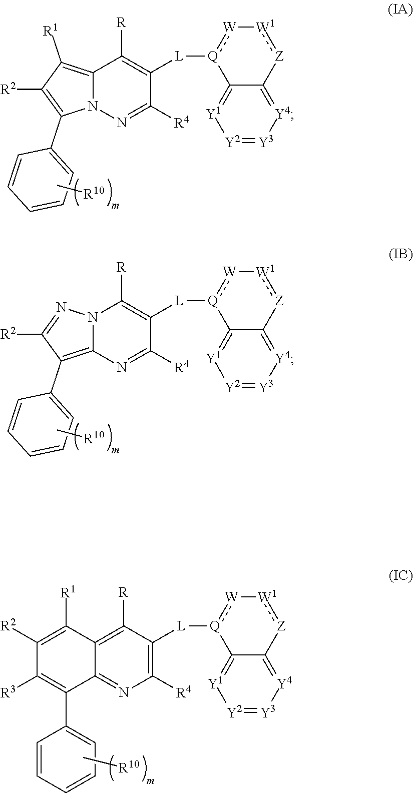

or salt thereof, wherein variables R, R¹, R², R³, R⁴, R¹⁰, L, Q, W, W¹, Z, Y¹, Y², Y³, Y⁴ and m are defined herein, and the dashed bonds ( [symbol] ) signifies a single or double bond in combination with one or more other antiparasitic active agents.

The invention also includes a composition comprising a compound of Formula (IA), Formula (IB) and/or Formula (IC), or a salt thereof, in combination with one or more additional antiparasitic active agents, and a pharmaceutically acceptable carrier. The antiparasitic combinations and compositions comprising the antiparasitic active agent combinations may be administered to animals, particularly mammals, fish and birds, to prevent, control and/or treat parasitic infections and/or infestations in animals. In particular, the antiparasitic combinations and compositions comprising the antiparasitic active agent combinations of the invention may be administered to cats, dogs, horses, chickens, pigs, sheep and cattle, with the aim of substantially ridding these hosts of endoparasites and/or ectoparasites.

In an embodiment, the antiparasitic active agent combinations comprising a compound of Formula (IA), Formula (IB) and/or Formula (IC), or a salt thereof, and compositions comprising said antiparasitic combinations are effective against endoparasites, such as filariae (e.g., Dirofilaria immitis), and nematodes including, but not limited to, hookworms, whipworms and roundworms of the digestive tract of animals and humans.

Another embodiment includes a method for the treatment, control and/or prevention of a parasitic infection and/or infestation in an animal comprising administering a compound of Formula (IA), Formula (IB) and/or Formula (IC), or a salt thereof, in combination with one or more additional antiparasitic active agents to the animal. Another embodiment includes the use of a compound of Formula (IA), Formula (IB) and/or Formula (IC), or a salt thereof, in combination with one or more additional antiparasitic active agents for the treatment, control and/or prevention of a parasitic infection and/or infestation in an animal. Also provided in another embodiment is the use of the compound of Formula (IA), Formula (IB) and/or Formula (IC), or a salt thereof, in combination with one or more additional antiparasitic active agents in the preparation of a medicament for the treatment, control and/or prevention of a parasitic infection and/or infestation in or on an animal.

Thus, the invention includes the following non-limiting embodiments:
(a) An antiparasitic combination of an antiparasitic compound of Formula (IA), Formula (IB) or Formula (IC), or a salt thereof, with one or more additional antiparasitic active agents;
(b) an antiparasitic combination of an antiparasitic compound of Formula (IA), Formula (IB) and/or Formula (IC), or a salt thereof, with one or more endoparasitic active agents;
(c) an antiparasitic combination of an antiparasitic compound of Formula (IA), Formula (IB) and/or Formula (IC), or a salt thereof, with one or more ectoparasitic active agents;
(d) an antiparasitic combination of an antiparasitic compound of Formula (IA), Formula (IB) and/or Formula (IC), or a salt thereof, with one or more endoparasitic active agents and one or more ectoparasitic active agents;
(e) a veterinary composition comprising an antiparasitic compound of Formula (IA), Formula (IB) and/or Formula (IC), or a salt thereof, in combination with one or more additional antiparasitic active agents and a pharmaceutically acceptable carrier or diluent;
(f) a veterinary composition comprising an antiparasitic compound of Formula (IA), Formula (IB) and/or Formula (IC), or a salt thereof, in combination with one or more additional endoparasitic active agents and a pharmaceutically acceptable carrier or diluent;
(g) a veterinary composition comprising an antiparasitic compound of Formula (IA), Formula (IB) and/or Formula (IC), or a salt thereof, in combination with one or more additional ectoparasitic active agents and a pharmaceutically acceptable carrier or diluent;
(h) a veterinary composition comprising an antiparasitic compound of Formula (IA), Formula (IB) and/or Formula (IC), or a salt thereof, in combination with one or more endoparasitic active agents and one or more ectoparasitic active agents, and a pharmaceutically acceptable carrier or diluent;
(i) a method for treating a parasitic infection and/or infestation in or on an animal comprising administering a parasiticidally effective amount of an antiparasitic compound of Formula (IA), Formula (IB) and/or Formula (IC), or a salt thereof, in combination with an effective amount of one or more additional antiparasitic active agents, to the animal in need thereof;
(j) a method for treating a parasitic infection and/or infestation in or on an animal comprising administering a parasiticidally effective amount of an antiparasitic compound of Formula (IA), Formula (IB) and/or Formula (IC), or a salt thereof, in combination with an effective amount of one or more additional ectoparasitic active agents, to the animal in need thereof;
(k) a method for treating a parasitic infection and/or infestation in or on an animal comprising administering a parasiticidally effective amount of an antiparasitic compound of Formula (IA), Formula (IB) and/or Formula (IC), or a salt thereof, in combination with an effective amount of one or more additional endoparasitic active agents, to the animal in need thereof;
(l) a method for treating a parasitic infection and/or infestation in or on an animal comprising administering a parasiticidally effective amount of an antiparasitic compound of Formula (IA), Formula (IB) and/or Formula (IC), or a salt thereof, in combination with one or more endoparasitic active agents and one or more ectoparasitic active agents, to the animal in need thereof,
(m) a method for the prevention of a parasitic infection and/or infestation of an animal, which comprises administering a parasiticidally effective amount of an antiparasitic compound of Formula (IA), Formula (IB) and/or Formula (IC), or a salt thereof, with an effective amount of one or more additional antiparasitic active agents to the animal in need thereof,
(n) a method for the prevention of a parasitic infection and/or infestation of an animal, which comprises administering a parasiticidally effective amount of an antiparasitic compound of Formula (IA), Formula (IB) and/or Formula (IC), or a salt thereof, with an effective amount of one or more additional endoparasitic active agents to the animal in need thereof,
(o) a method for the prevention of a parasitic infection and/or infestation of an animal, which comprises administering a parasiticidally effective amount of an antiparasitic compound of Formula (IA), Formula (IB) and/or Formula (IC), or a salt thereof, with an effective amount of one or more additional ectoparasitic active agents to the animal in need thereof,
(p) a method for the prevention of a parasitic infection and/or infestation of an animal, which comprises administering a parasiticidally effective amount of an antiparasitic compound of Formula (IA), Formula (IB) and/or Formula (IC), or a salt thereof, with an effective amount of one or more additional endoparasitic active agents and one or more additional ectoparasitic active agents to the animal in need thereof,
(q) an antiparasitic compound of Formula (IA), Formula (IB) and/or Formula (IC), or a salt thereof, with one or more additional antiparasitic active agents for use in the treatment, control and/or prevention of a parasitic infection and/or a parasitic infestation in an animal;
(r) an antiparasitic compound of Formula (IA), Formula (IB) and/or Formula (IC), or a salt thereof, with one or more additional endoparasitic active agents for use in the treatment, control and/or prevention of a parasitic infection and/or a parasitic infestation in an animal;
(s) an antiparasitic compound of Formula (IA), Formula (IB) and/or Formula (IC), or a salt thereof, with one or more additional ectoparasitic active agents for use in the treatment, control and/or prevention of a parasitic infection and/or a parasitic infestation in an animal;
(t) an antiparasitic compound of Formula (IA), Formula (IB) and/or Formula (IC), or a salt thereof, with one or more additional endoparasitic active agents and one or more additional ectoparasitic active agents for use in the treatment, control and/or prevention of a parasitic infection and possibly also a parasitic infestation in an animal;
(u) an antiparasitic compound of Formula (IA), Formula (IB) and/or Formula (IC), or a salt thereof, with one or more additional antiparasitic active agents for use in the treatment of a parasitic infection and possibly also a parasitic infestation in an animal;
(v) an antiparasitic compound of Formula (IA), Formula (IB) and/or Formula (IC), or a salt thereof, with one or more additional endoparasitic active agents for use in the treatment of a parasitic infection and possibly also a parasitic infestation in an animal;
(w) an antiparasitic compound of Formula (IA), Formula (IB) and/or Formula (IC), or a salt thereof, with one or more additional ectoparasitic active agents for use in the treatment of a parasitic infection and possibly also a parasitic infestation in an animal;
(x) an antiparasitic compound of Formula (IA), Formula (IB) and/or Formula (IC), or a salt thereof, with one or more additional endoparasitic active agents and one or more additional ectoparasitic active agents for use in the treatment of a parasitic infection and possibly also a parasitic infestation in an animal;
(y) an antiparasitic compound of Formula (IA), Formula (IB) and/or Formula (IC), or a salt thereof, with one or more additional antiparasitic active agents for use in the prevention of a parasitic infection and possibly also a parasitic infestation in an animal;
(z) an antiparasitic compound of Formula (IA), Formula (IB) and/or Formula (IC), or a salt thereof, with one or more additional endoparasitic active agents for use in the prevention of a parasitic infection and possibly also a parasitic infestation in an animal;
(aa) an antiparasitic compound of Formula (IA), Formula (IB) and/or Formula (IC), or a salt thereof, with one or more additional ectoparasitic active agents for use in the prevention of a parasitic infection and possibly also a parasitic infestation in an animal;
(bb) an antiparasitic compound of Formula (IA), Formula (IB) and/or Formula (IC), or a salt thereof, with one or more additional endoparasitic active agents and one or more additional ectoparasitic active agents for use in the prevention of a parasitic infection and possibly also a parasitic infestation in an animal;
(cc) use of an antiparasitic compound of Formula (IA), Formula (IB) and/or Formula (IC), or a salt thereof, in combination with one or more additional antiparasitic active agents for the manufacture of a veterinary medicament for the treatment, control and/or prevention of a parasitic infection and/or infestation in an animal;
(dd) use of an antiparasitic compound of Formula (IA), Formula (IB) and/or Formula (IC), or a salt thereof, in combination with one or more additional endoparasitic active agents for the manufacture of a veterinary medicament for the treatment, control and/or prevention of a parasitic infection and/or infestation in an animal
(ee) use of an antiparasitic compound of Formula (IA), Formula (IB) and/or Formula (IC), or a salt thereof, in combination with one or more additional ectoparasitic active agents for the manufacture of a veterinary medicament for the treatment, control and/or prevention of a parasitic infection and/or infestation in an animal;
(ff) use of an antiparasitic compound of Formula (IA), Formula (IB) and/or Formula (IC), or a salt thereof, in combination with one or more additional endoparasitic active agents and one or more ectoparasitic active agents for the manufacture of a veterinary medicament for the treatment, control and/or prevention of a parasitic infection and/or infestation in an animal;
(gg) use of an antiparasitic compound of Formula (IA), Formula (IB) and/or Formula (IC), or a salt thereof, in combination with one or more additional antiparasitic active agents for the manufacture of a veterinary medicament for the treatment of a parasitic infection and/or infestation in an animal;
(hh) use of an antiparasitic compound of Formula (IA), Formula (IB) and/or Formula (IC), or a salt thereof, in combination with one or more additional endoparasitic active agents for the manufacture of a veterinary medicament for the treatment of a parasitic infection and/or infestation in an animal
(ii) use of an antiparasitic compound of Formula (IA), Formula (IB) and/or Formula (IC), or a salt thereof, in combination with one or more additional ectoparasitic active agents for the manufacture of a veterinary medicament for the treatment of a parasitic infection and/or infestation in an animal;
(jj) use of an antiparasitic compound of Formula (IA), Formula (IB) and/or Formula (IC), or a salt thereof, in combination with one or more additional endoparasitic active agents and one or more ectoparasitic active agents for the manufacture of a veterinary medicament for the treatment of a parasitic infection and/or infestation in an animal;
(kk) use of an antiparasitic compound of Formula (IA), Formula (TB) and/or Formula (IC), or a salt thereof, in combination with one or more additional antiparasitic active agents for the manufacture of a veterinary medicament for the prevention of a parasitic infection and/or infestation in an animal;
(ll) use of an antiparasitic compound of Formula (IA), Formula (IB) and/or Formula (IC), or a salt thereof, in combination with one or more additional endoparasitic active agents for the manufacture of a veterinary medicament for the prevention of a parasitic infection and/or infestation in an animal
(mm) use of an antiparasitic compound of Formula (IA), Formula (IB) and/or Formula (IC), or a salt thereof, in combination with one or more additional ectoparasitic active agents for the manufacture of a veterinary medicament for the prevention of a parasitic infection and/or infestation in an animal; and
(nn) use of an antiparasitic compound of Formula (IA), Formula (IB) and/or Formula (IC), or a salt thereof, in combination with one or more additional endoparasitic active agents and one or more ectoparasitic active agents for the manufacture of a veterinary medicament for the prevention of a parasitic infection and/or infestation in an animal.

### INCORPORATION BY REFERENCE

All references cited herein are incorporated by reference herein, in their entirety.

### DETAILED DESCRIPTION

It is noted that in this disclosure and particularly in the claims and/or paragraphs, terms such as “comprises”, “comprised”, “comprising” and the like can be interpreted as “includes”, “included”, “including”, and the like; and that terms such as “consisting essentially of” and “consists essentially of” are interpreted as allowing for elements not explicitly recited, but excluding elements that are found in the prior art or that affect a basic or novel characteristic of the invention. The term “consist of” excludes any element, step, or ingredient not specified in the claim.

Unless defined otherwise, all technical and scientific terms used herein have the same meaning as is commonly understood by one of skill in the art to which this invention belongs at the time of filing. Terms not specifically defined herein should be given the meanings that would be given to them by one of skill in the art in light of the disclosure and the context. The meaning and scope of terms should be clear; however, in the event of any latent ambiguity, definitions provided herein take precedent over any dictionary or extrinsic definition. Further, unless otherwise required by context, singular terms shall include pluralities and plural terms shall include the singular. Herein, the use of “or” means “and/or” unless stated otherwise. Furthermore, the use of the term “including”, as well as other forms such as “includes” and “included” is not limiting. As used in the specification, however, unless specified to the contrary, the following terms have the meaning indicated and the following conventions are adhered to.

In the groups, radicals, or moieties defined herein, the number of carbon atoms is often specified preceding the group, for example, C₁₋₆-alkyl means an alkyl group or radical having 1 to 6 carbon atoms. In general, in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last-named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a chemical formula, in case of any discrepancy the chemical formula shall prevail. An asterisk may be used in sub-formulas to indicate the bond which is connected to the core molecule as defined.

The numeration of the atoms of a substituent starts with the atom which is closest to the core or to the group to which the substituent is attached. For example, the term “3-carboxypropyl-group” represents the following substituent:

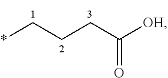 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

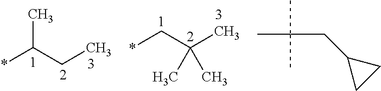

The asterisk or “ [symbol] ” may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless indicated otherwise for a specific embodiment of the invention, the term “optionally substituted” as used herein is meant to indicate that a given radical is optionally substituted by one or more of the following moieties: halogen, hydroxyl, oxo (C═O), alkyl, haloalkyl, cycloalkyl, cycloalkenyl, carboxyl, acyl, acyloxy, acetyl, alkylcarbonyl, haloalkylcarbonyl, alkoxycarbonyl, haloalkoxycarbonyl, aminocarbonyl, alkylaminocarbonyl, dialkylaminocarbonyl, haloalkylaminocarbonyl, dihaloalkylaminocarbonyl, amino, alkyl- or dialkylamino, amido, arylamino, alkoxy, haloalkoxy, aryloxy, alkylthio, haloalkylthio, nitro, cyano, azido, thiol, thioamido, imino, amidino, guanidino, carbonate, R₃Si— or R₃SiO— where R is C₁-C₆-alkyl, C₁-C₆-haloalkyl or aryl, SF₅, sulfate, alkylsulfonyl, haloalkylsulfonyl, alkoxysulfonyl, haloalkoxysulfonyl, arylsulfonyl, alkylsulfinyl, haloalkylsulfinyl, arylsulfinyl, sulfamoyl, sulfoximine, sulfinimine, sulfonimidamide, sulfonediimine, phosphonyl, phosphinyl, phosphoryl, phosphine, phosphonamidate, phosphinamidate, phosphinate, phosphine oxide, thioester, anhydride, oxime, hydrazine, carbamate, phosphate, phosphonate, aryl, heteroaryl and heterocyclyl.

In some embodiments, unless indicated otherwise for a specific embodiment of the invention, the term “optionally substituted” includes substitution of a core group with halogen (chloro, fluoro, bromo, iodo), hydroxyl, oxo, C₁-C₆-alkyl, C₁-C₆-haloalkyl, 3- to 8-membered cycloalkyl, 5- to 8-membered cycloalkenyl, carboxyl, amino, amido, thioamide, imino, amidino, guanidino, carbonate, tri-C₁-C₆-alkylsilyl, tri-C₁-C₆-alkylsiloxy,sulfate, C₁-C₆-alkylamino, C₁-C₆-dialkylamino, phenylamino, C₁-C₆-alkoxy, C₁-C₆-haloalkoxy, phenoxy, cyano, azido, thiol, nitro, SFS, acetyl, C₁-C₆-alkoxycarbonyl, C₁-C₆-alkoxycarbonyloxy, C₁-C₆-haloalkoxycarbonyl, C₁-C₆-alkylcarbonyl, C₁-C₆-alkylcarbonyloxy, C₁-C₆-haloalkylcarbonyl, aminocarbonyl, C₁-C₆-alkylaminocarbonyl, C₁-C₆-dialkylaminocarbonyl, aminocarbonyloxy, C₁-C₆-alkylaminocarbonyloxy, C₁-C₆-dialkylaminocarbonyloxy, C₁-C₆-haloalkylaminocarbonyl, C₁-C₆-dihaloalkylaminocarbonyl, C₁-C₆-alkylthio, C₁-C₆-alkylsulfinyl, phenylsulfinyl, C₁-C₆-alkylsulfonyl, phenylsulfonyl, C₁-C₆-alkoxysulfonyl, C₁-C₆-haloalkylthio, C₁-C₆-haloalkylsulfinyl, C₁-C₆-haloalkylsulfonyl, C₁-C₆-haloalkoxysulfonyl, phenyl, 5- or 6-membered heteroaryl, 5- or 6-membered heterocyclyl, sulfamoyl, sulfoximine, sulfinimine, sulfonimidamide, sulfonediimine, phosphonyl, phosphinyl, phosphoryl, phosphine, phosphonamidate, phosphinamidate, phosphinate, phosphine oxide, thioester, anhydride, oxime, hydrazine, carbamate, phosphate or phosphonate.

In other embodiments, unless indicated otherwise for a specific embodiment of the invention, the term “optionally substituted” includes substitution of a core group with halogen (chloro, fluoro, bromo, iodo), hydroxyl, C₁-C₃-alkyl, C₁-C₃-haloalkyl, 3- to 8-membered cycloalkyl, 5-8-membered cycloalkenyl, amino, C₁-C₃-alkylamino, C₁-C₃-dialkylamino, C₁-C₃-alkoxy, C₁-C₃-haloalkoxy, cyano, nitro, SF₅, acetyl, C₁-C₃-alkoxycarbonyl, C₁-C₃-haloalkoxycarbonyl, C₁-C₃-alkylcarbonyl, C₁-C₃-haloalkylcarbonyl, aminocarbonyl, C₁-C₃-alkylaminocarbonyl, C₁-C₃-dialkylaminocarbonyl, C₁-C₃-haloalkylaminocarbonyl, C₁-C₃-dihaloalkylaminocarbonyl, C₁-C₃-alkylthio, C₁-C₃-alkylsulfinyl, C₁-C₃-alkylsulfonyl, C₁-C₃-haloalkylthio, C₁-C₃-haloalkylsulfinyl, C₁-C₃-haloalkylsulfonyl, phenyl, 5- or 6-membered heteroaryl, 5- or 6-membered heterocyclyl.

In certain embodiments, unless indicated otherwise for a specific embodiment of the invention, the term “optionally substituted” includes substitution by halogen (chloro, fluoro, bromo and iodo), methyl, ethyl, n-propyl, iso-propyl, n-butyl, iso-butyl, sec-butyl, tert-butyl, cyclopropyl, cyclobutyl, cyclopentyl, cyclohexyl, hydroxyl, thiol, amino, methylamino, ethylamino, propylamino, butylamino, dimethylamino, diethylamino, methoxy, ethoxy, propoxy, CF₃, CF₂CF₃, —OCF₃, —OCF₂CF₃, —SCH₃, —SCF₃, —S(O)CH₃, —S(O)CF₃, —S(O)₂CH₃, —S(O)₂CF₃, aziridinyl, azetidinyl, morpholino, piperidinyl, pyridyl and phenyl.

Unless specifically indicated, throughout the specification and the appended claims, a given chemical formula or name shall encompass tautomers and all stereo, optical and geometrical isomers (e.g. enantiomers, diastereomers, E/Z isomers etc.) and racemates thereof as well as mixtures in different proportions of the separate enantiomers, mixtures of diastereomers, or mixtures of any of the foregoing forms where such isomers and enantiomers exist, as well as solvates thereof, such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of animals without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts) also comprise a part of the invention.

The term “halogen” denotes fluorine, chlorine, bromine and iodine.

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₁₋ₙ-alkylene”, wherein n is an integer selected from 2, 3, 4, 5 or 6, preferably 4, 5 or 6, either alone or in combination with another radical, denotes an acyclic, saturated, branched or linear chain divalent alkyl radical containing from 1 to n carbon atoms. For example, the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(C H₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH₃)—, —CH₂—CH(C H₂CH₃)—, —CH(CH₂CH₃)—CH₂—, —CH(CH₂CH₂CH₃)—, —CH(CH(CH₃))₂— and —C(CH₃)(CH₂CH₃)—.

The term “C₂₋ₘ-alkenyl” is used for a group “C₂₋ₘ-alkyl”, wherein m is an integer selected from 3, 4, 5 or 6, preferably 4, 5 or 6, if at least two carbon atoms of said group are bonded to each other by a double bond.

The term “C₂₋ₘ-alkenylene” is used for a group “C₂₋ₘ-alkylene”, wherein m is an integer selected from 3, 4, 5 or 6, preferably 4, 5 or 6, if at least two carbon atoms of said group are bonded to each other by a double bond.

The term “C₂₋ₘ-alkynyl” is used for a group “C₂₋ₘ-alkyl”, wherein m is an integer selected from 3, 4, 5 or 6, preferably 4, 5 or 6, if at least two carbon atoms of said group are bonded to each other by a triple bond.

The term “C₂₋ₘ-alkynylene” is used for a group “C₂₋ₘ-alkylene”, wherein m is an integer selected from 3, 4, 5 or 6, preferably 4, 5 or 6, if at least two of those carbon atoms of said group are bonded to each other by a triple bond.

The term “C₃₋ₖ-cycloalkyl”, wherein k is an integer selected from 4, 5, 6, 7 or 8, preferably 4, 5 or 6, either alone or in combination with another radical, denotes a cyclic, saturated, unbranched hydrocarbon radical with 3 to k C atoms. For example, the term C₃₋₇-cycloalkyl includes cyclopropyl, cyclobutyl, cyclopentyl, cyclohexyl and cycloheptyl.

The term “C₃₋ₖ-cycloalkenyl”, wherein k is an integer selected from 4, 5, 6, 7 or 8, preferably 4, 5 or 6, either alone or in combination with another radical, denotes a cyclic, unsaturated, but non-aromatic, unbranched hydrocarbon radical with 3 to k C atoms, at least two of which are bonded to each other by a double bond. For example, the term C₃₋₇-cycloalkenyl includes cyclopropenyl, cyclobutenyl, cyclopentenyl, cyclopentadienyl, cyclohexenyl, cyclohexadienyl, cycloheptenyl, cycloheptadienyl and cycloheptatrienyl.

The term “halo” added to an “alkyl”, “alkylene”, “alkenyl”, “alkenylene”, “alkynyl”, “alkynylene”, “cycloalkyl”, “cycloalkenyl” or “alkoxy” group defines an alkyl, alkylene, alkenyl, alkenylene, alkynyl, alkynylene, cycloalkyl, cycloalkenyl or alkoxy group, wherein one or more hydrogen atoms are replaced by a halogen atom selected from among fluorine, chlorine, bromine or iodine, preferably fluorine and chlorine, particularly preferred is fluorine. For example C₁-C₄-haloalkyl includes, but is not limited to, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “fluoroalkyl” as used herein refers to an alkyl in which one or more of the hydrogen atoms is replaced with fluorine atoms, for example difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “alkoxy” refers to an alkyl-O—, wherein alkyl is as defined above. Similarly, the terms “alkenyloxy”, “alkynyloxy”, “haloalkoxy”, “haloalkenyloxy”, “haloalkynyloxy”, “cycloalkoxy”, “cycloalkenyloxy”, “halocycloalkoxy”, and “halocycloalkenyloxy” refer to the groups alkenyl-O—, alkynyl-O—, haloalkyl-O—, haloalkenyl-O—, haloalkynyl-O—, cycloalkyl-O—, cycloalkenyl-O—, halocycloalkyl-O—, and halocycloalkenyl-O—, respectively, wherein alkenyl, alkynyl, haloalkyl, haloalkenyl, haloalkynyl, cycloalkyl, cycloalkenyl, halocycloalkyl, and halocycloalkenyl are as defined herein. Examples of C₁-C₆-alkoxy include, but are not limited to, methoxy, ethoxy, OCH₂—C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

The term “carbocyclyl” or “carbocycle”, either alone or in combination with another radical, means a mono-, bi- or tricyclic ring structure consisting of 3 to 14 carbon atoms. The term “carbocyclyl” or “carbocycle” refers to fully saturated, partially saturated and aromatic ring systems. The term “carbocyclyl” or “carbocycle” encompasses fused, bridged and spirocyclic systems. Examples include:

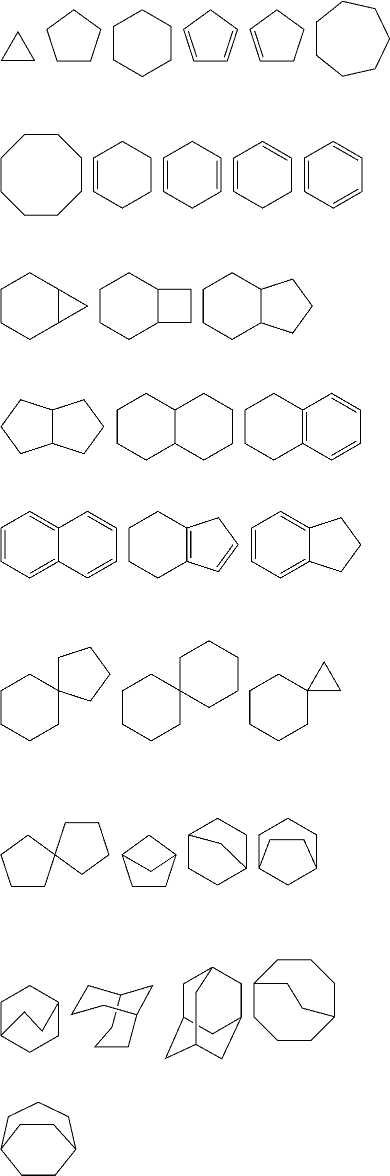

The term “aryl”, either alone or in combination with another radical, denotes a carbocyclic aromatic monocyclic group containing 6 carbon atoms, which is optionally further fused to a second five- or six-membered, carbocyclic group which is aromatic, fully saturated or partially saturated. The term “aryl” includes, but is not limited to, phenyl, indanyl, indenyl, naphthyl, anthracenyl, phenanthrenyl, tetrahydronaphthyl and dihydronaphthyl.

The term “aralkyl” refers to an aryl group that is bonded to the parent compound through a diradical alkylene bridge, (—CH₂—)ₙ, where n is 1-6 and where “aryl” is as defined herein.

The term “heterocyclyl” or “heterocycle” means a saturated or unsaturated mono- or polycyclic ring system optionally comprising aromatic rings, containing one or more heteroatoms selected from N, O, S, S(O) or S(O)₂ consisting of 3 to 14 ring atoms, wherein none of the heteroatoms is part of the aromatic ring. The term “heterocyclyl” or “heterocycle” is intended to include all possible isomeric forms. Thus, the term “heterocyclyl” or “heterocycle” includes the following exemplary structures (not depicted as radicals as each form is optionally attached through a covalent bond to any atom so long as appropriate valences are maintained):

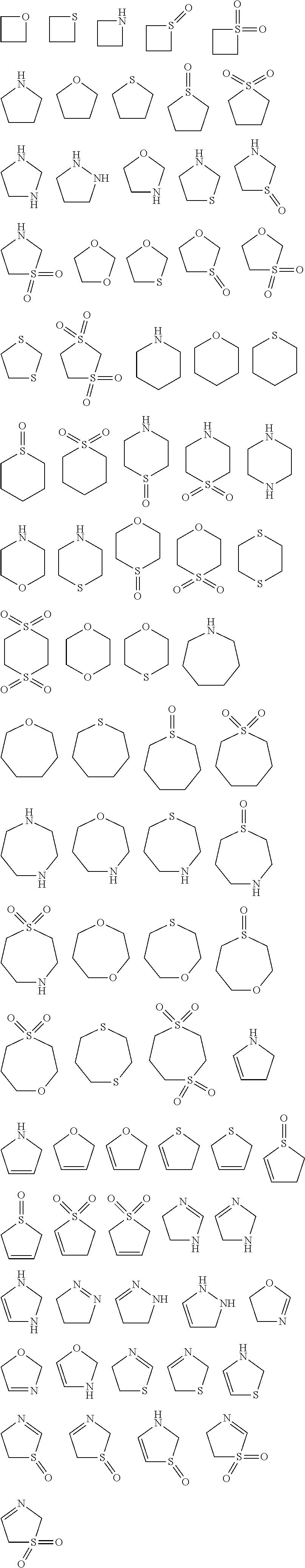

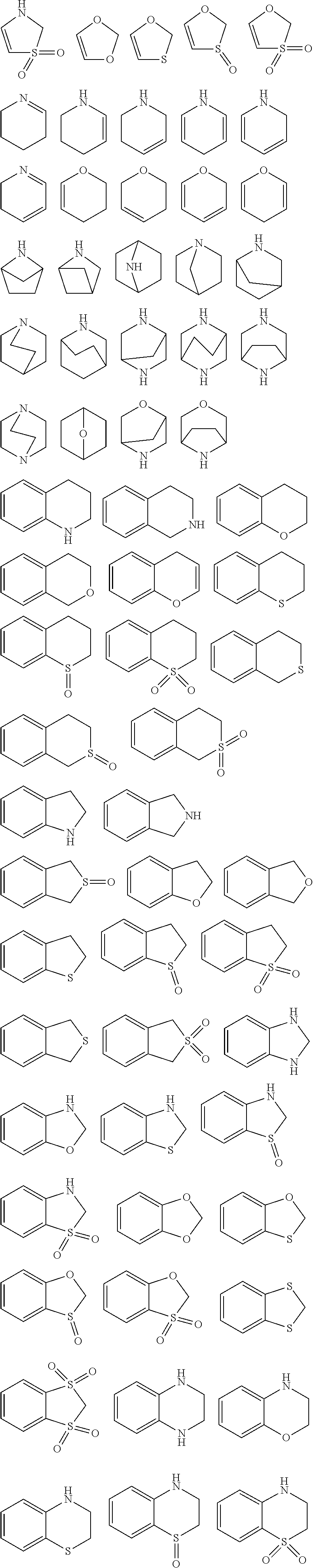

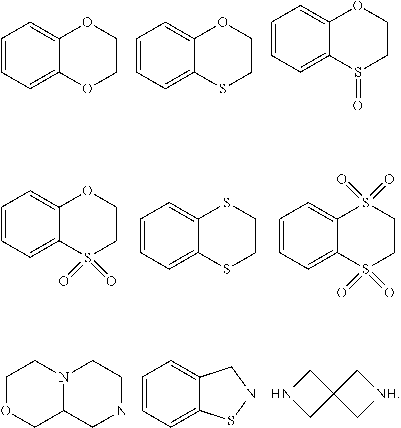

The term “heteroaryl” means a mono- or polycyclic ring system, comprising at least one aromatic ring, containing one or more heteroatoms selected from N, O, S, S(O) or S(O)₂, consisting of 5 to 14 ring atoms, wherein at least one of the heteroatoms is part of an aromatic ring. The term “heteroaryl” is intended to include all the possible isomeric forms. Thus, the term “heteroaryl” includes the following exemplary structures (not depicted as radicals as each form is optionally attached through a covalent bond to any atom so long as appropriate valences are maintained):

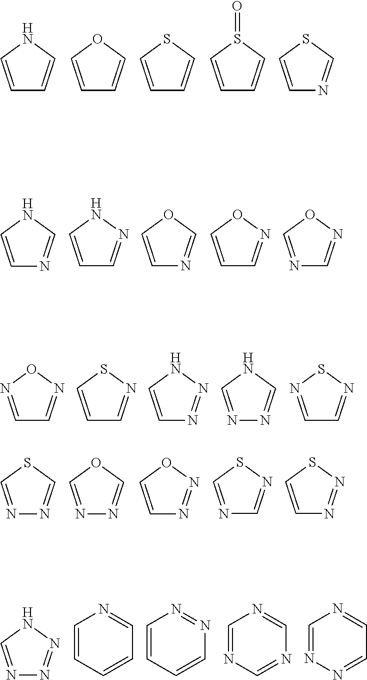

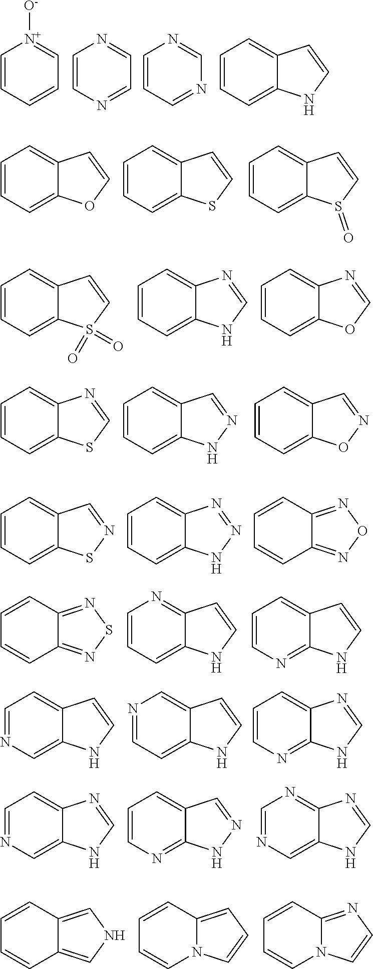

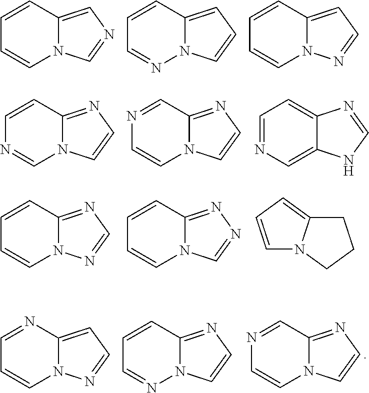

Many of the terms given herein may be used repeatedly in the definition of a formula or group and in each case have one of the meanings given herein, independently of one another.

The term “bicyclic ring systems” means groups consisting of 2 joined cyclic substructures including spirocyclic, fused, and bridged ring systems.

The term “tricyclic ring systems” means groups consisting of 3 joined cyclic substructures including spirocyclic, fused, and bridged ring systems.

As used herein, the term “control” in connection with “parasitic infections and/or infestations in animals” means that the parasitic infection and/or infestation is ameliorated or improved, sustainedly reduced in incidence and/or prevented from worsening as regards the animal.

A first embodiment of the present invention includes an antiparasitic combination of antiparasitic active agents comprising at least one antiparasitic compound of Formula (IA), Formula (IB) and/or Formula (IC), or a salt thereof, and at least one additional antiparasitic active agent.

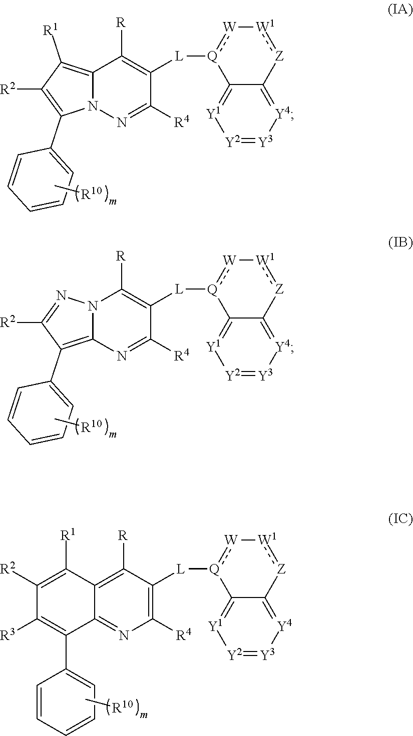

wherein:
L is L1 or L2:

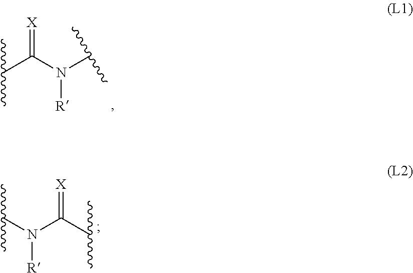

R′ is hydrogen, optionally substituted alkyl or optionally substituted haloalkyl;
R is optionally substituted alkyl, optionally substituted cycloalkyl, optionally substituted cycloalkenyl, optionally substituted alkenyl, optionally substituted alkynyl, optionally substituted heterocyclyl or —NR^{a}R^{b}, wherein R^{a} and R^{b} are independently H or optionally substituted alkyl;
R¹, R², R³ and R⁴ are independently hydrogen, cyano, halogen, optionally substituted alkyl, optionally substituted alkenyl, optionally substituted alkynyl, optionally substituted alkoxy;
R⁵ is independently each occurrence, hydrogen, halogen, cyano, optionally substituted alkyl, optionally substituted alkoxy, optionally substituted alkylcarbonyl, optionally substituted alkoxycarbonyl, optionally substituted aminocarbonyl, optionally substituted alkylaminocarbonyl, optionally substituted dialkylaminocarbonyl, optionally substituted alkylcarbonyloxy, optionally substituted alkylcarbonylamino, —SOₚ(optionally substituted alkyl), —SOₚ(optionally substituted haloalkyl);
R⁶ and R⁷ are independently hydrogen, halogen, optionally substituted alkyl, optionally substituted alkenyl, optionally substituted alkynyl, optionally substituted alkoxy, or R⁶ together with R⁷ for a carbonyl group (C═O);
R⁸ is hydrogen or optionally substituted alkyl;
R⁹ is hydrogen, halogen or optionally substituted alkyl;
R¹⁰ is cyano, halogen, optionally substituted alkyl or optionally substituted alkoxy;
Q is C, C—R⁹ or N;
X is O, S or N—R′;
Y¹, Y², Y³ and Y⁴ are each independently N or —CR⁵—;
W is CR⁶, CR⁶R⁷, O, SOₚ, N or N—R⁸, or W is absent;
W¹ is CR⁶, CR⁶R⁷, O, SOₚ, N or N—R⁸
Z is CR⁶, CR⁶R⁷, O, SOₚ, N or N—R⁸, and
wherein at most three of Y¹, Y², Y³ and Y⁴ are N;
m is 0, 1, 2, 3, 4 or 5;
p is independently in each occurrence is 0, 1, or 2;
the dashed bonds ( [symbol] ) signifies a single or double bond; and
wherein “optionally substituted” indicates that the group recited may be optionally substituted with halogen (independently chloro, fluoro, bromo or iodo), hydroxyl, thiol, oxo (C═O), C₁-C₆-alkyl, C₁-C₆-haloalkyl, C₃-C₈-cycloalkyl, C₁-C₆-alkoxy, C₁-C₆-haloalkoxy, —S(O)ₚ(C₁-C₆-alkyl) or —S(O)ₚ(C₁-C₆-haloalkyl) where p is 0, 1 or 2, carboxyl, acetyl, C₁-C₆-alkylcarbonyl, C₁-C₆-haloalkylcarbonyl, C₁-C₆-alkoxycarbonyl, C₁-C₆-haloalkoxycarbonyl, aminocarbonyl, C₁-C₆-alkylaminocarbonyl, di-C₁-C₆-alkylaminocarbonyl, C₁-C₆-haloalkylaminocarbonyl, di-C₁-C₆-haloalkylaminocarbonyl, amino, C₁-C₆-alkylamino, di-C₁-C₆-alkylamino, nitro, cyano, R₃Si— or R₃SiO— where R is C₁-C₆-alkyl, C₁-C₆-haloalkyl or phenyl; SF₅, phenyl, naphthyl, 5- to 10-membered heteroaryl containing from one to three N, O, Si or S heteroatoms; and 3- to 10-membered heterocyclyl containing from one to three N, O, Si or S heteroatoms.

In a further embodiment, the foregoing combination is for use in treating and/or preventing a parasitic infection and/or infestation in and/or on an animal.

In another embodiment, a method for the treatment and/or prevention of a parasitic infection and/or infestation in and/or on an animal is provided comprising administering the foregoing combination to an animal.

In another embodiment, the invention provides an antiparasitic combination of antiparasitic active agents comprising at least one antiparasitic compound of Formula (IA), Formula (IB) and/or Formula (IC), or a salt thereof, together with one or more additional antiparasitic active agents,
wherein:
R′ is hydrogen, optionally substituted C₁-C₆-alkyl or optionally substituted C₁-C₆-haloalkyl;
R is optionally substituted C₁-C₆-alkyl, optionally substituted C₁-C₆-haloalkyl, optionally substituted C₃-C₈-cycloalkyl, optionally substituted C₄-C₈-cycloalkenyl, optionally substituted C₂-C₆-alkenyl, optionally substituted C₂-C₆-alkynyl, optionally substituted 3- to 10-membered heterocyclyl or —NR^{a}R^{b}, wherein R^{a} and R are independently H or optionally substituted C₁-C₆-alkyl;
R¹, R², R³ and R⁴ are independently hydrogen, cyano, halogen, optionally substituted C₁-C₆-alkyl, optionally substituted C₁-C₆-haloalkyl, optionally substituted C₂-C₆-alkenyl, optionally substituted C₂-C₆-haloalkenyl, optionally substituted C₂-C₆-alkynyl, optionally substituted C₂-C₆-haloalkynyl, optionally substituted C₁-C₆-alkoxy, optionally substituted C₁-C₆-haloalkoxy;
R⁵ is independently in each occurrence, hydrogen, halogen, cyano, optionally substituted C₁-C₆-alkyl, optionally substituted C₁-C₆-haloalkyl, optionally substituted C₁-C₆-alkoxy, optionally substituted C₁-C₆-haloalkoxy, optionally substituted C₁-C₆-alkylcarbonyl, optionally substituted C₁-C₆-alkoxycarbonyl, optionally substituted aminocarbonyl, optionally substituted C₁-C₆-alkylaminocarbonyl, optionally substituted di-C₁-C₆-alkylaminocarbonyl, optionally substituted C₁-C₆-alkylcarbonyloxy, optionally substituted C₁-C₆-alkylcarbonylamino, —SOₚ(optionally substituted C₁-C₆-alkyl), —SOₚ(optionally substituted C₁-C₆-haloalkyl);
R⁶ and R⁷ are independently hydrogen, halogen, optionally substituted C₁-C₆-alkyl, optionally substituted C₁-C₆-haloalkyl, optionally substituted C₂-C₆-alkenyl, optionally substituted C₂-C₆-haloalkenyl, optionally substituted C₂-C₆-alkynyl, optionally substituted C₂-C₆-haloalkynyl, optionally substituted C₁-C₆-alkoxy or optionally substituted C₁-C₆-haloalkoxy;
R⁸ is hydrogen, optionally substituted C₁-C₆-alkyl or optionally substituted C₁-C₆-haloalkyl;
R⁹ is hydrogen, halogen, optionally substituted C₁-C₆-alkyl or optionally substituted C₁-C₆-haloalkyl;
R¹⁰ is cyano, halogen, optionally substituted C₁-C₆-alkyl, optionally substituted C₁-C₆-haloalkyl, optionally substituted C₁-C₆-alkoxy or optionally substituted C₁-C₆-haloalkoxy;
L, Q, X, Y¹, Y², Y³, Y⁴, W, W¹, Z, m, p and the dashed bonds ( [symbol] ) are as defined above for the compound of Formula (IA), Formula (IB) and Formula (IC) in the first embodiment;

In a further embodiment, the foregoing combination is for use in treating and/or preventing a parasitic infection and/or infestation in and/or on an animal.

In another embodiment, a method for the treatment and/or prevention of a parasitic infection and/or infestation in and/or on an animal is provided comprising administering the foregoing combination to an animal.

In one embodiment of the antiparasitic compound of Formula (IA), Formula (IB) or Formula (IC), L is L1. In another embodiment, L is L2.

In one embodiment of Formula (IA), Formula (IB) or Formula (IC), R′ is hydrogen.

In some embodiments, R is C₁-C₄-alkyl, C₁-C₄-haloalkyl, amino, C₁-C₄-alkylamino, or di-(C₁-C₄-alkyl) amino.

In some embodiments, R is dimethylamino.

In some embodiments, R is C₁-C₄-alkyl or C₁-C₄-haloalkyl.

In other embodiments, R is hydroxy-C₁-C₄-alkyl, C₁-C₄-alkoxy-C₁-C₄-alkyl, C₁-C₄-haloalkoxy-C₁-C₄-alkyl or C₁-C₄-haloalkoxy-C₁-C₄-haloalkyl.

In some embodiments, R is methyl, ethyl, n-propyl, isopropyl, n-butyl, sec-butyl, isobutyl or tert-butyl.

In other embodiments, R is isopropyl or tert-butyl.

In one embodiment, R is tert-butyl.

In some embodiments, R is C₂-C₄-alkenyl or C₂-C₄-haloalkenyl.

In some embodiments, R is optionally substituted cyclopentyl or optionally substituted cyclohexyl.

In another embodiment, R is optionally substituted cyclobutyl. In yet another embodiment, R is cyclobutyl substituted by halogen, C₁-C₆-alkyl or C₁-C₆-haloalkyl. In still another embodiment, R is cyclobutyl substituted by one or more fluoro, methyl or CF₃.

In another embodiment R is the following cyclobutyl group, wherein #is the point of attachment:

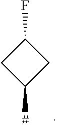

In another embodiment, R is optionally substituted cyclopropyl.

In one embodiment, R is cyclopropyl substituted by one or more halogen, C₁-C₆-alkyl or C₁-C₆-haloalkyl.

In another embodiment, R is cyclopropyl substituted by one or more fluoro, methyl or CF₃ groups.

In another embodiment R is a 3-, 4-, 5-, 6-, 7-, or 8-membered heterocyclyl group, which may include one to three heteroatoms selected from the group consisting of N, O, Si and S and may be optionally substituted.

In some embodiments, R is an optionally substituted, saturated or unsaturated 6-membered heterocyclyl group.

In another embodiment R is a 4- or 5-membered nitrogen-containing heterocyclyl group, which may be optionally substituted by one or more C₁-C₄-alkyl, C₁-C₄-haloalkyl, hydroxyl or halogen.

In another embodiment R is a 3- or 4-membered nitrogen-containing heterocyclyl group, which may be optionally substituted by one or more C₁-C₄-alkyl, C₁-C₄-haloalkyl, hydroxyl or halogen.

In some embodiments, R is aziridinyl, azetidinyl, oxetanyl, pyrrolidinyl or pyrrolyl, all of which are optionally substituted by one or more C₁-C₄-alkyl, C₁-C₄-haloalkyl, hydroxyl or halogen.

In some embodiments, R is morpholinyl, piperidinyl or piperazinyl, each of which are optionally substituted by one or more C₁-C₄-alkyl, C₁-C₄-haloalkyl, hydroxyl or halogen.

In some embodiments, R is aziridinyl or azetidinyl, optionally substituted by one or more C₁-C₄-alkyl, C₁-C₄-haloalkyl, hydroxyl or halogen.

In other embodiments, R is aziridinyl or azetidinyl, optionally substituted by one or more methyl, hydroxyl or fluoro.

In another embodiment, R is optionally substituted aziridin-1-yl, azetidine-1-yl, piperidin-1-yl, piperazin-1-yl or morpholin-1-yl.

In one embodiment, R¹ is hydrogen, halogen, C₁-C₄-alkyl or C₁-C₄-haloalkyl.

In one embodiment, R¹ is hydrogen.

In another embodiment, R¹ is C₁-C₄-alkyl.

In another embodiment, R¹ is methyl, ethyl, n-propyl, iso-propyl, n-butyl, sec-butyl, iso-butyl or tert-butyl.

In another embodiment, R¹ is methyl.

In another embodiment, R¹ is C₁-C₄-haloalkyl.

In another embodiment, R¹ is CF₃.

In another embodiment, R¹ is halogen.

In another embodiment, R¹ is fluoro, bromo or chloro.

In one embodiment, R¹ is fluoro.

In some embodiments, R² is hydrogen, halogen, C₁-C₄-alkyl or C₁-C₄-haloalkyl.

In one embodiment, R² is hydrogen.

In another embodiment, R² is C₁-C₄-alkyl.

In another embodiment, R² is methyl, ethyl, n-propyl, iso-propyl, n-butyl, sec-butyl, iso-butyl or tert-butyl.

In another embodiment, R² is methyl.

In another embodiment, R² is C₁-C₄-haloalkyl.

In another embodiment, R² is CF₃, —CH₂CF₃, —CHFCF₃ or —CF₂CF₃.

In one embodiment, R² is CF₃.

In some embodiments, R² is halogen.

In another embodiment, R² is fluoro, bromo or chloro.

In one embodiment, R² is fluoro.

In some embodiments, R³ is hydrogen, halogen, C₁-C₄-alkyl or C₁-C₄-haloalkyl.

In one embodiment, R³ is hydrogen.

In another embodiment, R³ is C₁-C₄-alkyl.

In another embodiment, R³ is methyl, ethyl, n-propyl, iso-propyl, n-butyl, sec-butyl, iso-butyl or tert-butyl.

In another embodiment, R³ is methyl.

In another embodiment, R³ is C₁-C₄-haloalkyl.

In another embodiment, R³ is CF₃, —CH₂CF₃, —CHFCF₃ or —CF₂CF₃.

In one embodiment, R³ is CF₃.

In some embodiments, R³ is halogen.

In another embodiment, R³ is fluoro, bromo or chloro.

In another embodiment, R³ is fluoro.

In some embodiments, R⁴ is hydrogen, halogen, C₁-C₄-alkyl or C₁-C₄-haloalkyl.

In one embodiment, R⁴ is hydrogen.

In another embodiment, R⁴ is C₁-C₄-alkyl.

In another embodiment, R⁴ is methyl, ethyl, n-propyl, iso-propyl, n-butyl, sec-butyl, iso-butyl or tert-butyl.

In another embodiment, R⁴ is methyl.

In another embodiment, R⁴ is C₁-C₄-haloalkyl.

In another embodiment, R⁴ is CF₃, —CH₂CF₃, —CHFCF₃ or —CF₂CF₃.

In one embodiment, R⁴ is CF₃.

In some embodiments, R⁴ is halogen.

In another embodiment, R⁴ is fluoro, bromo or chloro.

In another embodiment, R⁴ is fluoro.

In some embodiments, R⁵ is hydrogen, halogen, C₁-C₄-alkyl or C₁-C₄-haloalkyl.

In one embodiment, R⁵ is hydrogen.

In another embodiment, R⁵ is C₁-C₄-alkyl.

In another embodiment, R⁵ is methyl.

In another embodiment, R⁵ is C₁-C₄-haloalkyl.

In one embodiment, R⁵ is CF₃.

In some embodiments, R⁵ is halogen.

In another embodiment, R⁵ is fluoro, bromo or chloro.

In another embodiment, R⁵ is fluoro.

In one embodiment, R¹⁰ is halogen. In another embodiment, R¹⁰ is halogen and m is 2 or 3. In another embodiment, R¹⁰ is halogen and m is 2. In another embodiment, R¹⁰ is halogen and mis 3.

In another embodiment, m is 2 and R¹⁰ is chloro or fluoro substituted at the 2- and 3-positions. In another embodiment, m is 2 and R¹⁰ is chloro or fluoro substituted at the 2- and 4-positions. In another embodiment, m is 2 and R¹⁰ is chloro or fluoro substituted at the 2- and 5-positions. In another embodiment, m is 2 and R¹⁰ is chloro or fluoro substituted at the 2- and 6-positions. In another embodiment, m is 2 and R¹⁰ is chloro or fluoro substituted at the 3- and 4-positions. In one embodiment, m is 2 and R¹⁰ is chloro or fluoro substituted at the 3- and 5-positions.

In another embodiment, m is 3 and R¹⁰ is chlorine or fluorine substituted at the 2-, 3- and 4-positions of the phenyl ring. In other embodiments, m is 3 and R¹⁰ is chlorine or fluorine substituted at the 2-, 3- and 5-positions of the phenyl ring. In other embodiments, m is 3 and R¹⁰ is chlorine or fluorine substituted at the 2-, 4- and 5-positions of the phenyl ring. In other embodiments, m is 3 and R¹⁰ is chlorine or fluorine substituted at the 2-, 3- and 6-positions of the phenyl ring. In other embodiments, m is 3 and R¹⁰ is chlorine or fluorine substituted at the 3-, 4- and 5-positions of the phenyl ring. In other embodiments, m is 3 and R¹⁰ is chlorine or fluorine substituted at the 2-, 4- and 6-positions of the phenyl ring.

In another embodiment, m is 3 and R¹⁰ is chlorine substituted at the 2-, 3- and 4-positions of the phenyl ring. In other embodiments, m is 3 and R¹⁰ is chlorine substituted at the 2-, 3- and 5-positions of the phenyl ring. In other embodiments, m is 3 and R¹⁰ is chlorine substituted at the 2-, 4- and 5-positions of the phenyl ring. In other embodiments, m is 3 and R¹⁰ is chlorine substituted at the 2-, 3- and 6-positions of the phenyl ring. In other embodiments, m is 3 and R¹⁰ is chlorine substituted at the 3-, 4- and 5-positions of the phenyl ring. In other embodiments, m is 3 and R¹⁰ is chlorine substituted at the 2-, 4- and 6-positions of the phenyl ring.

In another embodiment, m is 3 and R¹⁰ is fluorine substituted at the 2-, 3- and 4-positions of the phenyl ring. In other embodiments, m is 3 and R¹⁰ is fluorine substituted at the 2-, 3- and 5-positions of the phenyl ring. In other embodiments, m is 3 and R¹⁰ is fluorine substituted at the 2-, 4- and 5-positions of the phenyl ring. In other embodiments, m is 3 and R¹⁰ is fluorine substituted at the 2-, 3- and 6-positions of the phenyl ring. In other embodiments, m is 3 and R¹⁰ is fluorine substituted at the 3-, 4- and 5-positions of the phenyl ring. In other embodiments, m is 3 and R¹⁰ is fluorine substituted at the 2-, 4- and 6-positions of the phenyl ring.

In some embodiments, each R⁵ is independently hydrogen or halogen.

In some embodiments, each R⁵ is independently hydrogen, C₁-C₄-alkyl or C₁-C₄-haloalkyl.

In some embodiments, each R⁵ is hydrogen.

In another embodiment, each R⁵ is independently hydrogen, fluoro or chloro.

In some embodiments, R⁶ and R⁷ are independently in each occurrence hydrogen, halogen, C₁-C₄-alkyl or C₁-C₄-haloalkyl. In another embodiment, R⁶ and R⁷ are each hydrogen. In another embodiment, R⁶ and R⁷ are independently hydrogen or methyl.

In one embodiment, R⁹ is H. In another embodiment, R⁹ is C₁-C₃-alkyl. In yet another embodiment, R⁹ is methyl.

In one embodiment, Q is N.

In another embodiment, Q is C.

In another embodiment, Q is C—R⁹.

In another embodiment, X is O.

In another embodiment, X is S.

In another embodiment, X is NR′.

In another embodiment, W is CH₂.

In another embodiment, W is CH.

In other embodiments, W is C(C₁-C₃-alkyl)₂ or C(C₁-C₃-haloalkyl)₂.

In other embodiments, W is CH₂, C(CH₃)₂ or C(CF₃)₂.

In some embodiments, W is absent.

In some embodiments, W¹ is CH₂.

In another embodiment, W¹ is CH, C—C₁-C₃-alkyl or C—C₁-C₃-haloalkyl.

In other embodiments, W¹ is CH₂, C(C₁-C₃-alkyl)₂ or C(C₁-C₃-haloalkyl)₂.

In other embodiments, W¹ is CH₂, C(CH₃)₂, C(C₂H₅)₂ or C(CF₃)₂.

In some embodiments, Z is CH₂.

In another embodiment, Z is CH, C—C₁-C₃-alkyl or C—C₁-C₃-haloalkyl.

In other embodiments, Z is C(C₁-C₃-alkyl)₂ or C(C₁-C₃-haloalkyl)₂.

In some embodiments, Z is O.

In one embodiment, the antiparasitic combinations of the invention comprise a compound of Formula (IA), wherein:
L is L1, wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is hydrogen, methyl, —CF₃, chloro or fluoro; R⁴ is hydrogen; R⁹ is hydrogen; R¹⁰ is chloro or fluoro and m is 2 or 3; W and W¹ are each CH₂; Z is O and Y¹, Y², Y³ and Y⁴ are each CH.

In another embodiment, the antiparasitic combinations comprise an antiparasitic compound of Formula (IA), wherein L is L1, wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro and m is 3, wherein the chloro substituents are substituted at the 2, 3 and 5 positions of the phenyl ring; W and W¹ are each CH₂; Z is O and Y¹, Y², Y³ and Y⁴ are each CH.

In another embodiment, the antiparasitic combinations comprise an antiparasitic compound of Formula (IA), wherein L is L1, wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is fluoro and m is 3, wherein the fluoro substituents are substituted at the 2, 3 and 5 positions of the phenyl ring; W and W¹ are each CH₂; Z is O and Y¹, y², Y³ and Y⁴ are each CH.

In another embodiment, the antiparasitic combinations comprise an antiparasitic compound of Formula (IA), wherein L is L1, wherein X is O and R′ is hydrogen; Q is C—R⁹; R is tert-butyl or iso-propyl; R¹ is hydrogen; R² is methyl; R⁴ is hydrogen; R⁹ is hydrogen; R¹⁰ is chloro or fluoro and m is 3, wherein the chloro or fluoro substituents are located at the 2, 3 and 5 positions of the phenyl ring; W and W¹ are each CH₂; Z is O and Y¹, Y², Y³ and Y⁴ are each CH.

In another embodiment, the antiparasitic combinations of the invention comprise an antiparasitic compound of Formula (IB), wherein:
L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R² is hydrogen, methyl, —CF₃, chloro or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH.

In another embodiment, the antiparasitic combinations comprise an antiparasitic compound of Formula (IB), wherein L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R² is hydrogen, methyl, —CF₃, chloro or fluoro; R⁴ is hydrogen; R⁹ is hydrogen; R¹⁰ is chloro or fluoro and m is 2 or 3; W and W¹ are each CH₂; Z is O and Y¹, Y², Y³ and Y⁴ are each CH.

In another embodiment, the antiparasitic combinations comprise an antiparasitic compound of Formula (IB), wherein L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R² is hydrogen, methyl, —CF₃, chloro or fluoro; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH.

In another embodiment, the antiparasitic combinations comprise an antiparasitic compound of Formula (IB), wherein L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R² is methyl; R⁴ is hydrogen; R⁹ is hydrogen; R¹⁰ is chloro and m is 3, wherein the chloro substituents are substituted at the 2, 3 and 5 positions of the phenyl ring; W and W¹ are each CH₂; Z is O and Y¹, Y², Y³ and Y⁴ are each CH.

In another embodiment, the antiparasitic combinations comprise an antiparasitic compound of Formula (IB), wherein L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R² is methyl; R⁴ is hydrogen; R⁹ is hydrogen; R¹⁰ is fluoro and m is 3, wherein the fluoro substituents are substituted at the 2, 3 and 5 positions of the phenyl ring; W and W¹ are each CH₂; Z is O and Y¹, Y², Y³ and Y⁴ are each CH.

In another embodiment, the antiparasitic combinations of the invention comprise an antiparasitic compound of Formula (IC), wherein:
L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen, methyl, —CF₃, chloro or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH.

In another embodiment of Formula (IC), L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH.

In another embodiment of Formula (IC), L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W^{I} are each CH₂; Z is O and Y¹, Y², Y^{a} and Y⁴ are each CH.

In another embodiment of Formula (IC), L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is chloro and m is 3, wherein the chloro substituents are substituted at the 2, 3 and 5 positions of the phenyl ring; W and W¹ are each CH₂; Z is O and Y¹, Y², Y³ and Y⁴ are each CH.

In another embodiment of Formula (IC), L is L1, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is fluoro and m is 3, wherein the fluoro substituents are substituted at the 2, 3 and 5 positions of the phenyl ring; W and W¹ are each CH₂; Z is O and Y¹, Y², Y³ and Y⁴ are each CH.

In another embodiment of Formula (IC), L is L1, wherein X is O and R′ is hydrogen; Q is C—R⁹; R is ethyl, cyclobutyl, azetidine-1-yl, morpholin-4-yl, oxetan-3-yl or tetrahydropyran-4-yl, each optionally substituted by one or more fluoro or methyl; R¹ is hydrogen; R² is hydrogen; R³ is hydrogen or fluoro; R⁴ is hydrogen; R⁹ is hydrogen; R¹⁰ is fluoro and m is 3, wherein the fluoro substituents are substituted at the 2, 3 and 5 positions of the phenyl ring; W and W¹ are each CH₂; Z is O and Y¹, Y², Y³ and Y⁴ are each CH.

In another embodiment, the antiparasitic combinations of the invention comprise an antiparasitic compound of Formula (IC), wherein:
L is L2, wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl; R¹ is hydrogen; R² is hydrogen; R³ is hydrogen or fluoro; R⁴ is hydrogen; R⁹ is hydrogen; R¹⁰ is CH₃, CF₃, OCH₃, OCF₃, chloro or fluoro and m is 2 or 3; W is CH; W¹ is CH or C—CH₃; Z is N and Y¹, Y², Y³ and Y⁴ are each CH.

In another embodiment, the antiparasitic combinations of the invention comprise a compound of Formula (IC), wherein: L is L2, wherein X is O and R′ is hydrogen; Q is C—R⁹; R is morpholinyl, optionally substituted by one or more fluoro or methyl; R¹ is hydrogen; R² is hydrogen; R³ is hydrogen or fluoro; R⁴ is hydrogen; R⁹ is hydrogen; R¹⁰ is CH₃, CF₃, OCH₃, OCF₃, chloro or fluoro and m is 2 or 3; W is CH; W¹ is CH or C—CH₃; Z is N and Y¹, Y², Y³ and Y⁴ are each CH.

In another embodiment, the antiparasitic combinations of the invention comprise an antiparasitic compound of Formula (IC), wherein: L is L2, wherein X is O and R′ is hydrogen; Q is C—R⁹; R is morpholinyl, optionally substituted by one or more fluoro or methyl; R¹ is hydrogen; R² is hydrogen; R³ is hydrogen or fluoro; R⁴ is hydrogen; R⁹ is hydrogen; R¹⁰ is chloro or fluoro and m is 2 or 3; W is CH; W¹ is CH or C—CH₃; Z is N and Y¹, y², Y³ and Y⁴ are each CH.

In another embodiment, the antiparasitic combinations of the invention comprise an antiparasitic compound of Formula (IC), wherein: L is L2, wherein X is O and R′ is hydrogen; Q is C—R⁹; R is morpholinyl, optionally substituted by one or more fluoro or methyl; R¹ is hydrogen; R² is hydrogen; R³ is fluoro; R⁴ is hydrogen; R⁹ is hydrogen; R¹⁰ is chloro or fluoro and m is 2, wherein the chloro or fluoro are substituted at the 2-, 3- or 5-positions of the phenyl ring; W is CH; W¹ is CH or C—CH₃; Z is N and Y¹, Y², Y³ and Y⁴ are each CH.

In other embodiments, the antiparasitic combinations of the invention comprise an antiparasitic compound of Formula (IA) shown above, wherein L, R, R¹, R², R⁴ and (R¹⁰)ₘ are defined in the Table 1 below, and the group

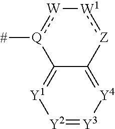

wherein #is the point of attachment to variable L, represents one of the following Ring Systems:

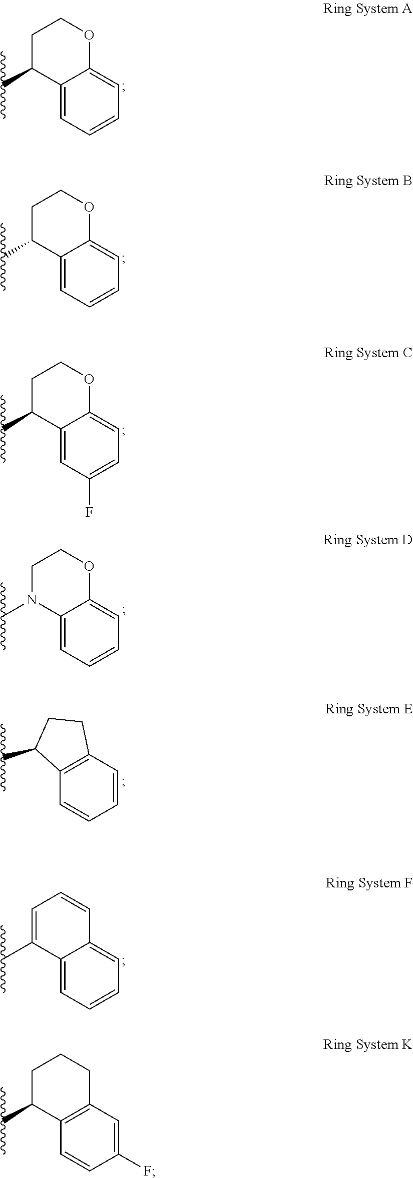

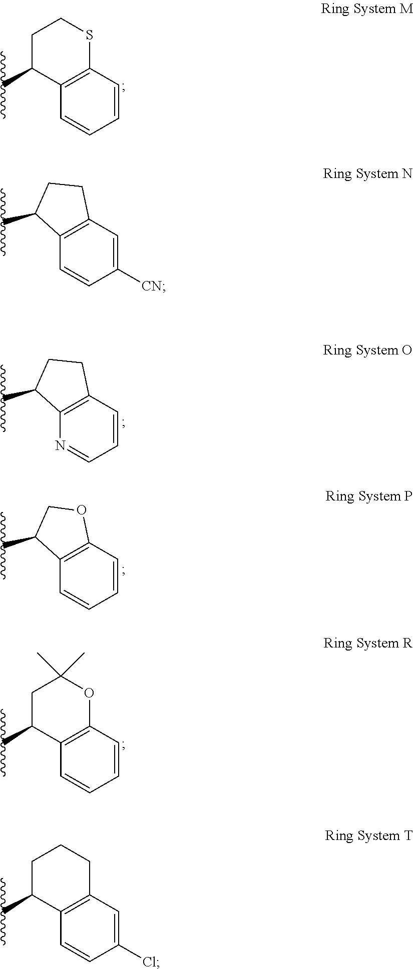

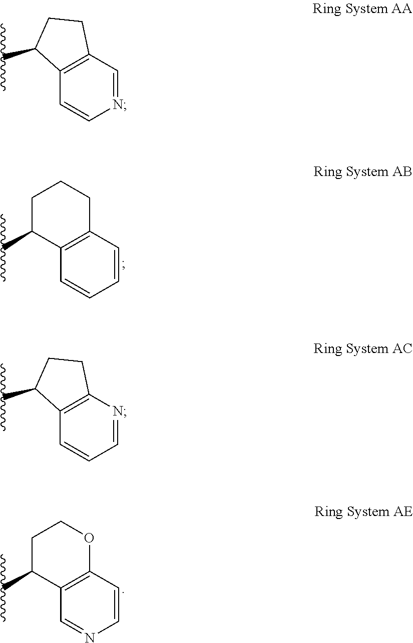 [SMILES: *[C@H]1CCc2cnccc21 ; *[C@H]1CCCc2ccccc21 ; *[C@H]1CCc2ncccc21 ; *[C@H]1CCOc2ccncc21]

In Table 1, “Me” represents methyl, “i-Pr” means isopropyl, “t-Bu” means tert-butyl; the expression “3,5-di-Cl” represents that the two chloro substituents are located at the 3- and 5-positions of the phenyl ring; “2,6-di-Cl-4-F” means the two chloro are substituted at the 2- and 6-positions and the fluoro is substituted at the 4-position of the ring; “2,4,6-tri-F” means that the three fluoro are located at the 2-, 4- and 6-positions of the phenyl ring; “2,3,5-tri-F” represents the three fluoro are located at the 2-, 3- and 5-positions of the phenyl ring, and so on.

TABLE 1

| | | | | | | | Ring | |
|---|---|---|---|---|---|---|---|---|
| Cmpd. # | L | R | R¹ | R² | R⁴ | (R¹⁰)ₘ | Sys. | ESI-MS |
| A461 | L2 | i-Pr | H | Me | H | 3,5-di-Cl-Ph | B | 494 [M + H]⁺ |
| A462 | L2 | i-Pr | H | Me | H | 3,5-di-Cl-Ph | A | 494 [M + H]⁺ |
| A463 | L1 | i-Pr | H | Cl | H | 3,5-di-Cl-Ph | A | 514 [M + H]⁺ |
| A464 | L1 | t-Bu | H | Me | H | 3,5-di-Cl-Ph | A | 508 [M + H]⁺ |
| A465 | L1 | i-Pr | H | Me | H | 3,5-di-Cl | A | 495 [M + H]⁺ |
| A466 | L1 | i-Pr | H | F | H | 3,5-di-Cl | A | 498 [M + H]⁺ |
| A467 | L1 | i-Pr | Cl | H | H | 3,5-di-Cl | A | 514 [M + H]⁺ |
| A468 | L1 | i-Pr | CF₃ | H | H | 3,5-di-Cl | A | 548 [M + H]⁺ |
| A469 | L1 | i-Pr | Me | H | H | 3,5-di-Cl | A | 494 [M + H]⁺ |
| A470 | L1 | i-Pr | H | H | H | 3,5-di-Cl | A | 480 [M + H]⁺ |
| A483 | L1 | t-Bu | H | CF₃ | H | 3,5-di-Cl | A | 562 [M + H]⁺ |
| A482 | L1 | t-Bu | H | Me | H | 3,5-di-Cl | D | 509 [M + H]⁺ |
| A484 | L1 | t-Bu | H | Cl | H | 2,4,6-tri-F | A | 514 [M + H]⁺ |
| A485 | L1 | t-Bu | H | Cl | H | 2,6-di-Cl-4-F | A | 547 [M + H]⁺ |
| A486 | L1 | t-Bu | H | Cl | H | 2,3,5-tri-F | A | 514 [M + H]⁺ |
| A472 | L1 | t-Bu | H | Me | H | 2,3,5-tri-Cl | A | 544 [M + H]⁺ |
| A471 | L1 | t-Bu | H | Me | H | 2,3,5-tri-F | A | 494 [M + H]⁺ |
| A476 | L1 | t-Bu | H | Me | H | 2,4,6-tri-F | A | 494 [M + H]⁺ |
| A487 | L1 | t-Bu | H | Me | H | 2,6-di-Cl-4-F | A | 526 [M + H]⁺ |
| A499 | L1 | 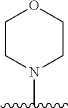 [SMILES: *N1CCOCC1] | H | Me | H | 3,5-di-Cl | A | 537 [M + H]⁺ |
| A475 | L1 | —N(CH₃)₂ | H | Cl | H | 3,5-di-Cl | A | 516 [M + H]⁺ |
| A473 | L1 | —N(CH₃)₂ | H | Cl | H | 2,3,5-tri-Cl | A | 550 [M + H]⁺ |
| A474 | L1 | —N(CH₃)₂ | H | Cl | H | 2,3,5-tri-F | A | 551 [M + H]⁺ |
| A477 | L1 | t-Bu | H | Cl | H | 3,5-di-Cl | A | 529 [M + H]⁺ |
| A478 | L1 | —N(CH₃)₂ | H | Cl | H | 2,3-di-Cl-5-F | A | 534 [M + H]⁺ |
| A490 | L1 | 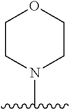 [SMILES: *N1CCOCC1] | H | Me | H | 2,3-di-Cl-5-F | A | 555 [M + H]⁺ |
| A491 | L1 | 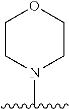 [SMILES: *N1CCOCC1] | H | Me | H | 2,3,5-tri-F | A | 523 [M + H]⁺ |
| A488 | L1 | t-Bu | H | Me | H | 2,3-di-Cl-5-F | C | 544 [M + H]⁺ |
| A479 | L1 | i-Pr | H | Cl | H | 3,5-di-Cl | D | 516 [M + H]⁺ |
| A489 | L1 | i-Pr | H | Me | H | 2,3-di-Cl-5-F | A | 512 [M + H]⁺ |
| A480 | L1 | t-Bu | H | Me | H | 3,5-di-Cl | E | 492 [M + H]⁺ |
| A481 | L1 | t-Bu | H | Me | H | 2,3-di-Cl-5-F | A | 526 [M + H]⁺ |
| A502 | L1 | t-Bu | H | Cl | H | 2,3-di-Cl-5-F | A | 547 [M + H]⁺ |
| A492 | L1 | t-Bu | H | Cl | H | 3,5-di-Cl | E | 513 [M + H]⁺ |
| A493 | L1 | —N(CH₃)₂ | H | Me | H | 2,3-di-Cl-5-F | A | 513 [M + H]⁺ |
| A516 | L1 | t-Bu | H | Cl | H | 3,5-di-Cl | D | 530 [M + H]⁺ |
| A494 | L1 | t-Bu | H | Cl | H | 2,3-di-Cl-5-F | D | 548 [M + H]⁺ |
| A482 | L1 | t-Bu | H | Me | H | 3,5-di-Cl | D | 509 [M + H]⁺ |
| A495 | L1 | t-Bu | H | Me | H | 2,3-di-Cl-5-F | D | 527 [M + H]⁺ |
| A496 | L1 | i-Pr | H | Cl | H | 2,3-di-Cl-5-F | D | 534 [M + H]⁺ |
| A497 | L1 | i-Pr | H | Me | H | 3,5-di-Cl-Ph | D | 495 [M + H]⁺ |
| A498 | L1 | i-Pr | H | Me | H | 2,3-di-Cl-5-F | D | 513 [M + H]⁺ |
| A501 | L1 | 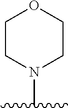 [SMILES: *N1CCOCC1] | H | Me | H | 2,3,5-tri-Cl | A | 572 [M + H]⁺ |
| A505 | L1 | 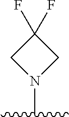 [SMILES: *N1CC(F)(F)C1] | H | Me | H | 2,3,5-tri-Cl | A | 578 [M + H]⁺ |
| A504 | L1 | 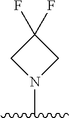 [SMILES: *N1CC(F)(F)C1] | H | Me | H | 2,3-di-Cl-5-F | A | 561 [M + H]⁺ |
| A511 | L1 | 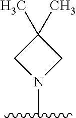 [SMILES: *N1CC(C)(C)C1] | H | Me | H | 2,3,5-tri-Cl | A | 570 [M + H]⁺ |
| A512 | L1 | 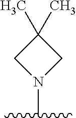 [SMILES: *N1CC(C)(C)C1] | H | Me | H | 2,3-di-Cl-5-F | A | 553 [M + H]⁺ |
| A508 | L1 | 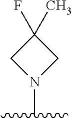 [SMILES: *N1CC(C)(F)C1] | H | Me | H | 2,3,5-tri-Cl | A | 574 [M + H]⁺ |
| A510 | L1 | 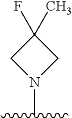 [SMILES: *N1CC(C)(F)C1] | H | Me | H | 2,3-di-Cl-5-F | A | 557 [M + H]⁺ |
| A506 | L1 | 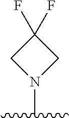 [SMILES: *N1CC(F)(F)C1] | H | Cl | H | 2,3,5-tri-Cl | A | 598 [M + H]⁺ |
| A507 | L1 | 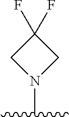 [SMILES: *N1CC(F)(F)C1] | H | Cl | H | 2,3-di-Cl-5-F | A | 582 [M + H]⁺ |
| A515 | L1 | 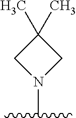 [SMILES: *N1CC(C)(C)C1] | H | Cl | H | 2,3,5-tri-Cl | A | 590 [M + H]⁺ |
| A520 | L1 | 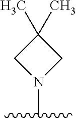 [SMILES: *N1CC(C)(C)C1] | H | Cl | H | 2,3-di-Cl-5-F | A | 574 [M + H]⁺ |
| A503 | L1 | 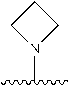 [SMILES: *N1CCC1] | H | Cl | H | 2,3-di-Cl-5-F | A | 546 [M + H]⁺ |
| A509 | L1 | 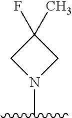 [SMILES: *N1CC(C)(F)C1] | H | Cl | H | 2,3,5-tri-Cl | A | 594 [M + H]⁺ |
| A514 | L1 | 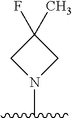 [SMILES: *N1CC(C)(F)C1] | H | Cl | H | 2,3-di-Cl-5-F | A | 578 [M + H]⁺ |
| A521 | L1 | i-Pr | H | Me | H | 3,5-di-Cl | F | 488 [M + H]⁺ |
| A513 | L1 | i-Pr | H | Me | H | 2,3,5-tri-F | A | 479 [M + H]⁺ |
| A517 | L1 | i-Pr | H | Me | H | 2,3,5-tri-Cl | A | 529 [M + H]⁺ |
| A518 | L1 | t-Bu | Br | Me | H | 2,3-di-Cl | A | 587 [M + H]⁺ |
| A519 | L1 | t-Bu | H | Me | H | 2,3-di-Cl | A | 508 [M + H]⁺ |
| A522 | L1 | t-Bu | H | Cl | H | 2,3,5-tri-Cl | A | 563 [M + H]⁺ |
| A523 | L1 | i-Pr | H | Cl | H | 2,3-di-Cl-5-F | A | 532 [M + H]⁺ |
| A500 | L1 | H | H | H | i-Pr | 3,5-di-Cl | A | 572 [M + H]⁺ |
| A525 | L1 | 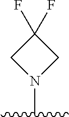 [SMILES: *N1CC(F)(F)C1] | H | Me | H | 2,3,5-tri-Cl | A | 578 [M + H]⁺ |
| A526 | L1 | 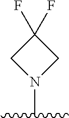 [SMILES: *N1CC(F)(F)C1] | H | Me | H | 2,3-di-Cl-5-F | A | 562 [M + H]⁺ |
| A530 | L1 | t-Bu | H | Me | H | 2,3,5-tri-Cl | A | 543 [M + H]⁺ |
| A534 | L1 | t-Bu | H | Cl | H | 2,3-di-Cl-5-F | A | 547 [M + H]⁺ |
| A538 | L1 | t-Bu | H | Me | H | 2,3-di-Cl-5-F | K | 543 [M + H]⁺ |
| A539 | L1; | i-Pr | H | Cl | H | 2,3-di-Cl-5-F | A | 548 [M + H]⁺ |
| | X = S | | | | | | | |
| A541 | L1 | t-Bu | H | Me | H | 2,3-di-Cl-5-F | M | 543 [M + H]⁺ |
| A542 | L1 | t-Bu | H | Me | H | 2,3-di-Cl-5-F | N | 536 [M + H]⁺ |
| A543 | L1; | t-Bu | H | Me | H | 2,3-di-Cl-5-F | A | 541 [M + H]⁺ |
| | R′ = Me | | | | | | | |
| A544 | L1 | t-Bu | H | Me | H | 2,3-di-Cl-5-F | O | 512 [M + H]⁺ |
| A545 | L1 | t-Bu | H | Me | H | 2,3-di-Cl-5-F | P | 513 [M + H]⁺ |
| A547 | L1 | t-Bu | H | Me | H | 2,3-di-Cl-5-F | R | 555 [M + H]⁺ |
| A549 | L1 | t-Bu | H | Me | H | 2,3-di-Cl-5-F | T | 559 [M + H]⁺ |
| A556 | L1 | t-Bu | H | Me | H | 2,3-di-Cl-5-F | AA | 512 [M + H]⁺ |
| A557 | L1 | t-Bu | H | Me | H | 2,3-di-Cl-5-F | AB | 525 [M + H]⁺ |
| A558 | L1 | t-Bu | H | Me | H | 2,3-di-Cl-5-F | E | 511 [M + H]⁺ |
| A559 | L1 | t-Bu | H | Me | H | 2,3-di-Cl-5-F | AC | 512 [M + H]⁺ |
| A561 | L1 | 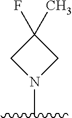 [SMILES: *N1CC(C)(F)C1] | H | Me | H | 2,3,5-tri-Cl | A | 575 [M + H]⁺ |
| A562 | L1 | 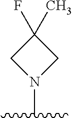 [SMILES: *N1CC(C)(F)C1] | H | Me | H | 2,3-di-Cl-5-F | A | 558 [M + H]⁺ |
| A563 | L1 | 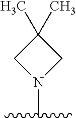 [SMILES: *N1CC(C)(C)C1] | H | Me | H | 2,3,5-tri-Cl | A | 571 [M + H]⁺ |
| A564 | L1 | 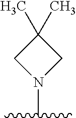 [SMILES: *N1CC(C)(C)C1] | H | Me | H | 2,3-di-Cl-5-F | A | 554 [M + H]⁺ |
| A565 | L1 | t-Bu | H | Cl | H | 3,5-di-Cl | D | 531 [M + H]⁺ |
| A566 | L1 | t-Bu | H | Me | H | 2,3,5-tri-F | A | 495 [M + H]⁺ |
| A567 | L1 | 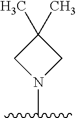 [SMILES: *N1CC(C)(C)C1] | H | Cl | H | 2,3-di-Cl-5-F | A | 575 [M + H]⁺ |
| A568 | L1 | —N(CH₃)₂ | H | Me | H | 2,3,5-tri-Cl | A | 531 [M + H]⁺ |
| A569 | L1 | t-Bu | H | Me | H | 2,3-di-Cl-5-F | AE | 528 [M + H]⁺ |

In one embodiment, the antiparasitic combinations of the invention include an antiparasitic compound of Formula (IA) that has the formula A472:

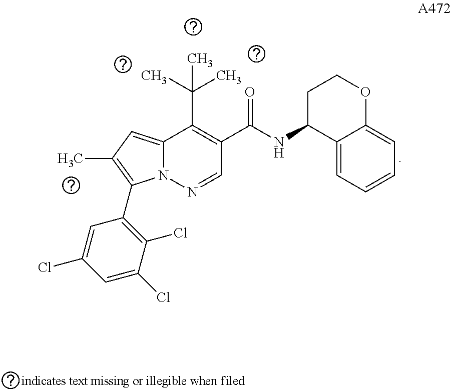 [SMILES: Cc1cc2c(C(C)(C)C)c(C(=O)N[C@H]3CCOc4ccccc43)cnn2c1-c1cc(Cl)cc(Cl)c1Cl]

In a further embodiment, such combinations are for use in treating and/or preventing a parasitic infection and/or infestation in and/or on an animal.

In another embodiment, a method for the treatment and/or prevention of a parasitic infection and/or infestation in and/or on an animal is provided comprising administering the foregoing combinations including compound A472 to an animal.

In another embodiment, the antiparasitic combinations of the description include a compound of Formula (IC) that has the formula IC-1:

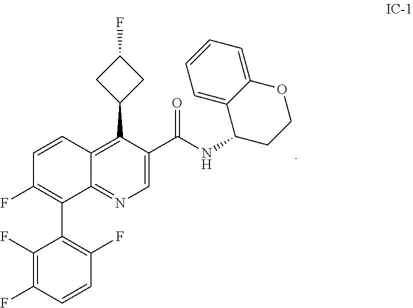 [SMILES: O=C(N[C@H]1CCOc2ccccc21)c1cnc2c(-c3c(F)ccc(F)c3F)c(F)ccc2c1[C@H]1C[C@H](F)C1]

In a further embodiment, such combinations are for use in treating and/or preventing a parasitic infection and/or infestation in and/or on an animal.

In another embodiment, a method for the treatment and/or prevention of a parasitic infection and/or infestation in and/or on an animal is provided comprising administering the foregoing combination including compound IC-1 to an animal.

### Pharmaceutical Compositions

The pharmaceutical compositions of the invention comprise an effective amount of an antiparasitic active agent combination of the invention, which includes at least one antiparasitic compound of Formula (IA), Formula (IB) and/or Formula (IC), or salts thereof, or combinations thereof, in combination with one or more additional antiparasitic active agents, and an acceptable carrier or diluent. The pharmaceutical compositions may be in a variety of solid and liquid forms which are suitable for various methods of application or administration to an animal. For example, the pharmaceutical compositions comprising the one or more antiparasitic compounds of the invention may be in compositions suitable for oral administration, injectable administration, including subcutaneous and parenteral administration, topical administration (e.g. spot-on or pour-on), including dermal or subdermal administration.

It is well known that combining two or more different compounds having different structures and physicochemical properties in a single oral dosage form presents significant challenges. Some compounds may be incompatible with the other active agents in the composition and/or excipients used with other compounds in the composition. Furthermore, some compounds will have unique stability requirements that may not be compatible with the other compounds and/or excipients used in the composition. These challenges are intensified with the addition of each additional active agent in the combination. Thus, the preparation of a stable composition comprising two or more antiparasitic active agents (e.g., three or more, four or more, etc.) that will deliver an effective amount of each active to the required location in and/or on the animal treated in order to treat and/or prevent a parasitic infection and/or infestation presents a significant challenge.

Suitable dosage forms for oral administration include dietary supplements, troches, lozenges, chewables (e.g. chewable tablets or soft chews), tablets, hard or soft capsules, boluses, emulsions, aqueous or oily suspensions, aqueous or oily solutions, oral drench compositions, dispersible powders or granules, premixes, syrups or elixirs, enteric compositions or pastes. Pharmaceutical compositions intended for oral use may be prepared according to any method known in the art for the manufacture of pharmaceutical compositions. Suitable tablets may be obtained, for example, by mixing the antiparasitic active agent combinations of the invention with known excipients, for example inert diluents, carriers, disintegrants, adjuvants, surfactants, binders and/or lubricants.

In one embodiment of the invention, a soft chewable veterinary composition is provided comprising an effective amount of an antiparasitic active agent combination of the invention and a pharmaceutically acceptable carrier.

In some embodiments, the compositions may be in the form of a sterile injectable composition. The injectable composition may be a solution or a suspension. This suspension may be formulated according to the known art using those suitable dispersing or wetting agents and suspending agents. The sterile injectable preparation may also be a sterile injectable solution or suspension in a non-toxic parenterally acceptable diluent or solvent.

Implant compositions that may be used to deliver the compounds of the invention are commonly subcutaneously delivered to an animal. In addition, external wearable devices such as collars, pendants, ear tags, and the like, may be used to deliver the compounds of the invention to an animal.

Suitable topical spot-on or pour-on pharmaceutical compositions comprise a pharmaceutically effective amount of an antiparasitic active agent combination in a pharmaceutically acceptable carrier. The pharmaceutically acceptable carrier for the topical compositions of the invention may include, but are not limited to, solvents and solvent mixtures, permeation enhancers, surfactants, antioxidants, pH stabilizers, preservatives and crystallization inhibitors known in the art.

### Methods of Treatment

The antiparasitic active agent combinations and compositions of the invention are intended to be administered to an animal including, but not limited to, mammals, birds and fish to treat and/or prevent parasitic infections and/or infestations in and/or on the animals. In one embodiment the antiparasitic active agent combinations and compositions of the invention are intended to be used to treat parasitic infections and/or infestations in and/or on the animals. In one embodiment the antiparasitic active agent combinations and compositions of the invention are intended to be used to prevent parasitic infections and/or infestations in and/or on the animals. In one embodiment the antiparasitic active agent combinations and compositions of the invention are intended to be used to prevent and treat parasitic infections and/or infestations in and/or on the animals. Examples of animals are mammals that include but are not limited to humans, cattle, sheep, goats, llamas, alpacas, pigs, horses, donkeys, dogs, cats and other livestock or domestic mammals. Further examples of animals are birds that include turkeys, chickens, ostriches and other livestock or domestic birds. In one embodiment, the invention provides a use of the antiparasitic active agent combinations and compositions comprising the combinations to protect companion animals such as dogs and cats from endoparasites and/or ectoparasites. In another embodiment, the antiparasitic active agent combinations and compositions comprising the combinations of the invention may be used to protect equine animals from parasites. The compound may also be used to protect livestock animals.

The present invention is directed to antiparasitic active agent combinations comprising at least one compound of Formula (IA), Formula (IB) and/or Formula (IC), or a salt thereof, and at least one additional antiparasitic active agent. These antiparasitic combinations are useful in the treatment, prevention and/or control of parasitic infections and/or infestations in and/or on animals. The antiparasitic combinations of the invention and compositions comprising the combinations treat and/or prevent parasitic infections and/or infestations from a broad scope of different endoparasites (e.g. heartworm, nematodes that infect the gastrointestinal tract of animals, etc.) and ectoparasites (fleas, ticks, mites, and other ectoparasites). It is well known in the field that the combination of different active agents in one dosage form is very challenging since some active agents may be incompatible with other active agents in the composition. For example, it is possible that the presence of one active agent in the composition may inhibit the absorption of another active agent in the same dosage form, leading to an insufficient concentration in the blood stream or at the target site. The addition of each additional active agent (e.g., three or four active agents) to the dosage form increases the likelihood of incompatibility between active agents. Thus, it is challenging to develop a medicinal product containing multiple active agents.

As noted in the Background of the Invention, WO 2023/073641 describes anthelmintic pyrrolopyridazine compounds, including compound A472, that are active against endoparasites such as Dirofilaria immitis and certain parasitic nematodes.

The biological examples of WO 2023/073641 demonstrated the efficacy of compound A472 against microfilaria and L4 larval stages of Dirofilaria immitis in motility screening assays (Biological Examples 1 and 3) and in vivo in immune-deficient mice (Biological Example 4) and dogs (Biological Example 8). Compound A472 was also found to be active against larvae of the gastrointestinal nematode Haemonchus contortus in a motility screening assay (Biological Example 2). Commonly known as the barber's pole worm, Haemonchus contortus is known to primarily infect small ruminants such as sheep and goats. WO 2023/073641 did not include any efficacy data against other gastrointestinal nematodes that commonly infect canines, such as roundworms and hookworms, let alone any in vivo efficacy data.

In the biological examples of the present invention, chewable tablets are administered to target a minimum efficacious dose of 0.8 mg/kg body weight Compound A472, 12 micrograms/kg body weight moxidectin (μg/kg), 2.5 mg/kg body weight afoxolaner and 5 mg/kg body weight praziquantel. The 4-way chewable tablet of the present invention is demonstrated to have excellent efficacy against Dirofilaria immitis (Biological Example 4) as well as Toxocara canis (Biological Example 2), Amblyomma americanum (Biological Example 1) and Dipylidium caninum (Biological Example 3).

Biological Example 2 demonstrates that compound A472 administered at the very low doses of 0.4 mg/kg and 0.8 mg/kg body weight is very effective against infections of Toxocara canis. Percent efficacies of 95.4% and 98.7% were observed when compound A472 was administered at doses of 0.4 mg/kg and 0.8 mg/kg, respectively. In contrast, the 3-way chewable tablet not containing compound A472 exhibited 0% efficacy, although it contained moxidectin administered at a dose of 12 μg/kg. In previous studies, moxidectin administered orally targeting a dose of 12 μg/kg has demonstrated a modest level of efficacy against this endoparasite. For example, a chewable tablet containing a combination of afoxolaner and moxidectin administered at a dose targeting 2.5 mg/kg afoxolaner and 12 micrograms/kg moxidectin exhibited a percent efficacy of 53.2% compared with a percent efficacy of 95.2% for NexGard® PLUS (see Table II.16. on page 24 of the Freedom of Information Summary for NADA 141-554, NexGard® PLUS).

Accordingly, the present invention relates to an antiparasitic combination of the invention for use as a medicament, including, but not limited to, for use as an antiparasitic medicament.

In one embodiment, the present invention provides an antiparasitic combination of the invention for use as a medicament that is administered orally. For example, in one embodiment the antiparastic combination of the invention is comprised in a pharmaceutical composition of the invention as described herein, including, but not limited to, the oral dosage forms described herein.

Furthermore, the present invention relates to the antiparasitic combinations of the invention for use in a method for the treatment, prevention and/or control of parasitic infections and/or infestations in and/or on animals.

In a further aspect the present invention relates to the antiparasitic combinations of the invention for use in the treatment, prevention and/or control of parasitic infections and/or infestations in and/or on animals. In one embodiment, the present invention relates to the antiparasitic combinations of the invention for use in the treatment of parasitic infections and/or infestations in and/or on animals. In another embodiment, the present invention relates to the antiparasitic combinations of the invention for use in the prevention of parasitic infections and/or infestations in and/or on animals.

In one embodiment, the present invention relates to the antiparasitic combinations of the invention for use in the treatment, prevention and/or control of parasitic infections and/or infestations in and/or on animals, wherein the antiparasitic combination is administered orally. In one embodiment, the present invention relates to the antiparasitic combinations of the invention for use in the treatment of parasitic infections and/or infestations in and/or on animals, wherein the antiparasitic combination is administered orally. In another embodiment, the present invention relates to the antiparasitic combinations of the invention for use in the prevention of parasitic infections and/or infestations in and/or on animals, wherein the antiparasitic combination is administered orally.

In a further aspect the present invention relates to the use of the antiparasitic combinations of the invention for the preparation of a medicament for the treatment, prevention and/or control of parasitic infections and/or infestations in and/or on animals. In one embodiment, the present invention relates to the use of the antiparasitic combinations of the invention for the preparation of a medicament for the treatment of parasitic infections and/or infestations in and/or on animals. In another embodiment the present invention relates to the use of the antiparasitic combinations of the invention for the preparation of a medicament for the prevention of parasitic infections and/or infestations in and/or on animals.

In a further aspect the present invention relates to the use of the antiparasitic combinations of the invention for the preparation of a medicament for the treatment, prevention and/or control of parasitic infections and/or infestations in and/or on animals, wherein the medicament is administered orally. In one embodiment, the present invention relates to the use of the antiparasitic combinations of the invention for the preparation of a medicament for the treatment of parasitic infections and/or infestations in animals, wherein the medicament is administered orally. In another embodiment the present invention relates to the use of the antiparasitic combinations of the invention for the preparation of a medicament for the prevention of parasitic infections and/or infestations in and/or on animals, wherein the medicament is administered orally.

In a further aspect of the present invention the present invention relates to methods for the treatment, prevention and/or control of parasitic infections and/or infestations in and/or on animals, which methods comprise the administration of a pharmaceutically effective amount of the antiparasitic combinations of the invention or pharmaceutical compositions of the invention to an animal/animal patient in need thereof. In one embodiment, the present invention relates to methods for the treatment of parasitic infections and/or infestations in and/or on animals, which methods comprise the administration of a pharmaceutically effective amount of the antiparasitic combinations of the invention or pharmaceutical compositions of the invention to an animal/animal patient in need thereof. In another embodiment, the present invention relates to methods for the prevention of parasitic infections and/or infestations in and/or on animals, which methods comprise the administration of a pharmaceutically effective amount of the antiparasitic combinations of the invention or pharmaceutical compositions of the invention to an animal/animal patient in need thereof.

In an embodiment of the present invention the present invention relates to methods for the treatment, prevention and/or control of parasitic infections and/or infestations in and/or on animals, which methods comprise the oral administration of a pharmaceutically effective amount of the antiparasitic combinations of the invention or pharmaceutical compositions of the invention to an animal/animal patient in need thereof. In one embodiment, the present invention relates to methods for the treatment of parasitic infections and/or infestations in and/or on animals, which methods comprise the oral administration of a pharmaceutically effective amount of the antiparasitic combinations of the invention or pharmaceutical compositions of the invention to an animal/animal patient in need thereof. In another embodiment, the present invention relates to methods for the prevention of parasitic infections and/or infestations in and/or on animals, which methods comprise the oral administration of a pharmaceutically effective amount of the antiparasitic combinations of the invention or pharmaceutical compositions of the invention to an animal/animal patient in need thereof.

The antiparasitic combinations of the present invention are highly effective for the treatment, prevention and/or control of external and/or internal parasites in and/or on animals including mammals, fish and birds. The antiparasitic combinations of the invention may be used to treat cats, dogs, horses, chicken, pigs, sheep and cattle, but also humans, with the aim of substantially ridding these hosts of ectoparasites and/or endoparasites. Mammals, which can be treated, include, but are not limited to, humans, cats, dogs, cattle, cows, bison, deer, goats, horses, llamas, camels, pigs, sheep and yaks.

In one embodiment of the present invention, the mammals treated are humans, cats or dogs. In another embodiment of the present invention, the mammals treated are horses. In yet another embodiment of the present invention, the mammals treated are livestock animals, such as cattle or sheep. In yet another embodiment, the compounds of the invention may be used to treat fish.

In one embodiment of the invention, any of the antiparasitic active agent combinations described herein, and compositions comprising the combinations, will be used to treat and/or prevent an endoparasitic infection in an animal.

In another embodiment of the invention, any of the antiparasitic active agent combinations described herein, and compositions comprising the combinations, will be used to treat and/or prevent an ectoparasitic infestation on an animal.

In one embodiment of the invention, any of the antiparasitic active agent combinations described herein, and compositions comprising the combinations, will be used to treat and/or prevent an endoparasitic infection and an ectoparasitic infestation in and on an animal.

In one embodiment of the invention, any of the antiparasitic active agent combinations described herein, and compositions comprising the combinations, will be used to treat and/or prevent a parasitic infection by Dirofilaria immitis in an animal.

In another embodiment of the invention, any of the antiparasitic active agent combinations described herein, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, any of the antiparasitic active agent combinations described herein, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, any of the active agent combinations described herein, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, any of the antiparasitic active agent combinations described herein, and compositions comprising the combinations, will be used to treat and/or prevent a parasitic infestation by fleas and/or ticks on an animal. Flea species treated include, but are not limited to, Ctenocephalides felis, Ctenocephalides canis, Pulex simulans and Echidnophaga gallinacea. Tick species treated by the antiparasitic active agent combinations and compositions comprising them include, but are not limited to, Ixodes scapularis, Ixodes Ricinus, Ixodes hexagonus, Rhipicephalus sanguineus, Hyalomma marginatum, Haemaphysalis longicornis, Dermacentor reticulatus, Dermacentor variabilis, Haemaphisalis longicornis, Amblyomma maculatum and Amblyomma Americanum.

In another embodiment of the invention, any of the antiparasitic active agent combinations described herein, and compositions comprising the combinations, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri) and/or adult lungworms (Dictyocaulus arnfieldi).

another embodiment of the invention, any of the antiparasitic active agent combinations described herein, and compositions comprising the combinations, will be used to treat and/or prevent parasitic infections of nematodes and cestodes in animals including, but not limited to, adult stages of large strongyles (Strongylus vulgaris), adult stages of small strongyles (Cyathostominae), adult ascarids (Parascaris equorum) and/or adult tapeworms (Anoplocephala perfoliata).

In one embodiment of the invention, any of the antiparasitic active agent combinations described herein, and compositions comprising the combinations, will be used to treat and/or prevent a parasitic infection by Dirofilaria immitis, hookworm, roundworms and/or cestodes, and/or infestations by ticks and/or fleas in and/or on an animal.

In one embodiment of the invention, any of the active agent combinations described herein, and compositions comprising the combinations, will be used to treat and/or prevent a parasitic infection by Dirofilaria immitis and one or more infections or infestations of hookworm, roundworms, cestodes, and ticks and fleas in and/or on an animal.

In another embodiment of the invention, any of the antiparasitic active agent combinations described herein, and compositions comprising the combinations, will be used to treat and/or prevent a parasitic infection by hookworm and one or more infections or infestations of Dirofilaria immitis, roundworms, cestodes, and ticks and/or fleas in and/or on an animal.

In another embodiment of the invention, any of the antiparasitic active agent combinations described herein, and compositions comprising the combinations, will be used to treat and/or prevent a parasitic infection by roundworms and one or more infections and/or infestations of Dirofilaria immitis, hookworm, cestodes, and ticks and/or fleas in and/or on an animal.

In another embodiment of the invention, any of the antiparasitic active agent combinations described herein, and compositions comprising the combinations, will be used to treat and/or prevent a parasitic infection by cestodes and one or more infections and/or infestations of Dirofilaria immitis, hookworms, roundworms, and ticks and/or fleas in and/or on an animal.

In another embodiment of the invention, any of the antiparasitic active agent combinations described herein, and compositions comprising the combinations, will be used to treat and/or prevent a parasitic infestation by ticks and/or fleas and one or more infections of Dirofilaria immitis, hookworm, roundworms and cestodes on and in an animal.

In one embodiment of the invention, any of the antiparasitic active agent combinations described herein, and compositions comprising the same, will be used to treat and/or prevent a parasitic infection by Dirofilaria immitis and hookworm, and optionally infestations by ticks and/or fleas, in and/or on an animal.

In another embodiment of the invention, any of the antiparasitic active agent combinations described herein, and compositions comprising the same, will be used to treat and/or prevent a parasitic infection by Dirofilaria immitis and roundworms, and optionally infestations by ticks and/or fleas, in and/or on an animal.

In another embodiment of the invention, any of the antiparasitic active agent combinations described herein, and compositions comprising the same, will be used to treat and/or prevent a parasitic infection by Dirofilaria immitis and cestodes, and optionally infestations by ticks and/or fleas, in and on an animal.

In another embodiment of the invention, any of the antiparasitic active agent combinations described herein, and compositions comprising the same, will be used to treat and/or prevent a parasitic infection by hookworm and roundworms, and optionally infestations by ticks and/or fleas, in and/on an animal.

In another embodiment of the invention, any of the antiparasitic active agent combinations described herein, and compositions comprising the same, will be used to treat and/or prevent a parasitic infection by hookworm and cestodes, and optionally infestations by ticks and/or fleas, in and/or on an animal.

In another embodiment of the invention, any of the antiparasitic active agent combinations described herein, and compositions comprising the same, will be used to treat and/or prevent a parasitic infection by roundworms and cestodes, and optionally infestations by ticks and/or fleas, in and/or on an animal.

In one embodiment of the invention, any of the antiparasitic active agent combinations described herein, and compositions comprising the same, will be used to treat and/or prevent a parasitic infection by Dirofilaria immitis, hookworm and roundworms, and optionally infestations by ticks and/or fleas, in and/or on an animal.

In another embodiment of the invention, any of the antiparasitic active agent combinations described herein, and compositions comprising the same, will be used to treat and/or prevent a parasitic infection by Dirofilaria immitis, hookworm and cestodes, and optionally infestations by ticks and/or fleas, in and/or on an animal.

In another embodiment of the invention, any of the antiparasitic active agent combinations described herein, and compositions comprising the same, will be used to treat and/or prevent a parasitic infection by Dirofilaria immitis, roundworms, cestodes, and optionally infestations by ticks and/or fleas, in and/or on an animal.

In another embodiment of the invention, any of the antiparasitic active agent combinations described herein, and compositions comprising the same, will be used to treat and/or prevent a parasitic infection by Dirofilaria immitis, hookworm and roundworms, and optionally infestations by ticks and/or fleas, in and/or on an animal.

In another embodiment of the invention, any of the antiparasitic active agent combinations described herein, and compositions comprising the same, will be used to treat and/or prevent a parasitic infection by hookworm, roundworms and cestodes, and optionally infestations by ticks and/or fleas, in and/or on an animal.

In one embodiment of the invention, each of the antiparasitic active agent combinations 1 to 80 described below, and compositions comprising the combinations, will be used to treat and/or prevent a parasitic infection by Dirofilaria immitis in an animal.

In another embodiment of the invention, each of the antiparasitic active agent combinations 1 to 80 described below, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, each of the antiparasitic active agent combinations 1 to 80 described below, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, each of the antiparasitic active agent combinations 1 to 80 described below, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, each of the antiparasitic active agent combinations 1 to 80 described below, and compositions comprising the combinations, will be used to treat and/or prevent parasitic infestations by fleas and ticks on an animal. Flea species treated include, but are not limited to, Ctenocephalides felis, Ctenocephalides canis, Pulex simulans and Echidnophaga gallinacea. Tick species treated by the antiparasitic active agent combinations and compositions comprising them include, but are not limited to, Ixodes scapularis, Ixodes Ricinus, Ixodes hexagonus, Rhipicephalus sanguineus, Hyalomma marginatum, Haemaphysalis longicornis, Dermacentor reticulatus, Dermacentor variabilis, Amblyomma maculatum and Amblyomma americanum.

In one embodiment of the invention, each of the antiparasitic active agent combinations 1 to 80 described below, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, any of the antiparasitic active agent combinations described herein, including active agent combinations 1 to 80 described below, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, each of the antiparasitic active agent combinations 1 to 80 described below, and compositions comprising the combinations, will be used to treat and/or prevent a parasitic infection by hookworm and one or more of Dirofilaria immitis, roundworms, cestodes, and infestations by ticks and fleas in and/or on an animal.

In another embodiment of the invention, each of the antiparasitic active agent combinations 1 to 80 described below, and compositions comprising the combinations, will be used to treat and/or prevent a parasitic infection by roundworms and one or more of Dirofilaria immitis, hookworm, cestodes, and infestations by ticks and fleas in and/or on an animal.

In another embodiment of the invention, each of the antiparasitic active agent combinations 1 to 80 described below, and compositions comprising the combinations, will be used to treat and/or prevent a parasitic infection by cestodes and one or more of Dirofilaria immitis, hookworms, roundworms, and infestations by ticks and/or fleas in and/or on an animal.

In another embodiment of the invention, each of the antiparasitic active agent combinations 1 to 80 described below, and compositions comprising the combinations, will be used to treat and/or prevent a parasitic infestation by ticks and/or fleas and one or more infections of Dirofilaria immitis, hookworm, roundworms and cestodes in and/or on an animal.

In one embodiment of the invention each of the antiparasitic active agent combinations 1 to 80 described below, and compositions comprising the same, will be used to treat and/or prevent a parasitic infection by Dirofilaria immitis, hookworm, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by Dirofilaria immitis, roundworms, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by Dirofilaria immitis, cestodes, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by hookworm, roundworms, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by hookworm and cestodes, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by roundworms and cestodes, and optionally infestations by ticks and/or fleas, in and/or on an animal.

In one embodiment of the invention, each of the antiparasitic active agent combinations 1 to 80 described below, and compositions comprising the same, will be used to treat and/or prevent a parasitic infection by Dirofilaria immitis, hookworm, roundworms, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by Dirofilaria immitis, hookworm, cestodes, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by Dirofilaria immitis, roundworms, cestodes, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by Dirofilaria immitis, hookworm, roundworms, and optionally infestations by ticks and/or fleas, in and/or on an animal.

In another embodiment of the invention, each of the antiparasitic active agent combinations 1 to 80 described below, and compositions comprising the same, will be used to treat and/or prevent a parasitic infection by hookworm, roundworms, cestodes, and optionally infestations by ticks and/or fleas, in and/or on an animal.

In another embodiment of the invention, each of the antiparasitic active agent combinations 1 to 80 described below, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the antiparasitic active agent combinations 1 to 80 described below, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata)

In one embodiment of the invention, each of the antiparasitic active agent combinations C81-1 to C81-153 described in Table 2 below, and compositions comprising the combinations, will be used to treat and/or prevent a parasitic infection by Dirofilaria immitis in an animal.

In another embodiment of the invention, each of the antiparasitic active agent combinations C81-1 to C81-153 described in Table 2 below, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, each of the antiparasitic active agent combinations C81-1 to C81-153 described in Table 2 below, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, each of the antiparasitic active agent combinations C81-1 to C81-153 described in Table 2 below, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, each of the antiparasitic active agent combinations C81-1 to C81-153 described in Table 2 below, and compositions comprising the combinations, will be used to treat and/or prevent parasitic infestations by fleas and/or ticks on an animal. Flea species treated include, but are not limited to, Ctenocephalides felis, Ctenocephalides canis, Pulex simulans and Echidnophaga gallinacea. Tick species treated by the antiparasitic active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, each of the antiparasitic active agent combinations C81-1 to C81-153 described in Table 2 below, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, each of the antiparasitic active agent combinations C81-1 to C81-153 described in Table 2 below, and compositions comprising the combinations, will be used to treat and/or prevent a parasitic infection by Dirofilaria immitis and one or more of hookworms, roundworms, cestodes, and infestations by ticks and/or fleas in and/or on an animal.

In another embodiment of the invention, each of the antiparasitic active agent combinations C81-1 to C81-153 described in Table 2 below, and compositions comprising the combinations, will be used to treat and/or prevent a parasitic infection by hookworms and one or more of Dirofilaria immitis, roundworms, cestodes, and infestations by ticks and/or fleas in and/or on an animal.

In another embodiment of the invention, each of the antiparasitic active agent combinations C81-1 to C81-153 described in Table 2 below, and compositions comprising the combinations, will be used to treat and/or prevent a parasitic infection by roundworms and one or more of Dirofilaria immitis, hookworms, cestodes, and infestations by ticks and/or fleas in and/or on an animal.

In another embodiment of the invention, each of the antiparasitic active agent combinations C81-1 to C81-153 described in Table 2 below, and compositions comprising the combinations, will be used to treat and/or prevent a parasitic infection by cestodes and one or more of Dirofilaria immitis, hookworms, roundworms, and infestations by ticks and/or fleas in and/or on an animal.

In another embodiment of the invention, each of the antiparasitic active agent combinations C81-1 to C81-153 described in Table 2 below, and compositions comprising the combinations, will be used to treat and/or prevent a parasitic infestation by ticks and/or fleas and one or more infections of Dirofilaria immitis, hookworms, roundworms and cestodes in and on an animal.

In one embodiment of the invention, each of the antiparasitic active agent combinations C81-1 to C81-153 described in Table 2 below, and compositions comprising the same, will be used to treat and/or prevent a parasitic infection by Dirofilaria immitis, hookworms, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by Dirofilaria immitis, roundworms, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by Dirofilaria immitis, cestodes, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by hookworms, roundworms, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by hookworms and cestodes, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by roundworms and cestodes, and optionally infestations by ticks and/or fleas, in and/or on an animal.

In one embodiment of the invention, each of the antiparasitic active agent combinations C81-1 to C81-153 described in Table 2 below, and compositions comprising the same, will be used to treat and/or prevent a parasitic infection by Dirofilaria immitis, hookworms, roundworms, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by Dirofilaria immitis, hookworms, cestodes, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by Dirofilaria immitis, roundworms, cestodes, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by Dirofilaria immitis, hookworms, roundworms, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infection by hookworms, roundworms, cestodes, and optionally infestations by ticks and/or fleas, in and/or on an animal.

In another embodiment of the invention, each of the antiparasitic active agent combinations C81-1 to C81-153 described in Table 2 below, and compositions comprising the same, will be used to treat and/or prevent a parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the antiparasitic active agent combinations C81-1 to C81-153 described in Table 2 below, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata).

In one embodiment of the invention, each of the antiparasitic active agent combinations C82-1 to C82-153 described in Table 3 below, and compositions comprising the combinations, will be used to treat and/or prevent a parasitic infection by Dirofilaria immitis in an animal.

In another embodiment of the invention, each of the antiparasitic active agent combinations C82-1 to C82-153 described in Table 3 below, and compositions comprising the combinations, will be used to treat and/or prevent a parasitic infection by hookworms (including, but not limited to, immature and mature stages of Ancylostoma caninum, Ancylostoma braziliense, Uncinaria stenocephala) in an animal.

In another embodiment of the invention, each of the antiparasitic active agent combinations C82-1 to C82-153 described in Table 3 below, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, each of the antiparasitic active agent combinations C82-1 to C82-153 described in Table 3 below, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, each of the antiparasitic active agent combinations C82-1 to C82-153 described in Table 3 below, and compositions comprising the combinations, will be used to treat and/or prevent parasitic infestations by fleas and ticks on an animal. Flea species treated include, but are not limited to, Ctenocephalides felis, Ctenocephalides canis, Pulex simulans and Echidnophaga gallinacea. Tick species treated by the antiparasitic active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, each of the antiparasitic active agent combinations C82-1 to C82-153 described in Table 3 below, and compositions comprising the combinations, will be used to treat and/or prevent a parasitic infection by Dirofilaria immitis, hookworms, roundworms, cestodes, and/or infestations by ticks and/or fleas in and/or on an animal.

In one embodiment of the invention, each of the antiparasitic active agent combinations C82-1 to C82-153 described in Table 3 below, and compositions comprising the combinations, will be used to treat and/or prevent a parasitic infection by Dirofilaria immitis and one or more of hookworms, roundworms, cestodes, and infestations by ticks and/or fleas in and/or on an animal.

In another embodiment of the invention, each of the antiparasitic active agent combinations C82-1 to C82-153 described in Table 3 below, and compositions comprising the combinations, will be used to treat and/or prevent a parasitic infection by hookworms and one or more of Dirofilaria immitis, roundworms, cestodes, and infestations by ticks and/or fleas in and/or on an animal.

In another embodiment of the invention, each of the antiparasitic active agent combinations C82-1 to C82-153 described in Table 3 below, and compositions comprising the combinations, will be used to treat and/or prevent a parasitic infection by roundworms and one or more of Dirofilaria immitis, hookworms, cestodes, and infestations by ticks and/or fleas in and/or on an animal.

In another embodiment of the invention, each of the antiparasitic active agent combinations C82-1 to C82-153 described in Table 3 below, and compositions comprising the combinations, will be used to treat and/or prevent a parasitic infection by cestodes and one or more of Dirofilaria immitis, hookworms, roundworms, and infestations by ticks and/or fleas in and/or on an animal.

In another embodiment of the invention, each of the antiparasitic active agent combinations C82-1 to C82-153 described in Table 3 below, and compositions comprising the combinations, will be used to treat and/or prevent parasitic infestations by ticks and/or fleas and one or more infections of Dirofilaria immitis, hookworms, roundworms and cestodes in and on an animal.

In one embodiment of the invention, each of the antiparasitic active agent combinations C82-1 to C82-153 described in Table 3 below, and compositions comprising the same, will be used to treat and/or prevent a parasitic infection by Dirofilaria immitis, hookworms, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by Dirofilaria immitis, roundworms, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by Dirofilaria immitis, cestodes, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by hookworms, roundworms, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by hookworms and cestodes, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by roundworms and cestodes, and optionally infestations by ticks and/or fleas, in and/or on an animal.

In one embodiment of the invention, each of the antiparasitic active agent combinations C82-1 to C82-153 described in Table 3 below, and compositions comprising the same, will be used to treat and/or prevent a parasitic infection by Dirofilaria immitis, hookworms, roundworms, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by Dirofilaria immitis, hookworms, cestodes, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by Dirofilaria immitis, roundworms, cestodes, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by Dirofilaria immitis, hookworms, roundworms, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent a parasitic infection by hookworms, roundworms, cestodes, and optionally infestations by ticks and/or fleas, in and/or on an animal.

In another embodiment of the invention, each of the antiparasitic active agent combinations C82-1 to C82-153 described in Table 3 below, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the antiparasitic active agent combinations C82-1 to C82-153 described in Table 3 below, and compositions comprising the same, will be used to treat and/or prevent a parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata).

In one embodiment of the invention, each of the antiparasitic active agent combinations C83-1 to C83-1224 described in Table 4 below, and compositions comprising the combinations, will be used to treat and/or prevent a parasitic infection by Dirofilaria immitis in an animal.

In another embodiment of the invention, each of the antiparasitic active agent combinations C83-1 to C83-1224 described in Table 4 below, and compositions comprising the combinations, will be used to treat and/or prevent a parasitic infection by hookworms (including, but not limited to, immature and mature stages of Ancylostoma caninum, Ancylostoma braziliense, Uncinaria stenocephala) in an animal.

In another embodiment of the invention, each of the antiparasitic active agent combinations C83-1 to C83-1224 described in Table 4 below, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, each of the antiparasitic active agent combinations C83-1 to C83-1224 described in Table 4 below, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, each of the antiparasitic active agent combinations C83-1 to C83-1224 described in Table 4 below, and compositions comprising the combinations, will be used to treat and/or prevent parasitic infestations by fleas and/or ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, each of the active agent combinations C83-1 to C83-1224 described in Table 4 below, and compositions comprising the combinations, will be used to treat and/or prevent a parasitic infection by Dirofilaria immitis, hookworms, roundworms, cestodes, and/or infestations by ticks and/or fleas in and/or on an animal.

In one embodiment of the invention, each of the antiparasitic active agent combinations C83-1 to C83-1224 described in Table 4 below, and compositions comprising the combinations, will be used to treat and/or prevent a parasitic infection by Dirofilaria immitis and one or more of hookworms, roundworms, cestodes, and infestations by ticks and/or fleas in and/or on an animal.

In another embodiment of the invention, each of the antiparasitic active agent combinations C83-1 to C83-1224 described in Table 4 below, and compositions comprising the combinations, will be used to treat and/or prevent a parasitic infection by hookworms and one or more of Dirofilaria immitis, roundworms, cestodes, and infestations by ticks and/or fleas in and/or on an animal.

In another embodiment of the invention, each of the antiparasitic active agent combinations C83-1 to C83-1224 described in Table 4 below, and compositions comprising the combinations, will be used to treat and/or prevent a parasitic infection by roundworms and one or more of Dirofilaria immitis, hookworms, cestodes, and infestations by ticks and/or fleas in and/or on an animal.

In another embodiment of the invention, each of the antiparasitic active agent combinations C83-1 to C83-1224 described in Table 4 below, and compositions comprising the combinations, will be used to treat and/or prevent a parasitic infection by cestodes and one or more of Dirofilaria immitis, hookworms, roundworms, and infestations by ticks and/or fleas in and/or on an animal.

In another embodiment of the invention, each of the antiparasitic active agent combinations C83-1 to C83-1224 described in Table 4 below, and compositions comprising the combinations, will be used to treat and/or prevent a parasitic infestations by ticks and/or fleas and one or more infections of Dirofilaria immitis, hookworms, roundworms and cestodes on and in an animal.

In one embodiment of the invention, each of the antiparasitic active agent combinations C83-1 to C83-1224 described in Table 4 below, and compositions comprising the same, will be used to treat and/or prevent a parasitic infection by Dirofilaria immitis, hookworms, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by Dirofilaria immitis, roundworms, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by Dirofilaria immitis, cestodes, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by hookworms, roundworms, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by hookworms and cestodes, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent parasitic infections by roundworms and cestodes, and optionally infestations by ticks and/or fleas, in and/or on an animal.

In one embodiment of the invention, each of the antiparasitic active agent combinations C83-1 to C83-1224 described in Table 4 below, and compositions comprising the same, will be used to treat and/or prevent a parasitic infection by Dirofilaria immitis, hookworms, roundworms, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by Dirofilaria immitis, hookworms, cestodes, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by Dirofilaria immitis, roundworms, cestodes, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by Dirofilaria immitis, hookworms, roundworms, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infection by hookworms, roundworms, cestodes, and optionally infestations by ticks and/or fleas, in and/or on an animal.

In another embodiment of the invention, each of the antiparasitic active agent combinations C83-1 to C83-1224 described in Table 4 below, and compositions comprising the same, will be used to treat and/or prevent a parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the antiparasitic active agent combinations C83-1 to C83-1224 described in Table 4 below, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata).

In one embodiment of the invention, each of the antiparasitic active agent combinations C84-1 to C84-1224 described in Table 5 below, and compositions comprising the combinations, will be used to treat and/or prevent a parasitic infection by Dirofilaria immitis in an animal.

In another embodiment of the invention, each of the antiparasitic active agent combinations C84-1 to C84-1224 described in Table 5 below, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, each of the antiparasitic active agent combinations C84-1 to C84-1224 described in Table 5 below, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, each of the antiparasitic active agent combinations C84-1 to C84-1224 described in Table 5 below, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, each of the antiparasitic active agent combinations C84-1 to C84-1224 described in Table 5 below, and compositions comprising the combinations, will be used to treat and/or prevent parasitic infestations by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, each of the antiparasitic active agent combinations C84-1 to C84-1224 described in Table 5 below, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, each of the antiparasitic active agent combinations C84-1 to C84-1224 described in Table 5 below, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, each of the antiparasitic active agent combinations C84-1 to C84-1224 described in Table 5 below, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, each of the antiparasitic active agent combinations C84-1 to C84-1224 described in Table 5 below, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, each of the antiparasitic active agent combinations C84-1 to C84-1224 described in Table 5 below, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, each of the antiparasitic active agent combinations C84-1 to C84-1224 described in Table 5 below, and compositions comprising the combinations, will be used to treat and/or prevent parasitic infestations by ticks and/or fleas and parasitic infections of one or more of Dirofilaria immitis, hookworm, roundworms and cestodes in and on an animal.

In one embodiment of the invention, each of the antiparasitic active agent combinations C84-1 to C84-1224 described in Table 5 below, and compositions comprising the same, will be used to treat and/or prevent a parasitic infection by Dirofilaria immitis, hookworm, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by Dirofilaria immitis, roundworms, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by Dirofilaria immitis, cestodes, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by hookworm, roundworms, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by hookworm and cestodes, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by roundworms and cestodes, and optionally infestations by ticks and/or fleas, in and/or on an animal.

In one embodiment of the invention, each of the antiparasitic active agent combinations C84-1 to C84-1224 described in Table 5 below, and compositions comprising the same, will be used to treat and/or prevent a parasitic infection by Dirofilaria immitis, hookworm, roundworms, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by Dirofilaria immitis, hookworm, cestodes, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by Dirofilaria immitis, roundworms, cestodes, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by Dirofilaria immitis, hookworm, roundworms, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infection by hookworm, roundworms, cestodes, and optionally infestations by ticks and/or fleas, in and/or on an animal.

In another embodiment of the invention, each of the antiparasitic active agent combinations C84-1 to C84-1224 described in Table 5 below, and compositions comprising the same, will be used to treat and/or prevent a parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the antiparasitic active agent combinations C84-1 to C84-1224 described in Table 5 below, and compositions comprising the same, will be used to treat and/or prevent a parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata).

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection and/or infestation in and/or on an animal.

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent an endoparasitic infection and/or ectoparasitic infestation in and/or on an animal.

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis in an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infestation by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, roundworms, cestodes, and/or infestations by ticks and fleas in and/or on an animal.

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infestation by ticks and/or fleas and one or more infections of Dirofilaria immitis, hookworm, roundworms and cestodes on and in an animal.

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, roundworms, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, cestodes, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm, roundworms, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm and cestodes, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by roundworms and cestodes, and optionally infestations by ticks and/or fleas, in and/or on an animal.

In one embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, cestodes, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, roundworms, cestodes, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising A472, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm, roundworms, cestodes, and optionally infestations by ticks and/or fleas, in and/or on an animal.

In one embodiment of the invention, the active agent combinations comprising compound A472, or a salt thereof, afoxolaner, moxidectin and praziquantel, and compositions comprising the combinations, will be used to treat and/or prevent an endoparasitic infection and/or ectoparasitic infestation in and/or on an animal.

In one embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis in an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, the active agent combinations comprising compound A472, or a salt thereof, afoxolaner, moxidectin and praziquantel, and compositions comprising the combinations, will be used to treat and/or prevent a parasitic infestation by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infestation by ticks and/or fleas and one or more infections of Dirofilaria immitis, hookworm, roundworms and cestodes on and in an animal.

In one embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, hookworm, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, roundworms, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, cestodes, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by hookworm, roundworms, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by hookworm and cestodes, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by roundworms and cestodes, and optionally infestations by ticks and/or fleas, in and/or on an animal.

In one embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, hookworm, cestodes, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, roundworms, cestodes, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising compound A472, or a salt thereof, afoxolaner, moxidectin and praziquantel, and compositions comprising the combinations, will be used to treat and/or prevent a parasitic infection by hookworm, roundworms, cestodes, and optionally infestations by ticks and/or fleas, in and/or on an animal.

In another embodiment of the invention, each of the active agent combinations comprising compound A472, or a salt thereof, and praziquantel, and compositions comprising the same, will be used to treat and/or prevent a parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the active agent combinations comprising compound A472, or a salt thereof, and praziquantel, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata).

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection and/or infestation in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent an endoparasitic infection and/or ectoparasitic infestation in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infestation by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infestation by ticks and/or fleas and one or more infection of Dirofilaria immitis, hookworm, roundworms and cestodes in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infections by Dirofilaria immitis, hookworm, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm and cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by roundworms and cestodes, and optionally infestations by ticks and/or fleas, in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, roundworms, cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally praziquantel, and compositions comprising the combinations, will be used to treat and/or prevent a parasitic infection by hookworm, roundworms, cestodes, and optionally infestations by ticks and/or fleas, in and/or on an animal.

In one embodiment of the invention, the active agent combinations comprising compound IC-1, or a salt thereof, afoxolaner, moxidectin and praziquantel, and compositions comprising the combinations, will be used to treat and/or prevent an endoparasitic and/or ectoparasitic infection or infestation in and/or on an animal.

In one embodiment of the invention, the active agent combinations comprising compound IC-1, or a salt thereof, lotilaner, moxidectin and praziquantel, and compositions comprising the combinations, will be used to treat and/or prevent an ectoparasitic and/or endoparasitic infestation or infection in and/or on an animal.

In one embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis in an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, the active agent combinations comprising compound IC-1, or a salt thereof, lotilaner, moxidectin and praziquantel, and compositions comprising the combinations, will be used to treat and/or prevent a parasitic infestation by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, the active agent combinations comprising compound IC-1, or a salt thereof, lotilaner, moxidectin and praziquantel, and compositions comprising the combinations, will be used to treat and/or prevent parasitic infestations by ticks and fleas and one or more parasitic infections of Dirofilaria immitis, hookworm, roundworms and cestodes on and in an animal.

In one embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, hookworm, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, roundworms, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, cestodes, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by hookworm, roundworms, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by hookworm and cestodes, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by roundworms and cestodes, and optionally infestations by ticks and/or fleas, in and/or on an animal.

In one embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, hookworm, cestodes, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, roundworms, cestodes, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising compound IC-1, or a salt thereof, lotilaner, moxidectin and praziquantel, and compositions comprising the combinations, will be used to treat and/or prevent a parasitic infection by hookworm, roundworms, cestodes, and optionally infestations by ticks and/or fleas, in and/or on an animal.

In another embodiment of the invention, each of the active agent combinations comprising compound IC-1, or a salt thereof, and praziquantel, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), adult tapeworms (Anoplocephala perfoliata), immature and encysted stages of small strongyles (Cyathostomines), adult hairworms(Trichostrongylus axei), adult pinworms (Oxyuris equi), microfilariae of neck threadworms (Onchocerca cervicalis), adult intestinal threadworms (Strongyloides westeri), and/or adult lungworms (Dictyocaulus arnfieldi).

In another embodiment of the invention, each of the active agent combinations comprising compound IC-1, or a salt thereof, and praziquantel, and compositions comprising the same, will be used to treat and/or prevent parasitic infections of nematodes and/or cestodes in animals including, but not limited to, adult stages of large strongyles (Strongylus vulgaris), adult stages of small strongyles (Cyathostominae), adult ascarids (Parascaris equorum), and/or adult tapeworms (Anoplocephala perfoliata).

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection and/or infestation in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent an endoparasitic and/or ectoparasitic infection or infestation in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent parasitic infestations by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent parasitic infestations by ticks and/or fleas and one or more parasitic infections of Dirofilaria immitis, hookworm, roundworms and cestodes on and in an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, hookworm, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by hookworm, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by hookworm and cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by roundworms and cestodes, and optionally infestations by ticks and/or fleas, in and/or on an animal.

In one embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, hookworm, cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, roundworms, cestodes, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising IC-1, or a salt thereof, a macrocyclic lactone active agent (including ML-1 to ML-17), an isoxazoline active agent (including Is-1 to Is-9), and optionally pyrantel, and compositions comprising the combinations, will be used to treat and/or prevent a parasitic infection by hookworm, roundworms, cestodes, and optionally infestations by ticks and/or fleas, in and/or on an animal.

In one embodiment of the invention, the active agent combinations comprising compound IC-1, or a salt thereof, afoxolaner, moxidectin and pyrantel, and compositions comprising the combinations, will be used to treat and/or prevent an endoparasitic and/or ectoparasitic infection or infestation in and/or on an animal.

In one embodiment of the invention, the active agent combinations comprising compound IC-1, or a salt thereof, lotilaner, moxidectin and pyrantel, and compositions comprising the combinations, will be used to treat and/or prevent an ectoparasitic and/or endoparasitic infestation or infection in and/or on an animal.

In one embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis in an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by hookworm (including, but not limited to, immature and mature stages of Ancylostoma caninum, Ancylostoma braziliense, Uncinaria stenocephala) in an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by roundworms (including, but not limited to, Toxocara canis, Toxascaris leonina, Toxocara cati, Toxocara malaysiensis) in an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by cestodes including, but not limited to, Dipylidium caninum, Echinococcus granulosus and Echinococcus multilocularis in an animal.

In another embodiment of the invention, the active agent combinations comprising compound IC-1, or a salt thereof, lotilaner, moxidectin and pyrantel, and compositions comprising the combinations, will be used to treat and/or prevent a parasitic infestation by fleas and ticks on an animal. Flea species treated include, but are not limited to, Ctenocephalides felis, Ctenocephalides canis, Pulex simulans and Echidnophaga gallinacea. Tick species treated by the active agent combinations and compositions comprising them include, but are not limited to, Ixodes scapularis, Rhipicephalus sanguineus, Dermacentor variabilis, Haemaphisalis longicornis, Amblyomma maculatum and Amblyomma Americanum.

In one embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, hookworm, roundworms, cestodes, and/or infestations by ticks and/or fleas in and/or on an animal.

In one embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis and one or more of hookworm, roundworms, cestodes, and infestations by ticks and/or fleas in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by hookworm and one or more of Dirofilaria immitis, roundworms, cestodes, and infestations by ticks and/or fleas in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by roundworms and one or more of Dirofilaria immitis, hookworm, cestodes, and infestations by ticks and/or fleas in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by cestodes and one or more of Dirofilaria immitis, hookworm, roundworms, and infestations by ticks and/or fleas in and/or on an animal.

In another embodiment of the invention, the active agent combinations comprising compound IC-1, or a salt thereof, lotilaner, moxidectin and pyrantel, and compositions comprising the combinations, will be used to treat and/or prevent parasitic infestations by ticks and/or fleas and one or more parasitic infections of Dirofilaria immitis, hookworm, roundworms and cestodes on and in an animal.

In one embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, hookworm, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, roundworms, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, cestodes, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by hookworm, roundworms, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by hookworm and cestodes, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by roundworms and cestodes, and optionally infestations by ticks and/or fleas, in and/or on an animal.

In one embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, hookworm, cestodes, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, roundworms, cestodes, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by Dirofilaria immitis, hookworm, roundworms, and optionally infestations by ticks and/or fleas, in and/or on an animal.

In another embodiment of the invention, the active agent combinations comprising compound IC-1, or a salt thereof, lotilaner, moxidectin and pyrantel, and compositions comprising the combinations, will be used to treat and/or prevent a parasitic infection by hookworm, roundworms, cestodes, and optionally infestations by ticks and/or fleas, in and/or on an animal.

The dose range of the compounds of Formula (IA), Formula (IB) or Formula (IC) applicable per day is usually from 0.001 mg/kg to 1,000 mg/kg of body weight for animals.

In one embodiment, the antiparasitic combinations of the invention will be effective against hookworm in dogs including, but not limited to, Ancylostoma caninum. The combinations of the invention would be evaluated for safety and efficacy in vivo against adult Ancylostoma caninum according to the following protocol. Beagle dogs receive a full physical examination prior to inclusion in the study. Each dog is inoculated orally with approximately 300 infective L₃ ofA. caninum on study day −24. Dogs will be randomly allocated to one of three treatment groups. Each study group will contain eight dogs. Dogs in Treatment Group 1 (positive control) are treated on study day 0 with a commercial product containing 5 mg/kg pyrantel (as pamoate salt) at the label dose according to instructions. Dogs in Treatment Group 2 (test group) are treated orally once on study day 0 with an oral chewable tablet containing a combination of compound A472, afoxolaner, moxidectin and praziquantel in concentrations prepared to deliver doses of 0.8 mg/kg compound A472, 2.5 mg/kg afoxolaner, 12 μg/kg moxidectin and 5 mg/kg praziquantel per body weight of dog treated. Dogs in Treatment Group 3 (negative control) would not receive any treatment. On study day 7 dogs in the study are euthanized and necropsied for evaluation of the presence of adult Ancylostoma caninum worms.

In one embodiment, the antiparasitic combinations of the invention will be effective against roundworms in dogs including, but not limited to, Toxocara canis. The combinations of the invention would be evaluated for safety and efficacy in vivo against adult Toxocara canis according to the following protocol. Beagle dogs receive a full physical examination prior to inclusion in the study. Each dog is infected with approximately 100-300 infective T. canis on Day −49. Dogs are ranked by decreasing body weight within sex to form blocks of eight dogs each with approximately equal numbers of each sex and randomly assigned to each treatment group. Each study group will contain eight dogs. Dogs in the negative control group (Group 1) are untreated. Dogs in Group 2 are treated orally once on study day 0 with an oral chewable tablet containing a combination of compound A472, afoxolaner, moxidectin and praziquantel in concentrations prepared to deliver doses of 0.8 mg/kg compound A472, 2.5 mg/kg afoxolaner, 12 μg/kg moxidectin and 5 mg/kg praziquantel per body weight of dog treated. Dogs in Group 3 are also treated once orally on study day 0 with an oral chewable tablet prepared to deliver a dose of 12 μg/kg moxidectin, 2.5 mg/kg afoxolaner and 5 mg/kg praziquantel per body weight of dog treated. On study day 7 dogs in the study are euthanized and necropsied for evaluation of the presence of adult Toxocara canis worms.

In another embodiment, the antiparasitic combinations of the invention would be effective for preventing infections of Dirofilaria immitis in dogs. In one embodiment, the antiparasitic combinations would be evaluated for safety and efficacy in vivo against Dirofilaria immitis according to the following protocol. Beagle dogs are tested for microfilaria and heartworm antigen and receive a full physical examination prior to inclusion in the study. Each dog is inoculated with 50 infective third-stage D. immitis larvae on Day −30. Antigen testing is performed on blood collected on Day 90 to confirm that animals had not been exposed to D. immitis prior to inducing the infection. Dogs are ranked by decreasing body weight within sex to form blocks of eight dogs each with approximately equal numbers of each sex and randomly assigned to each treatment group. Each study group will contain eight dogs. Dogs in the negative control group (Group 1) are untreated. Dogs in Group 2 are treated orally once on study day 0 with an oral chewable tablet containing a combination of compound A472, afoxolaner, moxidectin and praziquantel in concentrations prepared to deliver doses of 0.8 mg/kg compound A472, 2.5 mg/kg afoxolaner, 12 μg/kg moxidectin and 5 mg/kg praziquantel per body weight of dog treated. Dogs in Group 3 are also treated once orally on study day 0 with an oral chewable tablet containing a combination of compound A472, afoxolaner and praziquantel in concentrations prepared to deliver doses of 0.8 mg/kg compound A472, 2.5 mg/kg afoxolaner and 5 mg/kg praziquantel per body weight of dog treated. On study day 120 dogs in the study are euthanized and necropsied for evaluation of the presence of adult heartworms.

The actual pharmaceutically effective amount or therapeutic dosage will usually depend on factors known by those skilled in the art such as age and weight of the animal patient, route of administration and severity of disease. In any case the compounds of the invention will be administered at dosages and in a manner which allows a pharmaceutically effective amount to be delivered based upon the animal patient's unique condition.

### Veterinary Parasites

In one embodiment, the antiparasitic active agent combinations of the invention and pharmaceutical compositions comprising the antiparasitic active agent combinations may be used for treating, controlling and/or preventing an endoparasitic infection in an animal by one or more of the following parasite genera: Aelurostrongylus, Acanthocheilonema, Ancylostoma, Angiostrongylus, Anoplocephala, Ascaridia, Ascaris, Babesia, Besnoitia, Brugia, Bunostomum, Caballonema, Capillaria, Chabertia, Cooperia, Crenosoma, Coronocyclus, Craterostomum, Crenosoma, Cryptosporidium, Cyathostomum, Cylicocyclus, Cylicodontophorus, Cylicostephanus, Cylindropharynx, Cystocaulus, Cystoisospora, Cytauxzoon, Dictyocaulus, Dipetalonema, Diphyllobothrium (Dibothriocephalus), Diplopylidium, Dipylidium, Dioctophyme, Dirofilaria, Dracunculus, Draschia, Echinococcus, Eimeria, Enterobius, Fasciola, Filaroides, Giardia, Gyalocephalus, Habronema, Haemonchus, Hammondia, Heligmosomoides, Hepatozoon, Heterakis, Histomonas, Hsiungia, Hymenolepis, Hyostrongylus, Isospora (Cytoisospora), Joyeuxiella, Leishmania, Linguatula, Litomosoides, Macracanthorynchus, Mansonella, Mesocestoides, Metastrongylus, Moniezia, Necator, Nematodirus, Neospora, Nippostrongylus, Oesophagostomum, Onchocerca, Opisthorchis, Oslerus, Ostertagia, Oxyuris, Parafilaria, Parapoteriostomum, Parascaris, Petrovinema, Pneumonyssoides, Pneumonyssus, Poteriostomum, Probstmayria, Protostrongylus, Schistosoma, Skrjabinodentus, Spirocerca, Spirometra, Spirura, Strongyloides, Strongylus, Taenia, Teladorsagia, Theileria, Thelazia, Toxascaris, Toxocara, Toxoplasma, Trichinella, Trichostrongylus, Trichuris, Tridentoinfundibulum, Triodontophorus, Tritrichomonas, Troglostrongylus, Trypanosoma, Uncinaria, Wuchereria.

In one embodiment, the antiparasitic active agent combinations and compositions of the invention may be used to treat, control and/or prevent an endoparasitic infection in an animal by one or more of the following parasites: Aelurostrongylus abstrusus, Ancylostoma braziliense, Ancylostoma caninum, Ancylostoma ceylanicum, Ancylostoma tubaeforme, Angiostrongylus vasorum, Anoplocephala magna, Anoplocephala perfoliata, Ascaris suum, Babesia bigemina, Babesia bovis, Babesia canis, Babesia divergens, Babesia gisoni, Babesia major, Babesia rossi, Babesia vogeli, Babesia vulpes, Brugia buckleyi, Brugia ceylonensis, Brugia malayi, Brugia pahangi, Brugia patei, Bunostomum phlebotomum, Bunostomum trigonocephalum, Caballonema longicapsulatum, Capillaria aerophila, Capillaria anatis, Capillaria bovis, Capillaria contorta, Capillaria feliscati, Capillaria obsignata, Chabertia ovina, Clonorchis sinensis, Cooperia curticei, Cooperia oncophora, Cooperia pectinata, Cooperia punctata, Cooperia surnabada, Coronocyclus coronatus, Coronocyclus labiatus, Coronocyclus labratus, Craterostomum acuticaudatum, Crenosoma vulpis, Cyathostomum alveatum, Cyathostomum catinatum, Cyathostomum paternatum, Cyathostomum tetracanthum, Cylicocyclus ashworthi, Cylicocyclus brevicapsulatus, Cylicocyclus elongatus, Cylicocyclus insigniae, Cylicocyclus leptosomatum, Cylicocyclus nassatus, Cylicocyclus radiatus, Cylicocyclus ultrajectinus, Cylicodontophorus bicoronatus, Cylicodontophorus mettami, Cylicostephanus asymetricus, Cylicostephanus bidentatus, Cylicostephanus calicatus, Cylicostephanus goldi, Cylicostephanus hybridus, Cylicostephanus longibursatus, Cylicostephanus minutus, Cylindropharynx brevicauda, Cylindropharynx intermedia, Cylindropharynx longicauda, Cystocaulus oreatus, Cytauxzoon felis, Cytstoisispora burrowsi, Cytstoisispora canis, Cytstoisispora felis, Cytstoisispora ohioensis, Cytstoisispora rivolta, Cytstoisospora suis, Dictyocaulus arnfieldi, Dictyocaulus filaria, Dictyocaulus viviparus, Dipetalonema caudispina, Dipetalonema freitasi, Dipetalonema gracile, Dipetalonema robini, Dipetalonema yatesi, Diphyllobothrium latum, Dipylidium caninum, Dirofilaria hongkongensis, Dirofilaria immitis, Dirofilaria repens, Dracunculus insignis, Dracunculus lutrae, Dracunculus medinensis, Draschia megastoma, Echinococcus granulosus, Echinococcus multilocularis, Eimeria acervuline, Eimeria adenoids, Eimeria auburnensis, Eimeria bovis, Eimeria brunetti, Eimeria crandallis, Eimeria dispersa, Eimeria gallopavonis, Eimeria hagani, Eimeria maxima, Eimeria meleagrimitis, Eimeria mitavi, Eimeria necatrix, Eimeria ninakohlyakimovae, Eimeria ovina, Eimeria ovinoidalis, Eimeria praecox, Eimeria tenella, Eimeria zuernii, Fasciola gigantica, Fasciola hepatica, Fascioloides magna, Filaroides hirthi, Filaroides milksi, Filaroides osleri, Giardia duodenalis (syn. Giardia lamblia, Giardia intestinalis), Gyalocephalus capitatus, Habronema microstoma, Habronema muscae, Haemonchus contortus, Haemonchus placei, Haemonchus similis, Heterakis gallinarum, Histomonas meleagridis, Hsiungia triciliata, Hyostrongylus rubidus, Joyeuxiella pasquali, Leishmania infantum, Metastrongylus apri, Metastrongylus pudendotectus, Metastrongylus salmi, Moniezia benedeni, Moniezia expansa, Muellerius capillaris, Necator americanus, Nematodirus battus, Nematodirus filicollis, Nematodirus spathiger, Nippostrongylus brasiliensis, Nippostrongylos smalesae, Oesophagodontus robustus, Oesophagostomum bifurcum, Oesophagostomum columbianum, Oesophagostomum dendatum, Oesophagostomum quadrispinulatum, Oesophagostomum radiatum, Oesophagostomum venulosum, Onchocerca cervicalis, Onchocerca lupi, Opisthorchis felineus, Opisthorchis viverrine, Oslerus osleri, Ostertagia circumcincta, Ostertagia lyrata, Ostertagia ostertagi, Oxyuris equi, Oxyuris vermicularis, Paranoplocephala mamillana, Parascaris equorum, Parascaris univalens, Petrovinema poculatum, Poteriostomum imparidentatum, Probstmayria vivipara, Schistosoma indicum, Schistosoma leiperi, Schistosoma nasale, Schistosoma spindale, Spirocerca lupi, Spirometrafelis, Strongylus edentatus, Strongylus equinus, Strongylus vulgaris, Strongyoides papillosus, Strongyloides ransomi, Strongyloides westeri, Taenia crassiceps, Taenia hydatigena, Taenia multiceps, Taenia ovis, Taenia pisiformis, Taenia saginata, Taenia serialis, Taenia solium, Taenia taeniaeformis, Teladorsagia circumcincta, Teladorsagia davtiani, Teladorsagia trifurcate, Theileria annulate, Theileria parva, Thelazia callipaeda, Thelazia calforniensis, Toxascaris leonine, Toxocara canis, Toxocara cati (syn Toxocara mystax), Toxoplasma gondii, Trichostrongylus axei, Trichostrongylus colubriformis, Trichostrongylus rugatus, Trichostrongylus vitrinus, Triodontophorus brevicauda, Triodontophorus minor, Triodontophorus nipponicus, Triodontophorus serratus, Triodontophorus tenuicollis, Tritrichomonas blagburni (syn. Tritrichomonas foetus), Trichinella britovi, Trichinella papue, Trichinella spiralis, Trichuris campanula, Trichuris serrata, Trichuris vulpis, Trichuris suis, Troglostrongylus brevoir, Troglostrongylus subcrenatus, Troglostrongylus wilsoni, Trypanosoma brucei, Trypanosoma congolense, Trypanosoma evansi, Trypanosoma vivax and Uncinaria stenocephala.

In another embodiment, the antiparasitic active agent combinations and compositions of the invention may be used to treat, control and/or prevent an endoparasitic infection in a canine animal, including a dog, by one or more of Ancylostoma braziliense, Ancylostoma caninum, Angiostrongylus vasorum, Babesia canis, Babesia gisoni, Babesia rossi, Babesia vogeli, Babesia vulpes, Crenosoma vulpis, Cytstoisispora burrowsi, Cytstoisispora canis, Cytstoisispora ohioensis, Diphyllobothrium latum, Dipylidium caninum, Dirofilaria hongkongensis, Dirofilaria immitis, Dirofilaria repens, Echinococcus granulosus, Echinococcus multilocularis, Filaroides hirthi, Filaroides milksi, Filaroides osleri, Giardia duodenalis (syn. Giardia lamblia, Giardia intestinalis), Leishmania infantum, Necator americanus, Onchocerca lupi, Oslerus osleri, Spirocerca lupi, Taenia crassiceps, Taenia hydatigena, Taenia multiceps, Taenia ovis, Taenia pisiformis, Taenia serialis, Thelazia callipaeda, Thelazia californiensis, Toxascaris leonine, Toxocara canis, Trichinella britovi, Trichuris vulpis and Uncinaria stenocephala.

In another embodiment, the antiparasitic active agent combinations and compositions of the invention may be used to treat, control and/or prevent an endoparasitic infection in a feline animal, including a cat, by one or more of Aelurostrongylus abstrusus, Ancylostoma braziliense, Ancylostoma ceylanicum, Ancylostoma tubaeforme, Capillaria aerophila, Capillariafeliscati, Clonorchis sinensis, Cytauxzoonfelis, Cytstoisisporafelis, Cytstoisispora rivolta, Diphyllobothrium latum, Dipylidium caninum, Dirofilaria hongkongensis, Dirofilaria immitis, Dirofilaria repens, Echinococcus multilocularis, Giardia duodenalis (syn. Giardia lamblia, Giardia intestinalis), Joyeuxiella pasquali, Necator americanus, Opisthorchis felineus, Opisthorchis viverrine, Spirometrafelis, Taenia taeniaeformis, Toxascaris leonine, Toxocara cati, Trichuris campanula, Trichuris serrata, Tritrichomonas blagburni (syn. Tritrichomonas foetus), Troglostrongylus brevoir, Troglostrongylus subcrenatus, Troglostrongylus wilsoni and Uncinaria stenocephala.

In another embodiment, the antiparasitic active agent combinations and compositions of the invention may be used to treat, control and/or prevent an endoparasitic infection in an equine animal by one or more of Anoplocephala magna, Anoplocephala perfoliate, Cooperia curticei, Coronocyclus coronatus, Coronocyclus labiatus, Coronocyclus labratus, Craterostomum acuticaudatum, Cyathostomum alveatum, Cyathostomum catinatum, Cyathostomum paternatum, Cyathostomum tetracanthum, Cylicocyclus ashworthi, Cylicocyclus brevicapsulatus, Cylicocyclus elongatus, Cylicocyclus insigniae, Cylicocyclus leptosomatum, Cylicocyclus nassatus, Cylicocyclus radiatus, Cylicocyclus ultrajectinus, Dictyocaulus arnfieldi, Draschia megastoma, Gyalocephalus capitatus, Habronema microstoma, Habronema muscae, Haemonchus contortus, Nematodirus battus, Oesophagodontus robustus, Onchocerca cervicalis, Ostertagia circumcincta, Oxyuris equi, Paranoplocephala mamillana, Parascaris equorum, Parascaris univalens, Petrovinema poculatum, Poteriostomum imparidentatum, Probstmayria vivipara, Strongyloides westeri, Strongylus edentates, Strongylus equinus, Strongylus vulgaris, Trichinella britovi, Trichostrongylus axei, Trichostrongylus colubriformis, Triodontophorus brevicauda, Triodontophorus minor, Triodontophorus nipponicus, Triodontophorus serratus and Triodontophorus tenuicollis.

In one embodiment, the antiparasitic active agent combinations and compositions of the invention may be used to treat, control and/or prevent an endoparasitic infection in a ruminant animal by one or more of Babesia bigemina, Babesia bovis, Babesia divergens, Babesia major, Bunostomum phlebotomum, Bunostomum trigonocephalum, Capillaria bovis, Chabertia ovina, Cooperia curticei, Cooperia oncophora, Cooperia pectinata, Cooperia punctata, Cooperia surnabada, Cystocaulus oreatus, Dictyocaulus filaria, Dictyocaulus viviparus, Eimeria auburnensis, Eimeria bovis, Eimeria crandallis, Eimeria ninakohlyakimovae, Eimeria ovina, Eimeria ovinoidalis, Eimeria zuernii, Fasciola gigantica, Fasciola hepatica, Fascioloides magna, Haemonchus contortus, Haemonchus placei, Haemonchus similis, Hsiungia triciliata, Moniezia benedeni, Moniezia expansa, Muellerius capillaris, Nematodirus filicollis, Nematodirus spathiger, Oesophagostomum columbianum, Oesophagostomum radiatum, Oesophagostomum venulosum, Ostertagia lyrata, Ostertagia ostertagi, Schistosoma indicum, Schistosoma leiperi, Schistosoma nasale, Schistosoma spindale, Strongyoides papillosus, Taenia saginata, Teladorsagia circumcincta, Teladorsagia davtiani, Teladorsagia trifurcate, Theileria annulate, Theileria parva, Toxoplasma gondii, Trichostrongylus axei, Trichostrongylus colubriformis, Trichostrongylus rugatus, Trichostrongylus vitrinus, Trypanosoma brucei, Trypanosoma congolense, Trypanosoma evansi and Trypanosoma vivax.

In another embodiment, the antiparasitic active agent combinations and compositions of the invention may be used to treat, control and/or prevent an endoparasitic infection in poultry by one or more of Capillaria anatis, Capillaria contorta, Capillaria obsignata, Eimeria acervuline, Eimeria adenoids, Eimeria brunetti, Eimeria dispersa, Eimeria gallopavonis, Eimeria hagani, Eimeria maxima, Eimeria meleagrimitis, Eimeria mitavi, Eimeria necatrix, Eimeria praecox, Eimeria tenella, Heterakis gallinarum and Histomonas meleagridis.

In another embodiment, the antiparasitic active agent combinations and compositions of the invention may be used to treat, control and/or prevent an endoparasitic infection in swine by one or more of Ascaris suum, Cytstoisospora suis, Hyostrongylus rubidus, Metastrongylus apri, Metastrongylus pudendotectus, Metastrongylus salmi, Oesophagostomum bifurcum, Oesophagostomum dendatum, Oesophagostomum quadrispinulatum, Strongyloides ransomi, Taenia solium, Trichinella britovi, Trichinella papue, Trichinella spiralis and Trichuris suis.

In one embodiment, the antiparasitic active agent combinations and compositions of the invention may be used for treating, controlling and/or preventing an ectoparasitic infestation of one or more of the following parasite genera: Acarapis, Aedes, Alphitobius, Anopheles, Amblyomma, Bovicola, Calliphora, Ceratophyllus, Cheyletiella, Chorioptes, Chrysomya, Chrysops, Cochiomyia, Ctenocephalides, Culex, Culicoides, Damalinia, Demodex, Dermacentor, Dermanyssus, Dermatobia, Diachlorus, Echidnophaga, Felicola, Gasterophilus, Gliricola, Glossina, Gyropus, Haemagogus, Haemaphysalis, Haematobia, Haematopinus, Haematopota, Heterodoxus, Hippobosca, Holakartikos, Hyalomma, Hypoderma, Ixodes, Linognathus, Lucilia, Lutzomyia, Lynxacarus, Melophagus, Menopon, Musca, Notoedres, Oestrus, Ornithonyssus, Otobius, Otodectes, Pediculus, Phlebotomus, Pneumonyssoides, Psorergates, Psoroptes, Pulex, Rhipicephalus (syn. Boophilus), Sarcoptes, Simulium, Solenoptes, Stomoxys, Tabanus, Trichodectes, Trimenopan, Trixacarus, Trombicula, Tunga, Varroa, Werneckiella and Wohlfahrtia, Xenopsylla.

In another embodiment, the antiparasitic active agent combinations and compositions of the invention may be used to treat, control and/or prevent an infestation by one or more of the following ectoparasites: Aedes aegypti, Aedes albopictus, Aedes serratus, Alphitobius diaperinus, Anopheles gambiae, Amblyomma americanum, Ambylomma aureolatum, Amblyomma cajennense, Amblyomma maculatum, Ambylomma oblongoguttatum, Ambylomma ovale, Ambylomma parvum, Amblyomma sculptum, Ambylomma triste, Amblyomma variegatum, Bovicola bovis, Bovicola caprae, Bovicola limbatus, Bovicola ovis, Calliflora stygia, Ceratophyllus gallinae, Cheyletiella blakei, Cheyletiella parasitovorax, Cheyletiella yasguri, Chorioptes bovis, Chorioptes texanus, Chrysomya rufifacies, Cochliomyia hominivorax, Ctenocephalides canis, Ctenocephalides felis, Culex pipiens, Culicoides actoni, Culicoides brevitarsis, Culicoides fulvus, Culicoides imicola, Culicoides insignis, Culicoides nubeculosus, Culicoides variipennis, Damalinia bovis, Demodex brevis, Demodex canis, Demodex catis, Demodex cornei, Demodex follicolorum, Demodex gatoi, Demodex injai, Dermacentor andersoni, Dermacentor marginatus, Dermacentor occidentalis, Dermacentor reticulatus, Dermacentor variabilis, Dermanyssus gallinae, Dermatobia hominis, Echidnophaga gallinacea, Felicola subrostratus, Gasterophilus pecorum, Gasterophilus intestinalis, Gasterophilus nasalis, Gasterophilus haemorrhoidalis, Gasterophilus inermis, Gliricola porcelli, Glossina morsitans, Glossina brevipalpis, Glossina tachinoides, Glossinafuscipes, Glossina palpalis, Glossina pallidipes, Glossina austeni, Gyropus ovalis, Haemagogus janthinomys, Haemagogus leucocelaenus, Haemaphysalis longicornis, Haemaphysalis punctata, Haematobia irritans, Haematobia irritans exigua, Haematobia stimulans, Haematopinus eurysternus, Haematopinus quadripertusis, Holakartikos crassipes, Hyalomma anatolicum, Hyalomma dromedarii, Hyalomma lusitanicum, Hyalomma marginatum, Hyalomma truncatum, Hypoderma bovis, Hypoderma lineatum, Hypoderma diana, Hypoderma actaeon, Hypoderma tarandi, Ixodes gibbosus, Ixodes hexagonus, Ixodes holocyclus, Ixodes pacificus, Ixodes persulcatus, Ixodes ricinus, Ixodes scapularis, Linognathus africanus, Linognathus ovillus, Linognathus pedalis, Linognathus setosus, Linognathus stenopsis, Linognathus vituli, Lucilia cuprina, Lucilia sericata, Lutzomyia longipalpis, Lutzomyia shannoni, Lutzomyia anthophora, Lynxacarus radovskyi, Musca autumnalis, Notoedres cati, Oestrus ovis, Ornithonyssus bursae, Ornithonyssus sylviarum, Otobius megnini, Otodectes cynotis, Phlebotomus argentipes, Phlebotomus papatasi, Pneumonyssoides caninum, Przhevalskiana silenus, Psorergates ovis, Psoroptes cervinus, Psoroptes cuniculi, Psoroptes equi, Psoroptes natalensis, Psoroptes ovis, Pulex simulans, Pulex irritans, Rhipicephalus annulatus, Rhipicephalus appenidiculatus, Rhipicephalus bursa, Rhipicephalus decoloratus, Rhipicephalus microplus, Rhipicephalus sanguineus, Rhipicephalus turanicus, Sarcoptes anthracis, Sarcoptes scabiei, Sarcoptes scabiei var. canis, Solenopotes capillatus, Stomoxys calcitrans, Trichodectes canis, Trimenopan hispidum, Trixacarus caviae, Trixacarus diversus, Werneckiella equi, Wohlfahrtia magnifica and Xenopsylla cheopis.

In another embodiment, the antiparasitic active agent combinations and compositions of the invention may be used to treat, control and/or prevent an ectoparasitic infestation by one or more of the following flea species: Ceratophyllus gallinae, Ctenocephalides canis, Ctenocephalides felis, Echidnophaga gallinacea, Pulex simulans, Pulex irritans and Xenopsylla cheopis.

In another embodiment, the antiparasitic active agent combinations and compositions of the invention may be used to treat, control and/or prevent an ectoparasitic infestation by one or more of the following tick species: Amblyomma americanum, Ambylomma aureolatum, Amblyomma cajennense, Amblyomma maculatum, Ambylomma oblongoguttatum, Ambylomma ovale, Ambylomma parvum, Amblyomma sculptum, Ambylomma triste, Ambylomma triguttatum, Ambylomma triguttatum, Amblyomma variegatum, Dermacentor andersoni, Dermacentor marginatus, Dermacentor occidentalis, Dermacentor reticulatus, Dermacentor variabilis, Haemaphysalis elliptica, Haemaphysalis flava, Haemaphysalis elliptica, Haemaphysalisflava, Haemaphysalis longicornis, Haemaphysalis punctata, Hyalomma anatolicum, Hyalomma dromedarii, Hyalomma lusitanicum, Hyalomma marginatum, Hyalomma truncatum, Ixodes gibbosus, Ixodes hexagonus, Ixodes holocyclus, Ixodes, ovatus, Ixodes pacificus, Ixodes persulcatus, Ixodes ricinus, Ixodes scapularis, Otobius megnini, Rhipicephalus annulatus, Rhipicephalus appenidiculatus, Rhipicephalus bursa, Rhipicephalus decoloratus, Rhipicephalus microplus, Rhipicephalus sanguineus and Rhipicephalus turanicus.

In another embodiment, the antiparasitic active agent combinations and compositions of the invention may be used to treat, control and/or prevent an ectoparasitic infestation by one or more of the following fly species: Calliflora stygia, Cochliomyia hominivorax, Chrysomya rufifacies, Culicoides actoni, Culicoides brevitarsis, Culicoides fulvus, Culicoides imicola, Culicoides insignis, Culicoides nubeculosus, Culicoides variipennis, Dermatobia hominis, Gasterophilus pecorum, Gasterophilus intestinalis, Gasterophilus nasalis, Gasterophilus haemorrhoidalis, Gasterophilus inermis, Glossina morsitans, Glossina brevipalpis, Glossina tachinoides, Glossina fuscipes, Glossina palpalis, Glossina pallidipes, Glossina austeni, Haematobia irritans, Haematobia irritans exigua, Haematobia stimulans, Hypoderma bovis, Hypoderma lineatum, Hypoderma diana, Hypoderma actaeon, Hypoderma tarandi, Lucilia cuprina, Lucilia sericata, Lutzomyia longipalpis, Lutzomyia shannoni, Lutzomyia anthophora, Musca autumnalis, Oestrus ovis, Phlebotomus argentipes, Phlebotomus papatasi, Stomoxys calcitrans and Wohlfahrtia magnifica.

In another embodiment, the antiparasitic active agent combinations and compositions of the invention may be used to treat, control and/or prevent an ectoparasitic infestation by one or more of the following mite species: Chorioptes bovis, Chorioptes texanus, Cheyletiella blakei, Cheyletiella parasitovorax, Cheyletiella yasguri, Demodex brevis, Demodex canis, Demodex catis, Demodex cornei, Demodex follicolorum, Demodex gatoi, Dermanyssus gallinae, Demodex injai, Lynxacarus radovskyi, Notoedres cati, Ornithonyssus bursae, Ornithonyssus sylviarum, Otodectes cynotis, Pneumonyssoides caninum, Psorergates ovis, Psoroptes cervinus, Psoroptes cuniculi, Psoroptes equi, Psoroptes natalensis, Psoroptes ovis, Sarcoptes anthracis, Sarcoptes scabiei and Sarcoptes scabiei var. canis.

In another embodiment, the antiparasitic active agent combinations and compositions of the invention may be used to treat, control and/or prevent an ectoparasitic infestation by one or more of the following lice species: Bovicola bovis, Bovicola caprae, Bovicola limbatus, Bovicola ovis, Damalinia bovis, Felicola subrostratus, Gliricola porcelli, Gyropus ovalis, Haematopinus eurysternus, Haematopinus quadripertusis, Holakartikos crassipes, Linognathus africanus, Linognathus ovillus, Linognathus pedalis, Linognathus setosus, Linognathus stenopsis, Linognathus vituli, Solenopotes capillatus, Trichodectes canis, Trimenopan hispidum, Trixacarus caviae, Trixacarus diversus and Werneckiella equi.

In yet another embodiment, the antiparasitic active agent combinations and compositions of the invention may be used to treat, control and/or prevent an ectoparasitic infestation in an animal by one or more of the following mosquito species: Aedes aegypti, Aedes albopictus, Aedes serratus, Anopheles gambiae, Culex pipiens, Haemagogus janthinomys and Haemagogus leucocelaenus.

The list of ectoparasites disclosed above is not exhaustive and other ectoparasites are well known in the art to be harmful to animals and humans. These include, for example, migrating dipteran larvae.

### Additional Antiparasitic Active Agents

The antiparasitic active agent combinations of the invention comprising one or more Slo-1 agonist compounds, including one or more of the compounds of Formula (IA), Formula (IB) and/or Formula (IC), may include at least one other veterinary therapeutic agent. Veterinary pharmaceutical agents that may be included in the antiparasitic active agent combinations of the invention and compositions comprising the combinations are well-known in the art (see e.g. Plumb'Veterinary Drug Handbook, 5^{th} Edition, ed. Donald C. Plumb, Blackwell Publishing, (2005) or The Merck Veterinary Manual, 9^{th} Edition, (January 2005)) and include but are not limited to acarbose, acepromazine maleate, acetaminophen, acetazolamide, acetazolamide sodium, acetic acid, acetohydroxamic acid, acetylcysteine, acitretin, acyclovir, albendazole, albuterol sulfate, alfentanil, allopurinol, alprazolam, altrenogest, amantadine, amikacin sulfate, aminocaproic acid, aminopentamide hydrogen sulfate, aminophylline/theophylline, amiodarone, amitriptyline, amlodipine besylate, ammonium chloride, ammonium molybdenate, amoxicillin, clavulanate potassium, amphotericin B desoxycholate, amphotericin B lipid-based, ampicillin, amprolium, antacids (oral), antivenin, apomorphione, apramycin sulfate, ascorbic acid, asparaginase, aspiring, atenolol, atipamezole, atracurium besylate, atropine sulfate, aurnofin, aurothioglucose, azaperone, azathioprine, azithromycin, baclofen, barbituates, benazepril, betamethasone, bethanechol chloride, bisacodyl, bismuth subsalicylate, bleomycin sulfate, boldenone undecylenate, bromides, bromocriptine mesylate, budenoside, buprenorphine, buspirone, busulfan, butorphanol tartrate, cabergoline, calcitonin salmon, calcitrol, calcium salts, captopril, carbenicillin indanyl sodium, carbimazole, carboplatin, carnitine, carprofen, carvedilol, cefadroxil, cefazolin sodium, cefixime, clorsulon, cefoperazone sodium, cefotaxime sodium, cefotetan disodium, cefoxitin sodium, cefpodoxime proxetil, ceftazidime, ceftiofur sodium, ceftiofur, ceftiaxone sodium, cephalexin, cephalosporins, cephapirin, charcoal (activated), chlorambucil, chloramphenicol, chlordiazepoxide, chlordiazepoxide+/−clidinium bromide, chlorothiazide, chlorpheniramine maleate, chlorpromazine, chlorpropamide, chlortetracycline, chorionic gonadotropin (HCG), chromium, cimetidine, ciprofloxacin, cisapride, cisplatin, citrate salts, clarithromycin, clemastine fumarate, clenbuterol, clindamycin, clofazimine, clomipramine, claonazepam, clonidine, cloprostenol sodium, clorazepate dipotassium, clorsulon, cloxacillin, codeine phosphate, colchicine, corticotropin (ACTH), cosyntropin, cyclophosphamide, cydicloxacillin, diethylcarbamazine citrate, diethylstilbestrol (DES), difloxacin, digoxin, dihydrotachysterol (DHT), diltiazem, dimenhydrinate, dimercaprol/BAL, dimethyl sulfoxide, dinoprost tromethamine, diphenylhydramine, disopyramide phosphate, dobutamine, docusate/DSS, dolasetron mesylate, domperidone, dopamine, doramectin, doxapram, doxepin, doxorubicin, doxycycline, edetate calcium disodium.calcium EDTA, edrophonium chloride, enalapril/enalaprilat, enoxaparin sodium, enrofloxacin, ephedrine sulfate, epinephrine, epoetin/erythropoietin, eprinomectin, epsiprantel, erythromycin, esmolol, estradiol cypionate, ethacrynic acid/ethacrynate sodium, ethanol (alcohol), etidronate sodium, etodolac, etomidate, euthanasia agents w/pentobarbital, famotidine, fatty acids (essential/omega), felbamate, fentanyl, ferrous sulfate, filgrastim, finasteride, fipronil, florfenicol, fluconazole, flucytosine, fludrocortisone acetate, flumazenil, flumethasone, flunixin meglumine, fluorouracil (5-FU), fluoxetine, fluticasone propionate, fluvoxamine maleate, fomepizole (4-MP), furazolidone, furosemide, gabapentin, gemcitabine, gentamicin sulfate, glimepiride, glipizide, glucagon, glucocorticoid agents, glucosamine/chondroitin sulfate, glutamine, glyburide, glycerine (oral), glycopyrrolate, gonadorelin, grisseofulvin, guaifenesin, halothane, hemoglobin glutamer-200 (OXYGLOBIN®®), heparin, hetastarch, hyaluronate sodium, hydrazaline, hydrochlorothiazide, hydrocodone bitartrate, hydrocortisone, hydromorphone, hydroxyurea, hydroxyzine, ifosfamide, imidacloprid, imidocarb dipropinate, impenem-cilastatin sodium, imipramine, inamrinone lactate, insulin, interferon alfa-2a (human recombinant), iodide (sodium/potassium), ipecac (syrup), ipodate sodium, iron dextran, isoflurane, isoproterenol, isotretinoin, isoxsuprine, itraconazole, ivermectin, kaolin/pectin, ketamine, ketoconazole, ketoprofen, ketorolac tromethamine, lactulose, leuprolide, levamisole, levetiracetam, levothyroxine sodium, lidocaine, lincomycin, liothyronine sodium, lisinopril, lomustine (CCNU), lufenuron, lysine, magnesium, mannitol, marbofloxacin, mechlorethamine, meclizine, meclofenamic acid, medetomidine, medium chain triglycerides, medroxyprogesterone acetate, megestrol acetate, melarsomine, melatonin, meloxican, melphalan, meperidine, mercaptopurine, meropenem, metformin, methadone, methazolamide, methenamine mandelate/hippurate, methimazole, methionine, methocarbamol, methohexital sodium, methotrexate, methoxyflurane, methylene blue, methylphenidate, methylprednisolone, metoclopramide, metoprolol, metronidaxole, mexiletine, mibolerlone, midazolam milbemycin oxime, mineral oil, minocycline, misoprostol, mitotane, mitoxantrone, morphine sulfate, moxidectin, naloxone, mandrolone decanoate, naproxen, narcotic (opiate) agonist analgesics, neomycin sulfate, neostigmine, niacinamide, nitazoxanide, nitenpyram, nitrofurantoin, nitroglycerin, nitroprusside sodium, nizatidine, novobiocin sodium, nystatin, octreotide acetate, olsalazine sodium, omeprazole, ondansetron, opiate antidiarrheals, orbifloxacin, oxacillin sodium, oxazepam, oxibutynin chloride, oxymorphone, oxytretracycline, oxytocin, pamidronate disodium, pancreplipase, pancuronium bromide, paromomycin sulfate, parozetine, pencillamine, general information penicillins, penicillin G, penicillin V potassium, pentazocine, pentobarbital sodium, pentosan polysulfate sodium, pentoxifylline, pergolide mesylate, phenobarbital, phenoxybenzamine, pheylbutazone, phenylephrine, phenypropanolamine, phenytoin sodium, pheromones, parenteral phosphate, phytonadione/vitamin K-1, pimobendan, piperazine, pirlimycin, piroxicam, polysulfated glycosaminoglycan, ponazuril, potassium chloride, pralidoxime chloride, prazosin, prednisolone/prednisone, primidone, procainamide, procarbazine, prochlorperazine, propantheline bromide, Propionibacterium acnes injection, propofol, propranolol, protamine sulfate, pseudoephedrine, psyllium hydrophilic mucilloid, pyridostigmine bromide, pyrilamine maleate, pyrimethamine, quinacrine, quinidine, ranitidine, rifampin, s-adenosyl-methionine (SAMe), saline/hyperosmotic laxative, selamectin, selegiline/l-deprenyl, sertraline, sevelamer, sevoflurane, silymarin/milk thistle, sodium bicarbonate, sodium polystyrene sulfonate, sodium stibogluconate, sodium sulfate, sodum thiosulfate, somatotropin, sotalol, spectinomycin, spironolactone, stanozolol, streptokinase, streptozocin, succimer, succinylcholine chloride, sucralfate, sufentanil citrate, sulfachlorpyridazine sodium, sulfadiazine/trimethroprim, sulfamethoxazole/trimethoprim, sulfadimentoxine, sulfadimethoxine/ormetoprim, sulfasalazine, taurine, tepoxaline, terbinafline, terbutaline sulfate, testosterone, tetracycline, thiacetarsamide sodium, thiamine, thioguanine, thiopental sodium, thiotepa, thyrotropin, tiamulin, ticarcilin disodium, tiletamine/zolazepam, tilmocsin, tiopronin, tobramycin sulfate, tocainide, tolazoline, telfenamic acid, topiramate, tramadol, trimcinolone acetonide, trientine, trilostane, trimepraxine tartrate w/prednisolone, tripelennamine, tylosin, urdosiol, valproic acid, vanadium, vancomycin, vasopressin, vecuronium bromide, verapamil, vinblastine sulfate, vincristine sulfate, vitamin E/selenium, warfarin sodium, xylazine, yohimbine, zafirlukast, zidovudine (AZT), zinc acetate/zinc sulfate, zonisamide and mixtures thereof.

In one embodiment, the antiparasitic active agent combinations may include one or more compounds of Formula (IA), Formula (IB) and/or Formula (IC), or a salt thereof, together with an ectoparasiticide. In one embodiment, the one or more compounds of Formula (IA), Formula (IB) and/or Formula (IC) may be combined with one or more arylpyrazole compounds such as phenylpyrazoles. Examples of such arylpyrazole compounds include but are not limited to those described in U.S. Pat. Nos. 6,001,384; 6,010,710; 6,083,519; 6,096,329; 6,174,540; 6,685,954, 6,998,131 and 7,759,381 (all of which are incorporated herein by reference). Particularly preferred phenylpyrazole active agents include fipronil (Pp-1) or ethiprole (Pp-2).

In another embodiment of the invention, one or more compounds of Formula (IA), Formula (IB) and/or Formula (IC) may be combined with a class of acaricide or insecticides known as insect growth regulators (IGRs). Compounds belonging to this group are well known to the practitioner and represent a wide range of different chemical classes. These compounds all act by interfering with the development or growth of the insect pests. Insect growth regulators are described, for example, in U.S. Pat. Nos. 3,748,356, 3,818,047, 4,225,598, 4,798,837, 4,751,225, EP 0 179 022 or U.K. 2 140 010 as well as U.S. Pat. Nos. 6,096,329 and 6,685,954 (all incorporated herein by reference).

In one embodiment the IGR compound is one that mimics juvenile hormone or that modulates levels of juvenile hormones in insects. Examples of juvenile hormone mimics that may be combined with the compounds of Formula (IA), Formula (IB) and/or Formula (IC) include azadirachtin (Jh-1), diofenolan (Jh-2), fenoxycarb (Jh-3), hydroprene (Jh-4), kinoprene (Jh-5), methoprene (Jh-6), pyriproxyfen (Jh-7), tetrahydroazadirachtin (Jh-8) and 4-chloro-2(2-chloro-2-methyl-propyl)-5-(6-iodo-3-pyridylmethoxy)pyridazine-3(2H)-one (Jh-9). In another embodiment, the compound of Formula (IA), Formula (IB) or Formula (IC) may be combined with methoprene (Jh-6) or pyriproxyfen (Jh-7).

In another embodiment, the active agent combinations may include an IGR compound that is a chitin synthesis inhibitor. Chitin synthesis inhibitors include chlorofluazuron (Ci-1), cyromazine (Ci-2), diflubenzuron (Ci-3), fluazuron (Ci-4), flucycloxuron (Ci-5), flufenoxuron (Ci-6), hexaflumoron (Ci-7), lufenuron (Ci-8), tebufenozide (Ci-9), teflubenzuron (Ci-10), triflumuron (Ci-11), 1-(2,6-difluorobenzoyl)-3-(2-fluoro-4-(trifluoromethyl)phenylurea (Ci-12), 1-(2,6-difluoro-benzoyl)-3-(2-fluoro-4-(1,1,2,2-tetrafluoroethoxy)-phenylurea (Ci-13) and 1-(2,6-difluorobenzoyl)-3-(2-fluoro-4-trifluoromethyl)phenylurea (Ci-14).

Suitable organophosphate active agents for the active agent combinations that may be combined with a compound of Formula (IA), Formula (IB) and/or Formula (IC) include, but are not limited to, coumaphos (Op-1), trichlorfon (Op-2), haloxon (Op-3), naftalofos (Op-4) and dichlorvos (Op-5), heptenophos (Op-6), mevinphos (Op-7), monocrotophos (Op-8), TEPP (Op-9), and tetrachlorvinphos (Op-10).

In yet other embodiments, the antiparasitic active agent combinations of the invention may include other active agents that are effective against arthropod parasites including, but not limited to, bromocyclen, chlordane, DDT, endosulfan, lindane, methoxychlor, toxaphene, bromophos, bromophos-ethyl, carbophenothion, chlorfenvinphos, chlorpyrifos, crotoxyphos, cythioate, diazinon, dichlorenthion, diemthoate, dioxathion, ethion, famphur, fenitrothion, fenthion, fospirate, iodofenphos, malathion, naled, phosalone, phosmet, phoxim, propetamphos, ronnel, stirofos, allethrin, cyhalothrin, cypermethrin, deltamethrin, fenvalerate, flucythrinate, permethrin, phenothrin, pyrethrins, resmethrin, benzyl benzoate, carbon disulfide, crotamiton, diflubenzuron, diphenylamine, disulfiram, isobornyl thiocyanato acetate, methoprene, monosulfiram, pirenonylbutoxide, rotenone, triphenyltin acetate, triphenyltin hydroxide, deet, dimethyl phthalate, and the compounds 1,5a,6,9,9a,9b-hexahydro-4a(4H)-dibenzofurancarboxaldehyde (MGK-11), 2-(2-ethylhexyl)-3a,4,7,7a-tetrahydro-4,7-methano-1H-isoindole-1,3(2H)dione (MGK-264), dipropyl-2,5-pyridinedicarboxylate (MGK-326) and 2-(octylthio)ethanol (MGK-874).

In yet another embodiment of the invention, adulticide insecticides and acaricides can also be included in the antiparasitic active agent combinations. These include pyrethrins (which include cinerin I (Py-1), cinerin II (Py-2), jasmolin I (Py-3), jasmolin II (Py-4), pyrethrin I (Py-5), pyrethrin II (Py-6) and mixtures thereof) and synthetic pyrethroids. Pyrethroids are synthetic analogs of the pyrethrins with increased potency and stability. Pyrethroids that may be used in the compositions of the invention include, but are not limited to, permethrin (Py-7), alphacypermethrin (Py-8), cyfluthrin (Py-9), beta-cyfluthrin (Py-10), cyhalothrin (Py-11), cypermethrin (Py-12), cyphenothrin (Py-13), deltamethrin (Py-14), fenvalerate (Py-15), flucythrinate (Py-16), flumethrin (Py-17), tau-fluvalinate (Py-18), allethrin (Py-19), bioallethrin (Py-20), phenothrin (Py-21), resmethrin (Py-22), tetramethrin (Py-23), transfluthrin (Py-24) and etofenprox (Py-25).

Carbamate insecticides that may be used in the antiparasitic active agent combinations of the invention include, but are not limited to, benomyl (Ca-1), carbanolate (Ca-2), carbaryl (Ca-3), carbofuran (Ca-4), methiocarb (Ca-5), metolcarb (Ca-6), promacyl (Ca-7), propoxur (Ca-8), aldicarb (Ca-9), butocarboxim (Ca-10), oxamyl (Ca-11), thiocarboxime Ca-12) and thiofanox (Ca-13).

In another embodiment, the antiparasitic active agent combinations may comprise an active agent from the neonicotinoid class of parasiticides. The neonicotinoids bind and inhibit insect specific nicotinic acetylcholine receptors. In one embodiment, the neonicotinoid insecticidal agent that can be combined with a compound of Formula (IA), Formula (IB) and/or Formula (IC) in a composition of the invention is imidacloprid (Ne-1). Agents of this class are described, for example, in U.S. Pat. No. 4,742,060 or in EP 0 892 060 (both incorporated herein by reference). In other embodiments, the antiparasitic combinations of the invention may comprise nitenpyram (Ne-2), dinotefuran (Ne-3), thiamethoxam (Ne-4), acetamiprid (Ne-5) or clothianidin (Ne-6). The use of nitenpyram for controlling fleas is described in U.S. Pat. No. 5,750,548, which is incorporated herein by reference in its entirety.

In certain other embodiments of the invention, the compound of Formula (IA), Formula (IB) and/or Formula (IC) can be combined with antiparasitic semicarbazone active agents, such as metaflumizone (Se-1).

In another embodiment, the antiparasitic active agent combinations of the invention may advantageously include one or more isoxazoline compounds known in the art. Isoxazoline active agents are highly effective against a variety of ectoparasites. Particularly useful isoxazoline active agents that can be combined with the compound include afoxolaner (Is-1), including the substantially pure active enantiomer, esafoxolaner (Is-2), umifoxolaner (Is-3), sarolaner (Is-4), fluralaner (Is-5), including substantially pure active enantiomer (S)-fluralaner (Is-6), lotilaner (Is-7), mivorilaner (Is-8) and tigolaner (Is-9). These active agents are described in U.S. Pat. No. 7,964,204, US 2010/0254960 A1, US2011/0159107, US2012/0309620, US2012/0030841, US2010/0069247, WO 2007/125984, WO 2012/086462, U.S. Pat. Nos. 8,318,757, 8,466,115, 8,618,126, 8,822,466, 8,383,659, 8,853,186, 9,221,835, US 2011/0144349, U.S. Pat. No. 8,053,452; US 2010/0137612, U.S. Pat. No. 8,410,153, US 2011/152081, WO 2012/089623, WO 2012/089622, U.S. Pat. Nos. 8,119,671; 7,947,715; WO 2102/120135, WO 2012/107533, WO 2011/157748, US 2011/0245274, US 2011/0245239, US 2012/0232026, US 2012/0077765, US 2012/0035122, US 2011/0251247, WO 2011/154433, WO 2011/154434, US 2012/0238517, US 2011/0166193, WO 2011/104088, WO 2011/104087, WO 2011/104089, US 2012/015946, US 2009/0143410, WO 2007/123855 A2, US 2011/0118212, U.S. Pat. No. 7,951,828 & U.S. Pat. No. 7,662,972, US 2010/0137372 A1, US 2010/0179194 A2, US 2011/0086886 A2, US 2011/0059988 A1, US 2010/0179195 A1, US 2015/0126523, WO 2010/003923, WO 2010/003877, WO 2010/072602, WO 2014/134236, WO 2017/147352, U.S. Pat. Nos. 7,897,630, 7,951,828, WO 2020/007704 A1, WO 2021/028479 A1, WO 2014/122083 A1, WO 2016/177619 A1, WO 2014/012975 A1, WO 2015/078846 A1, WO 2015/078847 A1, WO 2015/150302 A1, WO 2015/181139 A1 and WO 2016/026789 A1, all of which are incorporated herein by reference in their entirety.

In another embodiment, the antiparasitic active agent combinations may include a meta diamide antiparasitic compound. Meta diamide antiparasitic compounds that may be combined with the compound of Formula (IA), Formula (IB) and/or Formula (IC) include modoflaner (Md-1).

In another embodiment of the invention, nodulisporic acid and its derivatives may be included in the antiparasitic active agent combinations of the invention. These compounds are used to treat or prevent infections in humans and animals and are described, for example, in U.S. Pat. Nos. 5,399,582, 5,962,499, 6,221,894 and 6,399,786, all of which are hereby incorporated by reference in their entirety. The compositions may include one or more of the known nodulisporic acid derivatives in the art, including all stereoisomers, such as those described in the literature cited above.

In some embodiments, the compound of Formula (IA), Formula (IB) and/or Formula (IC), or a salt thereof, may be combined with other endoparasiticide compounds. In one embodiment, the antiparasitic combinations of the invention include one or more macrocyclic lactone active agents, For the avoidance of doubt, the term “macrocyclic lactone” as used herein includes both naturally occurring and synthetic or semi-synthetic avermectin and milbemycin compounds.

The macrocyclic lactones that may be used in the antiparasitic active agent combinations of the invention include, but are not limited to, the naturally produced avermectins (e.g. including the components designated as A₁a, A₁b, A₂a, A₂b, B₁a, B₁b, B₂a and B₂b) and milbemycin compounds, semisynthetic avermectins and milbemycins, avermectin monosaccharide compounds and avermectin aglycone compounds. Examples of macrocyclic lactone compounds that may be used in the compositions include, but are not limited to, abamectin (ML-1), dimadectin (ML-2), doramectin (ML-3), emamectin (ML-4), eprinomectin (ML-5), ivermectin (ML-6), latidectin (ML-7), lepimectin (ML-8), selamectin (ML-9), ML-1,694,554 (ML-10) and milbemycins including, but not limited to, milbemectin (ML-11), milbemycin D (ML-12), milbemycin A₃ (ML-13), milbemycin A₄(ML-14), milbemycin oxime (ML-15), moxidectin (ML-16) and nemadectin (ML-17). Also included are the 5-oxo and 5-oxime derivatives of said avermectins and milbemycins.

The macrocyclic lactone active agents are known in the art and can easily be obtained commercially or through synthesis techniques known in the art. Reference is made to the widely available technical and commercial literature. For avermectins, ivermectin and abamectin, reference may be made, for example, to the work “Ivermectin and Abamectin”, 1989, by M. H. Fischer and H. Mrozik, William C. Campbell, published by Springer Verlag., or Albers-Schonberg et al. (1981), “Avermectins Structure Determination”, J. Am. Chem. Soc., 103, 4216-4221. For doramectin, “Veterinary Parasitology”, vol. 49, No. 1, July 1993, 5-15 may be consulted. For milbemycins, reference may be made, inter alia, to Davies H. G. et al., 1986, “Avermectins and Milbemycins”, Nat. Prod. Rep., 3, 87-121, Mrozik H. et al., 1983, Synthesis of Milbemycins from Avermectins, Tetrahedron Lett., 24, 5333-5336, U.S. Pat. No. 4,134,973 and EP 0 677 054, both incorporated herein by reference.

The structure of the avermectins and milbemycins are closely related, e.g., by sharing a complex 16-membered macrocyclic lactone ring. The natural product avermectins are disclosed in U.S. Pat. No. 4,310,519 and the 22,23-dihydro avermectin compounds are disclosed in U.S. Pat. No. 4,199,569. Mention is also made of U.S. Pat. Nos. 4,468,390, 5,824,653, EP 0 007 812 A1, U.K. Patent Specification 1 390 336, EP 0 002 916, and New Zealand Patent No. 237 086, inter alia. Naturally occurring milbemycins are described in U.S. Pat. No. 3,950,360 as well as in the various references cited in “The Merck Index” 12^{th} ed., S. Budavari, Ed., Merck & Co., Inc. Whitehouse Station, New Jersey (1996). Latidectin is described in the “International Nonproprietary Names for Pharmaceutical Substances (INN)”, WHO Drug Information, vol. 17, no. 4, pp. 263-286, (2003). Semisynthetic derivatives of these classes of compounds are well known in the art and are described, for example, in U.S. Pat. Nos. 5,077,308, 4,859,657, 4,963,582, 4,855,317, 4,871,719, 4,874,749, 4,427,663, 4,310,519, 4,199,569, 5,055,596, 4,973,711, 4,978,677, 4,920,148 and EP 0 667 054, all incorporated herein by reference.

In some embodiments, the antiparasitic active agent combinations of the invention may include one or more antinematodal agents including, but not limited to, active agents in the benzimidazoles, imidazothiazoles, tetrahydropyrimidines and the organophosphate class of compounds. In some embodiments, benzimidazoles including, but not limited to, thiabendazole (Bz-1), cambendazole (Bz-2), parbendazole (Bz-3), oxibendazole (Bz-4), mebendazole (Bz-5), flubendazole (Bz-6), fenbendazole (Bz-7), oxfendazole (Bz-8), albendazole (Bz-9), cyclobendazole (Bz-10), febantel (Bz-11), thiophanate (Bz-12) and its o,o-dimethyl analogue (Bz-13) may be included in the compositions.

In other embodiments, the antiparasitic active agent combinations of the invention may include an imidazothiazole compound including, but not limited to, tetramisole (Im-1), levamisole (Im-2) and butamisole (Im-3).

In still other embodiments, the antiparasitic active agent combinations of the invention may include tetrahydropyrimidine active agents including, but not limited to, pyrantel (Tp-1), oxantel (Tp-2), and morantel (Tp-3).

In other embodiments, the antiparasitic active agent combinations may include the antinematodal compounds phenothiazine (An-1), piperazine (An-2) as the neutral compound and in various salt forms, diethylcarbamazine (An-3), phenols such as disophenol (An-4), arsenicals such as arsenamide (An-5), ethanolamines such as bephenium (An-6), thenium closylate (An-7), and methyridine (An-8); cyanine dyes including pyrvinium chloride (An-9), pyrvinium pamoate (An-10) and dithiazanine iodide (An-11); isothiocyanates including bitoscanate (An-12), suramin sodium (An-13), phthalofyne (An-14), and various natural products including, but not limited to, hygromycin B (An-15), α-santonin (An-16) and kainic acid (An-17).

In other embodiments, the antiparasitic active agent combinations of the invention may include antitrematodal agents. Suitable antitrematodal agents include, but are not limited to, the miracils such as miracil D (At-1) and mirasan (At-2); clonazepam (At-3) and its 3-methyl derivative (At-4), oltipraz (At-5), lucanthone (At-6), hycanthone (At-7), oxamniquine (At-8), amoscanate (At-9), niridazole (At-10), nitroxinil (At-11), various bisphenol compounds known in the art including hexachlorophene (At-12), bithionol (At-13), bithionol sulfoxide (At-14) and menichlopholan (At-15); various salicylanilide compounds including tribromsalan (At-15), oxyclozanide (At-16), clioxanide (At-17), rafoxanide (At-18), nitroxinil (At-19), brotianide (At-20), bromoxanide (At-21) and closantel (At-22); triclabendazole (At-23), diamfenetide (At-24), clorsulon (At-25), hetolin (At-26) and emetine (At-27).

Anticestodal compounds may also be advantageously used in the antiparasitic active agent combinations of the invention including, but not limited to, arecoline (Ac-1) in various salt forms, bunamidine (Ac-2), niclosamide (Ac-3), nitroscanate (Ac-4), paromomycin (Ac-5), paromomycin II (Ac-6), praziquantel (Ac-7) and epsiprantel (Ac-8).

In another embodiment, an antiparasitic agent that can be included in the antiparasitic active agent combinations containing a compound of Formula (IA), Formula (IB) or Formula (IC) can be a biologically active peptide or protein including, but not limited to, cyclic depsipeptides such as cyclooctadepsipeptides. These include PF1022A (Dp-1) or analogs thereof and emodepside (Dp-2). Other cyclic depsipeptide compounds that may be included in the compositions comprising a compound of Formula (IA), Formula (IB) and/or Formula (IC) are those described in WO 2016/187534 A1 and WO 2017/116702 A1, both incorporated herein by reference. These compounds act at the neuromuscular junction by stimulating presynaptic receptors belonging to the secretin receptor family resulting in the paralysis and death of parasites. In one embodiment of the depsipeptide, the depsipeptide is emodepside (see Wilson et al., Parasitology, January 2003, 126(Pt 1):79-86).

In another embodiment, anthelmintic compounds of the amino acetonitrile class (AAD) of compounds such as monepantel (Aa-1) (ZOLVIX) and the like may be added to the antiparasitic active agent combinations of the invention. These compounds are described, for example, in U.S. Pat. No. 7,084,280 to Ducray et al. (incorporated herein by reference); Sager et al., Veterinary Parasitology, 2009, 159, 49-54; Kaminsky et al., Nature vol. 452, 13 Mar. 2008, 176-181.

The antiparasitic active agent combinations of the invention may also include aryloazol-2-yl cyanoethylamino compounds such as those described in U.S. Pat. No. 8,088,801 to Soll et al., which is incorporated herein by reference, and thioamide derivatives of these compounds, as described in U.S. Pat. No. 7,964,621, which is also incorporated herein by reference. Aryloazol-2-yl cyanoethylamino active agents, which are systemically-acting against endoparasites, may be used in combination with the compound in veterinary compositions of the invention.

The antiparasitic active agent combinations of the invention may also include paraherquamide compounds and derivatives of these compounds, including derquantel (Ph-1) (see Ostlind et al., Research in Veterinary Science, 1990, 48, 260-61; and Ostlind et al., Medical and Veterinary Entomology, 1997, 11, 407-408). The paraherquamide family of compounds is a known class of compounds that include a spirodioxepino indole core with activity against certain parasites (see Tett. Lett. 1981, 22, 135; J. Antibiotics 1990, 43, 1380, and J. Antibiotics 1991, 44, 492). In addition, the structurally related marcfortine family of compounds, such as marcfortines A (Ph-2), marcfortine B (Ph-3) and marcfortine C (Ph-4), are also known and may be combined with the compositions of the invention (see J. Chem. Soc.—Chem. Comm. 1980, 601 and Tet. Lett. 1981, 22, 1977). Further references to the paraherquamide derivatives can be found, for example, in WO 91/09961, WO 92/22555, WO 97/03988, WO 01/076370, WO 09/004432 and US 2010/0197624, U.S. Pat. Nos. 5,703,078 and 5,750,695, all of which are hereby incorporated by reference in their entirety.

In another embodiment of the invention, the antiparasitic active agent combinations may include a spinosyn active agent produced by the soil actinomycete Saccharopolyspora spinosa (see, for example Salgado V. L. and Sparks T. C., “The Spinosyns: Chemistry, Biochemistry, Mode of Action, and Resistance,” in Comprehensive Molecular Insect Science, vol. 6, pp. 137-173, 2005) or a semi-synthetic spinosoid active agent. The spinosyns are typically referred to as factors or components A, B, C, D, E, F, G, H, J, K, L, M, N, O, P, Q, R, S, T, U, V, W, or Y, and any of these components, or a combination thereof, may be used in the compositions of the invention. The spinosyn compound may be a 5,6,5-tricylic ring system, fused to a 12-membered macro cyclic lactone, a neutral sugar (rhamnose), and an amino sugar (forosamine). These and other natural spinosyn compounds, including 21-butenyl spinosyn produced by Saccharopolyspora pagona, which may be used in the compositions of the invention, may be produced via fermentation by conventional techniques known in the art. Other spinosyn compounds that may be used in the compositions of the invention are disclosed in U.S. Pat. Nos. 5,496,931; 5,670,364; 5,591,606; 5,571,901; 5,202,242; 5,767,253; 5,840,861; 5,670,486; 5,631,155 and 6,001,981, all incorporated by reference herein in their entirety. The spinosyn compounds may include, but are not limited to, spinosyn A (Sp-1), spinosyn D (Sp-2), Spinosad (Sp-3), spinetoram (Sp-4), or combinations thereof. Spinosad is a combination of spinosyn A and spinosyn D, and spinetoram is a combination of 3′-ethoxy-5,6-dihydro spinosyn J and 3′-ethoxy spinosyn L.

In other embodiments, the specific antiparasitic active agent combinations described in Combinations 1 to Combinations 80 below (abbreviated Comb 1 to Comb 80) are provided. For clarity, each numbered Combination (e.g. Combinations 1, Combinations 2, etc.) will include specific combinations with the compound noted (e.g. compound A472) and each specific combination partner listed. For example, Combinations 1 described below includes two specific antiparasitic combinations that are individually listed—Formula (IA)+fipronil (Pp-1) and Formula (IA)+ethiprole (Pp-2).
Combinations 1: In one embodiment, the antiparasitic active agent combinations comprise a compound of Formula (IA) with one or more phenylpyrazole active agents: Formula (IA)+Pp-1 (Comb 1-1), Formula (IA)+Pp-2 (Comb 1-2).
Combinations 2: In one embodiment, the antiparasitic active agent combinations comprise a compound of Formula (IA) with one or more juvenile hormone mimic active agents: Formula (IA)+Jh-1 (Comb 2-1), Formula (IA)+Jh-2 (Comb 2-2), Formula (IA)+Jh-3 (Comb 2-3), Formula (IA)+Jh-4 (Comb 2-4), Formula (IA)+Jh-5 (Comb 2-5), Formula (IA)+Jh-6 (Comb 2-6), Formula (IA)+Jh-7 (Comb 2-7), Formula (IA)+Jh-8 (Comb 2-8) or Formula (IA)+Jh-9 (Comb 2-9).
Combinations 3: In one embodiment, the antiparasitic active agent combinations comprise a compound of Formula (IA) with one or more chitin synthesis inhibitor active agents: Formula (IA)+Ci-1 (Comb 3-1), Formula (IA)+Ci-2 (Comb 3-2), Formula (IA)+Ci-3 (Comb 3-3), Formula (IA)+Ci-4 (Comb 3-4), Formula (IA)+Ci-5 (Comb 3-5), Formula (IA)+Ci-6 (Comb 3-6), Formula (IA)+Ci-7 (Comb 3-7), Formula (IA)+Ci-8 (Comb 3-8), Formula (IA)+Ci-9 (Comb 3-9), Formula (IA)+Ci-10 (Comb 3-10), Formula (IA)+Ci-11 (Comb 3-11), Formula (IA)+Ci-12 (Comb 3-12), Formula (IA)+Ci-13 (Comb 3-13) or Formula (IA)+Ci-14 (Comb 3-14).
Combinations 4: In one embodiment, the antiparasitic active agent combinations comprise a compound of Formula (IA) with one or more organophosphate active agents: Formula (IA)+Op-1 (Comb 4-1), Formula (IA)+Op-2 (Comb 4-2), Formula (IA)+Op-3 (Comb 4-3), Formula (IA)+Op-4 (Comb 4-4), Formula (IA)+Op-5 (Comb 4-5), Formula (IA)+Op-6 (Comb 4-6), Formula (IA)+Op-7 (Comb 4-7), Formula (IA)+Op-8 (Comb 4-8), Formula (IA)+Op-9 (Comb 4-9) or Formula (IA)+Op-10 (Comb 4-10).
Combinations 5: In one embodiment, the antiparasitic active agent combinations comprise a compound of Formula (IA) with one or more pyrethrin or pyrethroid active agents: Formula (IA)+Py-1 (Comb 5-1), Formula (IA)+Py-2 (Comb 5-2), Formula (IA)+Py-3 (Comb 5-3), Formula (IA)+Py-4 (Comb 5-4), Formula (IA)+Py-5 (Comb 5-5), Formula (IA)+Py-6 (Comb 5-6), Formula (IA)+Py-7 (Comb 5-7), Formula (IA)+Py-8 (Comb 5-8), Formula (IA)+Py-9 (Comb 5-9), Formula (IA)+Py-10 (Comb 5-10), Formula (IA)+Py-11 (Comb 5-11), Formula (IA)+Py-12 (Comb 5-12), Formula (IA)+Py-13 (Comb 5-13), Formula (IA)+Py-14 (Comb 5-14), Formula (IA)+Py-15 (Comb 5-15), Formula (IA)+Py-16 (Comb 5-16), Formula (IA)+Py-17 (Comb 5-17), Formula (IA)+Py-18 (Comb 5-18), Formula (IA)+Py-19 (Comb 5-19), Formula (IA)+Py-20 (Comb 5-20), Formula (IA)+Py-21 (Comb 5-21), Formula (IA)+Py-22 (Comb 5-22), Formula (IA)+Py-23 (Comb 5-23), Formula (IA)+Py-24 (Comb 5-24) or Formula (IA)+Py-25 (Comb 5-25).
Combinations 6: In one embodiment, the antiparasitic active agent combinations comprise a compound of Formula (IA) with one or more carbamate active agents: Formula (IA)+Ca-1 (Comb 6-1), Formula (IA)+Ca-2 (Comb 6-2), Formula (IA)+Ca-3 (Comb 6-3), Formula (IA)+Ca-4 (Comb 6-4), Formula (IA)+Ca-5 (Comb 6-5), Formula (IA)+Ca-6 (Comb 6-6), Formula (IA)+Ca-7 (Comb 6-7), Formula (IA)+Ca-8 (Comb 6-8), Formula (IA)+Ca-9 (Comb 6-9), Formula (IA)+Ca-10 (Comb 6-10), Formula (IA)+Ca-11 (Comb 6-11), Formula (IA)+Ca-12 (Comb 6-12) or Formula (IA)+Ca-13 (Comb 6-13).
Combinations 7: In one embodiment, the antiparasitic combinations comprise a compound of Formula (IA) with one or more neonicotinoid active agents: Formula (IA)+Ne-1 (Comb 7-1), Formula (IA)+Ne-2 (Comb 7-2), Formula (IA)+Ne-3 (Comb 7-3), Formula (IA)+Ne-4 (Comb 7-4), Formula (IA)+Ne-5 (Comb 7-5) or Formula (IA)+Ne-6 (Comb 7-6).
Combinations 8: In one embodiment, the antiparasitic combinations comprise a compound of Formula (IA) with one or more semicarbazone active agents: Formula (IA)+Se-1 (Comb 8-1).
Combinations 9: In one embodiment, the antiparasitic combinations comprise a compound of Formula (IA) with one or more isoxazoline active agents: Formula (IA)+Is-1 (Comb 9-1), Formula (IA)+Is-2 (Comb 9-2), Formula (IA)+Is-3 (Comb 9-3), Formula (IA)+Is-4 (Comb 9-4), Formula (IA)+Is-5 (Comb 9-5), Formula (IA)+Is-6 (Comb 9-6), Formula (IA)+Is-7 (Comb 9-7), Formula (IA)+Is-8 (Comb 9-8) or Formula (IA)+Is-9 (Comb 9-9).
Combinations 10: In one embodiment, the antiparasitic combinations comprise a compound of Formula (IA) with one or more macrocyclic lactone active agents: Formula (IA)+ML-1 (Comb 10-1), Formula (IA)+ML-2 (Comb 10-2), Formula (IA)+ML-3 (Comb 10-3), Formula (IA)+ML-4 (Comb 10-4), Formula (IA)+ML-5 (Comb 10-5), Formula (IA)+ML-6 (Comb 10-6), Formula (IA)+ML-7 (Comb 10-7), Formula (IA)+ML-8 (Comb 10-8), Formula (IA)+ML-9 (Comb 10-9), Formula (IA)+ML-10 (Comb 10-10), Formula (IA)+ML-11 (Comb 10-11), Formula (IA)+ML-12 (Comb 10-12), Formula (IA)+ML-13 (Comb 10-13), Formula (IA)+ML-14 (Comb 10-14), Formula (IA)+ML-15 (Comb 10-15), Formula (IA)+ML-16 (Comb 10-16) or Formula (IA)+ML-17 (Comb 10-17).
Combination 11: In one embodiment, the antiparasitic combinations comprise a compound of Formula (IA) with one or more benzimidazole active agents: Formula (IA)+Bz-1 (Comb 11-1), Formula (IA)+Bz-2 (Comb 11-2), Formula (IA)+Bz-3 (Comb 11-3), Formula (IA)+Bz-4 (Comb 11-4), Formula (IA)+Bz-5 (Comb 11-5), Formula (IA)+Bz-6 (Comb 11-6), Formula (IA)+Bz-7 (Comb 11-7), Formula (IA)+Bz-8 (Comb 11-8), Formula (IA)+Bz-9 (Comb 11-9), Formula (IA)+Bz-10 (Comb 11-10), Formula (IA)+Bz-11 (Comb 11-11), Formula (IA)+Bz-12 (Comb 11-12) or Formula (IA)+Bz-13 (Comb 11-13).
Combination 12: In one embodiment, the antiparasitic combinations comprise a compound of Formula (IA) with one or more imidazothiazole active agents: Formula (IA)+Im-1 (Comb 12-1), Formula (IA)+Im-2 (Comb 12-2) or Formula (IA)+Im-3 (Comb 12-3).
Combination 13: In one embodiment, the antiparasitic combinations comprise a compound of Formula (IA) with one or more tetrahydropyrimidines active agents: Formula (IA)+Tp-1 (Comb 13-1), Formula (IA)+Tp-2 (Comb 13-2) or Formula (IA)+Tp-3 (Comb 13-3).
Combination 14: In one embodiment, the antiparasitic combinations of the invention comprise Formula (IA) with one or more antitrematodal active agents: Formula (IA)+At-1 (Comb 14-1), Formula (IA)+At-2 (Comb 14-2), Formula (IA)+At-3 (Comb 14-3), Formula (IA)+At-4 (Comb 14-4), Formula (IA)+At-5 (Comb 14-5), Formula (IA)+At-6 (Comb 14-6), Formula (IA)+At-7 (Comb 14-7), Formula (IA)+At-8 (Comb 14-8), Formula (IA)+At-9 (Comb 14-9), Formula (IA)+At-10 (Comb 14-10), Formula (IA)+At-11 (Comb 14-11), Formula (IA)+At-12 (Comb 14-12), Formula (IA)+At-13 (Comb 14-13), Formula (IA)+At-14 (Comb 14-14), Formula (IA)+At-15 (Comb 14-15), Formula (IA)+At-16 (Comb 14-16), Formula (IA)+At-17 (Comb 14-17), Formula (IA)+At-18 (Comb 14-18), Formula (IA)+At-19 (Comb 14-19), Formula (IA)+At-20 (Comb 14-20), Formula (IA)+At-21 (Comb 14-21), Formula (IA)+At-22 (Comb 14-22), Formula (IA)+At-23 (Comb 14-23), Formula (IA)+At-24 (Comb 14-24), Formula (IA)+At-25 (Comb 14-25), Formula (IA)+At-26 (Comb 14-26) or Formula (IA)+At-27 (Comb 14-27).
Combination 15: In one embodiment, the antiparasitic combinations of the invention comprise a combination of the compound of Formula (IA) with one or more anticestodal active agents: Formula (IA)+Ac-1 (Comb 15-1), Formula (IA)+Ac-2 (Comb 15-2), Formula (IA)+Ac-3 (Comb 15-3), Formula (IA)+Ac-4 (Comb 15-4), Formula (IA)+Ac-5 (Comb 15-5), Formula (IA)+Ac-6 (Comb 15-6), Formula (IA)+Ac-7 (Comb 15-7) or Formula (IA)+Ac-8 (Comb. 15-8).
Combination 16: In one embodiment, the antiparasitic combinations of the invention comprise a combination of the compound of Formula (IA) with one or more antinematodal active agents: Formula (IA)+An-1 (Comb 16-1), Formula (IA)+An-2 (Comb 16-2), Formula (IA)+An-3 (Comb 16-3), Formula (IA)+An-4 (Comb 16-4), Formula (IA)+An-5 (Comb 16-5), Formula (IA)+An-6 (Comb 16-6), Formula (IA)+An-7 (Comb 16-7), Formula (IA)+An-8 (Comb 16-8), Formula (IA)+An-9 (Comb 16-9), Formula (IA)+An-10 (Comb 16-10), Formula (IA)+An-11 (Comb 16-11), Formula (IA)+An-12 (Comb 16-12), Formula (IA)+An-13 (Comb 16-13), Formula (IA)+An-14 (Comb 16-14), Formula (IA)+An-15 (Comb 16-15), Formula (IA)+An-16 (Comb 16-16) or Formula (IA)+An-17 (Comb 16-17).
Combination 17: In another embodiment, the antiparasitic combinations of the invention comprise a combination of the compound of Formula (IA) with cyclooctadepsipeptides active agents: Formula (IA)+Dp-1 (Comb 17-1) or Formula (IA)+Dp-2 (Comb 17-2).
Combination 18: In another embodiment, the antiparasitic combinations of the invention comprise a combination of the compound of Formula (IA) an amino acetonitrile (AAD) active agent such as Formula (IA)+Aa-1.
Combination 19: In another embodiment, the antiparasitic combinations of the invention comprise a combination of the compound of Formula (IA) with one or more paraherquamide active agents such as Formula (IA)-1+Ph-1 (Comb 19-1), Formula (IA)+Ph-2 (Comb 19-2), Formula (IA)+Ph-3 (Comb 19-3) or Formula (IA)+Ph-4 (Comb 19-4).
Combination 20: In one embodiment, the antiparasitic combinations of the invention comprise a combination of the compound of Formula (IA) with one or more spinosyn active agents: Formula (IA)+Sp-1 (Comb 20-1), Formula (IA)+Sp-2 (Comb 20-2), Formula (IA)+Sp-3 (Comb 20-3) or Formula (IA)+Sp-4 (Comb 20-4).
Combinations 21: In another embodiment, the antiparasitic combinations comprise compound A472 with one or more phenylpyrazole active agents: A472+Pp-1 (Comb 21-1), A472+Pp-2 (Comb 21-2).
Combination 22: In another embodiment, the antiparasitic combinations comprise compound A472 with one or more juvenile hormone mimics: A472+Jh-1 (Comb 22-1), A472+Jh-2 (Comb 22-2), A472+Jh-3 (Comb 22-3), A472+Jh-4 (Comb 22-4), A472+Jh-5 (Comb 22-5), A472+Jh-6 (Comb 22-6), A472+Jh-7 (Comb 22-7), A472+Jh-8 (Comb 22-8) or A472+Jh-9 (Comb 22-9).
Combination 23: In another embodiment, the antiparasitic combinations comprise compound A472 with one or more chitin synthesis inhibitors: A472+Ci-1 (Comb 23-1), A472+Ci-2 (Comb 23-2), A472+Ci-3 (Comb 23-3), A472+Ci-4 (Comb 23-4), A472+Ci-5 (Comb 23-5), A472+Ci-6 (Comb 23-6), A472+Ci-7 (Comb 23-7), A472+Ci-8 (Comb 23-8), A472+Ci-9 (Comb 23-9), A472+Ci-10 (Comb 23-10), A472+Ci-11 (Comb 23-11), A472+Ci-12 (Comb 23-12), A472+Ci-13 (Comb 23-13) or A472+Ci-14 (Comb 23-14).
Combination 24: In another embodiment, the antiparasitic combinations comprise compound A472 with one or more organophosphate active agents: A472+Op-1 (Comb 24-1), A472+Op-2 (Comb 24-2), A472+Op-3 (Comb 24-3), A472+Op-4 (Comb 24-4), A472+Op-5 (Comb 24-5), A472+Op-6 (Comb 24-6), A472+Op-7 (Comb 24-7), A472+Op-8 (Comb 24-8), A472+Op-9 (Comb 24-9) or A472+Op-10 (Comb 24-10).
Combination 25: In another embodiment, the antiparasitic combinations comprise a combination of the compound A472 with one or more pyrethrin or pyrethroid active agents: A472+Py-1 (Comb 25-1), A472+Py-2 (Comb 25-2), A472+Py-3 (Comb 25-3), A472+Py-4 (Comb 25-4), A472+Py-5 (Comb 25-5), A472+Py-6 (Comb 25-6), A472+Py-7 (Comb 25-7), A472+Py-8 (Comb 25-8), A472+Py-9 (Comb 25-9), A472+Py-10 (Comb 25-10), A472+Py-11 (Comb 25-11), A472+Py-12 (Comb 25-12), A472+Py-13 (Comb 25-13), A472+Py-14 (Comb 25-14), A472+Py-15 (Comb 25-15), A472+Py-16 (Comb 25-16), A472+Py-17 (Comb 25-17), A472+Py-18 (Comb 25-18), A472+Py-19 (Comb 25-19), A472+Py-20 (Comb 25-20), A472+Py-21 (Comb 25-21), A472+Py-22 (Comb 25-22), A472+Py-23 (Comb 25-23), A472+Py-24 (Comb 25-24) or A472+Py-25 (Comb 25-25).
Combination 26: In another embodiment, the antiparasitic combinations comprise compound A472 with one or more carbamate active agents: A472+Ca-1 (Comb 26-1), A472+Ca-2 (Comb 26-2), A472+Ca-3 (Comb 26-3), A472+Ca-4 (Comb 26-4), A472+Ca-5 (Comb 26-5), A472+Ca-6 (Comb 26-6), A472+Ca-7 (Comb 26-7), A472+Ca-8 (Comb 26-8), A472+Ca-9 (Comb 26-9), A472+Ca-10 (Comb 26-10), A472+Ca-11 (Comb 26-11), A472+Ca-12 (Comb 26-12) or A472+Ca-13 (Comb 26-13).
Combination 27: In another embodiment, the antiparasitic combinations comprise compound A472 with one or more neonicotinoid active agents: A472+Ne-1 (Comb 27-1), A472+Ne-2 (Comb 27-2), A472+Ne-3 (Comb 27-3), A472+Ne-4 (Comb 27-4), A472+Ne-5 (Comb 27-5) or A472+Ne-6 (Comb 27-6).
Combination 28: In another embodiment, the antiparasitic combinations comprise compound A472 with semicarbazone active agent Se-1: A472+Se-1 (Comb 28-1).
Combination 29: In another embodiment, the antiparasitic combinations comprise compound A472 with one or more isoxazoline active agents: A472+Is-1 (Comb 29-1), A472+Is-2 (Comb 29-2), A472+Is-3 (Comb 29-3), A472+Is-4 (Comb 29-4), A472+Is-5 (Comb 29-5), A472+Is-6 (Comb 29-6), A472+Is-7 (Comb 29-7), A472+Is-8 (Comb 29-8) or A472+Is-9 (Comb 29-9).
Combination 30: In another embodiment, the antiparasitic combinations comprise compound A472 with one or more macrocyclic lactone active agents: A472+ML-1 (Comb 30-1), A472+ML-2 (Comb 30-2), A472+ML-3 (Comb 30-3), A472+ML-4 (Comb 30-4), A472+ML-5 (Comb 30-5), A472+ML-6 (Comb 30-6), A472+ML-7 (Comb 30-7), A472+ML-8 (Comb 30-8), A472+ML-9 (Comb 30-9), A472+ML-10 (Comb 30-10), A472+ML-11 (Comb 30-11), A472+ML-12 (Comb 30-12), A472+ML-13 (Comb 30-13), A472+ML-14 (Comb 30-14), A472+ML-15 (Comb 30-15), A472+ML-16 (Comb 30-16) or A472+ML-17 (Comb 30-17).
Combination 31: In another embodiment, the antiparasitic combinations comprise compound A472 with one or more benzimidazole active agents: A472+Bz-1 (Comb 31-1), A472+Bz-2 (Comb 31-2), A472+Bz-3 (Comb 31-3), A472+Bz-4 (Comb 31-4), A472+Bz-5 (Comb 31-5), A472+Bz-6 (Comb 31-6), A472+Bz-7 (Comb 31-7), A472+Bz-8 (Comb 31-8), A472+Bz-9 (Comb 31-9), A472+Bz-10 (Comb 31-10), A472+Bz-11 (Comb 31-11), A472+Bz-12 (Comb 31-12) or A472+Bz-13 (Comb 31-13).
Combination 32: In another embodiment, the antiparasitic combinations comprise compound A472 with one or more imidazothiazole active agents: A472+Im-1 (Comb 32-1), A472+Im-2 (Comb 32-2) or A472+Im-3 (Comb 32-3).
Combination 33: In another embodiment, the antiparasitic combinations comprise compound A472 with one or more tetrahydropyrimidines active agents: A472+Tp-1 (Comb 33-1), A472+Tp-2 (Comb 33-2) or A472+Tp-3 (Comb 33-2).
Combination 34: In another embodiment, the antiparasitic combinations comprise compound A472 with one or more antitrematodal active agents: A472+At-1 (Comb 34-1), A472+At-2 (Comb 34-2), A472+At-3 (Comb 34-3), A472+At-4 (Comb 34-4), A472+At-5 (Comb 34-5), A472+At-6 (Comb 34-6), A472+At-7 (Comb 34-7), A472+At-8 (Comb 34-8), A472+At-9 (Comb 34-9), A472+At-10 (Comb 34-10), A472+At-11 (Comb 34-11), A472+At-12 (Comb 34-12), A472+At-13 (Comb 34-13), A472+At-14 (Comb 34-14), A472+At-15 (Comb 34-15), A472+At-16 (Comb 34-16), A472+At-17 (Comb 34-17), A472+At-18 (Comb 34-18), A472+At-19 (Comb 34-19), A472+At-20 (Comb 34-20), A472+At-21 (Comb 34-21), A472+At-22 (Comb 34-22), A472+At-23 (Comb 34-23), A472+At-24 (Comb 34-24), A472+At-25 (Comb 34-25), A472+At-26 (Comb 34-26) or A472+At-27 (Comb 34-27).
Combination 35: In another embodiment, the antiparasitic combinations comprise compound A472 with one or more anticestodal active agents: A472+Ac-1 (Comb 35-1), A472+Ac-2 (Comb 35-2), A472+Ac-3 (Comb 35-3), A472+Ac-4 (Comb 35-4), A472+Ac-5 (Comb 35-5), A472+Ac-6 (Comb 35-6), A472+Ac-7 (Comb 35-7) or A472+Ac-8 (Comb 35-8).
Combination 36: In another embodiment, the antiparasitic combinations comprise compound A472 with one or more antinematodal active agents: A472+An-1 (Comb 36-1), A472+An-2 (Comb 36-2), A472+An-3 (Comb 36-3), A472+An-4 (Comb 36-4), A472+An-5 (Comb 36-5), A472+An-6 (Comb 36-6), A472+An-7 (Comb 36-7), A472+An-8 (Comb 36-8), A472+An-9 (Comb 36-9), A472+An-10 (Comb 36-10), A472+An-11 (Comb 36-11), A472+An-12 (Comb 36-12), A472+An-13 (Comb 36-13), A472+An-14 (Comb 36-14), A472+An-15 (Comb 36-15), A472+An-16 (Comb 36-16) or A472+An-17 (Comb 36-17).
Combination 37: In another embodiment, the antiparasitic combinations comprise compound A472 with one or more cyclooctadepsipeptides active agents: A472+Dp-1 (Comb 37-1) or A472+Dp-2 (Comb 37-2).
Combination 38: In another embodiment, the antiparasitic combinations comprise compound A472 combined with an amino acetonitrile (AAD) active agent: such as A472+Aa-1 (Comb 38-1).
Combination 39: In another embodiment, the antiparasitic combinations comprise compound A472 with one or more paraherquamide active agents such as A472+Ph-1 (Comb 39-1), A472+Ph-2 (Comb 39-2), A472+Ph-3 (Comb 39-3) or A472+Ph-4 (Comb 39-4).
Combination 40: In one embodiment, the antiparasitic combinations of the invention comprise a combination of the compound A472 with one or more spinosyn active agents: A472+Sp-1 (Comb 40-1), A472+Sp-2 (Comb 40-2), A472+Sp-3 (Comb 40-3) or A472+Sp-4 (Comb 40-4).
Combinations 41: In one embodiment, the antiparasitic combinations comprise a compound of Formula (IC) with one or more phenylpyrazole active agents: Formula (IC)+Pp-1 (Comb 41-1), Formula (IC)+Pp-2 (Comb 41-2).
Combinations 42: In one embodiment, the antiparasitic combinations comprise a compound of Formula (IC) with one or more juvenile hormone mimic active agents: Formula (IC)+Jh-1 (Comb 42-1), Formula (IC)+Jh-2 (Comb 42-2), Formula (IC)+Jh-3 (Comb 42-3), Formula (IC)+Jh-4 (Comb 42-4), Formula (IC)+Jh-5 (Comb 42-5), Formula (IC)+Jh-6 (Comb 42-6), Formula (IC)+Jh-7 (Comb 42-7), Formula (IC)+Jh-8 (Comb 42-8) or Formula (IC)+Jh-9 (Comb 42-9).
Combinations 43: In one embodiment, the antiparasitic combinations comprise a compound of Formula (IC) with one or more chitin synthesis inhibitor active agents: Formula (IC)+Ci-1 (Comb 43-1), Formula (IC)+Ci-2 (Comb 43-2), Formula (IC)+Ci-3 (Comb 43-3), Formula (IC)+Ci-4 (Comb 43-4), Formula (IC)+Ci-5 (Comb 43-5), Formula (IC)+Ci-6 (Comb 43-6), Formula (IC)+Ci-7 (Comb 43-7), Formula (IC)+Ci-8 (Comb 43-8), Formula (IC)+Ci-9 (Comb 43-9), Formula (IC)+Ci-10 (Comb 43-10), Formula (IC)+Ci-11 (Comb 43-11), Formula (IC)+Ci-12 (Comb 43-12), Formula (IC)+Ci-13 (Comb 43-13) or Formula (IC)+Ci-14 (Comb 43-14).
Combinations 44: In one embodiment, the antiparasitic combinations comprise a compound of Formula (IC) with one or more organophosphate active agents: Formula (IC)+Op-1 (Comb 44-1), Formula (IC)+Op-2 (Comb 44-2), Formula (IC)+Op-3 (Comb 44-3), Formula (IC)+Op-4 (Comb 44-4), Formula (IC)+Op-5 (Comb 44-5), Formula (IC)+Op-6 (Comb 44-6), Formula (IC)+Op-7 (Comb 44-7), Formula (IC)+Op-8 (Comb 44-8), Formula (IC)+Op-9 (Comb 44-9) or Formula (IC)+Op-10 (Comb 44-10).
Combinations 45: In one embodiment, the antiparasitic combinations comprise a compound of Formula (IC) with one or more pyrethrin or pyrethroid active agents: Formula (IC)+Py-1 (Comb 45-1), Formula (IC)+Py-2 (Comb 45-2), Formula (IC)+Py-3 (Comb 45-3), Formula (IC)+Py-4 (Comb 45-4), Formula (IC)+Py-5 (Comb 45-5), Formula (IC)+Py-6 (Comb 45-6), Formula (IC)+Py-7 (Comb 45-7), Formula (IC)+Py-8 (Comb 45-8), Formula (IC)+Py-9 (Comb 45-9), Formula (IC)+Py-10 (Comb 45-10), Formula (IC)+Py-11 (Comb 45-11), Formula (IC)+Py-12 (Comb 45-12), Formula (IC)+Py-13 (Comb 45-13), Formula (IC)+Py-14 (Comb 45-14), Formula (IC)+Py-15 (Comb 45-15), Formula (IC)+Py-16 (Comb 45-16), Formula (IC)+Py-17 (Comb 45-17), Formula (IC)+Py-18 (Comb 45-18), Formula (IC)+Py-19 (Comb 45-19), Formula (IC)+Py-20 (Comb 45-20), Formula (IC)+Py-21 (Comb 45-21), Formula (IC)+Py-22 (Comb 45-22), Formula (IC)+Py-23 (Comb 45-23), Formula (IC)+Py-24 (Comb 45-24) or Formula (IC)+Py-25 (Comb 45-25).
Combinations 46: In one embodiment, the antiparasitic combinations comprise a compound of Formula (IC) with one or more carbamate active agents: Formula (IC)+Ca-1 (Comb 46-1), Formula (IC)+Ca-2 (Comb 46-2), Formula (IC)+Ca-3 (Comb 46-3), Formula (IC)+Ca-4 (Comb 46-4), Formula (IC)+Ca-5 (Comb 46-5), Formula (IC)+Ca-6 (Comb 46-6), Formula (IC)+Ca-7 (Comb 46-7), Formula (IC)+Ca-8 (Comb 46-8), Formula (IC)+Ca-9 (Comb 46-9), Formula (IC)+Ca-10 (Comb 46-10), Formula (IC)+Ca-11 (Comb 46-11), Formula (IC)+Ca-12 (Comb 46-12) or Formula (IC)+Ca-13 (Comb 46-13).
Combinations 47: In one embodiment, the antiparasitic combinations comprise a compound of Formula (IC) with one or more neonicotinoid active agents: Formula (IC)+Ne-1 (Comb 47-1), Formula (IC)+Ne-2 (Comb 47-2), Formula (IC)+Ne-3 (Comb 47-3), Formula (IC)+Ne-4 (Comb 47-4), Formula (IC)+Ne-5 (Comb 47-5) or Formula (IC)+Ne-6 (Comb 47-6).
Combinations 48: In one embodiment, the antiparasitic combinations comprise a compound of Formula (IC) with one or more semicarbazone active agents: Formula (IC)+Se-1 (Comb 48-1).
Combinations 49: In one embodiment, the antiparasitic combinations comprise a compound of Formula (IC) with one or more isoxazoline active agents: Formula (IC)+Is-1 (Comb 49-1), Formula (IC)+Is-2 (Comb 49-2), Formula (IC)+Is-3 (Comb 49-3), Formula (IC)+Is-4 (Comb 49-4), Formula (IC)+Is-5 (Comb 49-5), Formula (IC)+Is-6 (Comb 49-6), Formula (IC)+Is-7 (Comb 49-7), Formula (IC)+Is-8 (Comb 49-8) or Formula (IC)+Is-9 (Comb 49-9).
Combinations 50: In one embodiment, the antiparasitic combinations comprise a compound of Formula (IC) with one or more macrocyclic lactone active agents: Formula (IC)+ML-1 (Comb 50-1), Formula (IC)+ML-2 (Comb 50-2), Formula (IC)+ML-3 (Comb 50-3), Formula (IC)+ML-4 (Comb 50-4), Formula (IC)+ML-5 (Comb 50-5), Formula (IC)+ML-6 (Comb 50-6), Formula (IC)+ML-7 (Comb 50-7), Formula (IC)+ML-8 (Comb 50-8), Formula (IC)+ML-9 (Comb 50-9), Formula (IC)+ML-10 (Comb 50-10), Formula (IC)+ML-11 (Comb 50-11), Formula (IC)+ML-12 (Comb 50-12), Formula (IC)+ML-13 (Comb 50-13), Formula (IC)+ML-14 (Comb 50-14), Formula (IC)+ML-15 (Comb 50-15), Formula (IC)+ML-16 (Comb 50-16) or Formula (IC)+ML-17 (Comb 50-17).
Combination 51: In one embodiment, the antiparasitic combinations comprise a compound of Formula (IC) with one or more benzimidazole active agents: Formula (IC)+Bz-1 (Comb 51-1), Formula (IC)+Bz-2 (Comb 51-2), Formula (IC)+Bz-3 (Comb 51-3), Formula (IC)+Bz-4 (Comb 51-4), Formula (IC)+Bz-5 (Comb 51-5), Formula (IC)+Bz-6 (Comb 51-6), Formula (IC)+Bz-7 (Comb 51-7), Formula (IC)+Bz-8 (Comb 51-8), Formula (IC)+Bz-9 (Comb 51-9), Formula (IC)+Bz-10 (Comb 51-10), Formula (IC)+Bz-11 (Comb 51-11), Formula (IC)+Bz-12 (Comb 51-12) or Formula (IC)+Bz-13 (Comb 51-13).
Combination 52: In one embodiment, the antiparasitic combinations comprise a compound of Formula (IC) with one or more imidazothiazole active agents: Formula (IC)+Im-1 (Comb 52-1), Formula (IC)+Im-2 (Comb 52-2) or Formula (IC)+Im-3 (Comb 52-3).
Combination 53: In one embodiment, the antiparasitic combinations comprise a compound of Formula (IC) with one or more tetrahydropyrimidines active agents: Formula (IC)+Tp-1 (Comb 53-1), Formula (IC)+Tp-2 (Comb 53-2) or Formula (IC)+Tp-3 (Comb 53-3).
Combination 54: In one embodiment, the antiparasitic combinations of the invention comprise Formula (IC) with one or more antitrematodal active agents: Formula (IC)+At-1 (Comb 54-1), Formula (IC)+At-2 (Comb 54-2), Formula (IC)+At-3 (Comb 54-3), Formula (IC)+At-4 (Comb 54-4), Formula (IC)+At-5 (Comb 54-5), Formula (IC)+At-6 (Comb 54-6), Formula (IC)+At-7 (Comb 54-7), Formula (IC)+At-8 (Comb 54-8), Formula (IC)+At-9 (Comb 54-9), Formula (IC)+At-10 (Comb 54-10), Formula (IC)+At-11 (Comb 54-11), Formula (IC)+At-12 (Comb 54-12), Formula (IC)+At-13 (Comb 54-13), Formula (IC)+At-14 (Comb 54-14), Formula (IC)+At-15 (Comb 54-15), Formula (IC)+At-16 (Comb 54-16), Formula (IC)+At-17 (Comb 54-17), Formula (IC)+At-18 (Comb 54-18), Formula (IC)+At-19 (Comb 54-19), Formula (IC)+At-20 (Comb 54-20), Formula (IC)+At-21 (Comb 54-21), Formula (IC)+At-22 (Comb 54-22), Formula (IC)+At-23 (Comb 54-23), Formula (IC)+At-24 (Comb 54-24), Formula (IC)+At-25 (Comb 54-25), Formula (IC)+At-26 (Comb 54-26) or Formula (IC)+At-27 (Comb 54-27).
Combination 55: In one embodiment, the antiparasitic combinations of the invention comprise a combination of the compound of Formula (IC) with one or more anticestodal active agents: Formula (IC)+Ac-1 (Comb 55-1), Formula (IC)+Ac-2 (Comb 55-2), Formula (IC)+Ac-3 (Comb 55-3), Formula (IC)+Ac-4 (Comb 55-4), Formula (IC)+Ac-5 (Comb 55-5), Formula (IC)+Ac-6 (Comb 55-6), Formula (IC)+Ac-7 (Comb 55-7) or Formula (IC)+Ac-8 (Comb 55-8).
Combination 56: In one embodiment, the antiparasitic combinations of the invention comprise a combination of the compound of Formula (IC) with one or more antinematodal active agents: Formula (IC)+An-1 (Comb 56-1), Formula (IC)+An-2 (Comb 56-2), Formula (IC)+An-3 (Comb 56-3), Formula (IC)+An-4 (Comb 56-4), Formula (IC)+An-5 (Comb 56-5), Formula (IC)+An-6 (Comb 56-6), Formula (IC)+An-7 (Comb 56-7), Formula (IC)+An-8 (Comb 56-8), Formula (IC)+An-9 (Comb 56-9), Formula (IC)+An-10 (Comb 56-10), Formula (IC)+An-11 (Comb 56-11), Formula (IC)+An-12 (Comb 56-12), Formula (IC)+An-13 (Comb 56-13), Formula (IC)+An-14 (Comb 56-14), Formula (IC)+An-15 (Comb 56-15), Formula (IC)+An-16 (Comb 56-16) or Formula (IC)+An-17 (Comb 56-17).
Combination 57: In another embodiment, the antiparasitic combinations of the invention comprise a combination of the compound of Formula (IC) with cyclooctadepsipeptides active agents: Formula (IC)+Dp-1 (Comb 57-1) or Formula (IC)+Dp-2 Comb 57-2).
Combination 58: In another embodiment, the antiparasitic combinations of the invention comprise a combination of the compound of Formula (IC) an amino acetonitrile (AAD) active agent such as Formula (IC)+Aa-1 (Comb 58-1).
Combination 59: In another embodiment, the antiparasitic combinations of the invention comprise a combination of the compound of Formula (IC) with one or more paraherquamide active agents such as Formula (IC)+Ph-1 (Comb 59-1), Formula (IC)+Ph-2 (Comb 59-2), Formula (IC)+Ph-3 (Comb 59-3) or Formula (IC)+Ph-4 (Comb 59-4).
Combination 60: In one embodiment, the antiparasitic combinations of the invention comprise a combination of the compound of Formula (IC) with one or more spinosyn active agents: Formula (IC)+Sp-1 (Comb 60-1), Formula (IC)+Sp-2 (Comb 60-2), Formula (IC)+Sp-3 (Comb 60-3) or Formula (IC)+Sp-4 (Comb 60-4).
Combination 61: In another embodiment, the antiparasitic combinations comprise a combination of the compound IC-1 with phenylpyrazole: IC-1+Pp-1 (Comb 61-1) or IC-1+Pp-2 (Comb 61-2).
Combination 62: In another embodiment, the antiparasitic combinations comprise a combination of the compound IC-1 with one or more juvenile hormone mimics: IC-1+Jh-1 (Comb 62-1), IC-1+Jh-2 (Comb 62-2), IC-1+Jh-3 (Comb 62-3), IC-1+Jh-4 (Comb 62-4), IC-1+Jh-5 (Comb 62-5), IC-1+Jh-6 (Comb 62-6), IC-1+Jh-7 (Comb 62-7), IC-1+Jh-8 (Comb 62-8) or IC-1+Jh-9 (Comb 62-9).
Combination 63: In another embodiment, the antiparasitic combinations comprise a combination of the compound IC-1 with one or more chitin synthesis inhibitors: IC-1+Ci-1 (Comb 63-1), IC-1+Ci-2 (Comb 63-2), IC-1+Ci-3 (Comb 63-3), IC-1+Ci-4 (Comb 63-4), IC-1+Ci-5 (Comb 63-5), IC-1+Ci-6 (Comb 63-6), IC-1+Ci-7 (Comb 63-7), IC-1+Ci-8 (Comb 63-8), IC-1+Ci-9 (Comb 63-9), IC-1+Ci-10 (Comb 63-10), IC-1+Ci-11 (Comb 63-11), IC-1+Ci-12 (Comb 63-12), IC-1+Ci-13 (Comb 63-13) or IC-1+Ci-14 (Comb 63-14).
Combination 64: In another embodiment, the antiparasitic combinations comprise a combination of the compound IC-1 with one or more organophosphate active agents: IC-1+Op-1 (Comb 64-1), IC-1+Op-2 (Comb 64-2), IC-1+Op-3 (Comb 64-3), IC-1+Op-4 (Comb 64-4), IC-1+Op-5 (Comb 64-5), IC-1+Op-6 (Comb 64-6), IC-1+Op-7 (Comb 64-7), IC-1+Op-8 (Comb 64-8), IC-1+Op-9 (Comb 64-9) or IC-1+Op-10 (Comb 64-10).
Combination 65: In another embodiment, the antiparasitic combinations comprise a combination of the compound IC-1 with one or more pyrethrin or pyrethroid active agents: IC-1+Py-1 (Comb 65-1), IC-1+Py-2 (Comb 65-2), IC-1+Py-3 (Comb 65-3), IC-1+Py-4 (Comb 65-4), IC-1+Py-5 (Comb 65-5), IC-1+Py-6 (Comb 65-6), IC-1+Py-7 (Comb 65-7), IC-1+Py-8 (Comb 65-8), IC-1+Py-9 (Comb 65-9), IC-1+Py-10 (Comb 65-10), IC-1+Py-11 (Comb 65-11), IC-1+Py-12 (Comb 65-12), IC-1+Py-13 (Comb 65-13), IC-1+Py-14 (Comb 65-14), IC-1+Py-15 (Comb 65-15), IC-1+Py-16 (Comb 65-16), IC-1+Py-17 (Comb 65-17), IC-1+Py-18 (Comb 65-18), IC-1+Py-19 (Comb 65-19), IC-1+Py-20 (Comb 65-20), IC-1+Py-21 (Comb 65-21), IC-1+Py-22 (Comb 65-22), IC-1+Py-23 (Comb 65-23), IC-1+Py-24 (Comb 65-24) or IC-1+Py-25 (Comb 65-25.
Combination 66: In another embodiment, the antiparasitic combinations comprise a combination of the compound IC-1 with one or more carbamate active agents: IC-1+Ca-1 (Comb 66-1), IC-1+Ca-2 (Comb 66-2), IC-1+Ca-3 (Comb 66-3), IC-1+Ca-4 (Comb 66-4), IC-1+Ca-5 (Comb 66-5), IC-1+Ca-6 (Comb 66-6), IC-1+Ca-7 (Comb 66-7), IC-1+Ca-8 (Comb 66-8), IC-1+Ca-9 (Comb 66-9), IC-1+Ca-10 (Comb 66-10), IC-1+Ca-11 (Comb 66-11), IC-1+Ca-12 (Comb 66-12) or IC-1+Ca-13 (Comb 66-13).
Combination 67: In another embodiment, the antiparasitic combinations comprise a combination of the compound IC-1 with one or more neonicotinoid active agents: IC-1+Ne-1 (Comb 67-1), IC-1+Ne-2 (Comb 67-2), IC-1+Ne-3 (Comb 67-3), IC-1+Ne-4 (Comb 67-4), IC-1+Ne-5 (Comb 67-5) or IC-1+Ne-6 (Comb 67-6).
Combination 68: In another embodiment, the antiparasitic combinations comprise a combination of the compound IC-1 with semicarbazone active agent Se-1: IC-1+Se-1 (Comb 68-1).
Combination 69: In one embodiment, the antiparasitic combinations of the invention comprise a combination of compound IC-1 with one or more isoxazoline active agents: IC-1+Is-1 (Comb 69-1), IC-1+Is-2 (Comb 69-2), IC-1+Is-3 (Comb 69-3), IC-1+Is-4 (Comb 69-4), IC-1+Is-5 (Comb 69-5), IC-1+Is-6 (Comb 69-6), IC-1+Is-7 (Comb 69-7), IC-1+Is-8 (Comb 69-8) or IC-1+Is-9 (Comb 69-9).
Combination 70: In another embodiment, the antiparasitic combinations comprise a combination of the compound IC-1 with one or more macrocyclic lactone active agents: IC-1+ML-1 (Comb 70-1), IC-1+ML-2 (Comb 70-2), IC-1+ML-3 (Comb 70-3), IC-1+ML-4 (Comb 70-4), IC-1+ML-5 (Comb 70-5), IC-1+ML-6 (Comb 70-6), IC-1+ML-7 (Comb 70-7), IC-1+ML-8 (Comb 70-8), IC-1+ML-9 (Comb 70-9), IC-1+ML-10 (Comb 70-10), IC-1+ML-11 (Comb 70-11), IC-1+ML-12 (Comb 70-12), IC-1+ML-13 (Comb 70-13), IC-1+ML-14 (Comb 70-14), IC-1+ML-15 (Comb 70-15), IC-1+ML-16 (Comb 70-16) or IC-1+ML-17 (Comb 70-17).
Combination 71: In one embodiment, the antiparasitic combinations of the invention comprise a combination of the compound IC-1 with one or more benzimidazole active agents: IC-1+Bz-1 (Comb 71-1), IC-1+Bz-2 (Comb 71-2), IC-1+Bz-3 (Comb 71-3), IC-1+Bz-4 (Comb 71-4), IC-1+Bz-5 (Comb 71-5), IC-1+Bz-6 (Comb 71-6), IC-1+Bz-7 (Comb 71-7), IC-1+Bz-8 (Comb 71-8), IC-1+Bz-9 (Comb 71-9), IC-1+Bz-10 (Comb 71-10), IC-1+Bz-11 (Comb 71-11), IC-1+Bz-12 (Comb 71-12) or IC-1+Bz-13 (Comb 71-13).
Combination 72: In one embodiment, the antiparasitic combinations of the invention comprise a combination of the compound IC-1 with one or more imidazothiazole active agents: IC-1+Im-1 (Comb 72-1), IC-1+Im-2 (Comb 72-2) or IC-1+Im-3 (Comb 72-3).
Combination 73: In one embodiment, the antiparasitic combinations of the invention comprise a combination of the compound IC-1 with one or more tetrahydropyrimidines active agents: IC-1+Tp-1 (Comb 73-1), IC-1+Tp-2 (Comb 73-2) or IC-1+Tp-3 (Comb 73-3).
Combination 74: In one embodiment, the antiparasitic combinations of the invention comprise a combination of the compound IC-1 with one or more antitrematodal active agents: IC-1+At-1 (Comb 74-1), IC-1+At-2 (Comb 74-2), IC-1+At-3 (Comb 74-3), IC-1+At-4 (Comb 74-4), IC-1+At-5 (Comb 74-5), IC-1+At-6 (Comb 74-6), IC-1+At-7 (Comb 74-7), IC-1+At-8 (Comb 74-8), IC-1+At-9 (Comb 74-9), IC-1+At-10 (Comb 74-10), IC-1+At-11 (Comb 74-11), IC-1+At-12 (Comb 74-12), IC-1+At-13 (Comb 74-13), IC-1+At-14 (Comb 74-14), IC-1+At-15 (Comb 74-15), IC-1+At-16 (Comb 74-16), IC-1+At-17 (Comb 74-17), IC-1+At-18 (Comb 74-18), IC-1+At-19 (Comb 74-19), IC-1+At-20 (Comb 74-20), IC-1+At-21 (Comb 74-21), IC-1+At-22 (Comb 74-22), IC-1+At-23 (Comb 74-23), IC-1+At-24 (Comb 74-24), IC-1+At-25 (Comb 74-25), IC-1+At-26 (Comb 74-26) or IC-1+At-27 (Comb 74-27).
Combination 75: In one embodiment, the antiparasitic combinations of the invention comprise a combination of the compound IC-1 with one or more anticestodal active agents: IC-1+Ac-1 (Comb 75-1), IC-1+Ac-2 (Comb 75-2), IC-1+Ac-3 (Comb 75-3), IC-1+Ac-4 (Comb 75-4), IC-1+Ac-5 (Comb 75-5), IC-1+Ac-6 (Comb 75-6), IC-1+Ac-7 (Comb 75-7) or IC-1+Ac-8 (Comb 75-8).
Combination 76: In one embodiment, the antiparasitic combinations of the invention comprise a combination of the compound IC-1 with one or more antinematodal active agents: IC-1+An-1 (Comb 76-1), IC-1+An-2 (Comb 76-2), IC-1+An-3 (Comb 76-3), IC-1+An-4 (Comb 76-4), IC-1+An-5 (Comb 76-5), IC-1+An-6 (Comb 76-6), IC-1+An-7 (Comb 76-7), IC-1+An-8 (Comb 76-8), IC-1+An-9 (Comb 76-9), IC-1+An-10 (Comb 76-10), IC-1+An-11 (Comb 76-11), IC-1+An-12 (Comb 76-12), IC-1+An-13 (Comb 76-13), IC-1+An-14 (Comb 76-14), IC-1+An-15 (Comb 76-15), IC-1+An-16 (Comb 76-16) or IC-1+An-17 (Comb 76-17).
Combination 77: In another embodiment, the antiparasitic combinations of the invention comprise a combination of the compound IC-1 with cyclooctadepsipeptides active agents: IC-1+Dp-1 (Comb 77-1) or IC-1+Dp-2 (Comb 77-2).
Combination 78: In another embodiment, the antiparasitic combinations of the invention comprise a combination of the compound IC-1 an amino acetonitrile (AAD) active agent such as Aa-1: IC-1+Aa-1 (Comb 78-1).
Combination 79: In another embodiment, the antiparasitic combinations of the invention comprise a combination of the compound IC-1 with one or more paraherquamide active agents: IC-1+Ph-1 (Comb 79-1), IC-1+Ph-2 (Comb 79-2), IC-1+Ph-3 (Comb 79-3) or IC-1+Ph-4 (Comb 79-4).
Combination 80: In one embodiment, the antiparasitic combinations of the invention comprise a combination of the compound IC-1 with one or more spinosyn active agents: IC-1+Sp-1 (Comb 80-1), IC-1+Sp-2 (Comb 80-2), IC-1+Sp-3 (Comb 80-3) or IC-1+Sp-4 (Comb 80-4).

In further embodiments of the antiparasitic combinations of the invention, the antiparasitic combinations Comb 29-1 to Comb 29-9 described above, which contain combinations of compound A472 with isoxazoline active agents Is- to Is-9, are combined with macrocyclic lactone active agents ML-1 to ML-17 to provide further antiparasitic combinations C81-1 to C81-153 as shown in Table 2 below. In Table 2, the combinations Comb 29-1 to Comb 29-9 described above are abbreviated “C29-1 to C29-9” for brevity. For clarity, each antiparasitic combination C29-1 to C29-9 shown in the first row of the table are combined with macrocyclic lactones Ml-1 to M8-17 in the first column to provide new 3-way combinations C81-1 to C81-153. For example, combination Comb 29-1 (abbreviated C29-1 in Table 2) includes the combination of compound A472 with afoxolaner (Is-1). This combination is further combined with macrocyclic lactone abamectin (MIL-1) to produce 3-way combination C81-1 containing compound A472+afoxolaner+abamectin.

TABLE 2

| | C29-1 | C29-2 | C29-3 | C29-4 | C29-5 | C29-6 | C29-7 | C29-8 | C29-9 |
|---|---|---|---|---|---|---|---|---|---|
| ML-1 | C81-1 | C81-2 | C81-3 | C81-4 | C81-5 | C81-6 | C81-7 | C81-8 | C81-9 |
| ML-2 | C81-10 | C81-11 | C81-12 | C81-13 | C81-14 | C81-15 | C81-16 | C81-17 | C81-18 |
| ML-3 | C81-19 | C81-20 | C81-21 | C81-22 | C81-23 | C81-24 | C81-25 | C81-26 | C81-27 |
| ML-4 | C81-28 | C81-29 | C81-30 | C81-31 | C81-32 | C81-33 | C81-34 | C81-35 | C81-36 |
| ML-5 | C81-37 | C81-38 | C81-39 | C81-40 | C81-41 | C81-42 | C81-43 | C81-44 | C81-45 |
| ML-6 | C81-46 | C81-47 | C81-48 | C81-49 | C81-50 | C81-51 | C81-52 | C81-53 | C81-54 |
| ML-7 | C81-55 | C81-56 | C81-57 | C81-58 | C81-59 | C81-60 | C81-61 | C81-62 | C81-63 |
| ML-8 | C81-64 | C81-65 | C81-66 | C81-67 | C81-68 | C81-69 | C81-70 | C81-71 | C81-72 |
| ML-9 | C81-73 | C81-74 | C81-75 | C81-76 | C81-77 | C81-78 | C81-79 | C81-80 | C81-81 |
| ML-10 | C81-82 | C81-83 | C81-84 | C81-85 | C81-86 | C81-87 | C81-88 | C81-89 | C81-90 |
| ML-11 | C81-91 | C81-92 | C81-93 | C81-94 | C81-95 | C81-96 | C81-97 | C81-98 | C81-99 |
| ML-12 | C81-100 | C81-101 | C81-102 | C81-103 | C81-104 | C81-105 | C81-106 | C81-107 | C81-108 |
| ML-13 | C81-109 | C81-110 | C81-111 | C81-112 | C81-113 | C81-114 | C81-115 | C81-116 | C81-117 |
| ML-14 | C81-118 | C81-119 | C81-120 | C81-121 | C81-122 | C81-123 | C81-124 | C81-125 | C81-126 |
| ML-15 | C81-127 | C81-128 | C81-129 | C81-130 | C81-131 | C81-132 | C81-133 | C81-134 | C81-135 |
| ML-16 | C81-136 | C81-137 | C81-138 | C81-139 | C81-140 | C81-141 | C81-142 | C81-143 | C81-144 |
| ML-17 | C81-145 | C81-146 | C81-147 | C81-148 | C81-149 | C81-150 | C81-151 | C81-152 | C81-153 |

In other embodiments of the antiparasitic combinations of the invention, the antiparasitic combinations Comb 69-1 to Comb 69-9 described above (abbreviated in Table 3 as C69-1 to C69-9 for brevity) containing combinations of compound IC-1 and isoxazoline active agents Is-i to Is-9 are combined with macrocyclic lactone active agents MIL-1 to MIL-17 to provide further antiparasitic combinations C82-1 to C82-153 as shown in Table 3 below.

TABLE 3

| | C69-1 | C69-2 | C69-3 | C69-4 | C69-5 | C69-6 | C69-7 | C69-8 | C69-9 |
|---|---|---|---|---|---|---|---|---|---|
| ML-1 | C82-1 | C82-2 | C82-3 | C82-4 | C82-5 | C82-6 | C82-7 | C82-8 | C82-9 |
| ML-2 | C82-10 | C82-11 | C82-12 | C82-13 | C82-14 | C82-15 | C82-16 | C82-17 | C82-18 |
| ML-3 | C82-19 | C82-20 | C82-21 | C82-22 | C82-23 | C82-24 | C82-25 | C82-26 | C82-27 |
| ML-4 | C82-28 | C82-29 | C82-30 | C82-31 | C82-32 | C82-33 | C82-34 | C82-35 | C82-36 |
| ML-5 | C82-37 | C82-38 | C82-39 | C82-40 | C82-41 | C82-42 | C82-43 | C82-44 | C82-45 |
| ML-6 | C82-46 | C82-47 | C82-48 | C82-49 | C82-50 | C82-51 | C82-52 | C82-53 | C82-54 |
| ML-7 | C82-55 | C82-56 | C82-57 | C82-58 | C82-59 | C82-60 | C82-61 | C82-62 | C82-63 |
| ML-8 | C82-64 | C82-65 | C82-66 | C82-67 | C82-68 | C82-69 | C82-70 | C82-71 | C82-72 |
| ML-9 | C82-73 | C82-74 | C82-75 | C82-76 | C82-77 | C82-78 | C82-79 | C82-80 | C82-81 |
| ML-10 | C82-82 | C82-83 | C82-84 | C82-85 | C82-86 | C82-87 | C82-88 | C82-89 | C82-90 |
| ML-11 | C82-91 | C82-92 | C82-93 | C82-94 | C82-95 | C82-96 | C82-97 | C82-98 | C82-99 |
| ML-12 | C82-100 | C82-101 | C82-102 | C82-103 | C82-104 | C82-105 | C82-106 | C82-107 | C82-108 |
| ML-13 | C82-109 | C82-110 | C82-111 | C82-112 | C82-113 | C82-114 | C82-115 | C82-116 | C82-117 |
| ML-14 | C82-118 | C82-119 | C82-120 | C82-121 | C82-122 | C82-123 | C82-124 | C82-125 | C82-126 |
| ML-15 | C82-127 | C82-128 | C82-129 | C82-130 | C82-131 | C82-132 | C82-133 | C82-134 | C82-135 |
| ML-16 | C82-136 | C82-137 | C82-138 | C82-139 | C82-140 | C82-141 | C82-142 | C82-143 | C82-144 |
| ML-17 | C82-145 | C82-146 | C82-147 | C82-148 | C82-149 | C82-150 | C82-151 | C82-152 | C82-153 |

In other embodiments of the antiparasitic combinations of the invention, the antiparasitic combinations C81-1 to C81-153 described in Table 2 above containing combinations of compound A472, isoxazolines Is-1 to Is-9 and macrocyclic lactones ML-1 to ML-17 are further combined with anticestodal active agents Ac-1 to Ac-8 to provide further antiparasitic combinations C83-1 to C83-1224 as shown in Table 4 below.

TABLE 4

| | Ac-1 | Ac-2 | Ac-3 | Ac-4 | Ac-5 | Ac-6 | Ac-7 | Ac-8 |
|---|---|---|---|---|---|---|---|---|
| C81-1 | C83-1 | C83-2 | C83-3 | C83-4 | C83-5 | C83-6 | C83-7 | C83-8 |
| C81-2 | C83-9 | C83-10 | C83-11 | C83-12 | C83-13 | C83-14 | C83-15 | C83-16 |
| C81-3 | C83-17 | C83-18 | C83-19 | C83-20 | C83-21 | C83-22 | C83-23 | C83-24 |
| C81-4 | C83-25 | C83-26 | C83-27 | C83-28 | C83-29 | C83-30 | C83-31 | C83-32 |
| C81-5 | C83-33 | C83-34 | C83-35 | C83-36 | C83-37 | C83-38 | C83-39 | C83-40 |
| C81-6 | C83-41 | C83-42 | C83-43 | C83-44 | C83-45 | C83-46 | C83-47 | C83-48 |
| C81-7 | C83-49 | C83-50 | C83-51 | C83-52 | C83-53 | C83-54 | C83-55 | C83-56 |
| C81-8 | C83-57 | C83-58 | C83-59 | C83-60 | C83-61 | C83-62 | C83-63 | C83-64 |
| C81-9 | C83-65 | C83-66 | C83-67 | C83-68 | C83-69 | C83-70 | C83-71 | C83-72 |
| C81-10 | C83-73 | C83-74 | C83-75 | C83-76 | C83-77 | C83-78 | C83-79 | C83-80 |
| C81-11 | C83-81 | C83-82 | C83-83 | C83-84 | C83-85 | C83-86 | C83-87 | C83-88 |
| C81-12 | C83-89 | C83-90 | C83-91 | C83-92 | C83-93 | C83-94 | C83-95 | C83-96 |
| C81-13 | C83-97 | C83-98 | C83-99 | C83-100 | C83-101 | C83-102 | C83-103 | C83-104 |
| C81-14 | C83-105 | C83-106 | C83-107 | C83-108 | C83-109 | C83-110 | C83-111 | C83-112 |
| C81-15 | C83-113 | C83-114 | C83-115 | C83-116 | C83-117 | C83-118 | C83-119 | C83-120 |
| C81-16 | C83-121 | C83-122 | C83-123 | C83-124 | C83-125 | C83-126 | C83-127 | C83-128 |
| C81-17 | C83-129 | C83-130 | C83-131 | C83-132 | C83-133 | C83-134 | C83-135 | C83-136 |
| C81-18 | C83-137 | C83-138 | C83-139 | C83-140 | C83-141 | C83-142 | C83-143 | C83-144 |
| C81-19 | C83-145 | C83-146 | C83-147 | C83-148 | C83-149 | C83-150 | C83-151 | C83-152 |
| C81-20 | C83-153 | C83-154 | C83-155 | C83-156 | C83-157 | C83-158 | C83-159 | C83-160 |
| C81-21 | C83-161 | C83-162 | C83-163 | C83-164 | C83-165 | C83-166 | C83-167 | C83-168 |
| C81-22 | C83-169 | C83-170 | C83-171 | C83-172 | C83-173 | C83-174 | C83-175 | C83-176 |
| C81-23 | C83-177 | C83-178 | C83-179 | C83-180 | C83-181 | C83-182 | C83-183 | C83-184 |
| C81-24 | C83-185 | C83-186 | C83-187 | C83-188 | C83-189 | C83-190 | C83-191 | C83-192 |
| C81-25 | C83-193 | C83-194 | C83-195 | C83-196 | C83-197 | C83-198 | C83-199 | C83-200 |
| C81-26 | C83-201 | C83-202 | C83-203 | C83-204 | C83-205 | C83-206 | C83-207 | C83-208 |
| C81-27 | C83-209 | C83-210 | C83-211 | C83-212 | C83-213 | C83-214 | C83-215 | C83-216 |
| C81-28 | C83-217 | C83-218 | C83-219 | C83-220 | C83-221 | C83-222 | C83-223 | C83-224 |
| C81-29 | C83-225 | C83-226 | C83-227 | C83-228 | C83-229 | C83-230 | C83-231 | C83-232 |
| C81-30 | C83-233 | C83-234 | C83-235 | C83-236 | C83-237 | C83-238 | C83-239 | C83-240 |
| C81-31 | C83-241 | C83-242 | C83-243 | C83-244 | C83-245 | C83-246 | C83-247 | C83-248 |
| C81-32 | C83-249 | C83-250 | C83-251 | C83-252 | C83-253 | C83-254 | C83-255 | C83-256 |
| C81-33 | C83-257 | C83-258 | C83-259 | C83-260 | C83-261 | C83-262 | C83-263 | C83-264 |
| C81-34 | C83-265 | C83-266 | C83-267 | C83-268 | C83-269 | C83-270 | C83-271 | C83-272 |
| C81-35 | C83-273 | C83-274 | C83-275 | C83-276 | C83-277 | C83-278 | C83-279 | C83-280 |
| C81-36 | C83-281 | C83-282 | C83-283 | C83-284 | C83-285 | C83-286 | C83-287 | C83-288 |
| C81-37 | C83-289 | C83-290 | C83-291 | C83-292 | C83-293 | C83-294 | C83-295 | C83-296 |
| C81-38 | C83-297 | C83-298 | C83-299 | C83-300 | C83-301 | C83-302 | C83-303 | C83-304 |
| C81-39 | C83-305 | C83-306 | C83-307 | C83-308 | C83-309 | C83-310 | C83-311 | C83-312 |
| C81-40 | C83-313 | C83-314 | C83-315 | C83-316 | C83-317 | C83-318 | C83-319 | C83-320 |
| C81-41 | C83-321 | C83-322 | C83-323 | C83-324 | C83-325 | C83-326 | C83-327 | C83-328 |
| C81-42 | C83-329 | C83-330 | C83-331 | C83-332 | C83-333 | C83-334 | C83-335 | C83-336 |
| C81-43 | C83-337 | C83-338 | C83-339 | C83-340 | C83-341 | C83-342 | C83-343 | C83-344 |
| C81-44 | C83-345 | C83-346 | C83-347 | C83-348 | C83-349 | C83-350 | C83-351 | C83-352 |
| C81-45 | C83-353 | C83-354 | C83-355 | C83-356 | C83-357 | C83-358 | C83-359 | C83-360 |
| C81-46 | C83-361 | C83-362 | C83-363 | C83-364 | C83-365 | C83-366 | C83-367 | C83-368 |
| C81-47 | C83-369 | C83-370 | C83-371 | C83-372 | C83-373 | C83-374 | C83-375 | C83-376 |
| C81-48 | C83-377 | C83-378 | C83-379 | C83-380 | C83-381 | C83-382 | C83-383 | C83-384 |
| C81-49 | C83-385 | C83-386 | C83-387 | C83-388 | C83-389 | C83-390 | C83-391 | C83-392 |
| C81-50 | C83-393 | C83-394 | C83-395 | C83-396 | C83-397 | C83-398 | C83-399 | C83-400 |
| C81-51 | C83-401 | C83-402 | C83-403 | C83-404 | C83-405 | C83-406 | C83-407 | C83-408 |
| C81-52 | C83-409 | C83-410 | C83-411 | C83-412 | C83-413 | C83-414 | C83-415 | C83-416 |
| C81-53 | C83-417 | C83-418 | C83-419 | C83-420 | C83-421 | C83-422 | C83-423 | C83-424 |
| C81-54 | C83-425 | C83-426 | C83-427 | C83-428 | C83-429 | C83-430 | C83-431 | C83-432 |
| C81-55 | C83-433 | C83-434 | C83-435 | C83-436 | C83-437 | C83-438 | C83-439 | C83-440 |
| C81-56 | C83-441 | C83-442 | C83-443 | C83-444 | C83-445 | C83-446 | C83-447 | C83-448 |
| C81-57 | C83-449 | C83-450 | C83-451 | C83-452 | C83-453 | C83-454 | C83-455 | C83-456 |
| C81-58 | C83-457 | C83-458 | C83-459 | C83-460 | C83-461 | C83-462 | C83-463 | C83-464 |
| C81-59 | C83-465 | C83-466 | C83-467 | C83-468 | C83-469 | C83-470 | C83-471 | C83-472 |
| C81-60 | C83-473 | C83-474 | C83-475 | C83-476 | C83-477 | C83-478 | C83-479 | C83-480 |
| C81-61 | C83-481 | C83-482 | C83-483 | C83-484 | C83-485 | C83-486 | C83-487 | C83-488 |
| C81-62 | C83-489 | C83-490 | C83-491 | C83-492 | C83-493 | C83-494 | C83-495 | C83-496 |
| C81-63 | C83-497 | C83-498 | C83-499 | C83-500 | C83-501 | C83-502 | C83-503 | C83-504 |
| C81-64 | C83-505 | C83-506 | C83-507 | C83-508 | C83-509 | C83-510 | C83-511 | C83-512 |
| C81-65 | C83-513 | C83-514 | C83-515 | C83-516 | C83-517 | C83-518 | C83-519 | C83-520 |
| C81-66 | C83-521 | C83-522 | C83-523 | C83-524 | C83-525 | C83-526 | C83-527 | C83-528 |
| C81-67 | C83-529 | C83-530 | C83-531 | C83-532 | C83-533 | C83-534 | C83-535 | C83-536 |
| C81-68 | C83-537 | C83-538 | C83-539 | C83-540 | C83-541 | C83-542 | C83-543 | C83-544 |
| C81-69 | C83-545 | C83-546 | C83-547 | C83-548 | C83-549 | C83-550 | C83-551 | C83-552 |
| C81-70 | C83-553 | C83-554 | C83-555 | C83-556 | C83-557 | C83-558 | C83-559 | C83-560 |
| C81-71 | C83-561 | C83-562 | C83-563 | C83-564 | C83-565 | C83-566 | C83-567 | C83-568 |
| C81-72 | C83-569 | C83-570 | C83-571 | C83-572 | C83-573 | C83-574 | C83-575 | C83-576 |
| C81-73 | C83-577 | C83-578 | C83-579 | C83-580 | C83-581 | C83-582 | C83-583 | C83-584 |
| C81-74 | C83-585 | C83-586 | C83-587 | C83-588 | C83-589 | C83-590 | C83-591 | C83-592 |
| C81-75 | C83-593 | C83-594 | C83-595 | C83-596 | C83-597 | C83-598 | C83-599 | C83-600 |
| C81-76 | C83-601 | C83-602 | C83-603 | C83-604 | C83-605 | C83-606 | C83-607 | C83-608 |
| C81-77 | C83-609 | C83-610 | C83-611 | C83-612 | C83-613 | C83-614 | C83-615 | C83-616 |
| C81-78 | C83-617 | C83-618 | C83-619 | C83-620 | C83-621 | C83-622 | C83-623 | C83-624 |
| C81-79 | C83-625 | C83-626 | C83-627 | C83-628 | C83-629 | C83-630 | C83-631 | C83-632 |
| C81-80 | C83-633 | C83-634 | C83-635 | C83-636 | C83-637 | C83-638 | C83-639 | C83-640 |
| C81-81 | C83-641 | C83-642 | C83-643 | C83-644 | C83-645 | C83-646 | C83-647 | C83-648 |
| C81-82 | C83-649 | C83-650 | C83-651 | C83-652 | C83-653 | C83-654 | C83-655 | C83-656 |
| C81-83 | C83-657 | C83-658 | C83-659 | C83-660 | C83-661 | C83-662 | C83-663 | C83-664 |
| C81-84 | C83-665 | C83-666 | C83-667 | C83-668 | C83-669 | C83-670 | C83-671 | C83-672 |
| C81-85 | C83-673 | C83-674 | C83-675 | C83-676 | C83-677 | C83-678 | C83-679 | C83-680 |
| C81-86 | C83-681 | C83-682 | C83-683 | C83-684 | C83-685 | C83-686 | C83-687 | C83-688 |
| C81-87 | C83-689 | C83-690 | C83-691 | C83-692 | C83-693 | C83-694 | C83-695 | C83-696 |
| C81-88 | C83-697 | C83-698 | C83-699 | C83-700 | C83-701 | C83-702 | C83-703 | C83-704 |
| C81-89 | C83-705 | C83-706 | C83-707 | C83-708 | C83-709 | C83-710 | C83-711 | C83-712 |
| C81-90 | C83-713 | C83-714 | C83-715 | C83-716 | C83-717 | C83-718 | C83-719 | C83-720 |
| C81-91 | C83-721 | C83-722 | C83-723 | C83-724 | C83-725 | C83-726 | C83-727 | C83-728 |
| C81-92 | C83-729 | C83-730 | C83-731 | C83-732 | C83-733 | C83-734 | C83-735 | C83-736 |
| C81-93 | C83-737 | C83-738 | C83-739 | C83-740 | C83-741 | C83-742 | C83-743 | C83-744 |
| C81-94 | C83-745 | C83-746 | C83-747 | C83-748 | C83-749 | C83-750 | C83-751 | C83-752 |
| C81-95 | C83-753 | C83-754 | C83-755 | C83-756 | C83-757 | C83-758 | C83-759 | C83-760 |
| C81-96 | C83-761 | C83-762 | C83-763 | C83-764 | C83-765 | C83-766 | C83-767 | C83-768 |
| C81-97 | C83-769 | C83-770 | C83-771 | C83-772 | C83-773 | C83-774 | C83-775 | C83-776 |
| C81-98 | C83-777 | C83-778 | C83-779 | C83-780 | C83-781 | C83-782 | C83-783 | C83-784 |
| C81-99 | C83-785 | C83-786 | C83-787 | C83-788 | C83-789 | C83-790 | C83-791 | C83-792 |
| C81-100 | C83-793 | C83-794 | C83-795 | C83-796 | C83-797 | C83-798 | C83-799 | C83-800 |
| C81-101 | C83-801 | C83-802 | C83-803 | C83-804 | C83-805 | C83-806 | C83-807 | C83-808 |
| C81-102 | C83-809 | C83-810 | C83-811 | C83-812 | C83-813 | C83-814 | C83-815 | C83-816 |
| C81-103 | C83-817 | C83-818 | C83-819 | C83-820 | C83-821 | C83-822 | C83-823 | C83-824 |
| C81-104 | C83-825 | C83-826 | C83-827 | C83-828 | C83-829 | C83-830 | C83-831 | C83-832 |
| C81-105 | C83-833 | C83-834 | C83-835 | C83-836 | C83-837 | C83-838 | C83-839 | C83-840 |
| C81-106 | C83-841 | C83-842 | C83-843 | C83-844 | C83-845 | C83-846 | C83-847 | C83-848 |
| C81-107 | C83-849 | C83-850 | C83-851 | C83-852 | C83-853 | C83-854 | C83-855 | C83-856 |
| C81-108 | C83-857 | C83-858 | C83-859 | C83-860 | C83-861 | C83-862 | C83-863 | C83-864 |
| C81-109 | C83-865 | C83-866 | C83-867 | C83-868 | C83-869 | C83-870 | C83-871 | C83-872 |
| C81-110 | C83-873 | C83-874 | C83-875 | C83-876 | C83-877 | C83-878 | C83-879 | C83-880 |
| C81-111 | C83-881 | C83-882 | C83-883 | C83-884 | C83-885 | C83-886 | C83-887 | C83-888 |
| C81-112 | C83-889 | C83-890 | C83-891 | C83-892 | C83-893 | C83-894 | C83-895 | C83-896 |
| C81-113 | C83-897 | C83-898 | C83-899 | C83-900 | C83-901 | C83-902 | C83-903 | C83-904 |
| C81-114 | C83-905 | C83-906 | C83-907 | C83-908 | C83-909 | C83-910 | C83-911 | C83-912 |
| C81-115 | C83-913 | C83-914 | C83-915 | C83-916 | C83-917 | C83-918 | C83-919 | C83-920 |
| C81-116 | C83-921 | C83-922 | C83-923 | C83-924 | C83-925 | C83-926 | C83-927 | C83-928 |
| C81-117 | C83-929 | C83-930 | C83-931 | C83-932 | C83-933 | C83-934 | C83-935 | C83-936 |
| C81-118 | C83-937 | C83-938 | C83-939 | C83-940 | C83-941 | C83-942 | C83-943 | C83-944 |
| C81-119 | C83-945 | C83-946 | C83-947 | C83-948 | C83-949 | C83-950 | C83-951 | C83-952 |
| C81-120 | C83-953 | C83-954 | C83-955 | C83-956 | C83-957 | C83-958 | C83-959 | C83-960 |
| C81-121 | C83-961 | C83-962 | C83-963 | C83-964 | C83-965 | C83-966 | C83-967 | C83-968 |
| C81-122 | C83-969 | C83-970 | C83-971 | C83-972 | C83-973 | C83-974 | C83-975 | C83-976 |
| C81-123 | C83-977 | C83-978 | C83-979 | C83-980 | C83-981 | C83-982 | C83-983 | C83-984 |
| C81-124 | C83-985 | C83-986 | C83-987 | C83-988 | C83-989 | C83-990 | C83-991 | C83-992 |
| C81-125 | C83-993 | C83-994 | C83-995 | C83-996 | C83-997 | C83-998 | C83-999 | C83-1000 |
| C81-126 | C83-1001 | C83-1002 | C83-1003 | C83-1004 | C83-1005 | C83-1006 | C83-1007 | C83-1008 |
| C81-127 | C83-1009 | C83-1010 | C83-1011 | C83-1012 | C83-1013 | C83-1014 | C83-1015 | C83-1016 |
| C81-128 | C83-1017 | C83-1018 | C83-1019 | C83-1020 | C83-1021 | C83-1022 | C83-1023 | C83-1024 |
| C81-129 | C83-1025 | C83-1026 | C83-1027 | C83-1028 | C83-1029 | C83-1030 | C83-1031 | C83-1032 |
| C81-130 | C83-1033 | C83-1034 | C83-1035 | C83-1036 | C83-1037 | C83-1038 | C83-1039 | C83-1040 |
| C81-131 | C83-1041 | C83-1042 | C83-1043 | C83-1044 | C83-1045 | C83-1046 | C83-1047 | C83-1048 |
| C81-132 | C83-1049 | C83-1050 | C83-1051 | C83-1052 | C83-1053 | C83-1054 | C83-1055 | C83-1056 |
| C81-133 | C83-1057 | C83-1058 | C83-1059 | C83-1060 | C83-1061 | C83-1062 | C83-1063 | C83-1064 |
| C81-134 | C83-1065 | C83-1066 | C83-1067 | C83-1068 | C83-1069 | C83-1070 | C83-1071 | C83-1072 |
| C81-135 | C83-1073 | C83-1074 | C83-1075 | C83-1076 | C83-1077 | C83-1078 | C83-1079 | C83-1080 |
| C81-136 | C83-1081 | C83-1082 | C83-1083 | C83-1084 | C83-1085 | C83-1086 | C83-1087 | C83-1088 |
| C81-137 | C83-1089 | C83-1090 | C83-1091 | C83-1092 | C83-1093 | C83-1094 | C83-1095 | C83-1096 |
| C81-138 | C83-1097 | C83-1098 | C83-1099 | C83-1100 | C83-1101 | C83-1102 | C83-1103 | C83-1104 |
| C81-139 | C83-1105 | C83-1106 | C83-1107 | C83-1108 | C83-1109 | C83-1110 | C83-1111 | C83-1112 |
| C81-140 | C83-1113 | C83-1114 | C83-1115 | C83-1116 | C83-1117 | C83-1118 | C83-1119 | C83-1120 |
| C81-141 | C83-1121 | C83-1122 | C83-1123 | C83-1124 | C83-1125 | C83-1126 | C83-1127 | C83-1128 |
| C81-142 | C83-1129 | C83-1130 | C83-1131 | C83-1132 | C83-1133 | C83-1134 | C83-1135 | C83-1136 |
| C81-143 | C83-1137 | C83-1138 | C83-1139 | C83-1140 | C83-1141 | C83-1142 | C83-1143 | C83-1144 |
| C81-144 | C83-1145 | C83-1146 | C83-1147 | C83-1148 | C83-1149 | C83-1150 | C83-1151 | C83-1152 |
| C81-145 | C83-1153 | C83-1154 | C83-1155 | C83-1156 | C83-1157 | C83-1158 | C83-1159 | C83-1160 |
| C81-146 | C83-1161 | C83-1162 | C83-1163 | C83-1164 | C83-1165 | C83-1166 | C83-1167 | C83-1168 |
| C81-147 | C83-1169 | C83-1170 | C83-1171 | C83-1172 | C83-1173 | C83-1174 | C83-1175 | C83-1176 |
| C81-148 | C83-1177 | C83-1178 | C83-1179 | C83-1180 | C83-1181 | C83-1182 | C83-1183 | C83-1184 |
| C81-149 | C83-1185 | C83-1186 | C83-1187 | C83-1188 | C83-1189 | C83-1190 | C83-1191 | C83-1192 |
| C81-150 | C83-1193 | C83-1194 | C83-1195 | C83-1196 | C83-1197 | C83-1198 | C83-1199 | C83-1200 |
| C81-151 | C83-1201 | C83-1202 | C83-1203 | C83-1204 | C83-1205 | C83-1206 | C83-1207 | C83-1208 |
| C81-152 | C83-1209 | C83-1210 | C83-1211 | C83-1212 | C83-1213 | C83-1214 | C83-1215 | C83-1216 |
| C81-153 | C83-1217 | C83-1218 | C83-1219 | C83-1220 | C83-1221 | C83-1222 | C83-1223 | C83-1224 |

In other embodiments of the antiparasitic combinations of the invention, the antiparasitic combinations C82-1 to C82-153 described in Table 3 above containing combinations of compound IC-1, isoxazolines Is-1 to Is-9 and macrocyclic lactones MIL-1 to MIL-17 are further combined with anticestodal active agents Ac-1 to Ac-8 to provide further antiparasitic combinations C84-1 to C84-1224 as shown in Table 5 below.

TABLE 5

| | Ac-1 | Ac-2 | Ac-3 | Ac-4 | Ac-5 | Ac-6 | Ac-7 | Ac-8 |
|---|---|---|---|---|---|---|---|---|
| C82-1 | C84-1 | C84-2 | C84-3 | C84-4 | C84-5 | C84-6 | C84-7 | C84-8 |
| C82-2 | C84-9 | C84-10 | C84-11 | C84-12 | C84-13 | C84-14 | C84-15 | C84-16 |
| C82-3 | C84-17 | C84-18 | C84-19 | C84-20 | C84-21 | C84-22 | C84-23 | C84-24 |
| C82-4 | C84-25 | C84-26 | C84-27 | C84-28 | C84-29 | C84-30 | C84-31 | C84-32 |
| C82-5 | C84-33 | C84-34 | C84-35 | C84-36 | C84-37 | C84-38 | C84-39 | C84-40 |
| C82-6 | C84-41 | C84-42 | C84-43 | C84-44 | C84-45 | C84-46 | C84-47 | C84-48 |
| C82-7 | C84-49 | C84-50 | C84-51 | C84-52 | C84-53 | C84-54 | C84-55 | C84-56 |
| C82-8 | C84-57 | C84-58 | C84-59 | C84-60 | C84-61 | C84-62 | C84-63 | C84-64 |
| C82-9 | C84-65 | C84-66 | C84-67 | C84-68 | C84-69 | C84-70 | C84-71 | C84-72 |
| C82-10 | C84-73 | C84-74 | C84-75 | C84-76 | C84-77 | C84-78 | C84-79 | C84-80 |
| C82-11 | C84-81 | C84-82 | C84-83 | C84-84 | C84-85 | C84-86 | C84-87 | C84-88 |
| C82-12 | C84-89 | C84-90 | C84-91 | C84-92 | C84-93 | C84-94 | C84-95 | C84-96 |
| C82-13 | C84-97 | C84-98 | C84-99 | C84-100 | C84-101 | C84-102 | C84-103 | C84-104 |
| C82-14 | C84-105 | C84-106 | C84-107 | C84-108 | C84-109 | C84-110 | C84-111 | C84-112 |
| C82-15 | C84-113 | C84-114 | C84-115 | C84-116 | C84-117 | C84-118 | C84-119 | C84-120 |
| C82-16 | C84-121 | C84-122 | C84-123 | C84-124 | C84-125 | C84-126 | C84-127 | C84-128 |
| C82-17 | C84-129 | C84-130 | C84-131 | C84-132 | C84-133 | C84-134 | C84-135 | C84-136 |
| C82-18 | C84-137 | C84-138 | C84-139 | C84-140 | C84-141 | C84-142 | C84-143 | C84-144 |
| C82-19 | C84-145 | C84-146 | C84-147 | C84-148 | C84-149 | C84-150 | C84-151 | C84-152 |
| C82-20 | C84-153 | C84-154 | C84-155 | C84-156 | C84-157 | C84-158 | C84-159 | C84-160 |
| C82-21 | C84-161 | C84-162 | C84-163 | C84-164 | C84-165 | C84-166 | C84-167 | C84-168 |
| C82-22 | C84-169 | C84-170 | C84-171 | C84-172 | C84-173 | C84-174 | C84-175 | C84-176 |
| C82-23 | C84-177 | C84-178 | C84-179 | C84-180 | C84-181 | C84-182 | C84-183 | C84-184 |
| C82-24 | C84-185 | C84-186 | C84-187 | C84-188 | C84-189 | C84-190 | C84-191 | C84-192 |
| C82-25 | C84-193 | C84-194 | C84-195 | C84-196 | C84-197 | C84-198 | C84-199 | C84-200 |
| C82-26 | C84-201 | C84-202 | C84-203 | C84-204 | C84-205 | C84-206 | C84-207 | C84-208 |
| C82-27 | C84-209 | C84-210 | C84-211 | C84-212 | C84-213 | C84-214 | C84-215 | C84-216 |
| C82-28 | C84-217 | C84-218 | C84-219 | C84-220 | C84-221 | C84-222 | C84-223 | C84-224 |
| C82-29 | C84-225 | C84-226 | C84-227 | C84-228 | C84-229 | C84-230 | C84-231 | C84-232 |
| C82-30 | C84-233 | C84-234 | C84-235 | C84-236 | C84-237 | C84-238 | C84-239 | C84-240 |
| C82-31 | C84-241 | C84-242 | C84-243 | C84-244 | C84-245 | C84-246 | C84-247 | C84-248 |
| C82-32 | C84-249 | C84-250 | C84-251 | C84-252 | C84-253 | C84-254 | C84-255 | C84-256 |
| C82-33 | C84-257 | C84-258 | C84-259 | C84-260 | C84-261 | C84-262 | C84-263 | C84-264 |
| C82-34 | C84-265 | C84-266 | C84-267 | C84-268 | C84-269 | C84-270 | C84-271 | C84-272 |
| C82-35 | C84-273 | C84-274 | C84-275 | C84-276 | C84-277 | C84-278 | C84-279 | C84-280 |
| C82-36 | C84-281 | C84-282 | C84-283 | C84-284 | C84-285 | C84-286 | C84-287 | C84-288 |
| C82-37 | C84-289 | C84-290 | C84-291 | C84-292 | C84-293 | C84-294 | C84-295 | C84-296 |
| C82-38 | C84-297 | C84-298 | C84-299 | C84-300 | C84-301 | C84-302 | C84-303 | C84-304 |
| C82-39 | C84-305 | C84-306 | C84-307 | C84-308 | C84-309 | C84-310 | C84-311 | C84-312 |
| C82-40 | C84-313 | C84-314 | C84-315 | C84-316 | C84-317 | C84-318 | C84-319 | C84-320 |
| C82-41 | C84-321 | C84-322 | C84-323 | C84-324 | C84-325 | C84-326 | C84-327 | C84-328 |
| C82-42 | C84-329 | C84-330 | C84-331 | C84-332 | C84-333 | C84-334 | C84-335 | C84-336 |
| C82-43 | C84-337 | C84-338 | C84-339 | C84-340 | C84-341 | C84-342 | C84-343 | C84-344 |
| C82-44 | C84-345 | C84-346 | C84-347 | C84-348 | C84-349 | C84-350 | C84-351 | C84-352 |
| C82-45 | C84-353 | C84-354 | C84-355 | C84-356 | C84-357 | C84-358 | C84-359 | C84-360 |
| C82-46 | C84-361 | C84-362 | C84-363 | C84-364 | C84-365 | C84-366 | C84-367 | C84-368 |
| C82-47 | C84-369 | C84-370 | C84-371 | C84-372 | C84-373 | C84-374 | C84-375 | C84-376 |
| C82-48 | C84-377 | C84-378 | C84-379 | C84-380 | C84-381 | C84-382 | C84-383 | C84-384 |
| C82-49 | C84-385 | C84-386 | C84-387 | C84-388 | C84-389 | C84-390 | C84-391 | C84-392 |
| C82-50 | C84-393 | C84-394 | C84-395 | C84-396 | C84-397 | C84-398 | C84-399 | C84-400 |
| C82-51 | C84-401 | C84-402 | C84-403 | C84-404 | C84-405 | C84-406 | C84-407 | C84-408 |
| C82-52 | C84-409 | C84-410 | C84-411 | C84-412 | C84-413 | C84-414 | C84-415 | C84-416 |
| C82-53 | C84-417 | C84-418 | C84-419 | C84-420 | C84-421 | C84-422 | C84-423 | C84-424 |
| C82-54 | C84-425 | C84-426 | C84-427 | C84-428 | C84-429 | C84-430 | C84-431 | C84-432 |
| C82-55 | C84-433 | C84-434 | C84-435 | C84-436 | C84-437 | C84-438 | C84-439 | C84-440 |
| C82-56 | C84-441 | C84-442 | C84-443 | C84-444 | C84-445 | C84-446 | C84-447 | C84-448 |
| C82-57 | C84-449 | C84-450 | C84-451 | C84-452 | C84-453 | C84-454 | C84-455 | C84-456 |
| C82-58 | C84-457 | C84-458 | C84-459 | C84-460 | C84-461 | C84-462 | C84-463 | C84-464 |
| C82-59 | C84-465 | C84-466 | C84-467 | C84-468 | C84-469 | C84-470 | C84-471 | C84-472 |
| C82-60 | C84-473 | C84-474 | C84-475 | C84-476 | C84-477 | C84-478 | C84-479 | C84-480 |
| C82-61 | C84-481 | C84-482 | C84-483 | C84-484 | C84-485 | C84-486 | C84-487 | C84-488 |
| C82-62 | C84-489 | C84-490 | C84-491 | C84-492 | C84-493 | C84-494 | C84-495 | C84-496 |
| C82-63 | C84-497 | C84-498 | C84-499 | C84-500 | C84-501 | C84-502 | C84-503 | C84-504 |
| C82-64 | C84-505 | C84-506 | C84-507 | C84-508 | C84-509 | C84-510 | C84-511 | C84-512 |
| C82-65 | C84-513 | C84-514 | C84-515 | C84-516 | C84-517 | C84-518 | C84-519 | C84-520 |
| C82-66 | C84-521 | C84-522 | C84-523 | C84-524 | C84-525 | C84-526 | C84-527 | C84-528 |
| C82-67 | C84-529 | C84-530 | C84-531 | C84-532 | C84-533 | C84-534 | C84-535 | C84-536 |
| C82-68 | C84-537 | C84-538 | C84-539 | C84-540 | C84-541 | C84-542 | C84-543 | C84-544 |
| C82-69 | C84-545 | C84-546 | C84-547 | C84-548 | C84-549 | C84-550 | C84-551 | C84-552 |
| C82-70 | C84-553 | C84-554 | C84-555 | C84-556 | C84-557 | C84-558 | C84-559 | C84-560 |
| C82-71 | C84-561 | C84-562 | C84-563 | C84-564 | C84-565 | C84-566 | C84-567 | C84-568 |
| C82-72 | C84-569 | C84-570 | C84-571 | C84-572 | C84-573 | C84-574 | C84-575 | C84-576 |
| C82-73 | C84-577 | C84-578 | C84-579 | C84-580 | C84-581 | C84-582 | C84-583 | C84-584 |
| C82-74 | C84-585 | C84-586 | C84-587 | C84-588 | C84-589 | C84-590 | C84-591 | C84-592 |
| C82-75 | C84-593 | C84-594 | C84-595 | C84-596 | C84-597 | C84-598 | C84-599 | C84-600 |
| C82-76 | C84-601 | C84-602 | C84-603 | C84-604 | C84-605 | C84-606 | C84-607 | C84-608 |
| C82-77 | C84-609 | C84-610 | C84-611 | C84-612 | C84-613 | C84-614 | C84-615 | C84-616 |
| C82-78 | C84-617 | C84-618 | C84-619 | C84-620 | C84-621 | C84-622 | C84-623 | C84-624 |
| C82-79 | C84-625 | C84-626 | C84-627 | C84-628 | C84-629 | C84-630 | C84-631 | C84-632 |
| C82-80 | C84-633 | C84-634 | C84-635 | C84-636 | C84-637 | C84-638 | C84-639 | C84-640 |
| C82-81 | C84-641 | C84-642 | C84-643 | C84-644 | C84-645 | C84-646 | C84-647 | C84-648 |
| C82-82 | C84-649 | C84-650 | C84-651 | C84-652 | C84-653 | C84-654 | C84-655 | C84-656 |
| C82-83 | C84-657 | C84-658 | C84-659 | C84-660 | C84-661 | C84-662 | C84-663 | C84-664 |
| C82-84 | C84-665 | C84-666 | C84-667 | C84-668 | C84-669 | C84-670 | C84-671 | C84-672 |
| C82-85 | C84-673 | C84-674 | C84-675 | C84-676 | C84-677 | C84-678 | C84-679 | C84-680 |
| C82-86 | C84-681 | C84-682 | C84-683 | C84-684 | C84-685 | C84-686 | C84-687 | C84-688 |
| C82-87 | C84-689 | C84-690 | C84-691 | C84-692 | C84-693 | C84-694 | C84-695 | C84-696 |
| C82-88 | C84-697 | C84-698 | C84-699 | C84-700 | C84-701 | C84-702 | C84-703 | C84-704 |
| C82-89 | C84-705 | C84-706 | C84-707 | C84-708 | C84-709 | C84-710 | C84-711 | C84-712 |
| C82-90 | C84-713 | C84-714 | C84-715 | C84-716 | C84-717 | C84-718 | C84-719 | C84-720 |
| C82-91 | C84-721 | C84-722 | C84-723 | C84-724 | C84-725 | C84-726 | C84-727 | C84-728 |
| C82-92 | C84-729 | C84-730 | C84-731 | C84-732 | C84-733 | C84-734 | C84-735 | C84-736 |
| C82-93 | C84-737 | C84-738 | C84-739 | C84-740 | C84-741 | C84-742 | C84-743 | C84-744 |
| C82-94 | C84-745 | C84-746 | C84-747 | C84-748 | C84-749 | C84-750 | C84-751 | C84-752 |
| C82-95 | C84-753 | C84-754 | C84-755 | C84-756 | C84-757 | C84-758 | C84-759 | C84-760 |
| C82-96 | C84-761 | C84-762 | C84-763 | C84-764 | C84-765 | C84-766 | C84-767 | C84-768 |
| C82-97 | C84-769 | C84-770 | C84-771 | C84-772 | C84-773 | C84-774 | C84-775 | C84-776 |
| C82-98 | C84-777 | C84-778 | C84-779 | C84-780 | C84-781 | C84-782 | C84-783 | C84-784 |
| C82-99 | C84-785 | C84-786 | C84-787 | C84-788 | C84-789 | C84-790 | C84-791 | C84-792 |
| C82-100 | C84-793 | C84-794 | C84-795 | C84-796 | C84-797 | C84-798 | C84-799 | C84-800 |
| C82-101 | C84-801 | C84-802 | C84-803 | C84-804 | C84-805 | C84-806 | C84-807 | C84-808 |
| C82-102 | C84-809 | C84-810 | C84-811 | C84-812 | C84-813 | C84-814 | C84-815 | C84-816 |
| C82-103 | C84-817 | C84-818 | C84-819 | C84-820 | C84-821 | C84-822 | C84-823 | C84-824 |
| C82-104 | C84-825 | C84-826 | C84-827 | C84-828 | C84-829 | C84-830 | C84-831 | C84-832 |
| C82-105 | C84-833 | C84-834 | C84-835 | C84-836 | C84-837 | C84-838 | C84-839 | C84-840 |
| C82-106 | C84-841 | C84-842 | C84-843 | C84-844 | C84-845 | C84-846 | C84-847 | C84-848 |
| C82-107 | C84-849 | C84-850 | C84-851 | C84-852 | C84-853 | C84-854 | C84-855 | C84-856 |
| C82-108 | C84-857 | C84-858 | C84-859 | C84-860 | C84-861 | C84-862 | C84-863 | C84-864 |
| C82-109 | C84-865 | C84-866 | C84-867 | C84-868 | C84-869 | C84-870 | C84-871 | C84-872 |
| C82-110 | C84-873 | C84-874 | C84-875 | C84-876 | C84-877 | C84-878 | C84-879 | C84-880 |
| C82-111 | C84-881 | C84-882 | C84-883 | C84-884 | C84-885 | C84-886 | C84-887 | C84-888 |
| C82-112 | C84-889 | C84-890 | C84-891 | C84-892 | C84-893 | C84-894 | C84-895 | C84-896 |
| C82-113 | C84-897 | C84-898 | C84-899 | C84-900 | C84-901 | C84-902 | C84-903 | C84-904 |
| C82-114 | C84-905 | C84-906 | C84-907 | C84-908 | C84-909 | C84-910 | C84-911 | C84-912 |
| C82-115 | C84-913 | C84-914 | C84-915 | C84-916 | C84-917 | C84-918 | C84-919 | C84-920 |
| C82-116 | C84-921 | C84-922 | C84-923 | C84-924 | C84-925 | C84-926 | C84-927 | C84-928 |
| C82-117 | C84-929 | C84-930 | C84-931 | C84-932 | C84-933 | C84-934 | C84-935 | C84-936 |
| C82-118 | C84-937 | C84-938 | C84-939 | C84-940 | C84-941 | C84-942 | C84-943 | C84-944 |
| C82-119 | C84-945 | C84-946 | C84-947 | C84-948 | C84-949 | C84-950 | C84-951 | C84-952 |
| C82-120 | C84-953 | C84-954 | C84-955 | C84-956 | C84-957 | C84-958 | C84-959 | C84-960 |
| C82-121 | C84-961 | C84-962 | C84-963 | C84-964 | C84-965 | C84-966 | C84-967 | C84-968 |
| C82-122 | C84-969 | C84-970 | C84-971 | C84-972 | C84-973 | C84-974 | C84-975 | C84-976 |
| C82-123 | C84-977 | C84-978 | C84-979 | C84-980 | C84-981 | C84-982 | C84-983 | C84-984 |
| C82-124 | C84-985 | C84-986 | C84-987 | C84-988 | C84-989 | C84-990 | C84-991 | C84-992 |
| C82-125 | C84-993 | C84-994 | C84-995 | C84-996 | C84-997 | C84-998 | C84-999 | C84-1000 |
| C82-126 | C84-1001 | C84-1002 | C84-1003 | C84-1004 | C84-1005 | C84-1006 | C84-1007 | C84-1008 |
| C82-127 | C84-1009 | C84-1010 | C84-1011 | C84-1012 | C84-1013 | C84-1014 | C84-1015 | C84-1016 |
| C82-128 | C84-1017 | C84-1018 | C84-1019 | C84-1020 | C84-1021 | C84-1022 | C84-1023 | C84-1024 |
| C82-129 | C84-1025 | C84-1026 | C84-1027 | C84-1028 | C84-1029 | C84-1030 | C84-1031 | C84-1032 |
| C82-130 | C84-1033 | C84-1034 | C84-1035 | C84-1036 | C84-1037 | C84-1038 | C84-1039 | C84-1040 |
| C82-131 | C84-1041 | C84-1042 | C84-1043 | C84-1044 | C84-1045 | C84-1046 | C84-1047 | C84-1048 |
| C82-132 | C84-1049 | C84-1050 | C84-1051 | C84-1052 | C84-1053 | C84-1054 | C84-1055 | C84-1056 |
| C82-133 | C84-1057 | C84-1058 | C84-1059 | C84-1060 | C84-1061 | C84-1062 | C84-1063 | C84-1064 |
| C82-134 | C84-1065 | C84-1066 | C84-1067 | C84-1068 | C84-1069 | C84-1070 | C84-1071 | C84-1072 |
| C82-135 | C84-1073 | C84-1074 | C84-1075 | C84-1076 | C84-1077 | C84-1078 | C84-1079 | C84-1080 |
| C82-136 | C84-1081 | C84-1082 | C84-1083 | C84-1084 | C84-1085 | C84-1086 | C84-1087 | C84-1088 |
| C82-137 | C84-1089 | C84-1090 | C84-1091 | C84-1092 | C84-1093 | C84-1094 | C84-1095 | C84-1096 |
| C82-138 | C84-1097 | C84-1098 | C84-1099 | C84-1100 | C84-1101 | C84-1102 | C84-1103 | C84-1104 |
| C82-139 | C84-1105 | C84-1106 | C84-1107 | C84-1108 | C84-1109 | C84-1110 | C84-1111 | C84-1112 |
| C82-140 | C84-1113 | C84-1114 | C84-1115 | C84-1116 | C84-1117 | C84-1118 | C84-1119 | C84-1120 |
| C82-141 | C84-1121 | C84-1122 | C84-1123 | C84-1124 | C84-1125 | C84-1126 | C84-1127 | C84-1128 |
| C82-142 | C84-1129 | C84-1130 | C84-1131 | C84-1132 | C84-1133 | C84-1134 | C84-1135 | C84-1136 |
| C82-143 | C84-1137 | C84-1138 | C84-1139 | C84-1140 | C84-1141 | C84-1142 | C84-1143 | C84-1144 |
| C82-144 | C84-1145 | C84-1146 | C84-1147 | C84-1148 | C84-1149 | C84-1150 | C84-1151 | C84-1152 |
| C82-145 | C84-1153 | C84-1154 | C84-1155 | C84-1156 | C84-1157 | C84-1158 | C84-1159 | C84-1160 |
| C82-146 | C84-1161 | C84-1162 | C84-1163 | C84-1164 | C84-1165 | C84-1166 | C84-1167 | C84-1168 |
| C82-147 | C84-1169 | C84-1170 | C84-1171 | C84-1172 | C84-1173 | C84-1174 | C84-1175 | C84-1176 |
| C82-148 | C84-1177 | C84-1178 | C84-1179 | C84-1180 | C84-1181 | C84-1182 | C84-1183 | C84-1184 |
| C82-149 | C84-1185 | C84-1186 | C84-1187 | C84-1188 | C84-1189 | C84-1190 | C84-1191 | C84-1192 |
| C82-150 | C84-1193 | C84-1194 | C84-1195 | C84-1196 | C84-1197 | C84-1198 | C84-1199 | C84-1200 |
| C82-151 | C84-1201 | C84-1202 | C84-1203 | C84-1204 | C84-1205 | C84-1206 | C84-1207 | C84-1208 |
| C82-152 | C84-1209 | C84-1210 | C84-1211 | C84-1212 | C84-1213 | C84-1214 | C84-1215 | C84-1216 |
| C82-153 | C84-1217 | C84-1218 | C84-1219 | C84-1220 | C84-1221 | C84-1222 | C84-1223 | C84-1224 |

In one embodiment, the antiparasitic combinations of the invention include one or more compounds of Formula (IA), or a salt thereof, and one or more isoxazoline active agents, including afoxolaner, esafoxolaner, umifoxolaner, fluralaner, (S)-fluralaner, sarolaner, lotilaner, mivorilaner or tigolaner.

In another embodiment, the antiparasitic combinations of the invention include one or more compounds of Formula (TB), or a salt thereof, and one or more isoxazoline active agents, including afoxolaner, esafoxolaner, umifoxolaner, fluralaner, (S)-fluralaner, sarolaner, lotilaner, mivorilaner or tigolaner.

In another embodiment, the antiparasitic combinations of the invention include one or more compounds of Formula (IC), or a salt thereof, and one or more isoxazoline active agents, including afoxolaner, esafoxolaner, umifoxolaner, fluralaner, (S)-fluralaner, sarolaner, lotilaner, mivorilaner or tigolaner.

In one embodiment, the antiparasitic combinations of the invention include one or more compounds of Formula (IA), or a salt thereof, and one or macrocyclic lactones.

In one embodiment, the antiparasitic combinations of the invention include one or more compounds of Formula (IB), or a salt thereof, and one or macrocyclic lactones.

In one embodiment, the antiparasitic combinations of the invention include one or more compounds of Formula (IC), or a salt thereof, and one or macrocyclic lactones.

In one embodiment, the antiparasitic combinations of the invention include one or more compounds of Formula (IA), or a salt thereof, and milbemycin oxime.

In one embodiment, the antiparasitic combinations of the invention include one or more compounds of Formula (IB), or a salt thereof, and milbemycin oxime.

In one embodiment, the antiparasitic combinations of the invention include one or more compounds of Formula (IC), or a salt thereof, and milbemycin oxime.

In one embodiment, the antiparasitic combinations of the invention include one or more compounds of Formula (IA), or a salt thereof, and moxidectin.

In one embodiment, the antiparasitic combinations of the invention include one or more compounds of Formula (IB), or a salt thereof, and moxidectin.

In one embodiment, the antiparasitic combinations of the invention include one or more compounds of Formula (IC), or a salt thereof, and moxidectin.

In one embodiment, the antiparasitic combinations of the invention include one or more compounds of Formula (IA), or a salt thereof, and eprinomectin.

In one embodiment, the antiparasitic combinations of the invention include one or more compounds of Formula (IB), or a salt thereof, and eprinomectin.

In one embodiment, the antiparasitic combinations of the invention include one or more compounds of Formula (IC), or a salt thereof, and eprinomectin.

In one embodiment, the antiparasitic combinations of the invention include one or more compounds of Formula (IA), or a salt thereof, and selamectin.

In one embodiment, the antiparasitic combinations of the invention include one or more compounds of Formula (IB), or a salt thereof, and selamectin.

In one embodiment, the antiparasitic combinations of the invention include one or more compounds of Formula (IC), or a salt thereof, and selamectin.

In one embodiment, the antiparasitic combinations of the invention include one or more compounds of Formula (IA), or a salt thereof, and praziquantel.

In one embodiment, the antiparasitic combinations of the invention include one or more compounds of Formula (IB), or a salt thereof, and praziquantel.

In one embodiment, the antiparasitic combinations of the invention include one or more compounds of Formula (IC), or a salt thereof, and praziquantel.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with afoxolaner, esafoxolaner, umifoxolaner, fluralaner, (S)-fluralaner, sarolaner, lotilaner, mivorilaner or tigolaner.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with afoxolaner, esafoxolaner, umifoxolaner or fluralaner.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with afoxolaner.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with esafoxolaner.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with umifoxolaner.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with fluralaner.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with milbemycin oxime.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with moxidectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with eprinomectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with selamectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with praziquantel.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with afoxolaner and moxidectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with esafoxolaner and moxidectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with umifoxolaner and moxidectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with fluralaner and moxidectin.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with afoxolaner, moxidectin and praziquantel.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with esafoxolaner, moxidectin and praziquantel.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with umifoxolaner, moxidectin and praziquantel.

In another embodiment, the antiparasitic combinations comprise a compound of Formula (IA), L is L1 wherein X is O and R′ is hydrogen; Q is C—R⁹; R is iso-propyl, tert-butyl, cyclopropyl, cyclobutyl, —N(CH₃)₂, azetidinyl or morpholinyl, each optionally substituted by one or more fluoro or methyl; R¹ is hydrogen; R² is methyl; R⁴ is hydrogen; R⁹ is hydrogen; R¹⁰ is chloro or fluoro and m is 3, wherein the chloro or fluoro substituents are substituted at the 2, 3 and 5 positions of the phenyl ring; W and W¹ are each CH₂; Z is O and Y¹, Y², Y³ and Y⁴ are each CH, together with fluralaner, moxidectin and praziquantel.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with afoxolaner, esafoxolaner, umifoxolaner, fluralaner, (S)-fluralaner, sarolaner, lotilaner, mivorilaner or tigolaner.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with lotilaner.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with moxidectin.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with milbemycin oxime.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with eprinomectin.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with selamectin.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with praziquantel.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with lotilaner and moxidectin.

In another embodiment, the antiparasitic combinations of the invention comprise a compound of Formula (IC), wherein L is L1 wherein X is O and R′ is hydrogen; Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl; R¹ is hydrogen; R² is hydrogen; R³ is hydrogen or fluoro; R⁴ is hydrogen; R⁹ is hydrogen; R¹⁰ is CF₃, chloro or fluoro and m is 2 or 3; W and W¹ are each CH₂; Z is O and Y¹, Y², Y³ and Y⁴ are each CH, together with lotilaner, moxidectin and praziquantel.

In another embodiment, the antiparasitic combinations of the invention comprise a compound of Formula (IC), wherein: L is L2 wherein X is O and R′ is hydrogen; Q is C—R⁹; R is morpholinyl, optionally substituted by one or more fluoro or methyl; R¹ is hydrogen; R² is hydrogen; R³ is fluoro; R⁴ is hydrogen; R⁹ is hydrogen; R¹⁰ is chloro or fluoro and m is 2, wherein the chloro or fluoro are substituted at the 2-, 3- or 5-positions of the phenyl ring; W is CH; W¹ is CH or C—CH₃; Z is N and Y¹, Y², Y³ and Y⁴ are each CH, or a salt thereof, together with afoxolaner, esafoxolaner, umifoxolaner, fluralaner, (S)-fluralaner, sarolaner, lotilaner, mivorilaner or tigolaner.

In another embodiment, the antiparasitic combinations of the invention comprise a compound of Formula (IC), wherein: L is L2 wherein X is O and R′ is hydrogen; Q is C—R⁹; R is morpholinyl, optionally substituted by one or more fluoro or methyl; R¹ is hydrogen; R² is hydrogen; R³ is fluoro; R⁴ is hydrogen; R⁹ is hydrogen; R¹⁰ is chloro or fluoro and m is 2, wherein the chloro or fluoro are substituted at the 2-, 3- or 5-positions of the phenyl ring; W is CH; W¹ is CH or C—CH₃; Z is N and Y¹, Y², Y³ and Y⁴ are each CH, or a salt thereof, together with fluralaner.

In another embodiment, the antiparasitic combinations of the invention comprise a compound of Formula (IC), wherein: L is L2 wherein X is O and R′ is hydrogen; Q is C—R⁹; R is morpholinyl, optionally substituted by one or more fluoro or methyl; R¹ is hydrogen; R² is hydrogen; R³ is fluoro; R⁴ is hydrogen; R⁹ is hydrogen; R¹⁰ is chloro or fluoro and m is 2, wherein the chloro or fluoro are substituted at the 2-, 3- or 5-positions of the phenyl ring; W is CH; W¹ is CH or C—CH₃; Z is N and Y¹, Y², Y³ and Y⁴ are each CH, or a salt thereof, together with fluralaner and moxidectin.

In another embodiment, the antiparasitic combinations of the invention comprise a compound of Formula (IC), wherein: L is L2 wherein X is O and R′ is hydrogen; Q is C—R⁹; R is morpholinyl, optionally substituted by one or more fluoro or methyl; R¹ is hydrogen; R² is hydrogen; R³ is fluoro; R⁴ is hydrogen; R⁹ is hydrogen; R¹⁰ is chloro or fluoro and m is 2, wherein the chloro or fluoro are substituted at the 2-, 3- or 5-positions of the phenyl ring; W is CH; W¹ is CH or C—CH₃; Z is N and Y¹, Y², Y³ and Y⁴ are each CH, or a salt thereof, together with fluralaner, moxidectin and praziquantel.

In one embodiment, the antiparasitic combinations of the invention include the compound A472, or a salt thereof, and one or more isoxazoline active agents, including afoxolaner, esafoxolaner, umifoxolaner, fluralaner, (S)-fluralaner, sarolaner, lotilaner, mivorilaner or tigolaner.

In another embodiment, the antiparasitic combinations of the invention include the compound IC-1, or a salt thereof, and one or more isoxazoline active agents, including afoxolaner, esafoxolaner, umifoxolaner, fluralaner, (S)-fluralaner, sarolaner, lotilaner, mivorilaner or tigolaner.

In another embodiment, the antiparasitic combinations of the invention include the compound A472, or a salt thereof, and afoxolaner.

In another embodiment, the antiparasitic combinations of the invention include the compound A472, or a salt thereof, and esafoxolaner.

In another embodiment, the antiparasitic combinations of the invention include the compound A472, or a salt thereof, and umifoxolaner.

In another embodiment, the antiparasitic combinations of the invention include the compound A472, or a salt thereof, and fluralaner.

In another embodiment, the antiparasitic combinations of the invention include the compound A472, or a salt thereof, and moxidectin.

In another embodiment, the antiparasitic combinations of the invention include the compound A472, or a salt thereof, and milbemycin oxime.

In another embodiment, the antiparasitic combinations of the invention include the compound A472, or a salt thereof, and eprinomectin.

In another embodiment, the antiparasitic combinations of the invention include the compound A472, or a salt thereof, and selamectin.

In another embodiment, the antiparasitic combinations of the invention include the compound A472, or a salt thereof, and praziquantel.

In another embodiment, the antiparasitic combinations of the invention include the compound IC-1, or a salt thereof, and lotilaner.

In another embodiment, the antiparasitic combinations of the invention include the compound IC-1, or a salt thereof, and afoxolaner.

In another embodiment, the antiparasitic combinations of the invention include the compound IC-1, or a salt thereof, and esafoxolaner.

In another embodiment, the antiparasitic combinations of the invention include the compound IC-1, or a salt thereof, and umifoxolaner.

In another embodiment, the antiparasitic combinations of the invention include the compound IC-1, or a salt thereof, and fluralaner.

In another embodiment, the antiparasitic combinations of the invention include the compound IC-1, or a salt thereof, and moxidectin.

In another embodiment, the antiparasitic combinations of the invention include the compound IC-1, or a salt thereof, and milbemycin oxime.

In another embodiment, the antiparasitic combinations of the invention include the compound IC-1, or a salt thereof, and eprinomectin.

In another embodiment, the antiparasitic combinations of the invention include the compound IC-1, or a salt thereof, and selamectin.

In another embodiment, the antiparasitic combinations of the invention include the compound IC-1, or a salt thereof, and praziquantel.

In another embodiment, the antiparasitic combinations of the invention include the compound A472, or a salt thereof, afoxolaner and moxidectin.

In another embodiment, the antiparasitic combinations of the invention include the compound A472, or a salt thereof, esafoxolaner and moxidectin.

In another embodiment, the antiparasitic combinations of the invention include the compound A472, or a salt thereof, umifoxolaner and moxidectin.

In another embodiment, the antiparasitic combinations of the invention include the compound A472, or a salt thereof, fluralaner and moxidectin.

In another embodiment, the antiparasitic combinations of the invention include the compound IC-1, or a salt thereof, afoxolaner and moxidectin.

In another embodiment, the antiparasitic combinations of the invention include the compound IC-1, or a salt thereof, esafoxolaner and moxidectin.

In another embodiment, the antiparasitic combinations of the invention include the compound IC-1, or a salt thereof, umifoxolaner and moxidectin.

In another embodiment, the antiparasitic combinations of the invention include the compound IC-1, or a salt thereof, fluralaner and moxidectin.

In another embodiment, the antiparasitic combinations of the invention include the compound IC-1, or a salt thereof, lotilaner and moxidectin.

In one embodiment, the antiparasitic combinations of the invention include the compound A472, or a salt thereof, combined with afoxolaner, moxidectin and praziquantel.

In one embodiment, the antiparasitic combinations of the invention include the compound A472, or a salt thereof, combined with esafoxolaner, moxidectin and praziquantel.

In one embodiment, the antiparasitic combinations of the invention include the compound A472, or a salt thereof, combined with umifoxolaner, moxidectin and praziquantel.

In another embodiment, the antiparasitic combinations of the invention include the compound A472, or a salt thereof, combined with fluralaner, moxidectin and praziquantel.

In one embodiment, the antiparasitic combinations of the invention include the compound A472, or a salt thereof, combined with afoxolaner, moxidectin and pyrantel.

In one embodiment, the antiparasitic combinations of the invention include the compound A472, or a salt thereof, combined with esafoxolaner, moxidectin and pyrantel.

In one embodiment, the antiparasitic combinations of the invention include the compound A472, or a salt thereof, combined with umifoxolaner, moxidectin and pyrantel.

In another embodiment, the antiparasitic combinations of the invention include the compound A472, or a salt thereof, combined with fluralaner, moxidectin and pyrantel.

In one embodiment, the antiparasitic combinations of the invention include the compound IC-1, or a salt thereof, combined with lotilaner, moxidectin and praziquantel.

In one embodiment, the antiparasitic combinations of the invention include the compound IC-1, or a salt thereof, combined with lotilaner, moxidectin and pyrantel.

In one embodiment, the antiparasitic combinations of the invention include the compound IC-1, or a salt thereof, combined with afoxolaner, moxidectin and praziquantel.

In one embodiment, the antiparasitic combinations of the invention include the compound IC-1, or a salt thereof, combined with esafoxolaner, moxidectin and praziquantel.

In one embodiment, the antiparasitic combinations of the invention include the compound IC-1, or a salt thereof, combined with umifoxolaner, moxidectin and praziquantel.

In another embodiment, the antiparasitic combinations of the invention include the compound IC-1, or a salt thereof, combined with fluralaner, moxidectin and praziquantel.

In one embodiment, the antiparasitic combinations of the invention include the compound IC-1, or a salt thereof, combined with afoxolaner, moxidectin and pyrantel.

In one embodiment, the antiparasitic combinations of the invention include the compound IC-1, or a salt thereof, combined with esafoxolaner, moxidectin and pyrantel.

In one embodiment, the antiparasitic combinations of the invention include the compound IC-1, or a salt thereof, combined with umifoxolaner, moxidectin and pyrantel.

In another embodiment, the antiparasitic combinations of the invention include the compound IC-1, or a salt thereof, combined with fluralaner, moxidectin and pyrantel.

In general, additional active agents (other than the compound of Formula (IA), Formula (IB) or Formula (IC) described above) are included in the dosage units of the invention in an amount of between about 0.1 μg and about 1000 mg. Typically, the active agent may be included in an amount of about 10 μg to about 500 mg, about 10 μg to about 400 mg, about 1 mg to about 300 mg, about 10 mg to about 200 mg or about 10 mg to about 100 mg. More typically the additional active agent will be present in an amount of about 5 mg to about 50 mg in the compositions of the invention.

The concentration of the additional active agents in compositions comprising the antiparasitic active agent combinations of the invention will typically be from about 0.01% to about 30% (w/w) depending on the potency of the active agent. In certain embodiments for very potent additional active agents including, but not limited to a macrocyclic lactone active agent, the concentration of the additional active agent will typically be from about 0.01% to about 10% (w/w), from about 0.01 to about 1% (w/w), from about 0.01% to about 0.5% (w/w), from about 0.10% to about 0.50% (w/w) or from about 0.010% to about 0.10% (w/w). In other embodiments, the concentration of the additional active agent will typically be from about 0.10% to about 2% (w/w) or about 0.10% to about 10% (w/w).

In other embodiments, the additional active agent will typically be present at higher concentrations to achieve the desired efficacy. In some embodiments, the additional active agent will be present in a concentration of about 1% to about 30% (w/w), about 1% to about 20% (w/w), about 1% to about 15% (w/w) or about 0.5% to about 10%. In still other embodiments, the additional active agent will be present in a concentration of about 5% to about 20% (w/w) or about 5% to about 15% (w/w) in the composition.

In various embodiments of the invention, an additional active agent may be included in the composition to deliver a dose of about 0.001 mg/kg to about 50 mg/kg or about 0.5 mg/kg to about 50 mg/kg of body weight of the animal. In other embodiments, the additional active agent will typically be present in an amount sufficient to deliver a dose of about 0.01 mg/kg to about 5 mg/kg, about 0.01 mg/kg to about 1 mg/kg or about 0.01 mg/kg to about 0.05 mg/kg of body weight. In other embodiments, the additional active agent will typically be present in an amount sufficient to deliver a dose of about 0.05 mg/kg to about 30 mg/kg, about 0.1 mg/kg to about 20 mg/kg. In other embodiments, the additional active agent will be present in an amount sufficient to deliver a dose of about 0.1 mg/kg to about 10 mg/kg, about 0.1 mg/kg to about 5 mg/kg, about 0.1 mg/kg to about 1 mg/kg or about 0.5 mg/kg to about 50 mg/kg per body weight of the animal.

In some embodiments, the compounds of Formula (IA), Formula (IB) and/or Formula (IC), including compound A472 and IC-1, will be included an amount in dosage forms to deliver a dose of about 0.1 mg/kg to about 50 mg/kg of body weight. In other embodiments, the compounds of Formula (IA), Formula (IB) and/or Formula (IC), including compound A472 and IC-1, will be included in dosage forms in an amount to deliver a dose of about 0.4 mg/kg to about 40 mg/kg of body weight. In yet other embodiments, the compounds of Formula (IA), Formula (IB) and/or Formula (IC), including compound A472 and IC-1, will be included in dosage forms in an amount to deliver a dose of about 0.5 mg/kg to about 30 mg/kg of body weight. In other embodiments, the compounds of Formula (IA), Formula (IB) and/or Formula (IC), including compound A472 and IC-1, will be included in dosage forms in an amount to deliver a dose of about 0.5 mg/kg to about 20 mg/kg, about 0.5 mg/kg to about 10 mg/kg or about 0.5 mg/kg to about 5 mg/kg of body weight.

In one embodiment, the compound of formula A472 will be included in dosage forms in an amount to deliver a dose of about 0.1 mg/kg to about 30 mg/kg of body weight. In another embodiment, the compound of formula A472 will be included in dosage forms in an amount to deliver a dose of about 0.3 mg/kg to about 20 mg/kg of body weight. In another embodiment, the compound of formula A472 will be included in dosage forms in an amount to deliver a dose of about 0.1 mg/kg to about 10 mg/kg of body weight, about 0.5 mg/kg to about 5 mg/kg or about 0.5 mg/kg to about 3 mg/kg. In another embodiment, the compound of formula A472 will be included in dosage forms in an amount to deliver a dose of about 0.5 mg/kg to about 2 mg/kg of body weight. In another embodiment, the compound of formula A472 will be included in dosage forms in an amount to deliver a dose of about 0.5 mg/kg to about 1.5 mg/kg of body weight.

In other embodiments, the compound of formula A472 will be included in dosage forms in an amount to deliver a dose of about 10 mg/kg to about 50 mg/kg of body weight. In another embodiment, the compound of formula A472 will be included in dosage forms in an amount to deliver a dose of about 20 mg/kg to about 40 mg/kg of body weight or about 15 mg/kg to about 30 mg/kg of body weight.

In one embodiment, the compound of formula IC-1 will be included in dosage forms in an amount to deliver a dose of about 0.1 mg/kg to about 30 mg/kg of body weight. In another embodiment, the compound of formula IC-1 will be included in dosage forms in an amount to deliver a dose of about 0.3 mg/kg to about 20 mg/kg of body weight. In another embodiment, the compound of formula IC-1 will be included in dosage forms in an amount to deliver a dose of about 0.5 mg/kg to about 10 mg/kg of body weight, about 0.5 mg/kg to about 5 mg/kg or about 0.5 mg/kg to about 3 mg/kg. In another embodiment, the compound of formula IC-1 will be included in dosage forms in an amount to deliver a dose of about 0.5 mg/kg to about 2 mg/kg of body weight. In another embodiment, the compound of formula IC-1 will be included in dosage forms in an amount to deliver a dose of about 0.5 mg/kg to about 1 mg/kg of body weight.

In other embodiments, the compound of formula IC-1 will be included in dosage forms in an amount to deliver a dose of about 10 mg/kg to about 50 mg/kg of body weight. In another embodiment, the compound of formula IC-1 will be included in dosage forms in an amount to deliver a dose of about 20 mg/kg to about 40 mg/kg of body weight or about 15 mg/kg to about 30 mg/kg of body weight.

In certain embodiments of the invention where the additional active agent is a very potent compound such as a macrocyclic lactone or other potent compounds, the active agent will be present in a dosage form at a concentration to provide a dose of about 0.001 mg/kg to about 5 mg/kg, about 0.001 mg/kg to about 0.1 mg/kg or about 0.001 mg/kg to about 0.01 mg/kg of body weight. In another embodiment, the additional active agent will be present in a dosage form at a concentration to provide a dose of about 0.005 mg/kg to about 0.05 mg/kg of body weight. In still other embodiments, the additional active agent is present in an amount sufficient to deliver a dose of about 0.01 mg/kg to about 2 mg/kg or about 0.1 mg/kg to about 1 mg/kg per body weight of the animal. In still other embodiments, the additional active agent may be present in an amount to deliver a dose of about 1 μg/kg to about 200 μg/kg or about 0.1 mg/kg to about 1 mg/kg of weight of animal.

In an embodiment wherein the macrocyclic lactone is moxidectin, moxidectin will be present in a dosage form at a concentration sufficient to deliver a dose of about 5 μg/kg to about 25 μg/kg. In an embodiment wherein the macrocyclic lactone is moxidectin, moxidectin will be present in a dosage form at a concentration sufficient to deliver a dose of about 10 μg/kg to about 25 μg/kg. In another embodiment wherein the macrocyclic lactone is moxidectin, moxidectin will be present in a dosage form at a concentration sufficient to deliver a dose of about 15 μg/kg to about 25 μg/kg. In another embodiment wherein the macrocyclic lactone is moxidectin, moxidectin will be present in a dosage form at a concentration sufficient to deliver a dose of about 10 μg/kg to about 15 μg/kg. In another embodiment wherein the macrocyclic lactone is moxidectin, moxidectin will be present in a dosage form at a concentration sufficient to deliver a dose of about 12 μg/kg. In another embodiment wherein the macrocyclic lactone is moxidectin, moxidectin will be present in a dosage form at a concentration sufficient to deliver a dose of about 20 μg/kg. In another embodiment wherein the macrocyclic lactone is moxidectin, moxidectin will be present in a dosage form at a concentration sufficient to deliver a dose of about 24 μg/kg.

In some embodiments of the invention where the additional active agent is an isoxazoline active agent (e.g. Is-1 to Is-9), the isoxazoline active agent will be present in a dosage form at a concentration sufficient to deliver a dose of about 0.5 mg/kg to about 50 mg/kg of animal body weight. In other embodiments, the isoxazoline active agent will be present in a dosage form at a concentration sufficient to deliver a dose of about 1 mg/kg to about 40 mg/kg of animal body weight. In yet other embodiments, the isoxazoline active agent will be present in a dosage form at a concentration sufficient to deliver a dose of about 1 mg/kg to about 30 mg/kg of animal body weight. In another embodiment, the isoxazoline active agent will be present in a dosage form at a concentration to deliver a dose of about 1 mg/kg to about 20 mg/kg.

In some embodiments, the isoxazoline active agent will be present in a dosage form at a concentration sufficient to deliver a dose of about 0.5 mg/kg to about 10 mg/kg of animal body weight. In another embodiment, the isoxazoline active agent will be present in a dosage form at a concentration sufficient to deliver a dose of about 1 mg/kg to about 5 mg/kg. In yet another embodiment, the isoxazoline active agent will be present in a dosage form at a concentration sufficient to deliver a dose of about 1 mg/kg to about 3 mg/kg.

In other embodiments, the isoxazoline active agent will be present in a dosage form at a concentration sufficient to deliver a dose of about 5 mg/kg to about 30 mg/kg of animal body weight. In other embodiments, the isoxazoline active agent will be present in a dosage form at a concentration sufficient to deliver a dose of about 10 mg/kg to about 20 mg/kg of animal body weight. In another embodiment, the isoxazoline active agent will be present in a dosage form at a concentration sufficient to deliver a dose of about 15 mg/kg to about 30 mg/kg of animal body weight. In another embodiment, the isoxazoline active agent will be present in a dosage form at a concentration sufficient to deliver a dose of about 20 mg/kg to about 25 mg/kg of animal body weight.

In an embodiment wherein the isoxazoline active agent is fluralaner, fluralaner will be present in a dosage form at a concentration sufficient to deliver a dose of about 10 mg/kg. In another embodiment, fluralaner will be present in a dosage form at a concentration sufficient to deliver a dose of about 25 mg/kg of animal body weight.

In an embodiment wherein the isoxazoline active agent is afoxolaner, afoxolaner will be present in a dosage form at a concentration sufficient to deliver a dose of about 1 mg/kg to about 20 mg/kg, about 1 to about 10 mg/kg or about 1 to about 5 mg/kg of body weight.

In another embodiment wherein the isoxazoline active agent is afoxolaner, afoxolaner will be present in a dosage form at a concentration sufficient to deliver a dose of about 2.5 mg/kg.

In an embodiment wherein the isoxazoline active agent is sarolaner, sarolaner will be present in a dosage form at a concentration sufficient to deliver a dose of about 1.2 mg/kg.

In an embodiment wherein the isoxazoline active agent is lotilaner, lotilaner will be present in a dosage form at a concentration sufficient to deliver a dose of about 20 mg/kg.

In an embodiment of the antiparasitic combinations wherein the combination includes praziquantel, praziquantel will be present in a dosage form to deliver a dose of about 1 mg/kg to about 10 mg/kg of body weight. In an embodiment of the antiparasitic combinations wherein the combination includes praziquantel, praziquantel will be present in a dosage form to deliver a dose of about 2 mg/kg to about 7 mg/kg of body weight. In a particular embodiment of the antiparasitic combinations wherein the combination includes praziquantel, praziquantel will be present in a dosage form to deliver a dose of about 5 mg/kg of body weight.

In an embodiment of the antiparasitic combinations wherein the combination includes pyrantel, pyrantel will be present in a dosage form to deliver a dose of about 5 mg/kg of body weight as the pamoate salt.

In addition to the other active agents mentioned above, the antiparasitic combinations of the invention may be combined with other antiparasitic active agents in a composition to treat a desired spectrum of parasites. It would be well within the skill level of the practitioner to decide which individual compound can be used in the inventive composition to treat and/or prevent a particular parasitic infection or infestation.

In one particular embodiment, the antiparasitic combinations of the invention will include a combination of compound A472 in combination with afoxolaner. In another embodiment of the invention, the antiparasitic combinations include compound A472 and moxidectin. In another embodiment of the invention, the antiparasitic combinations include compound A472 and praziquantel.

In one embodiment, the antiparasitic combinations of the invention will include a combination of compound A472, afoxolaner and moxidectin.

In another embodiment, the antiparasitic combinations of the invention will include a combination of compound A472, afoxolaner, moxidectin and praziquantel.

In one embodiment, the antiparasitic combinations of the invention will be included in a dosage form to deliver a dose of about 0.1 mg/kg to about 10 mg/kg of compound A472, about 5 μg/kg to about 25 μg/kg moxidectin, about 1 mg/kg to about 20 mg/kg afoxolaner and about 0.1 mg/kg to about 10 mg/kg praziquantel.

In another embodiment, the antiparasitic combinations of the invention will be included in a dosage form to deliver a dose of about 0.5 mg/kg to about 5 mg/kg of compound A472, about 10 μg/kg to about 25 μg/kg moxidectin, about 0.5 mg/kg to about 10 mg/kg afoxolaner and about 2 mg/kg to about 7 mg/kg praziquantel.

In another embodiment, the antiparasitic combinations of the invention will be included in a dosage form to deliver a dose of about 0.5 mg/kg to about 3 mg/kg of compound A472, about 10 μg/kg to about 15 μg/kg moxidectin, about 0.5 mg/kg to about 3 mg/kg afoxolaner and about 5 mg/kg praziquantel.

In another embodiment, the antiparasitic combinations of the invention will be included in a dosage form to deliver a dose of about 0.5 mg/kg to about 2 mg/kg of compound A472, about 10 μg/kg to about 12 μg/kg moxidectin, about 2.5 mg/kg afoxolaner and about 5 mg/kg praziquantel.

In another embodiment, the antiparasitic combinations of the invention will be included in a dosage form to deliver a dose of about 0.5 mg/kg to about 1 mg/kg of compound A472, about 12 μg/kg moxidectin, about 2.5 mg/kg afoxolaner and about 5 mg/kg praziquantel.

In another embodiment, the antiparasitic combinations of the invention will include a combination of compound IC-1 in combination with lotilaner. In another embodiment of the invention, the antiparasitic combinations include compound IC-1 and moxidectin. In another embodiment of the invention, the antiparasitic combinations include compound IC-1 and praziquantel.

In one embodiment, the antiparasitic combinations of the invention will include a combination of compound IC-1, lotilaner and moxidectin.

In another embodiment, the antiparasitic combinations of the invention will include a combination of compound IC-1, lotilaner, moxidectin and praziquantel.

In one embodiment, the antiparasitic combinations of the invention will be included in a dosage form to deliver a dose of about 1 mg/kg to about 10 mg/kg of compound IC-1, about 10 μg/kg to about 25 μg/kg moxidectin, about 10 mg/kg to about 30 mg/kg lotilaner and about 2 mg/kg to about 7 mg/kg praziquantel.

In one embodiment, the antiparasitic combinations of the invention will be included in a dosage form to deliver a dose of about 10 mg/kg to about 20 mg/kg of compound IC-1, about 10 μg/kg to about 25 μg/kg moxidectin, about 10 mg/kg to about 30 mg/kg lotilaner and about 2 mg/kg to about 7 mg/kg praziquantel.

In one embodiment, the antiparasitic combinations of the invention will be included in a dosage form to deliver a dose of about 20 mg/kg to about 40 mg/kg of compound IC-1, about 10 μg/kg to about 25 μg/kg moxidectin, about 10 mg/kg to about 30 mg/kg lotilaner and about 2 mg/kg to about 7 mg/kg praziquantel.

In one embodiment, the antiparasitic combinations of the invention will be included in a dosage form to deliver a dose of about 25 mg/kg to about 35 mg/kg of compound IC-1, about 15 μg/kg to about 25 μg/kg moxidectin, about 15 mg/kg to about 25 mg/kg lotilaner and about 5 mg/kg praziquantel.

In one embodiment, the antiparasitic combinations of the invention will be included in a dosage form to deliver a dose of about 30 mg/kg of compound IC-1, about 20 μg/kg moxidectin, about 20 mg/kg lotilaner and about 5 mg/kg praziquantel.

In one embodiment, the antiparasitic combinations of the invention will be included in a dosage form to deliver a dose of about 20 mg/kg to about 40 mg/kg of compound IC-1, about 10 μg/kg to about 25 μg/kg moxidectin, about 10 mg/kg to about 30 mg/kg lotilaner and about 2 mg/kg to about 7 mg/kg pyrantel.

In one embodiment, the antiparasitic combinations of the invention will be included in a dosage form to deliver a dose of about 25 mg/kg to about 35 mg/kg of compound IC-1, about 15 μg/kg to about 25 μg/kg moxidectin, about 15 mg/kg to about 25 mg/kg lotilaner and about 5 mg/kg pyrantel.

In one embodiment, the antiparasitic combinations of the invention will be included in a dosage form to deliver a dose of about 30 mg/kg of compound IC-1, about 20 μg/kg moxidectin, about 20 mg/kg lotilaner and about 5 mg/kg pyrantel.

The invention will now be further described by way of the following non-limiting examples.

### Methods of Synthesis

The compounds of Formula (IA), including compound A472 and the compounds described in Table 1, or salts thereof, described herein may be prepared according to the process described in schemes 1 and 2 shown below. A person of skill in the art will be able to prepare a variety of compounds of the invention by adapting processes described below with alternate starting materials and/or reagents known in the art.

Compounds of the invention of Formula (IB) may be prepared by processes described in WO 2017/178416, which is incorporated herein by reference in its entirety.

Compounds of the invention of Formula (IC) may be prepared by processes described in WO 2018/087036, WO 2019/002132, WO 2019/115768, WO 2019/215182, WO 2021/204930, WO 2021/018839, WO 2021/122911 and WO 2025/027117, all of which are incorporated herein by reference in their entirety.

Isoxazoline active agents may be prepared by processes described in WO 2005/085216, U.S. Pat. No. 7,662,972, WO 2007/079162, WO 2009/002809, WO 2012/120399 and WO 2010/070068, all incorporated herein by reference in their entirety.

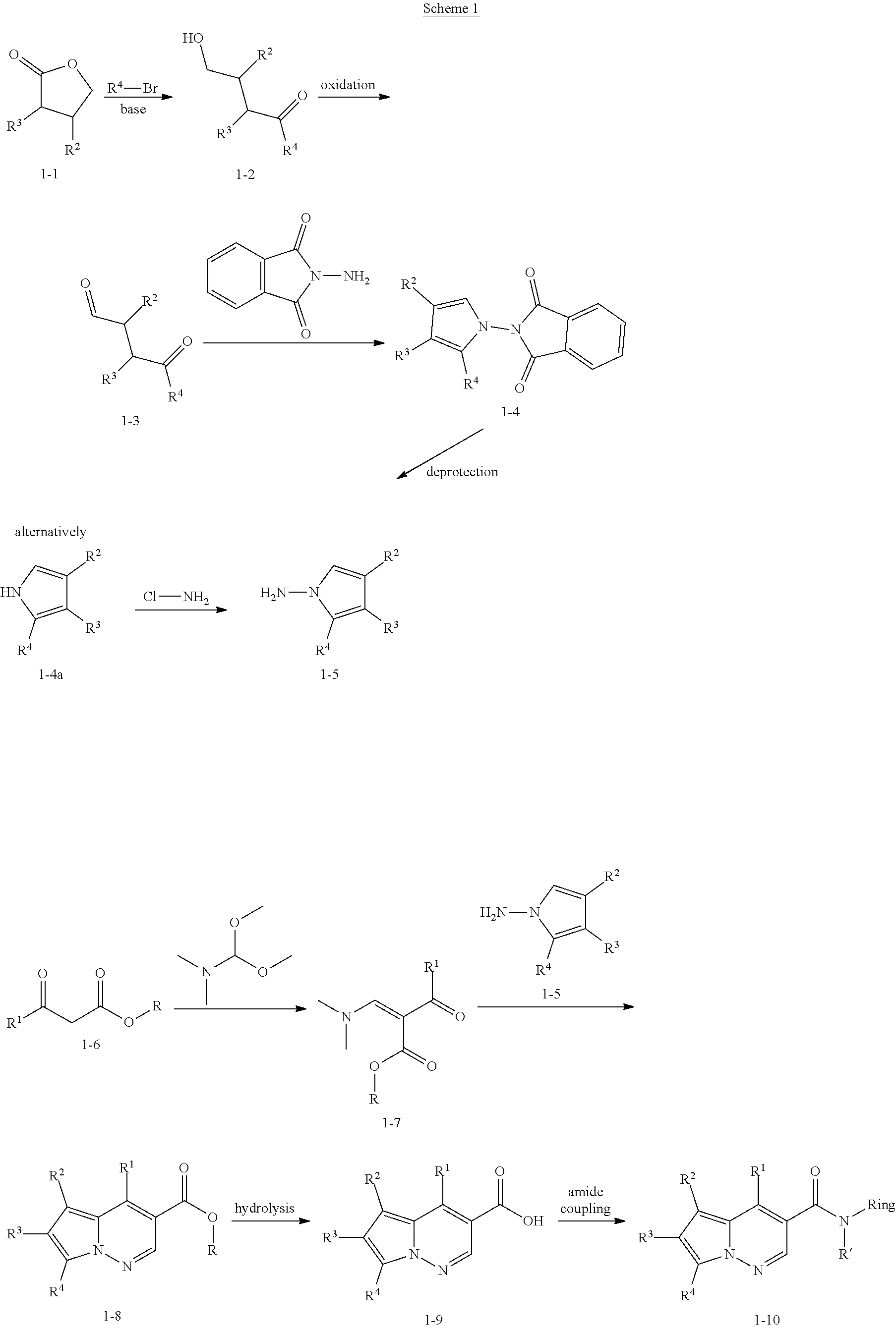

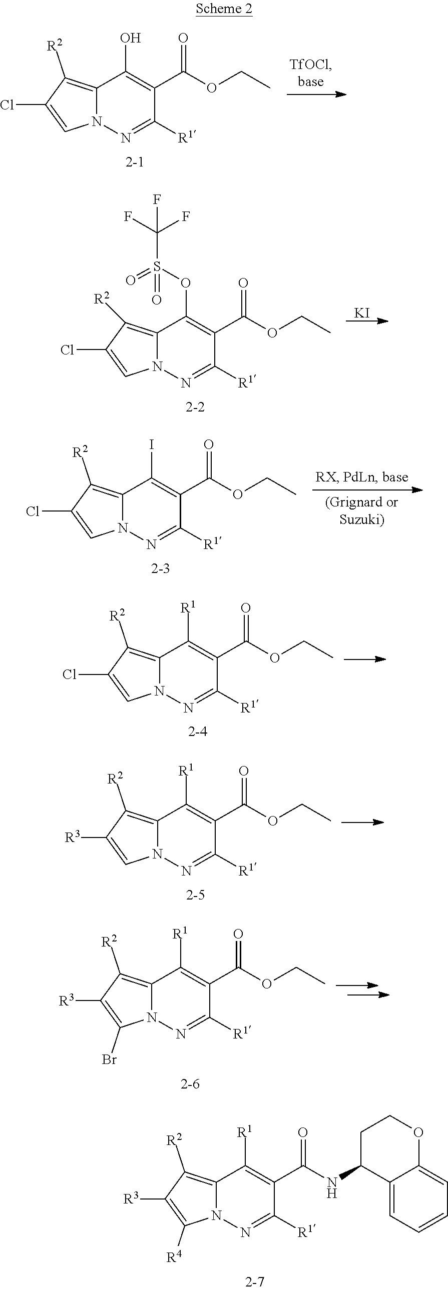

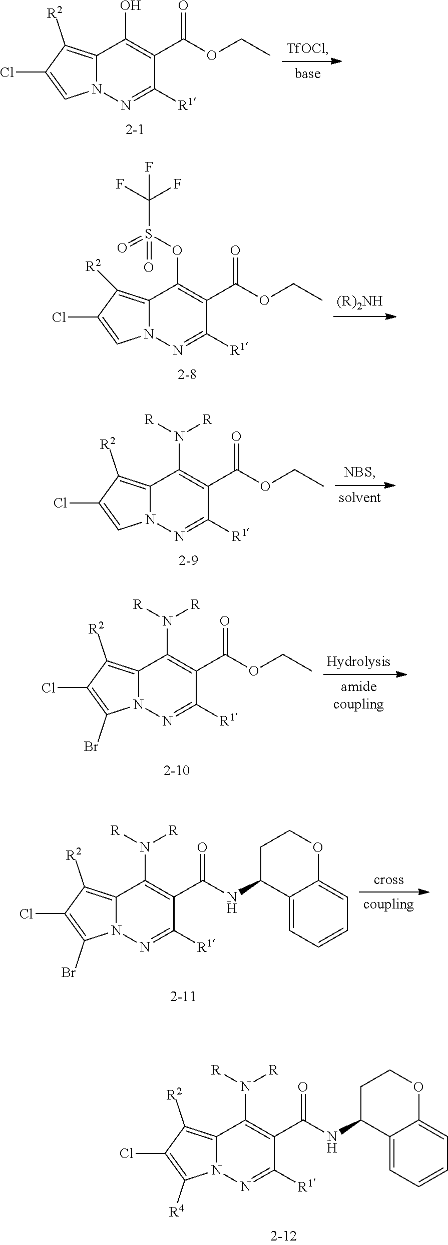

### EXAMPLES

### Preparation Examples

The Examples that follow are intended to only illustrate preparation of compounds of Formula (IA) of the present invention without restricting it. The compounds of the invention, including those of Formula (IA), or salts thereof, may be prepared by adopting one of the following reaction schemes. The starting materials for their preparation may be commercially available or can be prepared by methods known by persons of skill in the art and as described in the literature or may be an intermediate in any other of the schemes described herein. It will be appreciated that the following procedures may be modified by persons of skill in the art to prepare additional compounds according to the invention. For example, a person of skill in the art will understand that replacement of certain starting materials or the use of different intermediates will enable the preparation of different compounds of Formula (IA).

The terms “ambient temperature” and “room temperature” are used interchangeably and designate a temperature of about 20 °C. Although the following subject matter is described in some detail by way of illustration and example for purposes of clarity of understanding, it will be understood by those skilled in the art that certain changes and modifications can be practiced within the scope of the Examples.

Abbreviations:

| | |
|---|---|
| ACN | acetonitrile |
| Alox | aluminium oxide |
| Aq. | aqueous |
| Atm | atmosphere |
| °C | degree celsius |
| CyH/CH | cyclohexane |
| conc. | concentrated |
| DCC | N,N′-dicyclohexylmethanediimine |
| DCM | dichloromethane |
| DBU | 1,8-diazabicyclo[5.4.0]undec-7-ene |
| 1,2-DCE | 1,2-dichloroethane |
| DEE/Et₂O | diethyl ether |
| DIPE | diisopropyl ether |
| DIPEA | N,N-diisopropylethylamine |
| 1,2-DME | 1,2-dimethyl ether |
| DMF | N,N-dimethylformamide |
| DMSO | dimethyl sulfoxide |
| ESI-MS | electrospray ionisation mass spectrometry |
| EtOAc/EE/EA | ethyl acetate |
| ex | example |
| eq | equivalent |
| h | hour |
| HAc/HOAc | acetic acid |
| HATU | N,N,N′,N′-tetramethyl-O-(7-azabenzotriazol-1- |
| | yl)uranium hexafluorophosphate |
| HCl | hydrochloric acid |
| HNO₃ | nitric acid |
| HPLC | high performance liquid chromatography |
| iPrMgCl | isopropylmagnesium chloride |
| K₃PO₄ | tripotassium phosphate |
| LiHMDS | lithium-bis(trimethylsilyl)amide |
| LAH | lithium aluminum hydride |
| LiOH | lithium hydroxide |
| MeI | methyl iodide |
| MeOH | methanol |
| NaH | sodium hydride |
| NaHCO₃ | sodium bicarbonate |
| Na₂SO₄ | sodium sulfate |
| NBS | N-bromosuccinimide |
| NClS/NCS | N-chlorosuccinimide |
| NH₃ | ammonia |
| NH₄Cl | ammonium chloride |
| NBS | N-bromosuccinimide |
| NIS | N-iodosuccinimide |
| NMP | N-methyl-2-pyrrolidone |
| min | minute |
| mL | milliliter |
| mmol | millimole |
| Pd/C | palladium on activated carbon |
| Pd(DtBPF)Cl₂ | [1,1′-bis(di-tert- |
| | butylphosphino)ferrocene]dichloropalladium(II) |
| Pd(dppf)Cl₂ | [1,1′-bis(diphenyl- |
| | phosphino)ferrocene]dichloropalladium(II) |
| PE | petroleum ether |
| psi | pound per square inch |
| PtO₂ | platinum dioxide |
| RT | room temperature (about 20 °C) |
| Rₜ | retention time |
| sat. | saturated |
| TBAF | tetra-n-butylammonium fluoride |
| tBuOH | tert-butyl alcohol |
| tBuOK | potassium tert-butoxide |
| TEA | triethylamine |
| TFA | trifluoroacetic acid |
| THF | tetrahydrofuran |
| TLC | thin-layer chromatography on SiO₂ |
| TMSOK | potassium trimethylsilanolate |
| X-Phos | 2-dicyclohexylphosphino-2′,4′,6′-triisopropylbiphenyl |
| MCC | Microcrystalline cellulose; MCC 200 XLM is a grade |
| | of MCC with large particle size and extra low |
| | moisture manufactured by JRS Pharma |
| VitE TPGS | d-α-Tocopheryl polyethylene glycol 1000 succinate |
| BHT | Butylated hydroxytoluene |
| HPMC-AS | Hydroxypropyl methylcellulose acetate succinate |
| HPMC-AS | Hydroxypropyl methylcellulose acetate succinate |
| LG | LG grade |
| PVP | Polyvinylpyrrolidone |

Some examples of Formula (IA), or salts thereof, are derived after separation of racemic mixtures and obtained as enantiomerically pure products. The stereochemistry is in some cases arbitrarily assigned and the respective compound characterized by analytical methods.

Preparation Example 1: Compound A465. Compound A465 was prepared according to general Scheme 1 shown above as described below. In addition, the following compounds of the invention can be prepared by adapting general Scheme 1 and the reactions described below for the synthesis of example A465 as well as by employing and modifying reactions known in the literature (see for example WO2021030379, WO2012125886 and WO2012125893): A461, A462, A463, A464, A469, A466, A467, A468, A470, A471, A473, A474, A475, A476, A483, A484, A486, A487, A488, A489, A492, A494, A495, A496, A497, A498, A500.

### 1. Synthesis of 1-(3,5-dichlorophenyl)-4-hydroxy-2-methylbutan-1-one

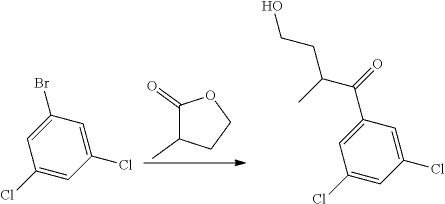

A mixture of 3.0 g (13.3 mmol) 1-bromo-3,5-dichlorobenzene in 50 mL THF anhydrous was cooled to −95 °C with a MeOH/nitrogen bath, 10.8 mL (17.3 mmol) tert-butyllithium was added dropwise, followed by 1.99 g (19.9 mmol) alpha-methyl-gamma-butyrolactone in 10 mL THF anhydrous. The reaction mixture was stirred at −85 °C for 1 h. The reaction mixture was quenched with sat. NH₄Cl solution at −90 °C and the solution warmed to RT and extracted twice with EE. The organic layers are collected, dried, filtered and the filtrate is evaporated to obtain 3.6 g (27%) of the crude product as yellow oil.

### 2. Synthesis of 4-(3,5-dichlorophenyl)-3-methyl-4-oxobutanal

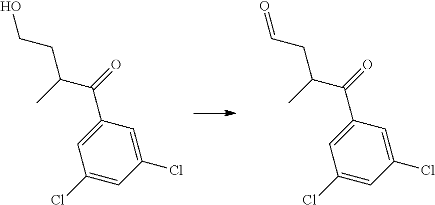

A mixture of 2.59 mL (26.42 mL) DMSO in 25 mL DCM was cooled to −78 °C. 1.6 mL (18.9 mmol) oxalyl chloride was added dropwise and the solution was stirred for 15 min. 3.6 g (3.6 mmol) 1-(3,5-dichlorophenyl)-4-hydroxy-2-methylbutan-1-one in 8 mL DCM was added and the reaction mixture was stirred at −78 °C for 1 h. 11.67 mL (83.76 mmol) TEA was added and the solution warmed to RT and stirred at RT for 1.5 h. The reaction mixture was quenched with water and extracted with DCM. The organic layers were collected, dried, filtered and the filtrate was evaporated. The residue was purified by column chromatography (silica gel; PE/EtOAc) and the solvents were removed in vacuo to obtain 1.10 g (92.0%) of the product as yellow oil.

### 3. Synthesis of 2-[2-(3,5-dichlorophenyl)-3-methyl-1H-pyrrol-1-yl]-2,3-dihydro-1H-isoindole-1,3-dione

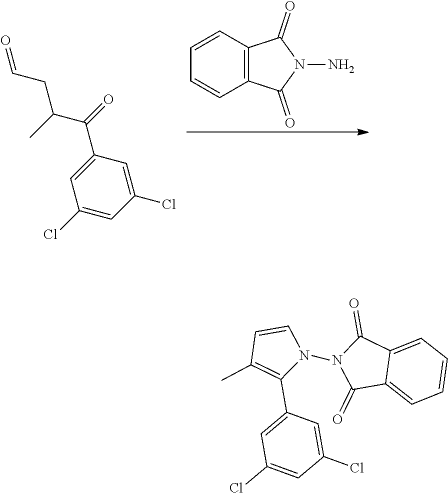 [SMILES: CC(CC=O)C(=O)c1cc(Cl)cc(Cl)c1 ; NN1C(=O)c2ccccc2C1=O ; Cc1ccn(N2C(=O)c3ccccc3C2=O)c1-c1cc(Cl)cc(Cl)c1]

A mixture of 1.10 g (3.36 mmol) 4-(3,5-dichlorophenyl)-3-methyl-4-oxobutanal, 600 mg (3.7 mmol)N-amino phthalimide and 9.26 mL (37.0 mmol) was stirred at 100 °C for 1 h. The reaction mixture was evaporated and the crude residue was crystallized with PE/EE to obtain 720 mg (57.0%) of 2-[2-(3,5-dichlorophenyl)-3-methyl-1H-pyrrol-1-yl]-2,3-dihydro-1H-isoindole-1,3-dione as a white solid.

### 4. Synthesis of 2-(3,5-dichlorophenyl)-3-methyl-1H-pyrrol-1-amine

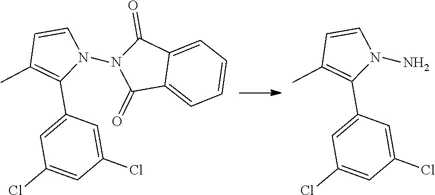 [SMILES: Cc1ccn(N2C(=O)c3ccccc3C2=O)c1-c1cc(Cl)cc(Cl)c1 ; Cc1ccn(N)c1-c1cc(Cl)cc(Cl)c1]

A mixture of 1.1 g (3.0 mmol) 2-[2-(3,5-dichlorophenyl)-3-methyl-1H-pyrrol-1-yl]-2,3-dihydro-1H-isoindole-1,3-dione and 0.2 mL (7.4 mmol) hydrazine monohydrate in 12 mL EtOH was stirred at reflux for 2 h. The reaction mixture was filtered and the filtrate was evaporated. The crude residue was purified by column chromatography (silica gel; PE/EtOAc) and the solvents were removed in vacuo to obtain 650 mg (91%) of the product as white solid

### 5. Synthesis of methyl 7-(3,5-dichlorophenyl)-6-methyl-4-(propan-2-yl)pyrrolo[1,2-b]pyridazine-3-carboxylate

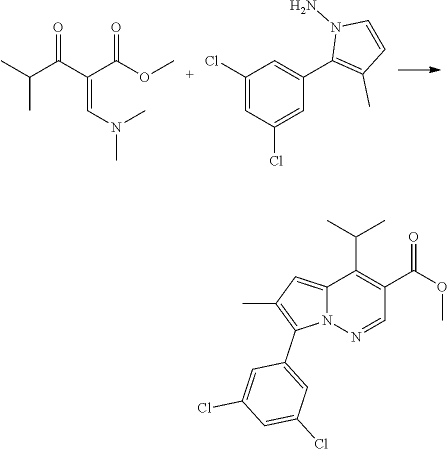

To a mixture of 1.lg (4.6 mmol) of 2-(3,5-dichlorophenyl)-3-methyl-1H-pyrrol-1-amine in 40 mL of methanol were added 820.0 mg (4.1 mmol) of methyl (2Z)-2-(dimethylamino)-methylidene]-4-methyl-3-oxopentanoate and 1.2 mL of 4M HCl in dioxane and the mixture was stirred at 80 °C for 3 h. After evaporation of the solvents, the crude residue was purified by column chromatography (silica gel; cyclohexane/EtOAc) and the solvents were removed in vacuo to obtain 380 mg (24%) of the product as a solid.

### 6. Synthesis of 7-(3,5-dichlorophenyl)-6-methyl-4-(propan-2-yl)pyrrolo[1,2-b]pyridazine-3-carboxylic acid

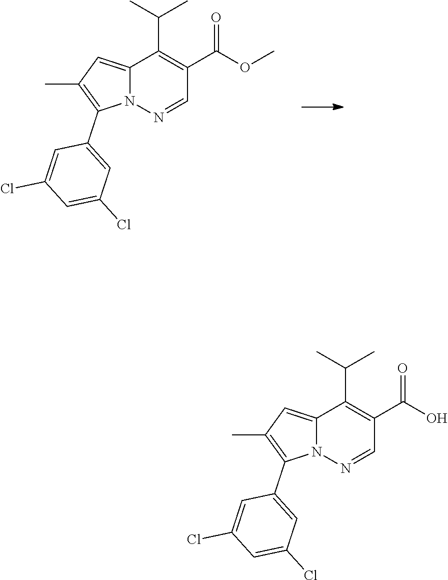 [SMILES: COC(=O)c1cnn2c(-c3cc(Cl)cc(Cl)c3)c(C)cc2c1C(C)C ; Cc1cc2c(C(C)C)c(C(=O)O)cnn2c1-c1cc(Cl)cc(Cl)c1]

To a mixture of 94.5 mg (0.2 mmol) of methyl 7-(3,5-dichlorophenyl)-6-methyl-4-(propan-2-yl)pyrrolo[1,2-b]pyridazine-3-carb oxyl ate and 1.5 mL solution of LiOH (1M) was added 1.5 mL of TH. After stirring for 2 h at 40 °C the reaction mixture was heated to 60 °C over night. Then, the solution was acidified with 4 M HCl at RT and the resulting residue was filtered off and washed with 2 mL of H₂O. After drying of the residue, 84 mg (93.0%) of 7-(3,5-dichlorophenyl)-6-methyl-4-(propan-2-yl)pyrrolo[1,2-b]pyridazine-3-carboxylic acid were obtained as a yellow solid.

### 7. Synthesis of 7-(3,5-dichlorophenyl)-N-[(4S)-3,4-dihydro-2H-1-benzopyran-4-yl]-6-methyl-4-(propan-2-yl)pyrrolo[1,2-b]pyridazine-3-carboxamide (A465)

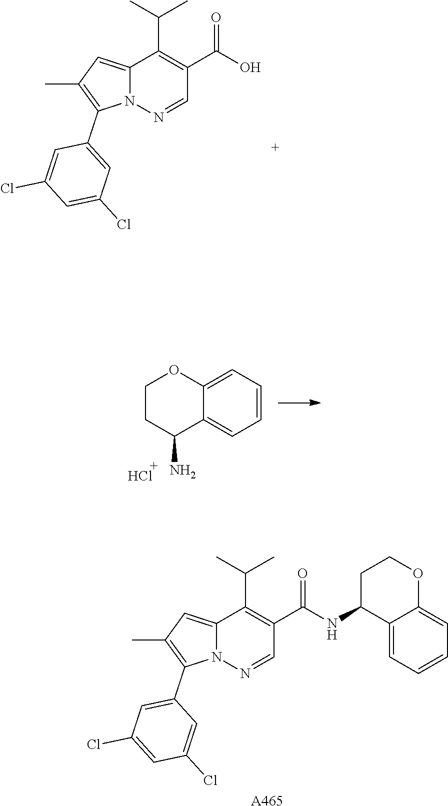 [SMILES: Cc1cc2c(C(C)C)c(C(=O)O)cnn2c1-c1cc(Cl)cc(Cl)c1 ; N[C@H]1CCOc2ccccc21 ; Cl ; Cc1cc2c(C(C)C)c(C(=O)N[C@H]3CCOc4ccccc43)cnn2c1-c1cc(Cl)cc(Cl)c1 ; "A465"]

A solution of 350 mg (0.9 mmol) of 7-(3,5-dichlorophenyl)-6-methyl-4-(propan-2-yl)pyrrolo[1,2-b]pyridazine-3-carboxylic acid in 8 mL of DMF was treated with 650 μl of DIPEA and 410 mg (1.7 mmol) of HATU and stirred for 10 min. After addition of 200 mg (1.1 mmol) of (S)-chroman-4-amine hydrochloride the mixture was stirred for 16 h at room temperature. The solvents were removed in vacuo and the residue was purified by column chromatography (silica gel; PE/EtOAc). After removal of the solvents in vacuo 310 mg (65.0%) of 7-(3,5-dichlorophenyl)-N-[(4S)-3,4-dihydro-2H-1-benzopyran-4-yl]-6-methyl-4-(propan-2-yl)pyrrolo[1,2-b]pyridazine-3-carboxamide (A465) were obtained as white solid. (ESI, m/z): 495 [M+H]⁺

### Preparation Example 2: Synthesis of 3,4-dihydro-2H-1,4-benzoxazin-4-amine

3,4-dihydro-2H-1,4-benzoxazin-4-amine, which is used in the synthesis of compounds of the invention containing Ring System D (see Table 1 above) such as compounds A479, A482, A494, A495, A496, A497, A498, A516 and A565, is prepared according to the process described below.

### 1. Synthesis of 4-nitroso-3,4-dihydro-2H-1,4-benzoxazine

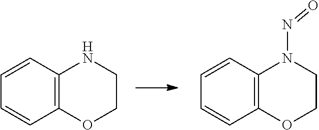 [SMILES: c1ccc2c(c1)NCCO2 ; O=NN1CCOc2ccccc21]

Into a 500 mL round-bottom flask. were added 3,4-dihydro-2H-1,4-benzoxazine (3.0 g, 22.2 mmol) and HCl (300 ml, 3M) and NaNO₂ (1.8 g, 26.1 mmol) was added in portions at 2 h at 0 °C under nitrogen atmosphere. The resulting mixture was concentrated under reduced pressure. The residue was purified by silica gel column chromatography, eluted with PE/EA (10:1) to afford 4-nitroso-3,4-dihydro-2H-1,4-benzoxazine (2.7 g, 75%) as a yellow oil.

### 2. Synthesis of 3,4-dihydro-2H-1,4-benzoxazin-4-amine

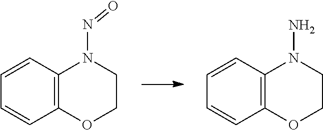 [SMILES: O=NN1CCOc2ccccc21 ; NN1CCOc2ccccc21]

Into a 500 mL round-bottom flask. were added 4-nitroso-3,4-dihydro-2H-1,4-benzoxazine (2.7 g, 19.2 mmol) and THF (300 ml) and LAH (1.35 g, 1.9 mmol) was added in portions at 2 h at 0 °C under nitrogen atmosphere. The resulting mixture was concentrated under reduced pressure. The residue was purified by silica gel column chromatography, eluted with PE/EA (10:1) to afford 3,4-dihydro-2H-1,4-benzoxazin-4-amine (2.2 g, 45%) as a yellow oil.

Preparation Example 3: Scheme 3 below describes the synthesis of intermediate compounds INT-1, INT-2 and INT-3, which correspond in Scheme 2 below, respectively, to compounds 2-1 wherein R^{1′} and R² are hydrogen; compound 2-4 wherein R¹ is tert-butyl, R² and R^{1′} are hydrogen and R³ is chloro; and compound 2-5 wherein R¹ is tert-butyl, R² and R^{1′} are hydrogen and R³ is methyl. These intermediates are used to prepare various compounds of the invention as depicted in Scheme 2 and described in detail below.

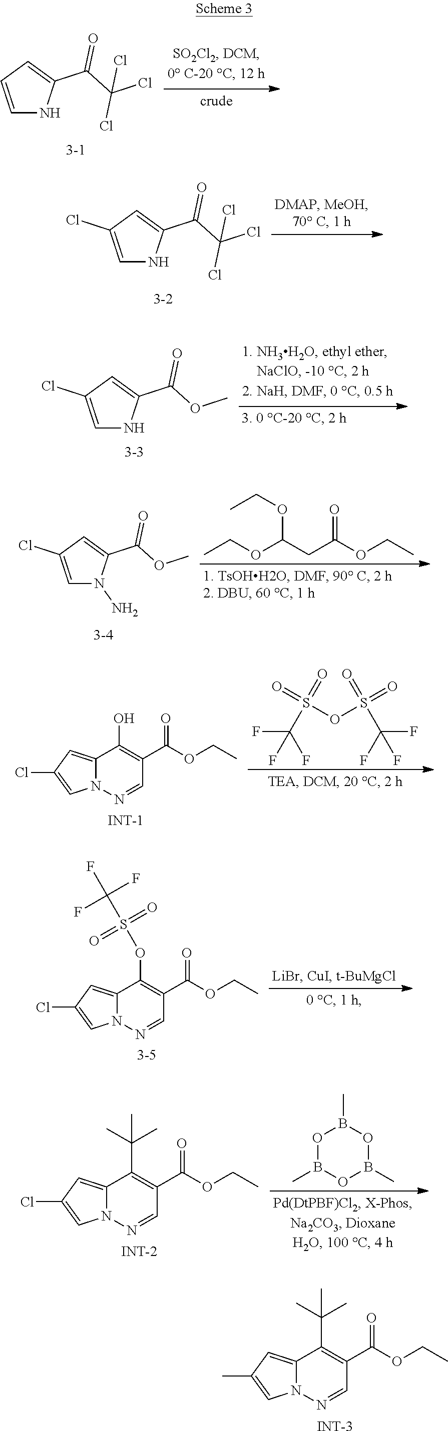

### 1. Synthesis of 2,2,2-trichloro-1-(4-chloro-1H-pyrrol-2-yl)ethanone (3-2)

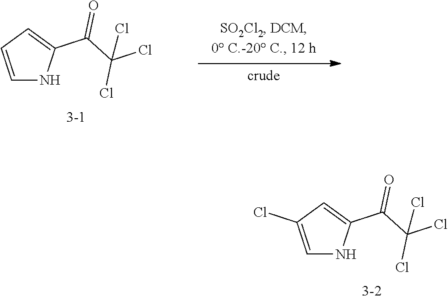 [SMILES: O=C(c1ccc[nH]1)C(Cl)(Cl)Cl ; O=C(c1cc(Cl)c[nH]1)C(Cl)(Cl)Cl]

Into a 5.0 L 3-necked round-bottom flask, was placed 2,2,2-trichloro-1-(1H-pyrrol-2-yl)ethanone (3-1, 600 g, 2824.2 mmol) in DCM (4.0 L). To this was added SO₂Cl₂ (419.30 g, 3106.6 mmol) dropwise at 0 °C. The reaction mixture was stirred for 12 h at 20 °C. The resulting mixture was adjusted to pH 8 with aqueous Na₂CO₃ (2 M). The resulting mixture was extracted with DCM (2.0 L x 3). The combined organic layers were dried over anhydrous Na₂SO₄ and concentrated under reduced pressure to afford 2,2,2-trichloro-1-(4-chloro-1H-pyrrol-2-yl)ethanone (3-2, 730 g, crude) as a black solid which was used directly in the next step.

### 2. Synthesis of methyl 4-chloro-1H-pyrrole-2-carboxylate (3-3)

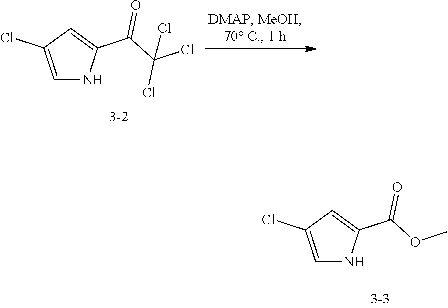

Into a 3.0 L 3-necked round-bottom flask, was placed 2,2,2-trichloro-1-(4-chloro-1H-pyrrol-2-yl)ethanone (3-2, 730 g, crude), MeOH (2.5 L) and DMAP (72.34 g, 592.1 mmol). The reaction mixture was stirred at 70 °C for 1 h. The mixture was allowed to cool down to room temperature and concentrated under reduced pressure. The residue was purified by silica gel column chromatography, eluted with PE/EA (10/1) to afford methyl 4-chloro-1H-pyrrole-2-carboxylate (3-3, 330 g, 73% 2 steps) as a white solid.

### 3. Synthesis of methyl 1-amino-4-chloropyrrole-2-carboxylate (3-4)

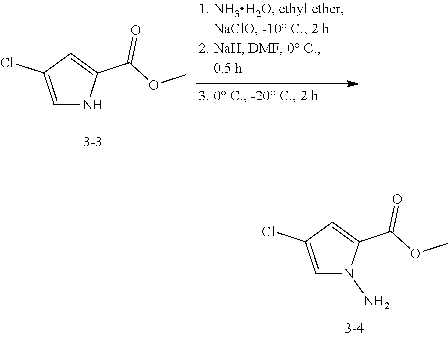

Into a 5 L 3-necked round-bottom flask, was placed NH₄Cl (80 g, 1495.6 mmol), ethyl ether (1.5 L) and NH₃·H₂O (120 mL). To this was added NaClO (1.7 L) dropwise at −10 °C. The reaction mixture was stirred at −10 °C for 2 h. Then the aqueous phase was removed by separation funnel. The organic layer was washed with 1.0 L of cooled brine. The organic layer (1.5 L) was dried over anhydrous Na₂SO₄ and stored at −40 °C for 1 h. Into another 2.0 L 3-necked round-bottom flask was placed methyl 4-chloro-1H-pyrrole-2-carboxylate (3-3, 80 g, 501.3 mmol) in DMF (800 mL). This was followed by addition of NaH (30.08 g, 752.0 mmol, 60%) in portion at 0 °C. The reaction mixture was stirred at 0 °C for 30 min. Then the above organic layers (1.5 L) were added. The combined reaction mixture was stirred at 20 °C for 2 h. The reaction was then quenched by the addition of 600 mL of Na₂S₂O₄ Saturated aqueous solution and the reaction mixture was stirred for 15 min. The resulting mixture was extracted with EA (3×1.0 L). The combined organic layers were dried over anhydrous Na₂SO₄ and concentrated under reduced pressure. The reaction was repeated one more time and the combined residue was purified by silica gel column chromatography, eluted with PE/EA (20/1) to afford methyl 1-amino-4-chloropyrrole-2-carboxylate (3-4, 188 g, 71%) as a white solid.

### 4. Synthesis of ethyl 6-chloro-4-hydroxypyrrolo[1,2-b]pyridazine-3-carboxylate (INT-1)

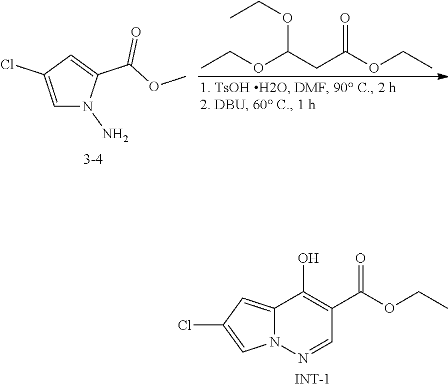

Into a 3.0 L round-bottom flask, was placed methyl 1-amino-4-chloropyrrole-2-carboxylate (3-4, 150 g, 859.2 mmol) in DMF (1.5 L) and ethyl 3,3-diethoxypropanoate (196.15 g, 1031.0 mmol) and PTSA (22.19 g, 128.9 mmol) were added. The reaction mixture was stirred at 90 °C for 2 h. The mixture was allowed to cool down to 60 °C. Then the DBU (261.61 g, 1718.4 mmol) was added at 60 °C. The reaction mixture was stirred at 60 °C for 1 h. The mixture was allowed to cool down to room temperature. The resulting mixture was diluted with EA (4.0 L) and washed with 3×1.0 L of water. The resulting solution was dried Na₂SO₄ and concentrated under reduced pressure. The crude product was stirred as a slurry with EtOH/water (3/1) to afford ethyl 6-chloro-4-hydroxypyrrolo[1,2-b]pyridazine-3-carboxylate (INT-1, 127 g, 61%) as a white solid after filtration. (ES, m/z): 239 [M−H]⁻; ¹H NMR (300 MHz, CDCl₃, ppm): δ 12.28 (s, 1H), 8.30 (s, 1H), 7.69 (d, J=2.0 Hz, 1H), 6.89 (d, J=1.6 Hz, 1H), 4.44 (q, J=7.2 Hz, 2H), 1.43 (t, J=7.2 Hz, 3H).

### 5. Synthesis of ethyl 6-chloro-4-(trifluoromethanesulfonyloxy)pyrrolo[1,2-b]pyridazine-3-carboxylate (3-5)

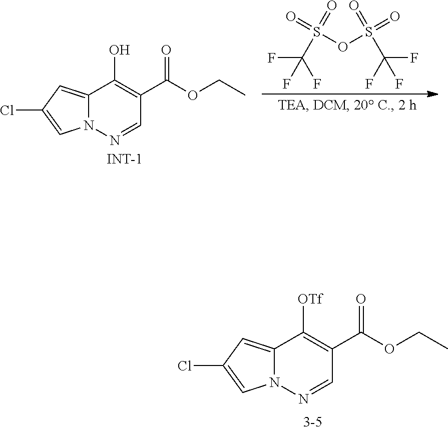

Into a 2.0 L round-bottom flask, was placed ethyl 6-chloro-4-hydroxypyrrolo[1,2-b]pyridazine-3-carboxylate (INT-1, 82 g, 340.8 mmol), DCM (800 mL), TEA (103.44 g, 1022.3 mmol). To this was added triflic anhydride (153.82 g, 545.2 mmol) dropwise at 0 °C. The reaction mixture was stirred at 20 °C for 2 h. The resulting mixture was diluted with DCM (1.0 L) and washed with 3×500 mL of water. The organic phase was collected and dried over Na₂SO₄ and concentrated under reduced pressure. The residue was purified by silica gel column chromatography, eluted with PE/EA (50/1) to afford ethyl 6-chloro-4-(trifluoromethanesulfonyloxy)pyrrolo[1,2-b]pyridazine-3-carboxylate (3-5, 90.5 g, 71%) as a yellow solid.

### 6. Synthesis of ethyl 4-tert-butyl-6-chloropyrrolo[1,2-b]pyridazine-3-carboxylate (INT-2)

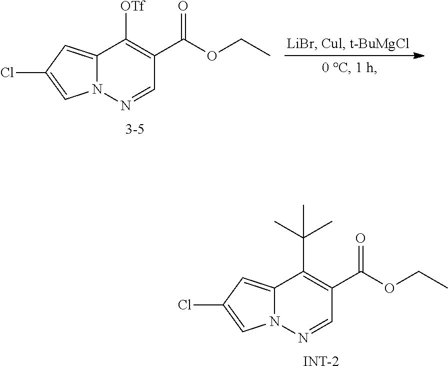

Into a 2.0 L three necks round-bottom flask, was placed ethyl 6-chloro-4-(trifluoromethanesulfonyloxy)pyrrolo[1,2-b]pyridazine-3-carboxylate (3-5, 62 g, 166.3 mmol), THF (1.2 L). To this was added LiBr (108.35 g, 1247.6 mmol) and CuI (237.62 g, 1247.6 mmol) at 0 °C. The reaction mixture was stirred at 0 °C for 30 min. This was followed by the addition of tert-butylmagnesium chloride (38.88 g, 332.7 mmol) at 0 °C. The reaction mixture was stirred at 0 °C for 30 min. The reaction was quenched by the addition of NH₄Cl (4 M in H₂O, 1.0 L) at 0 °C. The mixture was extracted with EA (3×500 mL). The organic phase was collected and dried over Na₂SO₄ and concentrated under reduced pressure. The residue was purified by silica gel column chromatography, eluted with PE/EA (25/1) to afford ethyl 4-tert-butyl-6-chloropyrrolo[1,2-b]pyridazine-3-carboxylate (INT-2, 42.5 g, 91%) as a yellow oil.

### 7. Synthesis of ethyl 4-tert-butyl-6-methylpyrrolo[1,2-b]pyridazine-3-carboxylate (INT-3)

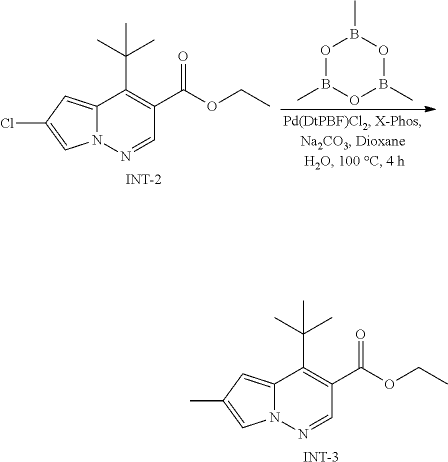

Into a 1.0 L round bottom flask, was placed ethyl 4-tert-butyl-6-chloropyrrolo[1,2-b]pyridazine-3-carboxylate (INT-2, 42 g, 149.6 mmol), dioxane (500 mL), H₂O (25 mL, 1387.7 mmol), Na₂CO₃ (39.64 g, 374.0 mmol), trimethylboroxine (65.73 g, 524.0 mmol), X-Phos (7.13 g, 14.7 mmol) and Pd(DtBPF)Cl₂ (9.75 g, 14.9 mmol) under nitrogen atmosphere. The reaction mixture was stirred at 100 °C for 4 h. The mixture was allowed to cool down to room temperature. The resulting mixture was diluted with EA (1.5 L) and washed with 3×500 mL of water. The organic phase was collected and dried over Na₂SO₄ and concentrated under reduced pressure. The residue was purified by silica gel column chromatography, eluted with PE/EA (20/1) to afford ethyl 4-tert-butyl-6-methylpyrrolo[1,2-b]pyridazine-3-carboxylate (INT-3, 31 g, 79%) as a yellow oil. (ES, m/z): 261 [M+H]⁺; ¹H NMR (300 MHz, CDCl₃, ppm): δ 7.75 (s, 1H), 7.54 (s, 1H), 6.68 (s, 1H), 4.36 (q, J=7.2 Hz, 2H), 2.37 (s, 3H), 1.58 (s, 9H), 1.40 (t, J=7.2 Hz, 3H).

Utilizing intermediates INT-1, INT-2 and INT-3 described above, the following compounds of the invention can be obtained by adapting the process shown in Scheme 2: A477, A478, A479, A480, A481, A482, A490, A491, A493, A499, A501, A502, A503, A504, A505, A506, A507, A508, A509, A510, A511, A512, A513, A514, A515, A516, A517, A518, A519, A520, A521, A522, A523, A525, A526, A530, A534, A538, A541, A542, A544, A545, A547, A549, A556, A557, A558, A559, A561, A562, A563, A564, A565, A566, A567, A568.

It is understood by persons of ordinary skill in the art that the order of reactions depicted in the chemical schemes described herein may be changed according to the reactivity of substituents. Furthermore, it will be understood that reactants known in the literature can be prepared according to published procedures. In addition, it is understood that in case racemic mixtures are obtained by the chemical reactions described herein, desired enantiomers may be isolated in pure form by known purification methods known in the art, including but not limited to chiral HPLC methods.

### Preparation Example 4: 7-(2,3-dichloro-5-fluorophenyl)-N-[(4S)-3,4-dihydro-2H-1-benzopyran-4-yl]-4-(dimethylamino)-6-methylpyrrolo[1,2-b]pyridazine-3-carboxamide (A493)

Compound A493 was prepared according to the general process described in Scheme 2. The specific synthesis details are provided below.

### 1. Synthesis of ethyl 6-chloro-4-(trifluoromethanesulfonyloxy)pyrrolo[1,2-b]pyridazine-3-carboxylate (3-5)

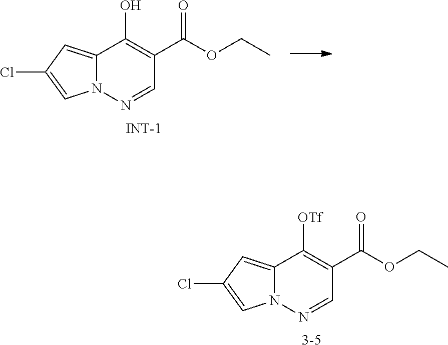

To a solution of 613.0 mg (2.5 mmol) of ethyl 6-chloro-4-hydroxypyrrolo[1,2-b]pyridazine-3-carboxylate (INT-1) and 776 μL (5.6 mmol) triethylamine in 5 mL of dichloromethane was added 471 μL (2.8 mmol) trifluoromethane anhydride at 0 °C. The solution was stirred over night at RT. Afterwards, water was added and the layers separated by phase transfer cartridge. The organic layer was dried over Na₂SO₄ and the solvent was evaporated under reduced pressure to yield 1.2 g of crude product as a brown oil. (3-5, ESI, m/z): 373 [M+H]⁺.

### 2. Synthesis of ethyl 6-chloro-4-(dimethylamino)pyrrolo[1,2-b]pyridazine-3-carboxylate (4-1)

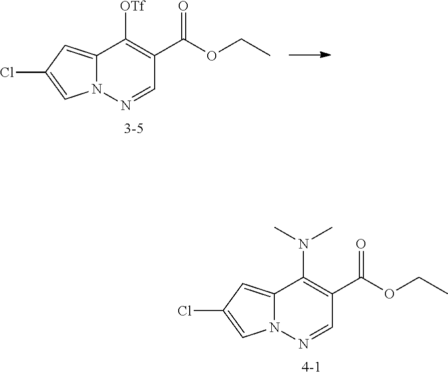

A solution of 31.0 g (83.1 mmol) of ethyl 6-chloro-4-(trifluoromethanesulfonyloxy)pyrrolo[1,2-b]pyridazine-3-carboxylate (3-5) in 300 mL of ethanol was added 40 mL (0.22 mol) of dimethylamine in ethanol (5.6 mol/L) and the solution heated to reflux for 15 min. After cooling to RT, the solution was diluted with ethyl acetate, washed with 1N HCl, 10% Na₂CO₃ solution and brine. After filtering through Celite, the solvent was evaporated under reduced pressure. Silica gel chromatography (CH/EA 9:1-2:8) yielded 22.3 g (70%) of 4-1 as yellow solid. (ESI, m/z): 268 [M+H]⁺.

### 3. Synthesis of ethyl 4-(dimethylamino)-6-methylpyrrolo[1,2-b]pyridazine-3-carboxylate (4-2)

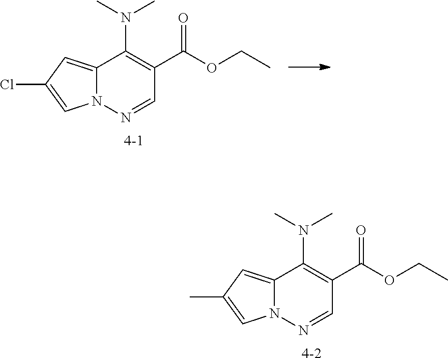

To a solution of 7.4 g (27.6 mmol) ethyl 6-chloro-4-(dimethylamino)pyrrolo[1,2-b]pyridazine-3-carboxylate (4-1) in 150 mL of dioxane in a three-necked flask was added 10 mL trimethylboroxine and 80 mL of 1M Na₂CO₃ solution. The mixture was purged with Argon for 5 min then heated to 50 °C and treated with 3.0 g (3.5 mmol) XPHOS Pd G3 under Argon and stirred for 1 h at 90 °C. After cooling to RT, the solution was diluted with ethyl acetate, washed with 10% Na₂CO₃ solution and brine. After filtering through Celite, the solvent was evaporated under reduced pressure. Silica gel chromatography (CH/EA 95:5-1:9) yielded 5.6 g (81%) of the title compound (4-2) as yellow oil that solidifies upon standing. (ESI, m/z): 248 [M+H]⁺.

### 4. Synthesis of ethyl 7-bromo-4-(dimethylamino)-6-methylpyrrolo[1,2-b]pyridazine-3-carboxylate (4-3)

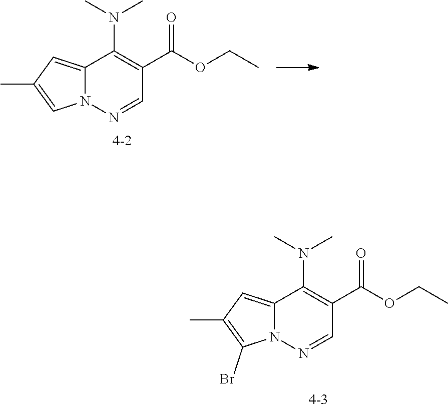

To a solution of 5.6 g (22.6 mmol) ethyl 4-(dimethylamino)-6-methylpyrrolo[1,2-b]pyridazine-3-carboxylate (4-2) in 120 mL DMF was added 3.9 g (21.9 mmol) NBS at 0 °C and the mixture stirred for 1 h at RT. The solution was diluted with ethyl acetate, washed with 10% Na₂CO₃ solution and brine. After filtering through Celite, the solvent was evaporated under reduced pressure. Silica gel chromatography (CH/EA 95:5-2:8) yielded 6.4 g (86%) of the title compound (4-3) as yellow solid. (ESI, m/z): 327 [M+H]⁺.

### 5. Synthesis of potassium 7-bromo-4-(dimethylamino)-6-methylpyrrolo[1,2-b]pyridazine-3-carboxylate (4-4)

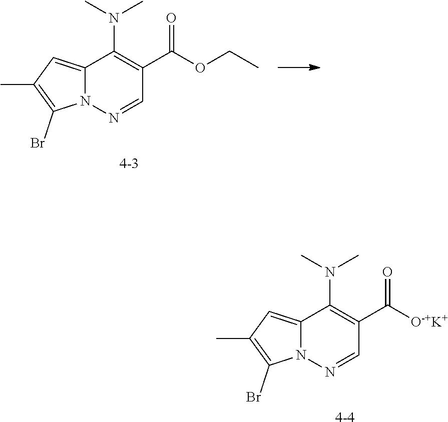

To a solution of 14.6 g (44.7 mmol) of ethyl 7-bromo-4-(dimethylamino)-6-methylpyrrolo[1,2-b]pyridazine-3-carboxylate (4-3) in 400 mL THF was added 12.7 g (89.0 mmol) TMSOK and the mixture stirred at 70 °C for 7 h. After evaporation of volatiles 15 g of crude product (4-4) was obtained and directly used in the next step. (ESI, m/z): 337 [M+H]⁺.

### 6. Synthesis of 7-bromo-N-[(4S)-3,4-dihydro-2H-1-benzopyran-4-yl]-4-(dimethylamino)-6-methylpyrrolo[1,2-b]pyridazine-3-carboxamide (4-5)

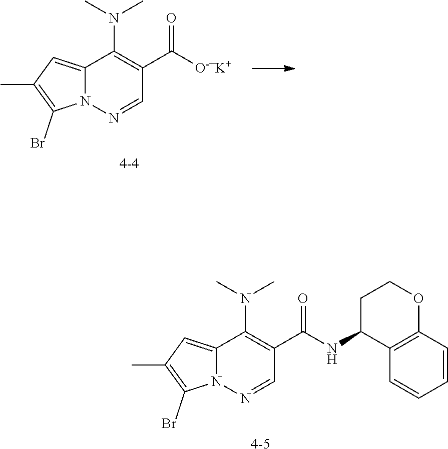

To a solution of 15 g (35.6 mmol) potassium 7-bromo-4-(dimethylamino)-6-methylpyrrolo[1,2-b]pyridazine-3-carboxylate (4-4) in 400 mL of DMF was added 28 mL (159.9 mmol) DIPEA and 24.0 g (63.1 mmol) HATU at RT. After stirring for 5 min at RT 13 g (70.0 mmol) (S)-chroman-4-amine hydrochloride were added and the mixture stirred for 30 min at 50 °C. After cooling to RT, the solution was diluted with ethyl acetate, washed with 1N HCl, 10% Na₂CO₃ solution and brine. After filtering through Celite, the solvent was evaporated under reduced pressure to about 200 mL and the product crystallized. The filtered solids were washed with little EA and dried to yield 10.6 g of product which was combined with the remaining mother liquor purified via silica gel chromatography (CH/EA 85:15-3:7) to yield a total of 11.9 g (77%) of the title compound (4-5) as beige solid. (ESI, m/z): 430 [M+H]⁺.

### 7. Synthesis of 7-(2,3-dichloro-5-fluorophenyl)-N-[(4S)-3,4-dihydro-2H-1-benzopyran-4-yl]-4-(dimethylamino)-6-methylpyrrolo[1,2-b]pyridazine-3-carboxamide (A493)

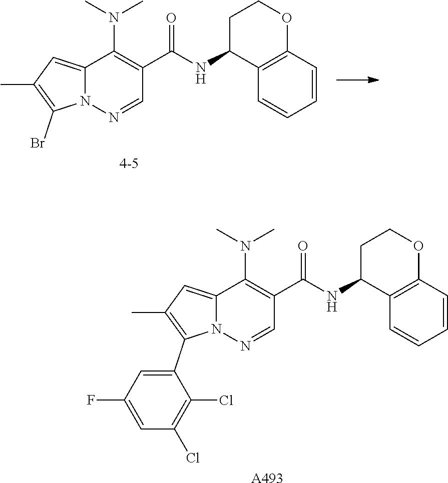

To a solution of 720 mg (1.5 mmol) of 7-bromo-N-[(4S)-3,4-dihydro-2H-1-benzopyran-4-yl]-4-(dimethylamino)-6-methylpyrrolo[1,2-b]pyridazine-3-carboxamide (4-5) in 25 mL of dioxane was added 430 mg (2.0 mmol) 2,3-dichloro-5-fluorophenyl)boronic acid under Argon followed by 200 mg (0.3 mmol) of 1,1′-Bis-(di-tert-butylphosphino-)ferrocen-palladiumdichloride and 3 mL Na₂CO₃ solution (2 mol/L). The mixture was stirred for 2 h at 80 °C in a sealed vessel. After cooling to RT, the solution was diluted with water and extracted with EA. After combining the organic layers, the solvent was evaporated under reduced pressure. Silica gel chromatography (CH to CH/EA 8:2) yielded 680 mg (51%) of the title compound (A493) as brown solid. (ESI, m/z): 514 [M+H]⁺.

### Preparation Example 5: Synthesis of 4-tert-butyl-6-chloro-7-(2,3-dichloro-5-fluorophenyl)-N-[(4S)-3,4-dihydro-2H-1-benzopyran-4-yl]pyrrolo[1,2-b]pyridazine-3-carboxamide (A502)

Compound A502 was prepared according to the general process described in Scheme 2. The specific synthesis details are provided below.

### 1. Synthesis of ethyl 7-bromo-4-tert-butyl-6-chloropyrrolo[1,2-b]pyridazine-3-carboxylate (5-1)

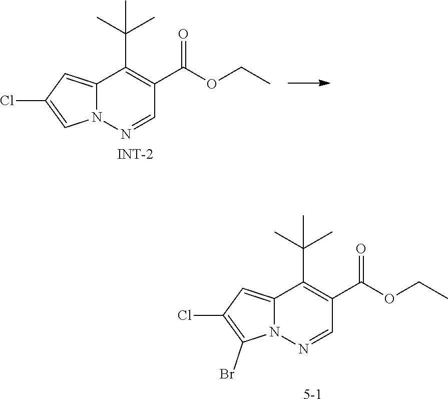

To a solution of 325 mg (1.0 mmol) ethyl 4-tert-butyl-6-chloropyrrolo[1,2-b]pyridazine-3-carboxylate (INT-2) in 5 mL DMF was added 166 mg (0.9 mmol) NBS at RT and the mixture stirred for 1 h at RT. The solution was diluted with water and extracted with ethyl acetate. The combined organic layers were dried over Na₂SO₄ and the solvent was evaporated under reduced pressure. Preparative HPLC (ACN/H₂O/TFA) yielded 323 mg (86%) of the title compound (5-1) as yellow solid. (5-1, ESI, m/z): 360 [M+H]⁺.

### 2. Synthesis of ethyl 4-tert-butyl-6-chloro-7-(2,3-dichloro-5-fluorophenyl)pyrrolo[1,2-b]pyridazine-3-carboxylate (5-2)

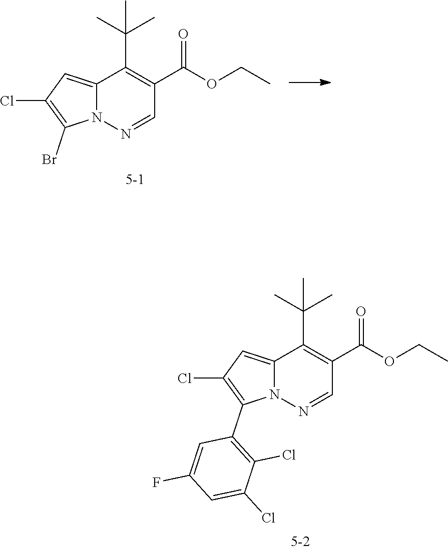

To a solution of 100 mg (0.27 mmol) of ethyl 7-bromo-4-tert-butyl-6-chloropyrrolo[1,2-b]pyridazine-3-carboxylate (5-1) in 3 mL of dioxane was added 63 mg (0.3 mmol) 2,3-dichloro-5-fluorophenyl)boronic acid under Argon followed by 0.5 mL Na₂CO₃ solution (2 mol/L) and 45 mg (0.05 mmol) of 1,1′-Bis-(di-tert-butylphosphino-)ferrocen-palladiumdichloride. The mixture was stirred for 2 h at 80 °C in a sealed vessel. After cooling to RT, the solution was diluted with water and extracted with EA. After combining the organic layers were dried over Na₂SO₄ and the solvent was evaporated under reduced pressure. Preparative HPLC (ACN/H₂O/TFA) yielded 45 mg (37%) of the title compound (5-2) as brown solid. (ESI, m/z): 444 [M+H]⁺.

### 3. Synthesis of 4-tert-butyl-6-chloro-7-(2,3-dichloro-5-fluorophenyl)pyrrolo[1,2-b]pyridazine-3-carboxylic acid (5-3)

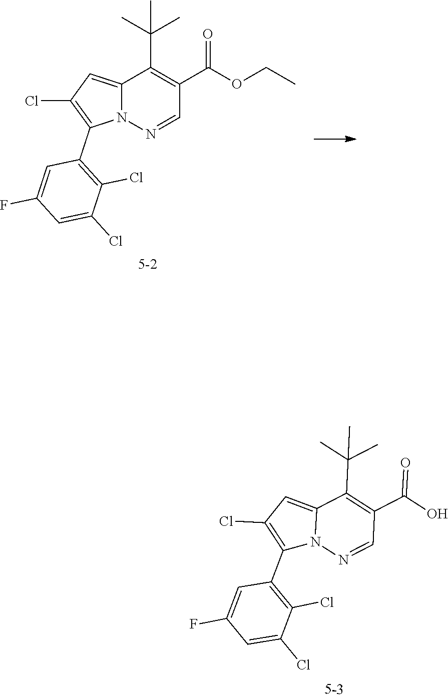 [SMILES: CCOC(=O)c1cnn2c(-c3cc(F)cc(Cl)c3Cl)c(Cl)cc2c1C(C)(C)C ; CC(C)(C)c1c(C(=O)O)cnn2c(-c3cc(F)cc(Cl)c3Cl)c(Cl)cc12]

To a solution of 45 mg (0.1 mmol) ethyl 4-tert-butyl-6-chloro-7-(2,3-dichloro-5-fluorophenyl)pyrrolo[1,2-b]pyridazine-3-carboxylate (5-2) in 1 mL dioxane/ethanol was added 0.5 mL NaOH solution (4 mol/L) at RT and the mixture stirred for 12 h at 90 °C. After cooling to RT, the solution was treated with HCl (4 mol/L) until acidic pH at 0 °C and diluted with DCM. After filtration through a phase separation cartridge, the organic phase was evaporated under reduced pressure and the crude title product (5-3) directly used in the subsequent step. (ESI, m/z): 416 [M+H]⁺.

### 4. Synthesis of 4-tert-butyl-6-chloro-7-(2,3-dichloro-5-fluorophenyl)-N-[(4S)-3,4-dihydro-2H-1-benzopyran-4-yl]pyrrolo[1,2-b]pyridazine-3-carboxamide (A502)

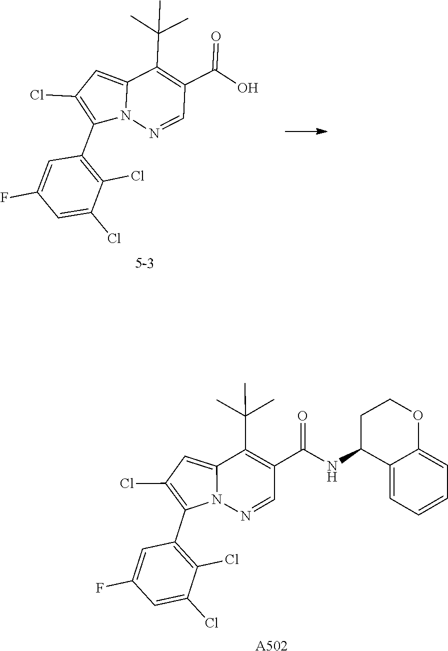 [SMILES: CC(C)(C)c1c(C(=O)O)cnn2c(-c3cc(F)cc(Cl)c3Cl)c(Cl)cc12 ; CC(C)(C)c1c(C(=O)N[C@H]2CCOc3ccccc32)cnn2c(-c3cc(F)cc(Cl)c3Cl)c(Cl)cc12 ; "A502"]

To a solution of 42 mg (0.1 mmol) 4-tert-butyl-6-chloro-7-(2,3-dichloro-5-fluorophenyl)pyrrolo[1,2-b]pyridazine-3-carboxylic acid (5-3) in 1.5 mL DMF was added 51 μL (0.3 mmol) DIPEA and 38 mg (0.1 mmol) HATU at RT. After stirring for 5 min at RT 28 mg (0.15 mmol) (S)-chroman-4-amine hydrochloride were added and the mixture stirred for 2 h at RT. After acidifying with TFA, the mixture was directly purified via preparative HPLC (ACN/H₂O/TFA) to yield 10 mg (18%) of the title compound (A502) as yellow solid. (ESI, m/z): 547 [M+H]⁺.

### Preparation Example 6: Synthesis of N-[(4S)-3,4-dihydro-2H-1-benzopyran-4-yl]-6-methyl-4-(propan-2-yl)-7-(2,3,5-trifluorophenyl)pyrrolo[1,2-b]pyridazine-3-carboxamide (A513)

### 1. Synthesis of ethyl 6-chloro-4-(prop-1-en-2-yl)pyrrolo[1,2-b]pyridazine-3-carboxylate (6-1)

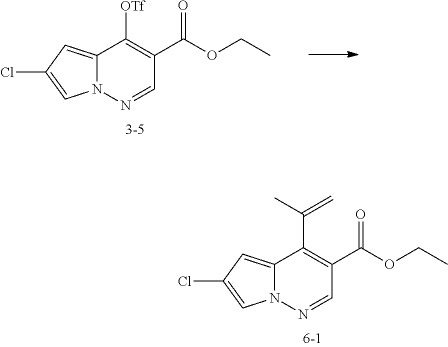 [SMILES: CCOC(=O)c1cnn2cc(Cl)cc2c1OS(=O)(=O)C(F)(F)F ; C=C(C)c1c(C(=O)OCC)cnn2cc(Cl)cc12]

A solution of 1.55 g (8.1 mmol) of ethyl 6-chloro-4-(trifluoromethanesulfonyloxy)pyrrolo[1,2-b]pyridazine-3-carboxylate (3-5) in 25 mL 1.2-dimethoxyethane was treated with 9.24 g (28.3 mmol) Cs₂CO₃ dissolved in 5 mL of water under Argon, followed by addition of 2.59 mL (13.7 mmol) of isopropenylboronic acid pinacol ester. After degassing for 5 min, 794 mg (1.0 mmol) of 1,1′-bis-(di-tert-butylphosphino-)ferrocen-palladiumdichloride were added under Argon and the sealed vessel stirred for 3 h at 80 °C. After cooling to RT, the solution was diluted with ethyl acetate and washed with 10% Na₂CO₃ solution and brine. The solvent was dried over Na₂SO₄ and the evaporated under reduced pressure. Preparative HPLC (ACN/H₂O/TFA) yielded 578 mg (27%) of the title compound (6-1) as solid. (ESI, m/z): 265 [M+H]⁺.

### 2. Synthesis of ethyl 6-chloro-4-(propan-2-yl)pyrrolo[1,2-b]pyridazine-3-carboxylate (6-2)

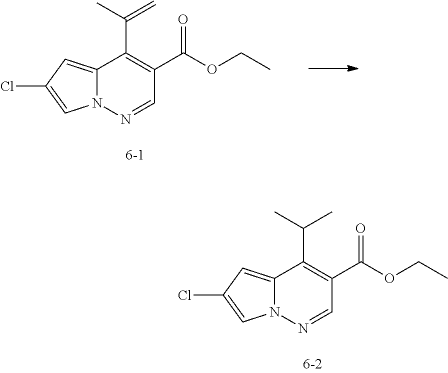 [SMILES: C=C(C)c1c(C(=O)OCC)cnn2cc(Cl)cc12 ; CCOC(=O)c1cnn2cc(Cl)cc2c1C(C)C]

A solution of 578 mg (2.1 mmol) ethyl 6-chloro-4-(prop-1-en-2-yl)pyrrolo[1,2-b]pyridazine-3-carboxylate (6-1) in 25 mL of acetic acid was treated with 50 mg of PtO₂ and stirred for 30 min at RT under 50 psi of hydrogen. The crude mixture was filtered, and the filtrate concentrated. Preparative HPLC (ACN/H₂O/TFA) yielded 337 mg (57%) of the title compound (6-2) as solid. (ESI, m/z): 267 [M+H]⁺.

### 3. Synthesis of ethyl 6-methyl-4-(propan-2-yl)pyrrolo[1,2-b]pyridazine-3-carboxylate (6-3)

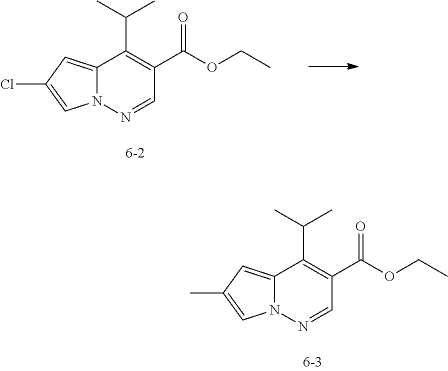 [SMILES: CCOC(=O)c1cnn2cc(Cl)cc2c1C(C)C ; CCOC(=O)c1cnn2cc(C)cc2c1C(C)C]

To a solution of 2.0 g (7.5 mmol) ethyl 6-chloro-4-(propan-2-yl)pyrrolo[1,2-b]pyridazine-3-carboxylate (6-2) in 22 mL of dioxane in a vial was added 505 mg (0.75 mmol) XPHOS Pd G3 under Argon, followed by 3.5 mL (24.7 mmol) trimethylboroxine and 11.2 mL of 2M Na₂CO₃ solution. The mixture was purged with Argon for 5 min then heated for 6 h at 80 °C. After cooling to RT, the solution was diluted with water and extracted with ethyl acetate. The combined organic layers were dried over Na₂SO₄ and the solvent was evaporated under reduced pressure. Silica gel chromatography (CH/EA 9:1) yielded 1.43 g (69%) of the title compound (6-3) as solid. (ESI, m/z): 247 [M+H]⁺.

### 4. Synthesis of ethyl 7-bromo-6-methyl-4-(propan-2-yl)pyrrolo[1,2-b]pyridazine-3-carboxylate (6-4)

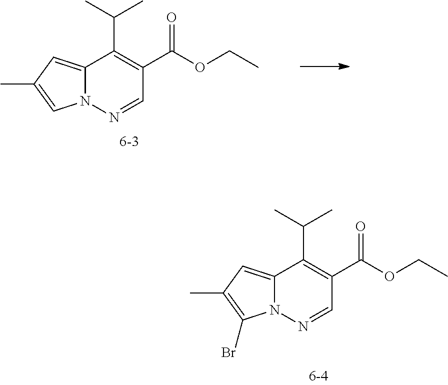 [SMILES: CCOC(=O)c1cnn2cc(C)cc2c1C(C)C ; CCOC(=O)c1cnn2c(Br)c(C)cc2c1C(C)C]

To a solution of 1.43 g (5.2 mmol) of ethyl 6-methyl-4-(propan-2-yl)pyrrolo[1,2-b]pyridazine-3-carboxylate (6-3) in 14 mL DMF was added 837 mg (4.7 mmol) NBS at RT and the mixture stirred for 30 min at RT. After acidifying with TFA, the mixture was directly purified via preparative HPLC (ACN/H₂O/TFA) to yield a total of 1.14 g (67%) of the title compound (6-4) as yellow oil. (ESI, m/z): 326 [M+H]⁺.

### 5. Synthesis of ethyl 6-methyl-4-(propan-2-yl)-7-(2,3,5-trifluorophenyl)pyrrolo[1,2-b]pyridazine-3-carboxylate (6-5)

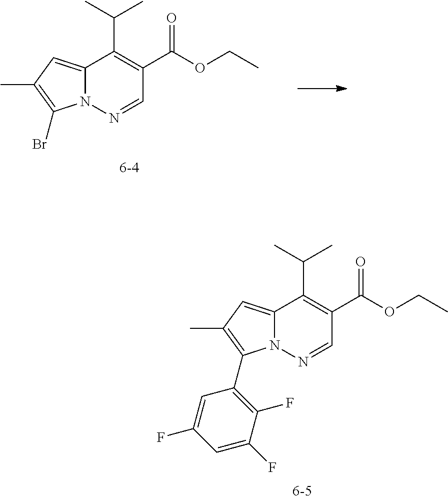 [SMILES: CCOC(=O)c1cnn2c(Br)c(C)cc2c1C(C)C ; CCOC(=O)c1cnn2c(-c3cc(F)cc(F)c3F)c(C)cc2c1C(C)C]

To a solution of 25 mg (0.1 mmol) of ethyl 7-bromo-6-methyl-4-(propan-2-yl)pyrrolo[1,2-b]pyridazine-3-carboxylate (6-4) in 1 mL of dioxane was added 20 mg (0.1 mmol) of 2,3,5-trifluorophenylboronic acid under Argon followed by 153 μL Na₂CO₃ solution (2 mol/L) and 10 mg (0.015 mmol) of 1,1′-Bis-(di-tert-butylphosphino-)ferrocen-palladiumdichloride. The mixture was stirred for 1 h at 80 °C in a sealed vessel. After cooling to RT, the solution was diluted with DCM and brine. After filtration through a phase separation cartridge, the organic phase was evaporated under reduced pressure and the mixture purified via preparative HPLC (ACN/H₂O/TFA) to yield 10 mg (34%) of the title compound (6-5) as brown solid. (ESI, m/z): 377 [M+H]⁺.

### 6. Synthesis of 6-methyl-4-(propan-2-yl)-7-(2,3,5-trifluorophenyl)pyrrolo[1,2-b]pyridazine-3-carboxylic acid (6-6)

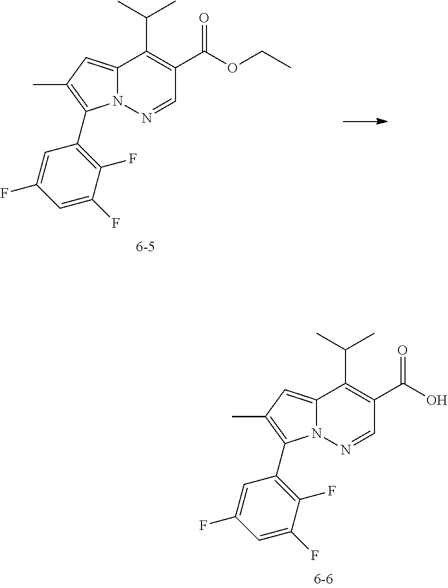 [SMILES: CCOC(=O)c1cnn2c(-c3cc(F)cc(F)c3F)c(C)cc2c1C(C)C ; Cc1cc2c(C(C)C)c(C(=O)O)cnn2c1-c1cc(F)cc(F)c1F]

To a solution of 10 mg (0.02 mmol) ethyl 6-methyl-4-(propan-2-yl)-7-(2,3,5-trifluorophenyl)pyrrolo[1,2-b]pyridazine-3-carboxylate (6-5) in 2 mL ethanol was added 0.13 mL NaOH solution (4 mol/L) at RT and the mixture stirred for 2 h at 90 °C. After cooling to RT, the solution was treated with HCl (4 mol/L) until acidic pH at 0 °C and diluted with DCM. After filtration through a phase separation cartridge, the organic phase was evaporated under reduced pressure and the crude title product (6-6) directly used in the subsequent step. (ESI, m/z): 349 [M+H]⁺.

### 7. Synthesis of N-[(4S)-3,4-dihydro-2H-1-benzopyran-4-yl]-6-methyl-4-(propan-2-yl)-7-(2,3,5-trifluorophenyl)pyrrolo[1,2-b]pyridazine-3-carboxamide (A513)

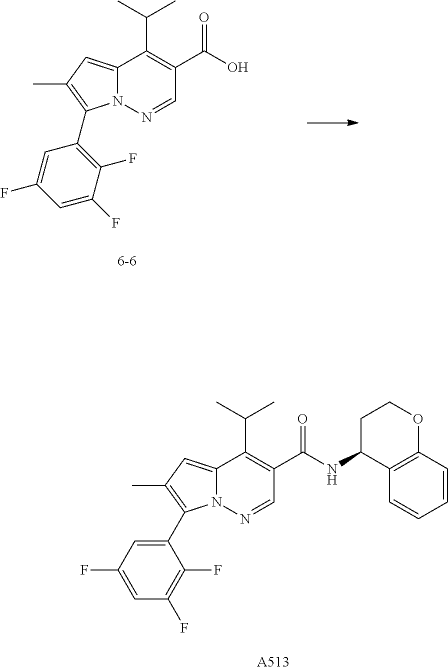 [SMILES: Cc1cc2c(C(C)C)c(C(=O)O)cnn2c1-c1cc(F)cc(F)c1F ; Cc1cc2c(C(C)C)c(C(=O)N[C@H]3CCOc4ccccc43)cnn2c1-c1cc(F)cc(F)c1F ; "A513"]

To a solution of 9 mg (0.02 mmol) 6-methyl-4-(propan-2-yl)-7-(2,3,5-trifluorophenyl)pyrrolo[1,2-b]pyridazine-3-carboxylic acid (6-6) in 1 mL DMF was added 13 μL (0.078 mmol) DIPEA and 9.8 mg (0.026 mmol) HATU at RT. After stirring for 10 min at RT 5.7 mg (0.03 mmol) (S)-chroman-4-amine hydrochloride were added and the mixture stirred at RT overnight. After diluting with ACN and acidifying with TFA, the mixture was directly purified via preparative HPLC (ACN/H₂O/TFA) to yield 9 mg (72%) of the title compound (A513) as solid. (ESI, m/z): 480 [M+H]⁺.

### Preparation Example 7: Synthesis of 6-chloro-7-(2,3-dichloro-5-fluorophenyl)-N-[(4S)-3,4-dihydro-2H-1-benzopyran-4-yl]-4-(propan-2-yl)pyrrolo[1,2-b]pyridazine-3-carbothioamide (A539)

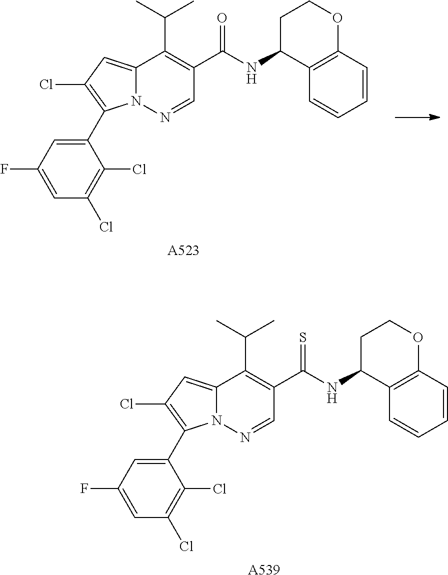 [SMILES: CC(C)c1c(C(=O)N[C@H]2CCOc3ccccc32)cnn2c(-c3cc(F)cc(Cl)c3Cl)c(Cl)cc12 ; "A523" ; CC(C)c1c(C(=S)N[C@H]2CCOc3ccccc32)cnn2c(-c3cc(F)cc(Cl)c3Cl)c(Cl)cc12 ; "A539"]

To as solution of 48 mg (0.1 mmol) of 6-chloro-7-(2,3-dichloro-5-fluorophenyl)-N-[(4S)-3,4-dihydro-2H-1-benzopyran-4-yl]-4-(propan-2-yl)pyrrolo[1,2-b]pyridazine-3-carboxamide (A523) in 3 mL of THF was added 54 mg (0.14 mmol) Lawesson's reagent. The mixture was stirred for 3 h at RT, then heated to 50 °C over night. After cooling to RT, the mixture was acidified with TFA and directly purified via preparative HPLC (ACN/H₂O/TFA) to yield 16.5 mg (33%) of the title compound (A539) as yellow solid. (ESI, m/z): 549 [M+H]⁺.

### Preparation Example 8: Synthesis of 4-tert-butyl-7-(2,3-dichloro-5-fluorophenyl)-N-[(4S)-3,4-dihydro-2H-1-benzopyran-4-yl]-N,6-dimethylpyrrolo[1,2-b]pyridazine-3-carboxamide (A543)

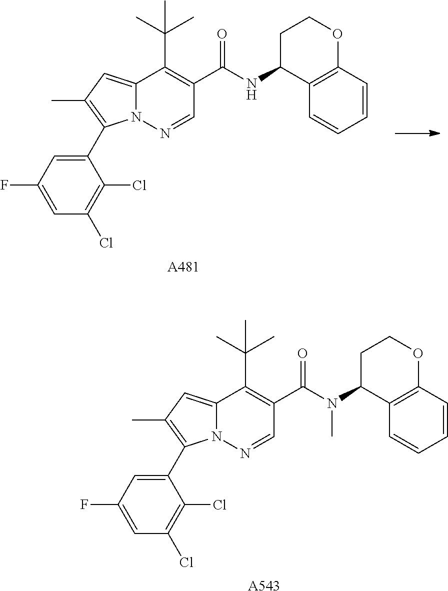 [SMILES: Cc1cc2c(C(C)(C)C)c(C(=O)N[C@H]3CCOc4ccccc43)cnn2c1-c1cc(F)cc(Cl)c1Cl ; "A481" ; Cc1cc2c(C(C)(C)C)c(C(=O)N(C)[C@H]3CCOc4ccccc43)cnn2c1-c1cc(F)cc(Cl)c1Cl ; "A543"]

To a solution of 20 mg (0.03 mmol) 4-tert-butyl-7-(2,3-dichloro-5-fluorophenyl)-N-[(4S)-3,4-dihydro-2H-1-benzopyran-4-yl]-6-methylpyrrolo[1,2-b]pyridazine-3-carboxamide (A481) in 3 mL of DMF was added 3.3 mg (0.077 mmol) of NaH. After addition of 2.6 μL (0.042 mmol) Mel the mixture was stirred for 1 h at RT and then directly purified via preparative HPLC (ACN/H₂O/TFA) to yield 9.5 mg (41%) of the title compound (A543) as yellow solid. (ESI, m/z): 541 [M+H]⁺.

### Preparation Example 9: Synthesis of 4-tert-butyl-N-[(4S)-3,4-dihydro-2H-1-benzopyran-4-yl]-6-methyl-7-(2,3,5-trichlorophenyl)pyrrolo[1,2-b]pyridazine-3-carboxamide (A472)

Scheme 4 below describes the synthesis of Compound A472:

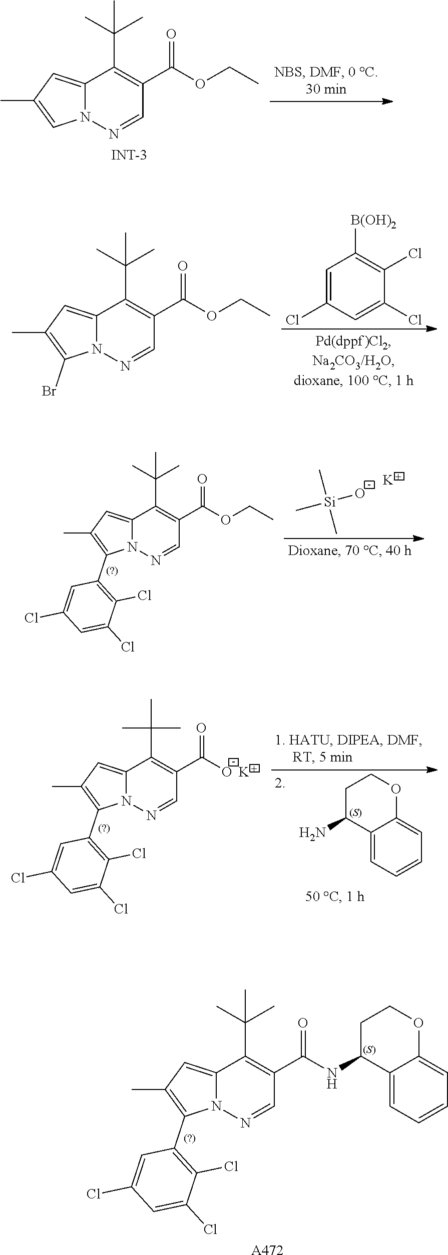

### Synthesis of ethyl 7-bromo-4-tert-butyl-6-methylpyrrolo[1,2-b]pyridazine-3-carboxylate

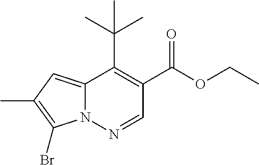 [SMILES: CCOC(=O)c1cnn2c(Br)c(C)cc2c1C(C)(C)C]

Into a 1.0 L round-bottom flask was added 8.2 g (31.5 mmol, 1 equiv.) of ethyl 4-tert-butyl-6-methylpyrrolo[1,2-b]pyridazine-3-carboxylate (INT-3) and 220 mL of DMF. At 0 °C, cooled with an ice-bath, NBS (5.5 g, 30.9 mmol, 0.98 equiv.) was added in portions. The mixture was warmed to RT and stirred for 30 min. The solution was diluted with ethyl acetate and washed with 10% Na₂CO₃ solution and brine. The combined organic layers were filtered over Celite and the solvent was evaporated under reduced pressure. Silica gel chromatography (CH/EA gradient) yielded 10.5 g (98%, 31.0 mmol) of the title compound as yellow oil. (ESI, m/z): 340 [M+H]⁺.

### 1. Synthesis of ethyl 4-tert-butyl-6-methyl-7-(2,3,5-trichlorophenyl)pyrrolo[1,2-b]pyridazine-3-carboxylate

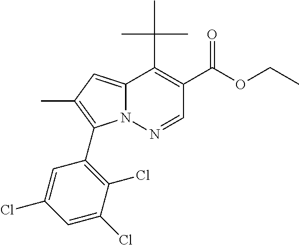 [SMILES: CCOC(=O)c1cnn2c(-c3cc(Cl)cc(Cl)c3Cl)c(C)cc2c1C(C)(C)C]

Into a 3-necked 500 mL flask was added 7-bromo-4-tert-butyl-6-methylpyrrolo[1,2-b]pyridazine-3-carboxylate (9.0 g, 85% purity, 22.6 mmol, 1 equiv.) and 180 mL of dioxane. The mixture was purged with argon while stirring for 10 min. The mixture was then treated with 2,3,5-trichlorophenylboronic acid (6.8 g, 30.2 mmol, 1.3 equiv.) followed by 40 mL Na₂CO₃ solution (2 mol/L) and purged for further 5 min with argon and subsequently heated to 50 °C. Then 1,1′-Bis-(di-tert-butylphosphino)-ferrocen-palladiumdichloride (2.0 g, 3.07 mmol, 0.13 equiv.) were added under argon and the mixture was stirred for 1 h at 100 °C. After cooling to RT, the solution was diluted with water and extracted with EA. After combining the organic layers and filtration over Celite, the solvent was evaporated under reduced pressure. Silica gel chromatography (CH to CH/EA 96:4) yielded 10.3 g (75% purity, 78%, 17.6 mmol) of the title compound as yellow oil. (ESI, m/z): 439 [M+H]⁺.

### 2. Synthesis of potassium 4-tert-butyl-6-methyl-7-(2,3,5-trichlorophenyl)pyrrolo[1,2-b]pyridazine-3-carboxylate

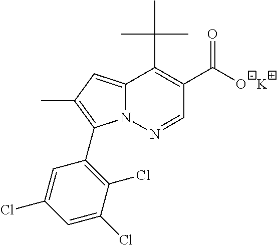

In a 500 mL flask ethyl 4-tert-butyl-6-methyl-7-(2,3,5-trichlorophenyl)pyrrolo[1,2-b]pyridazine-3-carboxylate (7.0 g, 15.9 mmol, 1 equiv.) was dissolved in 200 mL of tetrahydrofuran. After addition of potassium trimethylsilanolate (6.9 g, technical grade (90%), 48.8 mmol, 3.0 equiv.) the mixture was heated to 70 °C for 40 h. The solvent was evaporated, and the crude product directly used in the next step without any further purification. (ESI, m/z): 411 [M+H]⁺.

### 3. Synthesis of4-tert-butyl-N-[(4S)-3,4-dihydro-2H-1-benzopyran-4-yl]-6-methyl-7-(2,3,5-trichlorophenyl)pyrrolo[1,2-b]pyridazine-3-carboxamide (A472)

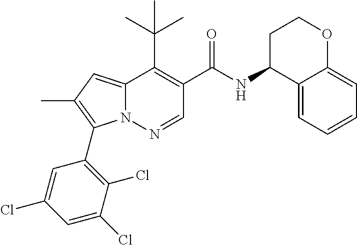 [SMILES: Cc1cc2c(C(C)(C)C)c(C(=O)N[C@H]3CCOc4ccccc43)cnn2c1-c1cc(Cl)cc(Cl)c1Cl]

To a solution of potassium 4-tert-butyl-6-methyl-7-(2,3,5-trichlorophenyl)pyrrolo[1,2-b]pyridazine-3-carboxylate (7.2 g (80%), 12.8 mmol, 1 equiv.) in 150 mL of DMF in a 250 mL flask was added DIPEA (7.9 mL, 46.2 mmol, 3.6 equiv.). After addition of HATU (7.0 g, 18.4 mmol, 1.4 equiv.) the mixture was stirred for 5 min at RT. Then (S)-chroman-4-amine hydrochloride (3.8 g, 20.5 mmol, 1.6 equiv.) were added and the mixture stirred for 1 h at 50 °C. After cooling to RT, the mixture was diluted with ethyl acetate and extracted with HCl solution (1 mol/L), 10% Na₂CO₃ solution and brine. The organic layer was filtered over Celite and the solvent was evaporated under reduced pressure. The residue was purified via preparative HPLC (ACN/H₂O/TFA) and the product fractions were evaporated to dryness, redissolved in EtOH (20 mL) to yield 3.5 g (6.45 mmol, 50%) of the title compound as light-yellow solid after drying. (ESI, m/z): 542 [M+H]⁺.

### Preparation Example 10: Synthesis of 2H,3H,4H-pyrano[3,2-c]pyridin-4-amine (12-2)

The bicyclic amine used in the preparation of Ring System AF present in compound A569 (see Table 1) is prepared from commercially available materials according to the process shown below.

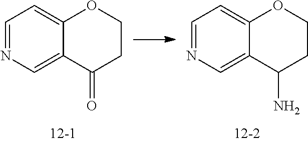 [SMILES: O=C1CCOc2ccncc21 ; NC1CCOc2ccncc21]

To a solution of 400 mg (2.6 mmol) 2H-Pyrano[3,2-c]pyridin-4(3H)-one (12-1) and 1.35 g (21.4 mmol) ammonium formate in 13.4 mL methanol were added 887 mg (13.4 mmol) sodium cyanoborohydride at RT and the mixture was then stirred at 60 °C over night. After addition of further same amount of ammonium formate and sodium cyanoborohydride and stirring for 3 h at 60 °C, the reaction mixture was cooled to RT, diluted with ethyl acetate and quenched with water. After phase separation the organic phase was dried over anhydrous Na₂SO₄ and the solvent was evaporated to yield 120 mg (23%) of the title compound (12-2) as crude oil. The crude material was used without further purification. (ESI, m/z): 151 [M+H]⁺.

Table 6 below includes ¹H NMR data for certain compounds shown in Table 1 and prepared according to the processes outlined in schemes 1, 2 and 3 and the preparation examples shown above:

TABLE 6
¹H NMR data for Compounds of the Invention

| Compound | ¹H NMR Spectra |
|---|---|
| A461 | (400 MHz, DMSO-d6, ppm) δ 9.93 (s, 1H), 7.99 (s, 1H), 7.68 (d, J = 2.03 Hz, 2H), |
| | 7.57 (t, J = 1.90 Hz, 1H), 6.75-7.26 (m, 5H), 4.30-4.43 (m, 1H), 4.17 (s, 1H), 3.99 |
| | (s, 1H), 3.34-3.47 (m, 1H), 2.52-2.55 (m, 3H), 2.11-2.32 (m, 2H), 1.36-1.46 (m, 6H) |
| A462 | (400 MHz, DMSO-d6, ppm) δ 9.93 (s, 1H), 7.99 (s, 1H), 7.68 (d, J = 2.03 Hz, 2H), |
| | 7.57 (t, J = 1.90 Hz, 1H), 6.75-7.26 (m, 5H), 4.30-4.43 (m, 1H), 4.17 (s, 1H), 3.99 |
| | (s, 1H), 3.34-3.47 (m, 1H), 2.52-2.55 (m, 3H), 2.11-2.32 (m, 2H), 1.36-1.46 (m, 6H) |
| A463 | (400 MHz, DMSO-d6) δ ppm 9.02 (d, J = 8.11 Hz, 1 H) 8.27 (s, 1 H) 7.77 (d, |
| | J = 1.90 Hz, 2 H) 7.68 (t, J = 1.90 Hz, 1 H) 7.31 (d, J = 7.37 Hz, 1 H) 7.20 (s, 1 H) |
| | 7.17 (dt, J = 7.80, 1.63 Hz, 1 H) 6.91 (t, J = 7.08 Hz, 1 H) 6.79 (dd, J = 8.17, 1.08 |
| | Hz, 1 H) 5.15-5.27 (m, 1 H) 4.25 (br d, J = 3.42 Hz, 2 H) 3.42-3.58 (m, 1 H) |
| | 2.12-2.24 (m, 1 H) 1.96-2.08 (m, 1 H) 1.44 (dd, J = 10.52, 7.10 Hz, 6 H) |
| A464 | (400 MHz, DMSO-d6) δ ppm 9.03 (d, J = 8.11 Hz, 1 H) 7.98 (s, 1 H) 7.65 (d, |
| | J = 1.90 Hz, 2 H) 7.58-7.62 (m, 1 H) 7.33 (d, J = 7.10 Hz, 1 H) 7.16 (t, J = 7.42 |
| | Hz, 1 H) 6.89-6.94 (m, 2 H) 6.79 (dd, J = 8.17, 0.95 Hz, 1 H) 5.13-5.19 (m, 1 |
| | H) 4.17-4.29 (m, 2 H) 2.33-2.44 (m, 3 H) 2.09-2.22 (m, 1 H) 1.95-2.09 |
| | (m, 1 H) 1.57 (s, 9 H) |
| A465 | (400 MHz, DMSO-d6) δ ppm 8.95 (d, J = 8.24 Hz, 1 H) 8.12 (s, 1 H) 7.68 (d, |
| | J = 1.90 Hz, 2 H) 7.60 (t, J = 1.90 Hz, 1 H) 7.30 (d, J = 7.22 Hz, 1 H) 7.16 (t, |
| | J = 7.69 Hz, 1 H) 6.88-6.94 (m, 2 H) 6.79 (d, J = 7.96 Hz, 1 H) 5.18-5.24 (m, 1 |
| | H) 4.20-4.30 (m, 2 H) 3.49-3.64 (m, 1 H) 2.39 (s, 3 H) 2.09-2.22 (m, 1 H) |
| | 1.98-2.09 (m, 1 H) 1.45 (dd, J = 10.27, 7.10 Hz, 6 H) |
| A466 | (400 MHz, DMSO-d6) δ ppm 9.04 (br d, J = 7.86 Hz, 1 H) 8.39 (s, 1 H) 8.01 (s, 2 H) |
| | 7.58-7.63 (m, 1 H) 7.32 (br d, J = 7.60 Hz, 1 H) 7.12-7.21 (m, 1 H) 7.09 (s, 1 H) 6.92 |
| | (t, J = 7.41 Hz, 1 H) 6.79 (d, J = 8.24 Hz, 1 H) 5.17-5.26 (m, 1 H) 4.17-4.32 (m, 2 H) |
| | 3.44-3.60 (m, 1 H) 2.11-2.22 (m, 1 H) 1.94-2.11 (m, 1 H) 1.44-1.53 (m, 6 H) |
| A467 | (400 MHz, DMSO-d6) δ ppm 9.10 (d, J = 8.11 Hz, 1 H) 8.32 (s, 1 H) 8.18 (d, |
| | J = 1.90 Hz, 2 H) 7.70 (s, 1 H) 7.57 (t, J = 1.90 Hz, 1 H) 7.36 (d, J = 7.48 Hz, 1 H) |
| | 7.16-7.22 (m, 1 H) 6.86-6.96 (m, 1 H) 6.80 (dd, J = 8.17, 0.82 Hz, 1 H) 5.15-5.23 |
| | (m, 1 H) 4.05-4.33 (m, 3 H) 2.11-2.24 (m, 1 H) 1.97-2.11 (m, 1 H) 1.42-1.52 (m, 6 H) |
| A468 | (400 MHz, DMSO-d6) δ ppm 9.14 (d, J = 7.98 Hz, 1 H) 8.54 (s, 1 H) 8.22 (d, |
| | J = 1.77 Hz, 2 H) 8.02 (s, 1 H) 7.62 (s, 1 H) 7.38 (d, J = 7.60 Hz, 1 H) 7.18 (t, |
| | J = 7.73 Hz, 1 H) 6.93 (t, J = 7.48 Hz, 1 H) 6.80 (d, J = 8.11 Hz, 1 H) 5.18-5.24 |
| | (m, 1 H) 4.18-4.32 (m, 2 H) 3.54 (dt, J = 13.97, 7.02 Hz, 1 H) 2.14-2.25 (m, 1 |
| | H) 2.02-2.14 (m, 1 H) 1.48 (t, J = 7.03 Hz, 6 H) |
| A469 | (400 MHz, DMSO-d6) δ ppm 9.02 (d, J = 7.98 Hz, 1 H) 8.16-8.24 (m, 3 H) |
| | 7.49 (t, J = 1.84 Hz, 1 H) 7.44 (s, 1 H) 7.36 (d, J = 7.48 Hz, 1 H) 7.15-7.27 (m, |
| | 1 H) 6.93 (t, J = 7.41 Hz, 1 H) 6.80 (d, J = 7.98 Hz, 1 H) 5.15-5.23 (m, 1 H) |
| | 4.18-4.33 (m, 2 H) 3.79 (dt, J = 14.04, 6.99 Hz, 1 H) 2.58 (s, 3 H) 2.11-2.25 |
| | (m, 1 H) 1.99-2.11 (m, 1 H) 1.23-1.52 (m, 6 H) |
| A470 | (400 MHz, DMSO-d6) δ ppm 9.00 (d, J = 8.11 Hz, 1 H) 8.34 (s, 1 H) 8.25 (d, |
| | J = 1.77 Hz, 2 H) 7.60 (d, J = 4.82 Hz, 1 H) 7.51 (t, J = 1.84 Hz, 1 H) 7.33 (d, |
| | J = 7.48 Hz, 1 H) 7.12-7.19 (m, 1 H) 7.07 (d, J = 4.82 Hz, 1 H) 6.90-7.00 (m, 1 |
| | H) 6.79 (d, J = 7.99 Hz, 1 H) 5.20-5.26 (m, 1 H) 4.21-4.31 (m, 2 H) 3.59 (spt, |
| | J = 7.03 Hz, 1 H) 2.12-2.24 (m, 1 H) 1.99-2.12 (m, 1 H) 1.41-1.52 (m, 6 H) |
| A472 | (400 MHz, DMSO-d6, ppm) δ 9.03-9.08 (m, 1H), 7.96 (d, 1H), 7.89-7.90 (d, |
| | 1H), 7.56-7.61 (dd, 1H), 7.29-7.33 (t, 1H), 7.13-7.17 (t, 1H), 6.89-6.92 (m, |
| | 2H), 6.77-6.79 (d, 1H), 5.13-5.17 (m, 1H), 4.17-4.27 (m, 2H), 2.21 (s, 3H), |
| | 1.99-2.14 (m, 2H), 1.58 (s, 9H) |
| A471 | (400 MHz, DMSO-d6) δ ppm 9.05 (d, J = 7.98 Hz, 1 H) 7.94 (s, 1 H) 7.56-7.69 |
| | (m, 1 H) 7.25-7.37 (m, 2 H) 7.16 (t, J = 7.67 Hz, 1 H) 6.86-6.96 (m, 2 |
| | H) 6.78 (d, J = 8.11 Hz, 1 H) 5.12-5.19 (m, 1 H) 4.15-4.30 (m, 2 H) 2.29 (s, 3 |
| | H) 2.09-2.25 (m, 1 H) 1.94-2.09 (m, 1 H) 1.58 (s, 9 H) |
| A475 | (400 MHz, DMSO-d6, ppm) δ 8.80-8.82 (d, 1H), 8.00 (s, 1H), 7.74-7.75 (d, 2H), |
| | 7.65-7.66 (t, 1H), 7.29-7.30 (d, 1H), 7.16-7.18 (t, 1H), 7.11 (s, 1H), 6.88-6.92 |
| | (t, 1H), 6.78-6.80 (d, 1H), 5.15-5.19 (m, 1H), 4.18-4.28 (m, 2H), 3.18 (s, |
| | 6H), 1.99-2.16 (m, 2H) |
| A473 | (400 MHz, DMSO-d6, ppm) δ 8.79-8.81 (dd, 1H), 8.01-8.02 (d, 1H), 7.90 (s, |
| | 1H), 7.60-7.62 (dd, 1H), 7.26-7.28 (m, 1H), 7.13-7.17 (t, 1H), 7.11 (s, 1H), |
| | 8.86-8.90 (t, 1H), 6.77-6.79 (d, 1H), 5.15-5.17 (m, 1H), 4.19-4.27 (m, 2H), |
| | 3.19 (d, 6H), 1.91-2.17 (m, 2H) |
| A474 | (400 MHz, DMSO-d6, ppm) δ 8.80-8.82 (d, 1H), 7.94 (s, 1H), 7.66-7.73 (m, |
| | 1H), 7.27-7.32 (m, 2H), 7.14-7.18 (m, 2H), 6.87-6.91 (t, 1H), 6.77-6.79 (d, |
| | 1H), 5.14-5.19 (m, 1H), 4.20-4.24 (m, 2H), 3.19 (s, 6H), 1.98-2.16 (m, 2H) |
| A522 | (400 MHz, DMSO-d6) δ ppm 9.12 (t, J = 9.08 Hz, 1 H) 8.07 (d, J = 7.10 Hz, 1 |
| | H) 8.04 (d, J = 2.53 Hz, 1 H) 7.69 (dd, J = 12.04, 2.41 Hz, 1 H) 7.32 (t, J = 8.17 |
| | Hz, 1 H) 7.20 (s, 1 H) 7.16 (t, J = 7.60 Hz, 1 H) 6.87-6.92 (m, 1 H) 6.78 (d, |
| | J = 7.35 Hz, 1 H) 5.11-5.18 (m, 1 H) 4.15-4.28 (m, 2 H) 2.52-2.65 (m, 3 H) |
| | 2.14 (br d, J = 3.42 Hz, 1 H) 1.98-2.07 (m, 1 H) 1.57 (s, 9 H) |
| A525 | (400 MHz, DMSO-d6) δ ppm 8.83 (dd, J = 8.11, 3.17 Hz, 1 H) 7.95 (d, J = 2.41 Hz, |
| | 1 H) 7.83 (d, J = 1.14 Hz, 1 H) 7.54 (dd, J = 8.17, 2.47 Hz, 1 H) 7.29 (d, J = 7.73 Hz, |
| | 1 H) 7.15 (t, J = 7.30 Hz, 1 H) 6.89 (td, J = 7.45, 1.08 Hz, 1 H) 6.78 (dd, J = 8.17, |
| | 0.82 Hz, 1 H) 6.75 (s, 1 H) 5.13 (br d, J = 5.70 Hz, 1 H) 4.75-4.93 (m, 4 H) 4.23 (br |
| | t, J = 4.50 Hz, 2 H) 2.05-2.23 (m, 4 H) 1.94-2.04 (m, 1 H) 1.40 (s, 1 H) |
| A526 | (400 MHz, DMSO-d6) δ ppm 8.84 (dd, J = 8.05, 4.12 Hz, 1 H) 7.79-7.85 (m, 2 |
| | H) 7.40 (td, J = 8.46, 2.98 Hz, 1 H) 7.29 (d, J = 7.73 Hz, 1 H) 7.15 (t, J = 7.73 Hz, |
| | 1 H) 6.89 (t, J = 7.45 Hz, 1 H) 6.78 (d, J = 7.98 Hz, 1 H) 6.75 (s, 1 H) 5.13 (br d, |
| | J = 5.45 Hz, 1 H) 4.72-4.94 (m, 4 H) 4.23 (br t, J = 4.44 Hz, 2 H) 2.07-2.23 |
| | (m, 4 H) 1.92-2.05 (m, 1 H) |
| A530 | 1H NMR (400 MHz, DMSO-d6) δ ppm 9.05 (dd, J = 10.46, 8.17 Hz, 1 H) 7.96 |
| | (d, J = 2.53 Hz, 1 H) 7.89 (d, J = 5.20 Hz, 1 H) 7.60 (d, J = 2.53 Hz, 1 H) 7.31 (t, |
| | J = 6.65 Hz, 1 H) 7.15 (t, J = 7.73 Hz, 1 H) 6.87-6.93 (m, 2 H) 6.78 (d, J = 7.95 |
| | Hz, 1 H) 5.12-5.18 (m, 1 H) 4.17-4.28 (m, 2 H) 2.67 (dt, J = 3.61, 1.74 Hz, 1 |
| | H) 2.31-2.34 (m, 1 H) 2.21 (s, 3 H) 1.58 (s, 9 H) |
| A534 | (400 MHz, DMSO-d6) δ ppm 9.12 (dd, J = 9.70, 8.30 Hz, 1 H) 8.06 (d, J = 6.59 Hz, |
| | 1 H) 7.90 (dd, J = 8.30, 2.98 Hz, 1 H) 7.55 (ddd, J = 11.94, 8.71, 3.04 Hz, 1 H) 7.32 |
| | (t, J = 7.86 Hz, 1 H) 7.20 (s, 1 H) 7.16 (t, J = 7.73 Hz, 1 H) 6.90 (tdd, 1 H) 6.78 (dd, |
| | J = 8.24, 0.89 Hz, 1 H) 5.11-5.18 (m, 1 H) 4.15-4.28 (m, 2 H) 2.52-2.55 (m, 1 H) |
| | 2.15 (ddd, J = 11.03, 5.45, 2.53 Hz, 1 H) 1.95-2.04 (m, 1 H) 1.57 (s, 9 H) |
| A538 | (400 MHz, DMSO-d6) δ ppm 8.92 (dd, J = 18.31, 8.43 Hz, 1 H) 7.87 (d, J = 4.31 |
| | Hz, 1 H) 7.81 (dd, J = 8.36, 2.91 Hz, 1 H) 7.44-7.51 (m, 1 H) 7.35-7.43 (m, 1 |
| | H) 6.90-7.07 (m, 2 H) 6.89 (s, 1 H) 5.06-5.13 (m, 1 H) 2.64-2.83 (m, 2 H) |
| | 2.21 (s, 3 H) 1.58 (s, 9 H) |
| A539 | (400 MHz, DMSO-d6) δ ppm 11.06 (br s, 2 H) 8.11 (s, 1 H) 8.07 (s, 1 H) 7.88-7.92 |
| | (m, 2 H) 7.51-7.59 (m, 2 H) 7.30 (t, J = 7.73 Hz, 2 H) 7.20 (t, J = 7.71 Hz, 2 H) |
| | 7.12 (d, J = 1.01 Hz, 2 H) 6.93 (t, J = 7.44 Hz, 2 H) 6.83 (d, J = 8.11 Hz, 2 H) 5.83 |
| | (br d, J = 4.44 Hz, 2 H) 4.26-4.33 (m, 2 H) 4.16-4.24 (m, 2 H) 3.33-3.48 (m, 2 H) |
| | 2.24-2.30 (m, 2 H) 2.05-2.19 (m, 2 H) 1.45 (dd, J = 6.97, 5.58 Hz, 6 H) 1.41 |
| | (dd, J = 7.03, 4.88 Hz, 6 H) |
| A541 | (400 MHz, DMSO-d6) δ ppm 9.08 (dd, J = 13.69, 8.24 Hz, 1 H) 7.86 (d, J = 2.15 |
| | Hz, 1 H) 7.82 (dd, J = 8.30, 2.98 Hz, 1 H) 7.44 (ddd, J = 13.94, 8.68, 2.98 Hz, 1 |
| | H) 7.34 (t, J = 6.72 Hz, 1 H) 7.04-7.17 (m, 3 H) 6.90 (s, 1 H) 5.19 (dq, J = 8.36, |
| | 4.35 Hz, 1 H) 3.08-3.20 (m, 1 H) 2.98-3.07 (m, 1 H) 2.18-2.29 (m, 4 H) 1.57 (s, 9 H) |
| A542 | (400 MHz, DMSO-d6) δ ppm 9.02 (dd, J = 11.60, 8.05 Hz, 1 H) 7.93 (d, J = 7.73 |
| | Hz, 1 H) 7.82 (dd, J = 8.36, 2.91 Hz, 1 H) 7.73 (s, 1 H) 7.66 (d, J = 7.86 Hz, 1 H) |
| | 7.56 (t, J = 7.35 Hz, 1 H) 7.44 (ddd, J = 16.51, 8.65, 2.98 Hz, 1 H) 6.91 (s, 1 H) |
| | 5.48 (quin, J = 7.45 Hz, 1 H) 2.96-3.05 (m, 1 H) 2.84-2.95 (m, 1 H) 2.52-2.53 |
| | (m, 1 H) 2.22 (s, 3 H) 1.88-1.99 (m, 1 H) 1.57 (s, 9 H) |
| A543 | (400 MHz, DMSO-d6) δ ppm 8.03 (s, 1 H) 7.77-7.85 (m, 1 H) 7.47-7.54 |
| | (m, 1 H) 7.22-7.27 (m, 1 H) 7.12-7.21 (m, 1 H) 6.89-6.97 (m, 2 H) 6.78-6.86 |
| | (m, 1 H) 5.95-6.01 (m, 1 H) 4.15-4.36 (m, 2 H) 2.68-2.71 (m, 1 H) |
| | 2.62-2.66 (m, 1 H) 2.52-2.56 (m, 2 H) 2.20-2.28 (m, 4 H) 1.50-1.61 (m, 8 H) |
| A544 | (400 MHz, DMSO-d6) δ ppm 8.89 (dd, J = 8.24, 5.83 Hz, 1 H) 8.38-8.42 (m, 1 |
| | H) 7.79-7.83 (m, 2 H) 7.66 (d, J = 7.60 Hz, 1 H) 7.44 (ddd, J = 11.60, 8.68, 3.04 |
| | Hz, 1 H) 7.21 (ddd, J = 7.38, 5.04, 2.03 Hz, 1 H) 6.90 (s, 1 H) 5.36 (quin, |
| | J = 8.02 Hz, 1 H) 2.93-3.01 (m, 1 H) 2.81-2.90 (m, 1 H) 2.52-2.56 (m, 1 H) |
| | 2.19-2.23 (m, 3 H) 1.88-2.05 (m, 1 H) 1.60 (s, 9 H) |
| A545 | (400 MHz, DMSO-d6) δ ppm 9.19 (d, J = 6.94 Hz, 1 H) 7.85 (s, 1 H) 7.81 (dd, |
| | J = 8.05, 2.85 Hz, 1 H) 7.44 (d, J = 8.75 Hz, 1 H) 7.39 (t, J = 7.03 Hz, 1 H) 7.22 (t, |
| | J = 7.73 Hz, 1 H) 6.88-6.94 (m, 2 H) 6.85 (d, J = 8.11 Hz, 1 H) 5.62-5.70 (m, 1 |
| | H) 4.73 (dd, J = 9.57, 8.81 Hz, 1 H) 4.36 (ddd, J = 9.73, 8.02, 4.82 Hz, 1 H) 2.21 |
| | (s, 3 H) 1.56 (s, 9 H) |
| A547 | (400 MHz, DMSO-d6) δ ppm 8.95 (dd, J = 21.42, 8.36 Hz, 1 H) 7.96 (d, J = 6.97 |
| | Hz, 1 H) 7.83 (dd, J = 8.30, 2.98 Hz, 1 H) 7.46 (ddd, J = 20.56, 8.65, 2.98 Hz, 1 |
| | H) 7.35 (t, J = 6.17 Hz, 1 H) 7.14 (t, J = 7.67 Hz, 1 H) 6.92 (s, 1 H) 6.86-6.91 |
| | (m, 1 H) 6.73 (dd, J = 8.11, 0.89 Hz, 1 H) 5.18-5.28 (m, 1 H) 3.57 (s, 2 H) |
| | 2.11-2.18 (m, 1 H) 1.75-1.83 (m, 1 H) 1.60 (s, 9 H) 1.40 (s, 3 H) 1.29 (s, 3 H) |
| A549 | (400 MHz, DMSO-d6) δ ppm 8.94 (dd, J = 18.76, 8.36 Hz, 1 H) 7.87 (d, J = 4.31 |
| | Hz, 1 H) 7.82 (dd, J = 8.36, 3.04 Hz, 1 H) 7.47 (dd, J = 8.62, 2.92 Hz, 1 H) |
| | 7.34-7.43 (m, 2 H) 7.17-7.23 (m, 2 H) 6.90 (s, 1 H) 5.06-5.12 (m, 1 H) |
| | 2.68-2.79 (m, 2 H) 2.52-2.52 (m, 1 H) 2.21 (s, 3 H) 1.58 (s, 9 H) |
| A556 | (400 MHz, DMSO-d6) δ ppm 9.11 (dd, J = 11.85, 7.92 Hz, 1 H) 8.64 (br s, 1 H) |
| | 8.55 (br s, 1 H) 7.97 (d, J = 9.00 Hz, 1 H) 7.83 (dd, J = 8.36, 3.04 Hz, 1 H) 7.66 |
| | (dd, J = 12.67, 5.20 Hz, 1 H) 7.45 (ddd, J = 17.87, 8.62, 3.04 Hz, 1 H) 6.93 (s, 1 |
| | H) 5.52 (quin, J = 8.52 Hz, 1 H) 3.03-3.15 (m, 1 H) 2.91-3.01 (m, 1 H) 2.52-2.57 |
| | (m, 1 H) 2.22 (s, 3 H) 1.58 (s, 9 H) |
| A557 | (400 MHz, DMSO-d6) δ ppm 8.93 (dd, J = 16.98, 8.49 Hz, 1 H) 7.86 (d, J = 3.68 |
| | Hz, 1 H) 7.81 (dd, J = 8.30, 2.98 Hz, 1 H) 7.44 (ddd, J = 22.21, 8.65, 2.98 Hz, 1 |
| | H) 7.31-7.37 (m, 1 H) 7.12-7.20 (m, 2 H) 7.07-7.12 (m, 1 H) 6.89 (s, 1 H) |
| | 5.12 (br d, J = 5.83 Hz, 1 H) 2.65-2.82 (m, 2 H) 2.21 (s, 3 H) 1.87-2.03 (m, 2 |
| | H) 1.70-1.85 (m, 2 H) 1.59 (s, 9 H) |
| A558 | (400 MHz, DMSO-d6) δ ppm 8.91 (t, J = 8.43 Hz, 1 H) 7.88 (d, J = 4.82 Hz, 1 H) 7.82 |
| | (dd, J = 8.36, 2.91 Hz, 1 H) 7.44 (ddd, J = 13.56, 8.68, 2.98 Hz, 1 H) 7.36 (t, J = 5.87 |
| | Hz, 1 H) 7.17-7.27 (m, 3 H) 6.90 (s, 1 H) 5.40-5.48 (m, 1 H) 2.91-3.07 (m, 1 H) |
| | 2.78-2.87 (m, 1 H) 2.37-2.48 (m, 1 H) 2.21 (s, 3 H) 1.84-1.95 (m, 1 H) 1.58 (s, 9 H) |
| A559 | (400 MHz, DMSO-d6) δ ppm 9.10 (dd, J = 10.84, 7.79 Hz, 1 H) 8.56 (br s, 1 H) 8.09 |
| | (dd, J = 10.90, 7.86 Hz, 1 H) 7.94 (d, J = 7.10 Hz, 1 H) 7.82 (dd, J = 8.30, 2.98 Hz, |
| | 1 H) 7.35-7.58 (m, 2 H) 6.92 (s, 1 H) 5.50 (quin, J = 7.76 Hz, 1 H) 3.01-3.19 (m, 2 |
| | H) 2.52-2.62 (m, 1 H) 2.22 (s, 3 H) 1.99-2.10 (m, 1 H) 1.57 (s, 9 H) |
| A561 | (400 MHz, DMSO-d6) δ ppm 8.73 (dd, J = 8.11, 3.80 Hz, 1 H) 7.94 (d, J = 2.41 |
| | Hz, 1 H) 7.77 (d, J = 1.14 Hz, 1 H) 7.52 (dd, J = 8.17, 2.47 Hz, 1 H) 7.27 (d, |
| | J = 7.22 Hz, 1 H) 7.15 (t, J = 7.32 Hz, 1 H) 6.71-6.91 (m, 3 H) 5.13 (br d, |
| | J = 5.70 Hz, 1 H) 4.37-4.62 (m, 4 H) 4.20-4.26 (m, 2 H) 2.09-2.16 (m, 4 H) |
| | 1.95-2.03 (m, 1 H) 1.60-1.65 (m, 3 H) |
| A562 | (400 MHz, DMSO-d6) δ ppm 8.74 (dd, J = 8.17, 4.88 Hz, 1 H) 7.80 (dd, J = 8.36, |
| | 2.92 Hz, 1 H) 7.77 (d, J = 0.63 Hz, 1 H) 7.38 (td, J = 8.52, 2.98 Hz, 1 H) 7.27 (d, |
| | J = 7.48 Hz, 1 H) 7.15 (t, J = 7.74 Hz, 1 H) 6.89 (td, J = 7.48, 1.14 Hz, 1 H) 6.78 (d, |
| | J = 7.82 Hz, 1 H) 6.71 (s, 1 H) 5.10-5.17 (m, 1 H) 4.37-4.61 (m, 4 H) 4.17-4.28 (m, |
| | 2 H) 2.52-2.52 (m, 1 H) 2.07-2.19 (m, 4 H) 1.94-2.04 (m, 1 H) 1.60-1.65 (m, 3 H) |
| A563 | (400 MHz, DMSO-d6) δ ppm 8.66 (br d, J = 7.10 Hz, 1 H) 7.93 (d, J = 2.41 Hz, |
| | 1 H) 7.70 (s, 1 H) 7.50 (dd, J = 6.97, 2.53 Hz, 1 H) 7.25 (d, J = 7.35 Hz, 1 H) |
| | 7.14 (t, J = 7.37 Hz, 1 H) 6.88 (t, J = 7.13 Hz, 1 H) 6.77 (d, J = 8.11 Hz, 1 H) 6.67 |
| | (s, 1 H) 5.09-5.16 (m, 1 H) 4.19-4.30 (m, 2 H) 4.08-4.17 (m, 4 H) 2.52-2.58 |
| | (m, 1 H) 2.08-2.15 (m, 4 H) 1.89-2.03 (m, 1 H) 1.30 (s, 6 H) |
| A564 | (400 MHz, DMSO-d6) δ ppm 8.66 (dd, J = 8.24, 2.15 Hz, 1 H) 7.78 (dd, |
| | J = 8.36, 2.91 Hz, 1 H) 7.70 (s, 1 H) 7.30-7.44 (m, 1 H) 7.25 (d, J = 7.60 Hz, 1 |
| | H) 7.14 (t, J = 7.76 Hz, 1 H) 6.88 (t, J = 7.09 Hz, 1 H) 6.77 (d, J = 8.24 Hz, 1 H) |
| | 6.67 (s, 1 H) 5.09-5.16 (m, 1 H) 4.23 (br t, J = 5.01 Hz, 2 H) 4.05-4.18 (m, 4 |
| | H) 2.52-2.58 (m, 1 H) 2.12 (s, 3 H) 1.93-2.02 (m, 1 H) 1.30 (s, 6 H) |
| A565 | (400 MHz, DMSO-d6) δ ppm 10.55 (s, 1 H) 8.25 (s, 1 H) 7.79 (d, J = 1.90 Hz, 2 H) |
| | 7.71 (t, J = 1.90 Hz, 1 H) 7.29 (s, 1 H) 7.00 (dd, J = 8.05, 1.20 Hz, 1 H) 6.85 (t, |
| | J = 7.16 Hz, 1 H) 6.69-6.79 (m, 2 H) 4.30-4.40 (m, 2 H) 3.61 (br s, 2 H) 1.60 (s, 9 H) |
| A566 | (400 MHz, DMSO-d6) δ ppm 9.05 (d, J = 7.98 Hz, 1 H) 7.94 (s, 1 H) 7.53-7.71 |
| | (m, 1 H) 7.24-7.39 (m, 2 H) 7.16 (t, J = 7.67 Hz, 1 H) 6.87-6.96 (m, 2 |
| | H) 6.78 (d, J = 8.11 Hz, 1 H) 5.12-5.19 (m, 1 H) 4.15-4.30 (m, 2 H) 2.29 (s, 3 |
| | H) 2.09-2.20 (m, 1 H) 1.94-2.05 (m, 1 H) 1.58 (s, 9 H) |
| A567 | (400 MHz, DMSO-d6) δ ppm 8.74 (dd, J = 8.17, 2.60 Hz, 1 H) 7.87 (dd, |
| | J = 8.30, 2.98 Hz, 1 H) 7.81 (s, 1 H) 7.45 (ddd, J = 8.68, 5.96, 2.98 Hz, 1 H) 7.25 |
| | (d, J = 7.48 Hz, 1 H) 7.15 (t, J = 7.66 Hz, 1 H) 6.92 (s, 1 H) 6.86-6.90 (m, 1 H) |
| | 6.78 (dd, J = 8.24, 1.01 Hz, 1 H) 5.09-5.16 (m, 1 H) 4.09-4.28 (m, 6 H) 2.89 |
| | (s, 1 H) 2.68-2.78 (m, 1 H) 2.05-2.16 (m, 1 H) 1.92-2.02 (m, 1 H) 1.31 (s, 6 H) |
| A568 | 1H NMR (400 MHz, DMSO-d6) δ ppm 8.73 (dd, J = 8.24, 3.04 Hz, 1 H) 7.94 |
| | (d, J = 2.41 Hz, 1 H) 7.80 (s, 1 H) 7.52 (dd, J = 8.36, 2.41 Hz, 1 H) 7.27 (dd, |
| | J = 6.97, 4.69 Hz, 1 H) 7.14 (t, J = 7.72 Hz, 1 H) 6.88 (t, J = 7.48 Hz, 1 H) 6.84 (s, |
| | 1 H) 6.78 (d, J = 7.98 Hz, 1 H) 5.13-5.20 (m, 1 H) 4.43 (q, J = 7.10 Hz, 1 H) |
| | 3.18 (d, J = 2.15 Hz, 6 H) 2.06-2.21 (m, 4 H) 1.93-2.04 (m, 1 H) |
| A569 | (400 MHz, DMSO-d6) δ ppm 9.20 (dd, J = 17.81, 7.41 Hz, 1 H) 8.80 (s, 1 H) |
| | 8.56 (d, J = 6.72 Hz, 1 H) 8.05 (d, J = 9.13 Hz, 1 H) 7.83 (dd, J = 8.36, 3.04 Hz, 1 |
| | H) 7.36-7.49 (m, 2 H) 6.95 (s, 1 H) 5.29 (quin, J = 6.18 Hz, 1 H) 4.61-4.73 |
| | (m, 1 H) 4.50-4.60 (m, 1 H) 2.21-2.31 (m, 4 H) 2.07-2.18 (m, 1 H) 1.57 (s, 9 H) |

### Preparation Example 11: Preparation of 140 mg 4-Way Chewable Tablets Containing a Combination of Compound A472, Afoxolaner, Moxidectin and Praziquantel

The preparation of 140 mg 4-way chewable tablets was achieved by blending pre-sieved granules containing afoxolaner, moxidectin, praziquantel and compound A472, as described in the examples below, with sieved microcrystalline cellulose (MCC), meat flavor, sodium croscarmellose, ferric oxide red and magnesium stearate in a free-fall blender; adding magnesium stearate and colloidal silicon dioxide to the blender and continuing to blend the mixture, followed by compressing the blended composition into 140 mg chewable tablets.

An exemplary composition of a final blend for a 500 gram batch is shown in Table 7:

TABLE 7
Exemplary composition of a 500 gram batch blend
for preparation of 4-way chewable tablets

| | % in | | Quantity |
|---|---|---|---|
| Ingredient | blend | Function | [g] |
| Praziquantel granules | 28.571 | Active Agent | 142.86 |
| | | Intermediate | |
| Compound A472 granules | 22.768 | Active Agent | 113.84 |
| | | Intermediate | |
| Moxidectin granules | 18.095 | Active Agent | 90.48 |
| | | Intermediate | |
| Afoxolaner granules | 14.286 | Active Agent | 71.43 |
| | | Intermediate | |
| Microcrystalline | 6.780 | Filler | 33.90 |
| Cellulose (MCC) | | | |
| Meat Flavor | 5.000 | Flavor | 25.00 |
| Sodium-Croscarmellose | 2.000 | Disintegrant | 10.00 |
| Ferric oxide red | 1.000 | Colorant | 5.00 |
| Magnesium Stearate | 1.000 | Lubricant | 5.00 |
| Colloidal Silicon | 0.500 | Flow aid | 2.50 |
| Dioxide (Aerosil ®) | | | |
| Total | 100.000 | | 500.00 |

The amount of the active agent intermediate granules in the batch is based on 100.000 potency. If the potency of the active agent in the granules differs from 100.00%, the amount of the granules is adjusted to account for the assay. If the quantities of granules are modified to compensate for the assay value, the amount of MCC is adjusted to compensate for the modified amount of the granule(s) according to the formula 100−sum of all granules (0%)−9.5000%.

### Screening/Blending:

Depending on the assay of the four active agent intermediate granule batches used, the assay-adjusted percentage (0%) of each granule batch and the MCC (filler correction) was calculated as follows:
Praziquantel granules (%)=28.571(%)/(assay (%)/100)
Compound A472 granules (%)=22.768(%)/(assay (%)/100)
Moxidectin granules (%)=18.095(%)/(assay (%)/100)
Afoxolaner granules (%)=24.286(%)/(assay (%)/100)
MCC (%)=100−sum of all granules (%)−9.500%

The assay values of the intermediate granules by HPLC analysis were determined to be:
Compound A472 granules: Assay=100.26%
Praziquantel granules: Assay=99.80%
Moxidectin granules: Assay=97.30%
Afoxolaner granules: Assay=94.60%

Accordingly, the percentage of each intermediate granule component and the MCC component were adjusted to provide the concentrations and weights shown in Table 8 below:

TABLE 8
Adjusted composition and weights of an exemplary 500 gram batch

| Ingredients | %/Dose | Weight [g] |
|---|---|---|
| Compound A472 Granules | 22.709 | 113.5 |
| Praziquantel - Granules | 28.629 | 143.1 |
| Moxidectin - Granules | 18.597 | 92.9 |
| Afoxolaner - Granules | 15.101 | 75.5 |
| MCC 200 XLM | 5.464 | 27.3 |
| Flavor | 5.000 | 25.0 |
| Sodium Croscarmellose | 2.000 | 10.0 |
| Magnesium Stearate | 1.000 | 5.0 |
| Ferric Oxide red | 1.000 | 5.0 |
| Colloidal Silicon | 0.500 | 2.5 |
| Dioxide (Aerosil ®) | | |
| | 100.000 | 499.8 |

The component granules, flavor, sodium croscarmellose an ferric oxide were sieved through a 1.0 mm sieve into a free-fall blender. The components were then blended at 32 revolutions per minute (rpm) for 15 minutes (480 U).

Aerosil® and magnesium stearate were weighted and sieved through a 0.5 mm sieve into the blender and the contents were mixed at 32 rpm for an additional 5 minutes (160 U). The resulting blended powder was used for tableting.

The final blend was compacted into tablets on an Korsch EK2 eccentric tablet compactor with round bi-convex punches of 8 mm, obtaining 140 mg tablets with tensile strengths of 2.0±0.5 N/mm².

It will be appreciated by persons of skill in the art that tablets of different sizes may be prepared using the same procedure but adapting the tablet compacting equipment to obtain the desired tablet size including corresponding different dosages of each active agent.

### Preparation Example 12: Preparation of 108 mg and 431 mg 3-Way Chewable Tablets Containing Afoxolaner, Moxidectin and Praziquantel

The preparation of 3-way chewable tablets comprising a combination of afoxolaner, moxidectin and praziquantel, each in the form of intermediate granule compositions described below, weighing 108 mg instead of 140 mg, was achieved similarly to the process described above in Preparation Example 11. Thus, pre-sieved granule compositions containing afoxolaner, moxidectin and praziquantel were blended with sieved microcrystalline cellulose (MCC), meat flavor, sodium croscarmellose, ferric oxide red and magnesium stearate. Magnesium stearate and colloidal silicon dioxide were added to the blender and the mixture was further blended followed by compressing the blended composition into chewable tablets of two sizes weighing 108 mg and 431 mg containing the 3-way combination of afoxolaner, moxidectin and praziquantel.

An exemplary composition of a final blend for a 386 gram batch is shown in Table 9:

TABLE 9
Exemplary composition of a 386 gram batch blend
for preparation of 3-way chewable tablets comprising
praziquantel, moxidectin and afoxolaner

| | % in | | |
|---|---|---|---|
| Ingredient | granulate | Function | Quantity [g] |
| Praziquantel granules | 36.994 | Active Agent | 142.86 |
| | | Intermediate | |
| Moxidectin granules | 23.430 | Active Agent | 90.48 |
| | | Intermediate | |
| Afoxolaner granules | 18.497 | Active Agent | 71.43 |
| | | Intermediate | |
| Microcrystalline | 8.778 | Filler | 33.90 |
| Cellulose (MCC) | | | |
| Meat Flavor | 6.474 | Flavor | 25.00 |
| Sodium-Croscarmellose | 2.590 | Disintegrant | 10.00 |
| Ferric oxide red | 1.295 | Colorant | 5.00 |
| Magnesium Stearate | 1.295 | Lubricant | 5.00 |
| Colloidal Silicon | 0.647 | Flow aid | 2.50 |
| Dioxide (Aerosil ®) | | | |
| Total | 100.000 | | 386.16 |

As noted above for Preparation Example 11, the amount of the active agent intermediate granules in the batch is based on 100.000 potency. The amount of the granules is adjusted to account for the assay. If the quantities of granules are modified to compensate for the assay value, the amount of MCC is adjusted to compensate for the modified amount of the granule(s) according to the formula 100−sum of all granules (%) −12.301%.

### Screening/Blending:

The assay-adjusted percentage (0%) of each granule batch and the MCC (filler correction) was calculated in the same manner as in Preparation Example 11.

The assay values of the intermediate granules by HPLC analysis were determined to be:
Praziquantel granules: Assay=99.06%
Moxidectin granules: Assay=98.49%
Afoxolaner granules: Assay=98.79%

Accordingly, the percentage of each active agent intermediate granule component and the MCC component were adjusted to provide the concentrations and weights shown in Table 10 below:

TABLE 10
Adjusted composition and weights of an exemplary 385 gram batch

| | % in | Weight |
|---|---|---|
| Ingredients | granulate | [g] |
| Praziquantel - Granules | 37.442 | 144.2 |
| Moxidectin - Granules | 23.851 | 91.9 |
| Afoxolaner - Granules | 18.772 | 72.4 |
| MCC 200 XLM | 7.602 | 29.3 |
| Flavor | 6.491 | 25.0 |
| Sodium Croscarmellose | 2.596 | 10.0 |
| Magnesium Stearate | 1.298 | 5.0 |
| Ferric Oxide red | 1.298 | 5.0 |
| Colloidal Silicon | 0.649 | 2.5 |
| Dioxide (Aerosil ®) | | |
| | 100.000 | 385.3 |

The active agent intermediate granules, flavor, MCC, sodium croscarmellose an ferric oxide were weighted and sieved through a 1.0 mm sieve into a 2-liter beaker. The beaker was placed into a turbula mixer, and the components were mixed at 32 revolutions per minute (rpm) for 15 minutes (480 U).

Aerosil® and magnesium stearate were weighed and sieved through a 0.5 mm sieve and added to the beaker, and the contents were mixed at 32 rpm for an additional 5 minutes (160 U). The resulting blended powder was used for tableting.

The final blend was compacted into tablets on an Korsch EK2 eccentric tablet compactor to obtain 3-way chewable tablets comprising afoxolaner, moxidectin and praziquantel weighing 108 mg and 431 mg with tensile strengths of 1.5±0.5 N/mm².

It will be appreciated by persons of skill in the art that tablets of different sizes may be prepared using the same procedure but adapting the tablet compacting equipment to obtain the desired tablet size including corresponding different dosages of each active agent.

### Preparation Example 13: Preparation of 114 mg and 457 mg 3-Way Chewable Tablets Containing Compound A472, Afoxolaner and Praziquantel

The preparation of 114 mg and 457 mg 3-way chewable tablets comprising a combination of compound A472, afoxolaner and praziquantel, each in the form of intermediate granule compositions as described in the examples below, was achieved similarly to the process described above in Preparation Examples 11 and 12.

An exemplary composition of a final blend for a 409 gram batch to produce the 3-way tablets is shown in Table 11:

TABLE 11
Exemplary composition of a 409 gram batch blend
for preparation of 3-way chewable tablets comprising
compound A472, afoxolaner and praziquantel

| | % in | | Quantity |
|---|---|---|---|
| Ingredient | granulate | Function | [g] |
| Compound A472 granules | 27.798 | Active Agent | 113.84 |
| | | Intermediate | |
| Praziquantel granules | 34.884 | Active Agent | 142.86 |
| | | Intermediate | |
| Afoxolaner granules | 17.442 | Active Agent | 71.43 |
| | | Intermediate | |
| Microcrystalline | 8.278 | Filler | 33.90 |
| Cellulose (MCC) | | | |
| Meat Flavor | 6.105 | Flavor | 25.00 |
| Sodium-Croscarmellose | 2.442 | Disintegrant | 10.00 |
| Ferric oxide red | 1.221 | Colorant | 5.00 |
| Magnesium Stearate | 1.221 | Lubricant | 5.00 |
| Colloidal Silicon | 0.610 | Flow aid | 2.50 |
| Dioxide (Aerosil ®) | | | |
| Total | 100.000 | | 409.52 |

As noted above for Preparation Examples 11 and 12, the amount of the active agent intermediate granules in the batch is based on 100.000 potency. The amount of the granules is adjusted to account for the assay. If the quantities of granules are modified to compensate for the assay value, the amount of MCC is adjusted to compensate for the modified amount of the granule(s) according to the formula 100−sum of all granules (0%)−11.5990%.

### Screening/Blending:

The assay-adjusted percentage (0%) of each granule batch and the MCC (filler correction) was calculated in the same manner as in Preparation Example 11.

The assay values of the intermediate granules by TIPLC analysis were determined to be:
Compound A472 granules: Assay=99.13%
Praziquantel granules: Assay=99.06%
Afoxolaner granules: Assay=98.79%

Accordingly, the percentage of each active agent intermediate granule component and the MCC component were adjusted to provide the concentrations and weights shown in Table 12 below:

TABLE 12
Adjusted composition and weights of an exemplary 408 gram batch

| Ingredients | %/Dose | Weight [g] |
|---|---|---|
| Compound A472 - Granules | 28.137 | 114.9 |
| Praziquantel - Granules | 35.334 | 144.2 |
| Afoxolaner - Granules | 17.716 | 72.3 |
| MCC 200 XLM | 7.175 | 29.3 |
| Flavor | 6.125 | 25.0 |
| Sodium Croscarmellose | 2.450 | 10.0 |
| Magnesium Stearate | 1.225 | 5.0 |
| Ferric Oxide red | 1.225 | 5.0 |
| Colloidal Silicon | 0.613 | 2.5 |
| Dioxide (Aerosil ®) | | |
| | 100.000 | 408.2 |

The component active agent intermediate granules, flavor, MCC, sodium croscarmellose an ferric oxide were weighed and sieved through a 1.0 mm sieve into a 2-liter beaker. The beaker was placed into a turbula mixer, and the components were mixed at 32 revolutions per minute (rpm) for 15 minutes (480 U).

Aerosil® and magnesium stearate were weighed and sieved through a 0.5 mm sieve and added to the beaker, and the contents were mixed at 32 rpm for an additional 5 minutes (160 U). The resulting blended powder was used for tableting.

The final blend was compacted into tablets on a StyleOne eccentric tablet compactor to obtain 3-way chewable tablets comprising compound A472, afoxolaner and praziquantel weighing 114 mg and 457 mg with tensile strengths of 1.5±0.3 N/mm².

It will be appreciated by persons of skill in the art that tablets of different sizes may be prepared using the same procedure but adapting the tablet compacting equipment to obtain the desired tablet size including corresponding different dosages of each active agent.

### Preparation Example 14: Preparation of 120 mg 3-Way Chewable Tablets Containing Compound A472, Moxidectin and Praziquantel

The preparation of 120 mg 3-way chewable tablets comprising a combination of compound A472, moxidectin and praziquantel, each in the form of intermediate granule compositions as described in the examples below, was achieved similarly to the process described above in Preparation Examples 11, 12 and 13.

An exemplary composition of a final blend for a 750 gram batch to produce the 3-way tablets is shown in Table 13:

TABLE 13
Exemplary composition of a 750 gram batch blend
for preparation of 3-way chewable tablets comprising
compound A472, moxidectin and praziquantel

| | % in | | Quantity |
|---|---|---|---|
| Ingredient | granulate | Function | [g] |
| Compound A472 granules | 26.563 | Active Agent | 199.22 |
| | | Intermediate | |
| Praziquantel granules | 33.333 | Active Agent | 250.00 |
| | | Intermediate | |
| Moxidectin granules | 21.111 | Active Agent | 158.33 |
| | | Intermediate | |
| Microcrystalline | 7.910 | Filler | 59.32 |
| Cellulose (MCC) | | | |
| Meat Flavor | 5.833 | Flavor | 43.75 |
| Sodium-Croscarmellose | 2.333 | Disintegrant | 17.50 |
| Ferric oxide red | 1.167 | Colorant | 8.75 |
| Magnesium Stearate | 1.167 | Lubricant | 8.75 |
| Colloidal Silicon | 0.583 | Flow aid | 4.38 |
| Dioxide (Aerosil ®) | | | |
| Total | 100.000 | | 750.00 |

As noted above for Preparation Examples 11-13, the amount of the active agent intermediate granules in the batch is based on 100.000 potency. The amount of the granules is adjusted to account for the assay. If the quantities of granules are modified to compensate for the assay value, the amount of MCC is adjusted to compensate for the modified amount of the granule(s) according to the formula 100−sum of all granules (0%)−11.0830%.

### Screening/Blending:

The assay-adjusted percentage (0%) of each granule batch and the MCC (filler correction) was calculated in the same manner as in Preparation Example 11.

The assay values of the intermediate granules by HPLC analysis were determined to be:
Compound A472 granules: Assay=100.0%
Praziquantel granules: Assay=99.8%
Moxidectin granules: Assay=98.7%

Accordingly, the percentage of each intermediate granule component and the MCC component were adjusted to provide the concentrations and weights shown in Table 14 below:

TABLE 14
Adjusted composition and weights of an exemplary 750 gram batch

| Ingredients | %/Dose | Weight [g] |
|---|---|---|
| Compound A472 - Granules | 26.563 | 199.1 |
| Praziquantel - Granules | 33.417 | 250.6 |
| Moxidectin - Granules | 21.396 | 160.4 |
| MCC 200 XLM | 7.541 | 56.5 |
| Flavor | 5.833 | 43.8 |
| Sodium Croscarmellose | 2.333 | 17.5 |
| Magnesium Stearate | 1.167 | 8.7 |
| Ferric Oxide red | 1.167 | 8.7 |
| Colloidal Silicon | 0.583 | 4.3 |
| Dioxide (Aerosil ®) | | |
| | 100.000 | 749.6 |

The active agent intermediate granules, flavor, MCC, sodium croscarmellose an ferric oxide were weighed and sieved through a 1.0 mm sieve into a 2-liter beaker. The beaker was placed into a turbula mixer and the components were mixed at 32 revolutions per minute (rpm) for 15 minutes (480 U).

Aerosil® and magnesium stearate were weighed and sieved through a 0.5 mm sieve and added to the beaker and the contents were mixed at 32 rpm for an additional 5 minutes (160 U). The resulting blended powder was used for tableting.

The final blend was compacted into tablets on an Korsch EK2 eccentric tablet compactor to obtain 3-way chewable tablets comprising compound A472, moxidectin and praziquantel weighing 120 mg with tensile strengths of 1.5±0.3 N/mm².

It will be appreciated by persons of skill in the art that tablets of different sizes may be prepared using the same procedure but adapting the tablet compacting equipment to obtain the desired tablet size including corresponding different dosages of each active agent.

### Preparation Example 15: Preparation of 100 mg 3-Way Chewable Tablets Containing Compound A472, Afoxolaner and Moxidectin

The preparation of 100 mg 3-way chewable tablets comprising a combination of compound A472, afoxolaner and moxidectin, each in the form of intermediate granule compositions as described in the examples below, was achieved similarly to the process described above in Preparation Examples 11-14 above.

An exemplary composition of a final blend for a 750 gram batch to produce the 3-way tablets is shown in Table 15:

TABLE 15
Exemplary composition of a 750 gram batch blend
for preparation of 3-way chewable tablets comprising
compound A472, afoxolaner and moxidectin

| | % in | | Quantity |
|---|---|---|---|
| Ingredient | granulate | Function | [g] |
| Compound A472 granules | 31.875 | Active Agent | 239.06 |
| | | Intermediate | |
| Afoxolaner granules | 20.000 | Active Agent | 150.00 |
| | | Intermediate | |
| Moxidectin granules | 25.333 | Active Agent | 190.00 |
| | | Intermediate | |
| Microcrystalline | 9.492 | Filler | 71.19 |
| Cellulose (MCC) | | | |
| Meat Flavor | 7.000 | Flavor | 52.50 |
| Sodium-Croscarmellose | 2.800 | Disintegrant | 21.00 |
| Ferric oxide red | 1.400 | Colorant | 10.50 |
| Magnesium Stearate | 1.400 | Lubricant | 10.50 |
| Colloidal Silicon | 0.700 | Flow aid | 5.25 |
| Dioxide (Aerosil ®) | | | |
| Total | 100.000 | | 750.00 |

As noted above for Preparation Examples 11-14, the amount of the active agent intermediate granules in the batch is based on 100.000 potency. The amount of the granules is adjusted to account for the assay. If the quantities of granules are modified to compensate for the assay value, the amount of MCC is adjusted to compensate for the modified amount of the granule(s) according to the formula 100−sum of all granules (%) −13.300%.

### Screening/Blending:

The assay-adjusted percentage (0%) of each granule batch and the MCC (filler correction) was calculated in the same manner as in Preparation Examples 11-14.

The assay values of the intermediate granules by HPLC analysis were determined to be:
Compound A472 granules: Assay=100.0%
Afoxolaner granules: Assay=98.0%
Moxidectin granules: Assay=98.7%

Accordingly, the percentage of each active agent intermediate granule component and the MCC component were adjusted to provide the concentrations and weights shown in Table 16 below:

TABLE 16
Adjusted composition and weights of an exemplary 750 gram batch

| Ingredients | %/Dose | Weight [g] |
|---|---|---|
| Compound A472 - Granules | 31.875 | 239.0 |
| Afoxolaner - Granules | 20.408 | 153.0 |
| Moxidectin - Granules | 25.676 | 192.5 |
| MCC 200 XLM | 8.741 | 65.5 |
| Flavor | 7.000 | 52.5 |
| Sodium Croscarmellose | 2.800 | 21.2 |
| Magnesium Stearate | 1.400 | 10.5 |
| Ferric Oxide red | 1.400 | 10.5 |
| Colloidal Silicon | 0.700 | 5.1 |
| Dioxide (Aerosil ®) | | |
| | 100.000 | 749.8 |

The active agent intermediate granules, flavor, MCC, sodium croscarmellose an ferric oxide were weighed and sieved through a 1.0 mm sieve into a 2-liter beaker. The beaker was placed into a turbula mixer and the components were mixed at 32 revolutions per minute (rpm) for 15 minutes (480 U).

Aerosil® and magnesium stearate were weighed and sieved through a 0.5 mm sieve and added to the beaker and the contents were mixed at 32 rpm for an additional 5 minutes (160 U). The resulting blended powder was used for tableting.

The final blend was compacted into tablets on an Korsch EK2 eccentric tablet compactor to obtain 3-way chewable tablets comprising compound A472, afoxolaner and moxidectin weighing 100 mg with tensile strengths of 1.5±0.3 N/mm².

It will be appreciated by persons of skill in the art that tablets of different sizes may be prepared using the same procedure but adapting the tablet compacting equipment to obtain the desired tablet size including corresponding different dosages of each active agent.

### Preparation Example 16: Preparation of Exemplary Amorphous Solid Dispersion (ASD) Granules Containing Compound A472

The exemplary composition of a 1.8 kilogram batch of ASD granules containing Compound A472 is shown in Table 17 below. The granules were prepared by using a Glatt 3.1 Fluid-Bed-Granulator (FBG) with a Schlick 940 two-fluid nozzle.

TABLE 17
Exemplary composition of a 1.8 kg
batch of Compound A472 ASD granules

| Composition | %/Dose | Function | Weight [g] |
|---|---|---|---|
| Compound A472 | 8.000 | Active Agent | 144.0 |
| VitE TPGS | 2.800 | Solubilizer | 50.4 |
| HPMC-AS LG | 29.200 | Binder | 525.6 |
| MCC 302 | 60.000 | Filler | 1080.2 |
| | 100.000 | | 1800.2 |

A spray solution of Compound A472, Vitamin E TPGS and HPMC-AS LG according to Table 18 below was prepared:

TABLE 18
Exemplary composition of Acetone Spraying Solution

| Spray Solution 1 (Slo-1) | % (w/w) | g |
|---|---|---|
| Compound A472 | 3.800 | 144.0 |
| VitE TPGS | 1.330 | 50.4 |
| HPMC-AS LG | 13.870 | 525.6 |
| Acetone | 81.000 | 3070.7 |
| Total: | 100.000 | 3790.7 |

Acetone was weighed into a light-protected glass bottle and Compound A472 was added in portions with stirring until a clear solution was obtained, without the presence of particles or turbidity.

Vitamin E TPGS was added to the solution in portions with stirring until the resulting slightly turbid solution was free of particles.

HPMC-AS LG was then added in portions with stirring until the slightly turbid solution was free of particles. The resulting solution was filtered through a 457 μm metal filter and used directly for the spray granulation.

MCC 302 was sieved through a 0.8 mm sieve and placed into the FBG. Fluidization was adjusted to obtain a suitable fluidized bed. The nozzle in the granulator was adjusted “position 2” and the process gas pressure was set to 0.8 bar.

The process gas (air) was set to 40 °C and flow of the gas was started to to allow the FBG to warm with pre-mixing.

Acetone was sprayed into the granulator for approximately 2 minutes to cool the nozzle tip by evaporation. The granulation process was then started by spraying the acetone solution of Compound A472, Vitamin E TPGS and HPMC-AS LG on top of the fluidized bed of MCC.

After the solution was sprayed onto the MCC, the line was flushed with acetone. The mixture was then dried at 60 °C for 30 minutes.

During the spraying process, the inlet air volume flow was set to approximately 40-100 Nm³/h to ensure proper fluidization, the inlet gas temperature was maintained at 40 °C, the temperature of the granulate product was maintained at approximately 25-36 °C by adjusting the spraying rate accordingly to approximately 15-45 g/min.

After the mixture was dried, the granules were equilibrated for at least 30 minutes at ambient room conditions and screened via a 0.8 mm sieve before packaging. 1643.4 grams of the Compound A472 ASD granules were isolated.

### Preparation Example 17: Exemplary Preparation of Amorphous Solid Dispersion (ASD) Granules Containing Moxidectin

The exemplary composition of a 1.5 kilogram ASD batch containing moxidectin is shown in table 19. The granules were prepared using a Glatt 3.1 Fluid-Bed-Granulator (FBG) with a Schlick 940 two-fluid nozzle.

TABLE 19
Exemplary composition of a 1.5 kilogram
batch of moxidectin ASD granules

| Composition | %/Dose | Function | Weight [g] |
|---|---|---|---|
| Moxidectin | 0.150 | Active Agent | 2.2550 |
| BHT | 0.150 | Antioxidant | 2.2556 |
| NaOH 1M | q.s. pH = 7.5 in | pH Modifier | pH = 7.62 |
| | spray solution | | |
| PVP K25 | 14.700 | Binder | 220.5 |
| MCC 302 | 85.000 | Filler | 1274.97 |
| Total: | 100.000 | | 1499.9806 |

A spray solution containing the components shown in Table 20 was prepared for the granulation process.

TABLE 20
Exemplary composition of spraying solution
for moxidectin ASD granules

| Spray Solution | % (w/w) | Weight [g] |
|---|---|---|
| Moxidectin | 0.150 | 2.2550 |
| BHT | 0.150 | 2.2556 |
| 1M NaOH | q.s. | 10.47 (pH = 7.62) |
| | pH = 7.5 | |
| PVP K25 | 14.700 | 220.5 |
| water | 17.000 | 255.1 |
| ethanol | 68.000 | 1020.1 |
| Total: | 100.000 | 1510.68 |

Water and ethanol were weighed into a light-protected glass bottle and the amount of BHT and moxidectin shown in Table 20 were added in portions with stirring until a clear solution was obtained, without the presence of any particles or turbidity.

The amount of PVP K25 shown in Table 20 was added in portions with stirring until no particles were evident, resulting in a clear solution.

The apparent pH of the solution was measured and adjusted to 7.5±0.3 with 1M NaOH.

The MCC 302 was sieved through a 0.8 mm sieve and placed into the FBG. The nozzle in the granulator was adjusted to “position 2” and the process gas pressure was set to 0.8 bar.

The process gas (air) was set to 50 °C and flow of the gas was started to allow the FBG to warm up with pre-mixing in order to obtain an adequate bed fluidization.

Ethanol was sprayed into the FBG for approximately 2 minutes to cool the nozzle tip by evaporation. The granulation process was then started by spraying the pH-adjusted aqueous ethanol solution of moxidectin, BHT and PVP K25 on top of the fluidized bed of MCC.

During the spraying process, the inlet air volume flow was set to approximately 40-100 Nm³/h, to ensure proper fluidization, the inlet gas temperature was maintained at 50 °C the temperature of the granulate product was maintained at approximately 30-40 °C by adjusting the spraying rate accordingly to approximately 10-45 g/minute.

After the solution was sprayed onto the MCC, the line was flushed with ethanol. The mixture was then dried at 60 °C for 30 minutes.

After the mixture was dried, the granules were equilibrated for at least 30 minutes at ambient room conditions and screened via a 0.8 mm sieve before packaging. 1393.7 grams of the moxidectin ASD granules were isolated.

### Preparation Example 18: Exemplary Preparation of Praziquantel High Shear Granules

The exemplary composition of a 1.0 kilogram batch of praziquantel high shear granules is shown in Table 21, not including 3×93.5 grams of water used as granulation liquid. The granules were prepared by using a Diosna High-Shear-Granulator (HSG) P1/6 with the 2 liter vessel. Three wet granulation batches were prepared and then combined for wet screening, drying and dry screening to provide the 1.0 kilogram batch of praziquantel high shear granules.

TABLE 21
Exemplary composition of a 1 kg batch
of praziquantel high shear granules

| | | | Weight | Weight | Weight |
|---|---|---|---|---|---|
| Composition | %/Dose | Function | part 1 [g] | part 2 [g] | part 3 [g] |
| Praziquantel | 40.000 | Active Agent | 133.6 | 133.6 | 133.6 |
| PVP K25 | 5.000 | Binder | 16.7 | 16.8 | 16.7 |
| MCC101 | 55.000 | Filler | 183.7 | 183.7 | 183.7 |
| Total: | 100.000 | | 334.0 | 334.1 | 334.0 |

For each part, the amounts of praziquantel, MCC 101 and PVP K25 shown in Table 21 were weighed and sieved through a 0.8 mm sieve into the high shear granulator (HSG).

The HSG was closed and the main rotator was started at 250 revolutions per minute (rpm) and mixed for 5 minutes.

The cutter was started at a rate of 1000 rpm.

The liquid pump was started to transfer the water into the powder within approximately 3-4 minutes. Granulation was continued for a total time of 6 minutes from the start of liquid addition.

The granulation process was stopped and the wet granules were transferred into a suitable container.

This granulation procedure was carried out for all 3 parts in a row.

The 3 wet powder parts were combined and sieved through a 4.0 mm screen in a suitable screening mill at 350 rpm in the oscillation mode (Frewitt MA Free Drive-Lab Mill) and the moisture content (loss on drying, LoD) was measured. The sieved powder was then transferred into a dryer and dried at 60 °C for approximately 10 minutes until an LoD of 1-2% was achieved.

The dried powder was sieved through a 1.25 mm screen at 350 rpm in the oscillation mode (Frewitt MA Free Drive-Lab Mill) and transferred to a suitable container. 843.9 grams of the praziquantel high-shear granules were isolated.

### Preparation Example 20: Exemplary Preparation of Afoxolaner High Shear Granules

The composition of a 1.0 kilogram batch of afoxolaner high shear granules is shown in table 22 below, not including the 280.0 gram water used as granulation liquid. The granules were prepared by using a Diosna High-Shear-Granulator (HSG) P1/6 with the 6 liter vessel.

TABLE 22
Exemplary composition of a 1 kilogram
batch of afoxolaner high shear granules

| Composition | %/Dose | Function | Weight [g] |
|---|---|---|---|
| Afoxolaner | 40.000 | Active Agent | 400.0 |
| PVP K25 | 13.333 | Binder | 133.4 |
| MCC101 | 43.667 | Filler | 436.7 |
| Sodium | 3.000 | Disintegrant | 30.0 |
| Croscarmellose | | | |
| Total: | 100.000 | | 1000.1 |

The amounts of afoxolaner, MCC 101 and PVP K25 and Sodium Croscarmellose were weighed and sieved through a 0.8 mm sieve into the high shear granulator (HSG).

The HSG was closed, the main rotator was started at 550 revolutions per minute (rpm) and mixed for 5 minutes to pre-mix the components.

The cutter was started at a rate of 2200 rpm.

The liquid pump was started to transfer the water into the powder within approximately 2 minutes. Granulation was continued for a total time of 210 seconds from the start of liquid addition.

The granulation process was stopped and the wet granules were transferred into a suitable container.

The wet powder was sieved through a 4.0 mm screen at 350 rpm in the oscillation mode (Frewitt MA Free Drive-Lab Mill) and the loss on drying (LoD) was measured. The sieved powder was then transferred into a dryer and dried at 60 °C for approximately 13 minutes until an LoD of 1-2% was obtained.

The dried powder was sieved through a 1.25 mm screen at 350 rpm in the oscillation mode (Frewitt MA Free Drive-Lab Mill) and transferred to a suitable container. 832.2 g of the afoxolaner high-shear granules were isolated.

### BIOLOGICAL EFFICACY EXAMPLES

Biological Example 1: Evaluation of the efficacy of a 4-way chewable tablet composition comprising a combination of compound A472, afoxolaner, moxidectin and praziquantel against induced infections of Amblyomma americanum on dogs.

Two Treatment Groups containing eight beagle dogs each were set up for the study. Dogs in Treatment Group 1 were untreated and dogs in Treatment Group 2 were treated with a 4-way chewable tablet composition according to Preparation Example 11 to target a minimum efficacious dose of 0.8 mg/kg body weight Compound A472, 12 micrograms/kg body weight moxidectin (μg/kg), 2.5 mg/kg body weight afoxolaner and 5 mg/kg body weight praziquantel on Treatment Day 0. Dogs were treated in the fed prandial state.

On Day −2, each dog in the study was infested with 50 (±5) Amblyomma americanum ticks. On Day 0, dogs in Treatment Group 2 were fed approximately 15-60 minutes before treatment and then treated with a combination of tablets to reach the target dose. Dogs in Treatment Group 1 were not treated.

On Day 3 (72±2 hours post treatment), the dogs were examined, and the number of live and dead ticks were counted. Dogs were re-infested with 50 (±5) Amblyomma americanum ticks on Days 7, 21, 28, 42, 56 and 70 of the study. Dogs were examined on Days 10, 24, 31, 45, 59 and 73 (72±2 hours post last treatment) to count dead and live ticks.

The percent reduction of ticks in Treatment Group 2 compared with Treatment Group 1 (untreated control group) was 100% at Days 3, 10, 24 and 31. The percent reduction on Day 45 was 84% compared to the untreated control group. The percent efficacy on Day 59 was 77.1% compared to the untreated control and the percent efficacy on Day 73 was 69.1% compared with the control.
Biological Example 2: Evaluation of the efficacy of a 4-way chewable tablet composition containing a combination of compound A472, afoxolaner, moxidectin and praziquantel; a 3-way chewable tablet containing a combination of compound A472, afoxolaner and praziquantel, and a 3-way chewable tablet containing a combination of afoxolaner, moxidectin and praziquantel against induced infections of Toxocara canis (roundworm) in dogs.

Four Treatment Groups containing six Beagle dogs each were set up. Dogs in Treatment Group 1 served as an untreated control group. Dogs in Treatment Group 2 were treated with chewable tablets containing a 4-way combination of compound A472, afoxolaner, moxidectin and praziquantel at concentrations to deliver minimum efficacious doses close to the target dose of 0.8 mg/kg body weight compound A472, 2.5 mg/kg body weight afoxolaner, 12 micrograms/kg moxidectin body weight and 5 mg/kg body weight praziquantel prepared according to Preparation Example 11. Dogs in Treatment Group 3 were treated with chewable tablets containing a 3-way combination of afoxolaner, moxidectin and praziquantel prepared according to Preparation Example 12 to deliver doses close to the target dose of 12 micrograms/kg moxidectin, 2.5 mg/kg afoxolaner and 5 mg/kg praziquantel. Dogs in Treatment Group 4 were treated with chewable tablets containing a 3-way combination of Compound A472, afoxalaner, and praziquantel prepared according to Preparation Example 13 to deliver doses of approximately 0.4 mg/kg compound A472, 1.25 mg/kg afoxalaner, and 2.5 mg/kg praziquantel.

On Day −49 of the study, dogs were inoculated with Toxocara canis. On Day −6, fecal sample collection was conducted to qualitatively verify the presence of Toxocara canis eggs. On Day −2, dogs were allocated to treatment groups 1, 2, 3 or 4 described above. Dogs in Treatment Group 2 were treated with the 4-way chewable tablets and dogs in Treatment Groups 3 and 4 were treated with the appropriate 3-way chewable tablets described above once on Day 0 of the study. Dogs were treated in the fed prandial state.

On Day 7, dogs were euthanized for parasite recovery and count. The percent reduction of Toxocara canis worms in Treatment Group 2 compared with Treatment Group 1 (untreated control group) was 98.7%. The percent reduction of Toxocara canis worms in Treatment Group 3 compared compared with Treatment Group 1 (untreated control group) was 0%, and the percent reduction of Toxocara canis worms in Treatment Group 4 compared with Treatment Group 1 (untreated control group) was 95.5%.
Biological Example 3: Evaluation of the efficacy of a chewable tablet composition comprising a 4-way combination of compound A472, afoxolaner, moxidectin and praziquantel against infections of Dipylidium caninum (tapeworm) in dogs using the experimental flea (Ctenocephalides felis) infestation model.

Two Treatment Groups of eight dogs each were set up for the study. Dogs in Treatment Group 1 served as an untreated control group and dogs in Treatment Group 2 were treated with chewable tablets containing a 4-way combination of compound A472, afoxolaner, moxidectin and praziquantel at concentrations to deliver doses of 0.8 mg/kg body weight compound A472, 2.5 mg/kg body weight afoxolaner, 12 micrograms/kg body weight moxidectin and 5 mg/kg body weight praziquantel prepared according to Preparation Example 11 once on Day 0 of the study in the fed prandial state.

Each dog enrolled in the study was topically infested with 100 Dipylidium caninum-infected Ctenocephalides felis (EU isolate) on study Days −49, −42 and −35 and allowed to groom freely. Five days after infestations on Days −44, −37 and −30, dogs were combed to remove any remaining fleas, which were collected for oral infestation. Macroscopic fecal examinations were carried out daily from Day −28 to Day −1 to confirm the presence of proglottids and to confirm success of the experimental infection.

On Day 0, all dogs are fed within approximately 15-60 minutes before oral administration of the chewable tablet containing the 4-way combination of active agents to dogs in Treatment Group 2.

On Day 10, the dogs in the study were euthanized, and necropsy and recovery of helminths from the gastrointestinal tract was performed in order to assess the worm burdens for dogs in both groups. The percent reduction of Dipylidium caninum helminths in dogs in Treatment Group 2 compared with Treatment Group 1 was determined to be 100%.
Biological Example 4: Evaluation of the efficacy of a chewable tablet composition comprising a 4-way combination of compound A472, afoxolaner, moxidectin and praziquantel and a chewable tablet composition comprising a 3-way combination of compound A472, afoxolaner and praziquantel against infections of Dirofilaria immitis (heartworm) in dogs.

Three Treatment Groups of six beagle dogs each were set up for the study. Dogs in Treatment Group 1 were not treated and serve as a control group. Dogs in Treatment Group 2 were treated with chewable tablets containing a 4-way active agent combination prepared according to Preparation Example 11 to deliver minimum efficacious doses of 0.8 mg/kg body weight compound A472, 12 microgram/kg body weight moxidectin, 2.5 mg/kg body weight afoxolaner and 5 mg/kg body weight praziquantel on Day 0 of the study. Dogs in Treatment Group 3 were treated with chewable tablets containing a 3-way active agent combination prepared according to Preparation Example 13 to deliver doses of 0.8 mg/kg body weight compound A472, 2.5 mg/kg body weight afoxolaner and 5 mg/kg body weight praziquantel on Day 0 of the study.

On Day −30 of the study, all dogs in the study were infected with 50 L3 larvae of Dirofilaria immitis. On Day 0, dogs in Treatment Groups 2 and 3 were fed approximately 15-60 minutes prior to treatment. Dogs in Treatment Groups 2 were administered chewable tablets containing the 4-way active agent combination, and dogs in Treatment Group 3 were administered chewable tablets containing the 3-way active agent combination described above.

On Day 119 of the study, all the dogs in the study were euthanized, and necropsy and determination of the number and sex of Dirofilaria immits worms was determined. The percent reduction of Dirofilaria immitis worms in dogs in Treatment Group 2 was determined to be 100% compared with the control group, Treatment Group 1. The percent reduction of Dirofilaria immitis worms in Treatment Group 3 was determined to be 32.4% compared with Treatment Group 1.
Biological Example 5: Evaluation of the efficacy of a 4-way chewable tablet composition comprising a combination of compound A472, afoxolaner, moxidectin and praziquantel, and a 3-way chewable tablet composition comprising a combination of compound A472, moxidectin and praziquantel against induced infections of Amblyomma americanum on dogs.

The study is conducted in accordance with A. A. Marchiondo, et al. World Association for the Advancement of Veterinary Parasitology (W. A. A.V.P.) second edition: Guidelines for evaluating the efficacy of parasiticides for the treatment, prevention and control of flea and tick infestations on dogs and cats. Veterinary Parasitology 194 (2013) 84-97. Three Treatment Groups containing ten beagle dogs each are set up for the study. Dogs in Treatment Group 1 are untreated and serve as a control group. Dogs in Treatment Group 2 are treated with a 4-way chewable tablet composition according to Preparation Example 11 to target a minimum efficacious dose of 0.8 mg/kg body weight Compound A472, 12 micrograms/kg body weight moxidectin (μg/kg), 2.5 mg/kg body weight afoxolaner and 5 mg/kg body weight praziquantel on Treatment Day 0. Dogs in Treatment Group 3 are treated with a 3-way chewable tablet composition prepared similarly to Preparation Example 14 to target a minimum efficacious dose of 0.8 mg/kg body weight Compound A472, 12 micrograms/kg body weight moxidectin (μg/kg), and 5 mg/kg body weight praziquantel on Treatment Day 0. Dogs are treated in the fed prandial state.

On Day −1, each dog in the study is infested with 50 (±5) Amblyomma americanum ticks. On Day 0, dogs in Treatment Groups 2 and 3 are fed approximately 15-60 minutes before treatment and then treated with a combination of 4-way (Treatment Group 2) and 3-way (Treatment Group 3) tablets to reach the target dose. Dogs in Treatment Group 1 are not treated.

On Day 3 (72±2 hours post treatment), the dogs are examined, and the number of live and dead ticks are counted. Dogs are re-infested with 50 (±5) Amblyomma americanum ticks on Days 7, 14, 21 and 30 of the study. Dogs are further examined on Days 10, 17, 24 and 33 (72±2 hours post last treatment) after re-infestations to count dead and live ticks.

The percent efficacy of live tick counts in Treatment Group 2 compared with Treatment Group 1 (untreated control group) is expected to be 90% or greater up to at least Day 33. The percent efficacy of live tick counts in Treatment Group 3, which does not contain afoxolaner, is expected to be negligible compared with Treatment Group 1 (untreated control group).
Biological Example 6: Evaluation of the efficacy of a 4-way chewable tablet composition comprising a combination of compound A472, afoxolaner, moxidectin and praziquantel against induced infections of Ancylostoma caninum in dogs.

The study is conducted in accordance with VICH GL7 Effectiveness of Anthelmintics: General Requirements, 2025; and World Association for the Advancement of Veterinary Parasitology (WAAVP), second edition: Guidelines for evaluating the efficacy of anthelmintics for dogs and cats, 2024.

Two Treatment Groups containing ten Beagle dogs each are set up. Dogs in Treatment Group 1 serve as an untreated control group. Dogs in Treatment Group 2 are treated with chewable tablets containing a 4-way combination of compound A472, afoxolaner, moxidectin and praziquantel at concentrations to deliver minimum efficacious doses close to the target dose of 0.8 mg/kg body weight compound A472, 2.5 mg/kg body weight afoxolaner, 12 micrograms/kg body weight moxidectin and 5 mg/kg body weight praziquantel prepared similarly to Preparation Example 11.

On Day −21 to −28 of the study, all dogs are orally inoculated with approximately 300 infective Ancylostoma caninum L3 larvae. On Day −1, dogs are randomly allocated to treatment groups 1 or 2 described above. Dogs in Treatment Group 2 are treated with the 4-way chewable tablets once on Day 0 of the study. Dogs in Treatment Group 1 are untreated. Dogs are treated in the fed prandial state approximately 15-60 minutes after feeding.

On Day 7 to 10, dogs are euthanized for parasite recovery and counting. The percent reduction of Ancylostoma caninum worms in Treatment Group 2 is expected to be greater or equal to a 90% reduction in the geometric mean counts compared with Treatment Group 1 (untreated control).

The following numbered paragraphs are also part of the disclosure and comprise the spirit of the invention:
1. A composition comprising a combination of two or more antiparasitic active agents, wherein one of the antiparasitic active agents is an anthelmintic compound of Formula (IA), Formula (IB) or Formula (IC):

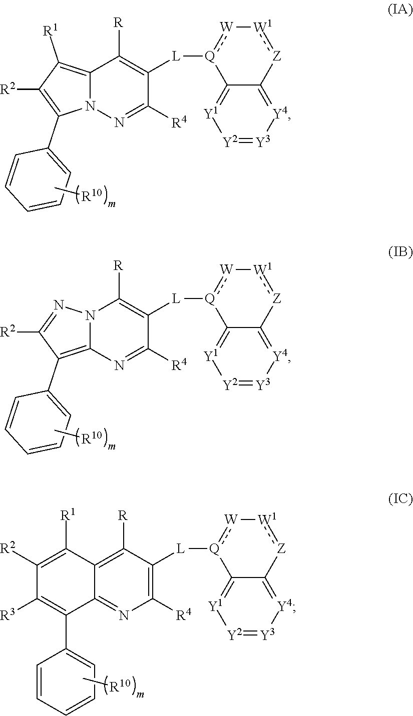

wherein:
L is L1 or L2:

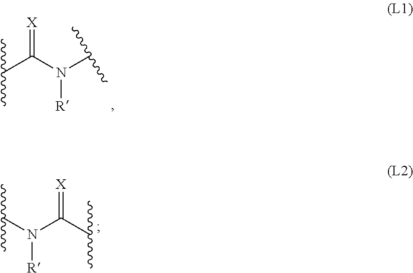

R′ is hydrogen, optionally substituted alkyl or optionally substituted haloalkyl;
R is optionally substituted alkyl, optionally substituted cycloalkyl, optionally substituted cycloalkenyl, optionally substituted alkenyl, optionally substituted alkynyl, optionally substituted heterocyclyl or —NR^{a}R^{b}, wherein R^{a} and R^{b} are independently H or optionally substituted alkyl;
R¹, R², R³ and R⁴ are independently hydrogen, cyano, halogen, optionally substituted alkyl, optionally substituted alkenyl, optionally substituted alkynyl, optionally substituted alkoxy;
R⁵ is independently each occurrence, hydrogen, halogen, cyano, optionally substituted alkyl, optionally substituted alkoxy, optionally substituted alkylcarbonyl, optionally substituted alkoxycarbonyl, optionally substituted aminocarbonyl, optionally substituted alkylaminocarbonyl, optionally substituted dialkylaminocarbonyl, optionally substituted alkylcarbonyloxy, optionally substituted alkylcarbonylamino, —SOₚ(optionally substituted alkyl), —SOₚ(optionally substituted haloalkyl);
R⁶ and R⁷ are independently hydrogen, halogen, optionally substituted alkyl, optionally substituted alkenyl, optionally substituted alkynyl, optionally substituted alkoxy, or R⁶ together with R⁷ for a carbonyl group (C═O);
R⁸ is hydrogen or optionally substituted alkyl;
R⁹ is hydrogen, halogen or optionally substituted alkyl;
R¹⁰ is cyano, halogen, optionally substituted alkyl or optionally substituted alkoxy;
Q is C, C—R⁹ or N;
X is O, S or N—R′;
Y¹, Y², Y³ and Y⁴ are each independently N or —CR⁵—;
W is CR⁶, CR⁶R⁷, O, SOₚ, N or N—R⁸, or W is absent;
W¹ is CR⁶, CR⁶R⁷, O, SOₚ, N or N—R⁸
Z is CR⁶, CR⁶R⁷, O, SOₚ, N or N—R⁸, and
wherein at most three of Y¹, Y², Y³ and Y⁴ are N;
m is 0, 1, 2, 3, 4 or 5;
p is independently in each occurrence is 0, 1, or 2;
the dashed bonds ( [symbol] ) signifies a single or double bond; and

wherein “optionally substituted” indicates that the group recited may be optionally substituted with halogen (independently chloro, fluoro, bromo or iodo), hydroxyl, thiol, oxo (C═O), C₁-C₆-alkyl, C₁-C₆-haloalkyl, C₃-C₈-cycloalkyl, C₁-C₆-alkoxy, C₁-C₆-haloalkoxy, —S(O)ₚ(C₁-C₆-alkyl) or —S(O)ₚ(C₁-C₆-haloalkyl) where p is 0, 1 or 2, carboxyl, acetyl, C₁-C₆-alkylcarbonyl, C₁-C₆-haloalkylcarbonyl, C₁-C₆-alkoxycarbonyl, C₁-C₆-haloalkoxycarbonyl, aminocarbonyl, C₁-C₆-alkylaminocarbonyl, di-C₁-C₆-alkylaminocarbonyl, C₁-C₆-haloalkylaminocarbonyl, di-C₁-C₆-haloalkylaminocarbonyl, amino, C₁-C₆-alkylamino, di-C₁-C₆-alkylamino, nitro, cyano, R₃Si— or R₃SiO— where R is C₁-C₆-alkyl, C₁-C₆-haloalkyl or phenyl, SF₅, phenyl, naphthyl, 5- to 10-membered heteroaryl containing from one to three N, O, Si or S heteroatoms; and 3- to 10-membered heterocyclyl containing from one to three N, O, Si or S heteroatoms; and
the one or more additional antiparasitic active agent is a macrocyclic lactone, an isoxazoline, praziquantel or pyrantel.
2. The composition of paragraph 1, wherein Ris optionally substituted alkyl, optionally substituted haloalkyl, optionally substituted cycloalkyl or optionally substituted saturated or unsaturated heterocyclyl.
3. The composition of paragraphs 1 or 2, wherein R¹, R², R³ and R⁴ are each independently hydrogen, halogen, optionally substituted alkyl or optionally substituted haloalkyl.
4. The composition of any one of paragraphs 1 to 3, wherein R¹⁰ is halogen or optionally substituted alkyl.
5. The composition of any one of paragraphs 1 to 4, wherein the one or more additional antiparasitic active agent is a macrocyclic lactone selected from the group consisting of moxidectin, milbemycin oxime, ivermectin, eprinomectin or selamectin, or a combination thereof.
6. The composition of any one of paragraphs 1 to 4, wherein the one or more additional antiparasitic active agent is an isoxazoline active agent selected from the group consisting of afoxolaner, esafoxolaner, umifoxolaner, fluralaner, lotilaner or sarolaner.
7. The composition of any one of paragraphs 1 to 4, wherein the one or more additional antiparasitic active agent is praziquantel.
8. The composition of any one of paragraphs 1 to 4, wherein the one or more additional antiparasitic active agent is pyrantel.
9. The composition of any one of paragraphs 1 to 4, wherein the one or more additional antiparasitic active agent is a combination of a macrocyclic lactone and an isoxazoline active agent.
10. The composition of any one of paragraphs 1 to 4, wherein the one or more additional antiparastic active agent is a combination of a macrocyclic lactone, an isoxazoline active agent and praziquantel.
11. The composition of any one of paragraphs 1 to 4, wherein the one or more additional antiparastic active agent is a combination of a macrocyclic lactone, an isoxazoline active agent and pyrantel.
12. The composition of any one of paragraphs 9 to 11, wherein the macrocyclic lactone is selected from the group consisting of moxidectin, milbemycin oxime, ivermectin, eprinomectin and selamectin, or a combination thereof, and/or
wherein the isoxazoline active agent is selected from the group consisting of afoxolaner, esafoxolaner, umifoxolaner, fluralaner, lotilaner or sarolaner, or a combination thereof.
13. The composition of any one of paragraphs 1 to 8, wherein the one or more additional antiparasitic active agent is a combination of a macrocyclic lactone selected from the group consisting of moxidectin, milbemycin oxime, ivermectin, eprinomectin and selamectin, or a combination thereof, and an isoxazoline active agent selected from the group consisting of afoxolaner, esafoxolaner, umifoxolaner, fluralaner, lotilaner or sarolaner, or a combination thereof.
14. The composition of any one of paragraphs 1 to 8, wherein the one or more additional antiparasitic active agent is a combination of a macrocyclic lactone selected from the group consisting of moxidectin, milbemycin oxime, ivermectin, eprinomectin or selamectin, or a combination thereof, an isoxazoline active agent selected from the group consisting of afoxolaner, esafoxolaner, umifoxolaner, fluralaner, lotilaner or sarolaner, or a combination thereof, and praziquantel.
15. The composition of any one of paragraphs 1 to 8, wherein the one or more additional antiparasitic active agent is a combination of a macrocyclic lactone selected from the group consisting of moxidectin, milbemycin oxime, ivermectin, eprinomectin or selamectin, or a combination thereof, an isoxazoline active agent selected from the group consisting of afoxolaner, esafoxolaner, umifoxolaner, fluralaner, lotilaner or sarolaner, or a combination thereof, and pyrantel.
16. The composition of any one of paragraphs 1 to 8, wherein the one or more additional antiparasitic active agent is a combination of moxidectin or milbemycin oxime; afoxolaner, esafoxolaner, fluralaner or lotilaner; and praziquantel.
17. The composition of any one of paragraphs 1 to 8, wherein the one or more additional antiparasitic active agent is a combination of moxidectin, afoxolaner and praziquantel.
18. The composition of any one of paragraphs 1 to 17, wherein the anthelmintic compound is the compound of Formula (IA), wherein:
L is L1, wherein X is O and R′ is hydrogen;
Q is C—R⁹;
R is iso-propyl, tert-butyl, cyclopropyl, cyclobutyl, —N(CH₃)₂, azetidinyl or morpholinyl, each optionally substituted by one or more fluoro or methyl;
R¹, R⁴ and R⁹ are each hydrogen;
R² is hydrogen, methyl, —CF₃, chloro or fluoro;
R¹⁰ is chloro or fluoro and m is 2 or 3;
W and W¹ are each CH₂;
Z is O; and
Y¹, Y², Y³ and Y⁴ are each CH.
19. The composition of any one of paragraphs 1 to 17, wherein the anthelmintic compound is Compound A472, or a salt thereof:

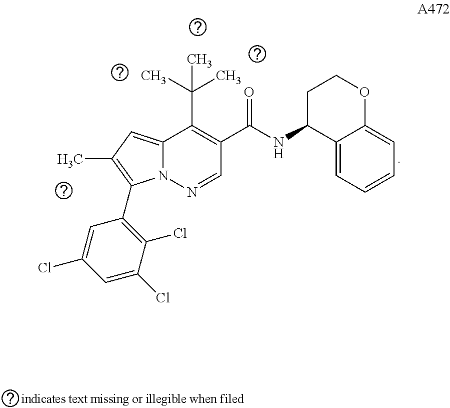 [SMILES: Cc1cc2c(C(C)(C)C)c(C(=O)N[C@H]3CCOc4ccccc43)cnn2c1-c1cc(Cl)cc(Cl)c1Cl]

20. The composition of any one of paragraphs 1 to 17, wherein the anthelmintic compound is the compound of Formula (IC), wherein:
L is L1 wherein X is O and R′ is hydrogen;
Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl;
R¹, R², R⁴ and R⁹ are each hydrogen;
R³ is hydrogen, methyl, —CF₃, chloro or fluoro;
R¹⁰ is CF₃, chloro or fluoro and m is 2 or 3;
W and W¹ are each CH₂;
Z is O; and
Y¹, Y², Y³ and Y⁴ are each CH.
21. The composition of any one of paragraphs 1 to 17, wherein the anthelmintic compound is Compound IC-1, or a salt thereof:

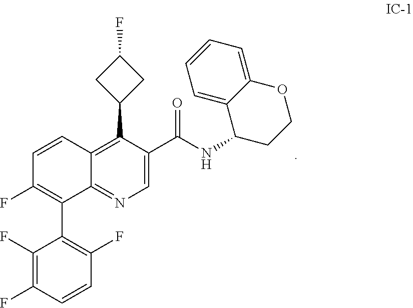 [SMILES: O=C(N[C@H]1CCOc2ccccc21)c1cnc2c(-c3c(F)ccc(F)c3F)c(F)ccc2c1[C@H]1C[C@H](F)C1]

22. A composition as defined in any one of paragraphs 1 to 21 for use in treating and/or preventing a parasitic infection and/or infestation in an animal.
23. A method of treating and/or preventing a parasitic infection and/or infestation in an animal, comprising administering a composition as defined in any one of paragraphs 1 to 21 to the animal.
24. The composition for use according to paragraphs 22, or the method according to paragraph 23, wherein the animal is a human, cat, dog, horse, cow, sheep or fish.
25. The composition for use according to paragraph 22 or paragraph 24, or the method according to paragraph 23 or paragraph 24, wherein the parasitic infection and/or infestation is an ectoparasitic and/or endoparasitic infection and/or infestation.
26. The composition for use according to any one of paragraphs 22, 24 and 25, or the method according to any one of paragraphs 23 to 25, wherein the parasitic infection and/or infestation is by Dirofilaria immitis, hookworm, roundworms, cestodes, ticks and fleas, or a combination thereof 0.27. A combination of antiparasitic active agents or a composition comprising a combination of antiparasitic active agents, wherein one of the antiparasitic active agents is an anthelmintic compound of Formula (IA):

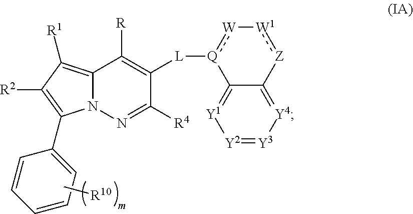

wherein:
L is L1:

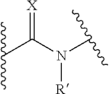

R′ is hydrogen;
R is iso-propyl, tert-butyl, cyclopropyl, cyclobutyl, —N(CH₃)₂, azetidinyl or morpholinyl, each optionally substituted by one or more fluoro or methyl;
R¹, R⁴ and R⁹ are each hydrogen;
R² is hydrogen, methyl, —CF₃, chloro or fluoro;
R¹⁰ is chloro or fluoro;
Q is C—R⁹;
X is O;
Y¹, Y², Y³ and Y⁴ are each CH;
W and W¹ are each CH₂;
Z is O and
m is 2 or 3;
the dashed bonds ( [symbol] ) signifies a single or double bond; and
wherein “optionally substituted” indicates that the group recited may be optionally substituted with halogen (independently chloro, fluoro, bromo or iodo), hydroxyl, thiol, oxo (C═O), C₁-C₆-alkyl, C₁-C₆-haloalkyl, C₃-C₈-cycloalkyl, C₁-C₆-alkoxy, C₁-C₆-haloalkoxy, —S(O)ₚ(C₁-C₆-alkyl) or —S(O)ₚ(C₁-C₆-haloalkyl) where p is 0, 1 or 2, carboxyl, acetyl, C₁-C₆-alkylcarbonyl, C₁-C₆-haloalkylcarbonyl, C₁-C₆-alkoxycarbonyl, C₁-C₆-haloalkoxycarbonyl, aminocarbonyl, C₁-C₆-alkylaminocarbonyl, di-C₁-C₆-alkylaminocarbonyl, C₁-C₆-haloalkylaminocarbonyl, di-C₁-C₆-haloalkylaminocarbonyl, amino, C₁-C₆-alkylamino, di-C₁-C₆-alkylamino, nitro, cyano, R₃Si— or R₃SiO— where R is C₁-C₆-alkyl, C₁-C₆-haloalkyl or phenyl, SF₅, phenyl, naphthyl, 5- to 10-membered heteroaryl containing from one to three N, O, Si or S heteroatoms; and 3- to 10-membered heterocyclyl containing from one to three N, O, Si or S heteroatoms; and
wherein the additional antiparasitic active agents are a macrocyclic lactone, an isoxazoline, and praziquantel,
preferably the anthelmintic compound of Formula (IA) is Compound A472, or a salt thereof:

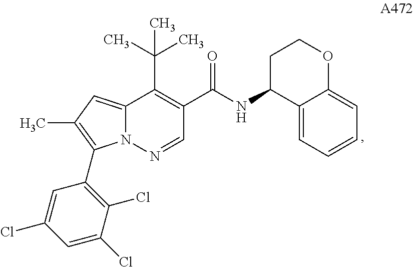 [SMILES: Cc1cc2c(C(C)(C)C)c(C(=O)N[C@H]3CCOc4ccccc43)cnn2c1-c1cc(Cl)cc(Cl)c1Cl]

preferably the macrocyclic lactone is moxidectin, and
preferably the isoxazoline is afoxolaner.
28. A composition comprising a combination of two or more antiparasitic active agents, wherein one of the antiparasitic active agents is an anthelmintic compound of Formula (IA):

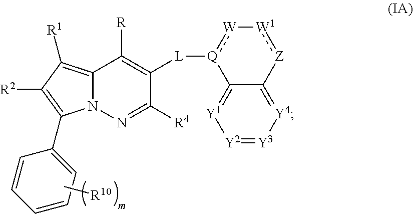

wherein:
L is L1:

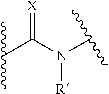

R′ is hydrogen;
R is iso-propyl, tert-butyl, cyclopropyl, cyclobutyl, —N(CH₃)₂, azetidinyl or morpholinyl, each optionally substituted by one or more fluoro or methyl;
R¹, R⁴ and R⁹ are each hydrogen;
R² is hydrogen, methyl, —CF₃, chloro or fluoro;
R¹⁰ is chloro or fluoro;
Q is C—R⁹;
X is O;
Y¹, Y², Y³ and Y⁴ are each CH;
W and W¹ are each CH₂;
Z is O, and
m is 2 or 3;
the dashed bonds ( [symbol] ) signifies a single or double bond; and
wherein “optionally substituted” indicates that the group recited may be optionally substituted with halogen (independently chloro, fluoro, bromo or iodo), hydroxyl, thiol, oxo (C═O), C₁-C₆-alkyl, C₁-C₆-haloalkyl, C₃-C₈-cycloalkyl, C₁-C₆-alkoxy, C₁-C₆-haloalkoxy, —S(O)ₚ(C₁-C₆-alkyl) or —S(O)ₚ(C₁-C₆-haloalkyl) where p is 0, 1 or 2, carboxyl, acetyl, C₁-C₆-alkylcarbonyl, C₁-C₆-haloalkylcarbonyl, C₁-C₆-alkoxycarbonyl, C₁-C₆-haloalkoxycarbonyl, aminocarbonyl, C₁-C₆-alkylaminocarbonyl, di-C₁-C₆-alkylaminocarbonyl, C₁-C₆-haloalkylaminocarbonyl, di-C₁-C₆-haloalkylaminocarbonyl, amino, C₁-C₆-alkylamino, di-C₁-C₆-alkylamino, nitro, cyano, R₃Si— or R₃SiO— where R is C₁-C₆-alkyl, C₁-C₆-haloalkyl or phenyl, SF₅, phenyl, naphthyl, 5- to 10-membered heteroaryl containing from one to three N, O, Si or S heteroatoms; and 3- to 10-membered heterocyclyl containing from one to three N, O, Si or S heteroatoms; and
wherein the one or more additional antiparasitic active agent is a macrocyclic lactone, an isoxazoline, or praziquantel.
29. The composition of paragraph 28, wherein the one or more additional antiparasitic active agent is a macrocyclic lactone.
30. The composition of paragraphs 28 or 29, wherein the one or more additional antiparasitic active agent is an isoxazoline.
31. The composition of any one of paragraphs 28 to 30, wherein the one or more additional antiparasitic active agent is praziquantel.
32. The composition of any one of paragraphs 28 to 31, wherein the one or more additional antiparasitic active agent is a combination of a macrocyclic lactone and an isooxazoline, and optionally praziquantel.
33. The composition or combination of any one of paragraphs 27 to 32, wherein the macrocyclic lactone is selected from the group consisting of moxidectin, milbemycin oxime, ivermectin, eprinomectin or selamectin, or a combination thereof.
34. The composition or combination of any one of paragraphs 27 to 33, wherein the isoxazoline is selected from the group consisting of afoxolaner, esafoxolaner, umifoxolaner, fluralaner, lotilaner or sarolaner.
35. The composition of any one of paragraphs 28 to 34, wherein the one or more additional antiparasitic active agent comprises or is a combination of (i) a macrocyclic lactone selected from the group consisting of moxidectin, milbemycin oxime, ivermectin, eprinomectin and selamectin, or a combination thereof, and (ii) an isoxazoline active agent selected from the group consisting of afoxolaner, esafoxolaner, umifoxolaner, fluralaner, lotilaner or sarolaner, or a combination thereof, and (iii) optionally praziquantel.
36. A combination of antiparasitic active agents or the composition of any one of paragraphs 1 to 35 comprising a combination of antiparasitic active agents, wherein said combination of antiparasitic active agents consists of (i) the anthelmintic compound of Formula (IA) as described in paragraph 27 or 28, (ii) a macrocyclic lactone selected from the group consisting of moxidectin, milbemycin oxime, ivermectin, eprinomectin or selamectin, or a combination thereof, preferably moxidectin, (iii) an isoxazoline active agent selected from the group consisting of afoxolaner, esafoxolaner, umifoxolaner, fluralaner, lotilaner or sarolaner, or a combination thereof, preferably afoxolaner, and (iv) praziquantel.
37. The composition of any one of paragraphs 2 to 36, wherein the one or more additional antiparasitic active agent comprises or is a combination of moxidectin or milbemycin oxime; afoxolaner, esafoxolaner, fluralaner or lotilaner; and praziquantel, preferably the one or more
additional antiparasitic active agent is a combination consisting of moxidectin, afoxolaner and praziquantel.
38. The composition or combination of any one of paragraphs 1 to 37, wherein the anthelmintic compound of Formula (IA) is Compound A472, or a salt thereof:

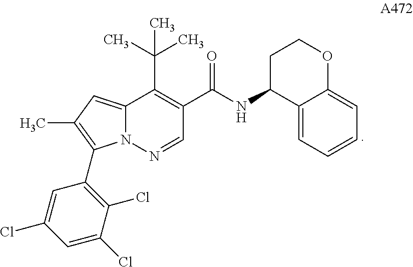 [SMILES: Cc1cc2c(C(C)(C)C)c(C(=O)N[C@H]3CCOc4ccccc43)cnn2c1-c1cc(Cl)cc(Cl)c1Cl]

39. A composition or combination as defined in any one of paragraphs 1 to 38 for use in treating and/or preventing a parasitic infection and/or infestation in an animal.
40. The composition or combination for use according to paragraph 39, wherein the animal is a human, cat, dog, horse, cow, sheep or fish, preferably a dog or a cat.
41. The composition or combination for use according to any one of paragraphs 39 or 40, wherein the parasitic infection and/or infestation is by Dirofilaria immitis, hookworm, roundworms, cestodes, ticks and fleas, or a combination thereof.
42. An antiparasitic combination of two or more antiparasitic active agents, wherein a first antiparasitic active agent in the combination is an anthelmintic compound of Formula (IA), Formula (IB) or Formula (IC), or a combination thereof:

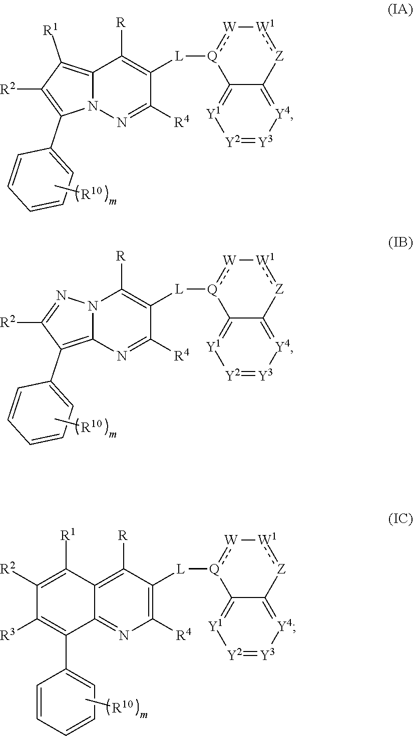

or salts thereof,
wherein:
L is L1 or L2:

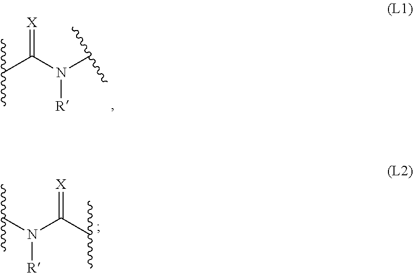

R′ is hydrogen, optionally substituted alkyl or optionally substituted haloalkyl;
R is optionally substituted alkyl, optionally substituted cycloalkyl, optionally substituted cycloalkenyl, optionally substituted alkenyl, optionally substituted alkynyl, optionally substituted heterocyclyl or —NR^{a}R^{b}, wherein R^{a} and R^{b} are independently H or optionally substituted alkyl;
R¹, R², R³ and R⁴ are independently hydrogen, cyano, halogen, optionally substituted alkyl, optionally substituted alkenyl, optionally substituted alkynyl, optionally substituted alkoxy;
R⁵ is independently each occurrence, hydrogen, halogen, cyano, optionally substituted alkyl, optionally substituted alkoxy, optionally substituted alkylcarbonyl, optionally substituted alkoxycarbonyl, optionally substituted aminocarbonyl, optionally substituted alkylaminocarbonyl, optionally substituted dialkylaminocarbonyl, optionally substituted alkylcarbonyloxy, optionally substituted alkylcarbonylamino, —SOₚ(optionally substituted alkyl), —SOₚ(optionally substituted haloalkyl);
R⁶ and R⁷ are independently hydrogen, halogen, optionally substituted alkyl, optionally substituted alkenyl, optionally substituted alkynyl, optionally substituted alkoxy, or R⁶ together with R⁷ for a carbonyl group (C═O);
R⁸ is hydrogen or optionally substituted alkyl;
R⁹ is hydrogen, halogen or optionally substituted alkyl;
R¹⁰ is cyano, halogen, optionally substituted alkyl or optionally substituted alkoxy;
Q is C, C—R⁹ or N;
X is O, S or N—R′;
Y¹, Y², Y³ and Y⁴ are each independently N or —CR⁵—;
W is CR⁶, CR⁶R⁷, O, SOₚ, N or N—R⁸, or W is absent;
W¹ is CR⁶, CR⁶R⁷, O, SOₚ, N or N—R⁸
Z is CR⁶, CR⁶R⁷, O, SOₚ, N or N—R⁸, and
wherein at most three of Y¹, Y², Y³ and Y⁴ are N;
m is 0, 1, 2, 3, 4 or 5;
p is independently in each occurrence is 0, 1, or 2;
the dashed bonds ( [symbol] ) signifies a single or double bond; and
wherein “optionally substituted” indicates that the group recited may be optionally substituted with halogen (independently chloro, fluoro, bromo or iodo), hydroxyl, thiol, oxo (C═O), C₁-C₆-alkyl, C₁-C₆-haloalkyl, C₃-C₈-cycloalkyl, C₁-C₆-alkoxy, C₁-C₆-haloalkoxy, —S(O)ₚ(C₁-C₆-alkyl) or —S(O)ₚ(C₁-C₆-haloalkyl) where p is 0, 1 or 2, carboxyl, acetyl, C₁-C₆-alkylcarbonyl, C₁-C₆-haloalkylcarbonyl, C₁-C₆-alkoxycarbonyl, C₁-C₆-haloalkoxycarbonyl, aminocarbonyl, C₁-C₆-alkylaminocarbonyl, di-C₁-C₆-alkylaminocarbonyl, C₁-C₆-haloalkylaminocarbonyl, di-C₁-C₆-haloalkylaminocarbonyl, amino, C₁-C₆-alkylamino, di-C₁-C₆-alkylamino, nitro, cyano, R₃Si— or R₃SiO— where R is C₁-C₆-alkyl, C₁-C₆-haloalkyl or phenyl, SF₅, phenyl, naphthyl, 5- to 10-membered heteroaryl containing from one to three N, O, Si or S heteroatoms; and 3- to 10-membered heterocyclyl containing from one to three N, O, Si or S heteroatoms; and
the second or more additional antiparasitic active agent(s) in the combination is a macrocyclic lactone, an isoxazoline, praziquantel or pyrantel, or a combination thereof.
43. The antiparasitic combination of paragraph 42, wherein Ris optionally substituted alkyl, optionally substituted haloalkyl, optionally substituted cycloalkyl or optionally substituted saturated or unsaturated heterocyclyl.
44. The antiparasitic combination of paragraph 42 or 43, wherein R¹, R², R³ and R⁴ are each independently hydrogen, halogen, optionally substituted alkyl or optionally substituted haloalkyl.
45. The antiparasitic combination of any one of paragraphs 42 to 44, wherein R¹⁰ is halogen or optionally substituted alkyl.
46. The antiparasitic combination of any one of paragraphs 42 to 45, wherein the second or more additional antiparasitic active agent(s) comprises, consists essentially of, or consists of, a macrocyclic lactone selected from the group consisting of moxidectin, milbemycin oxime, ivermectin, eprinomectin and selamectin, or a combination thereof.
47. The antiparasitic combination of any one of paragraphs 42 to 45, wherein the second or more additional antiparasitic active agent(s) comprises, consists essentially of, or consists of, an isoxazoline active agent selected from the group consisting of afoxolaner, esafoxolaner, umifoxolaner, fluralaner, lotilaner and sarolaner.
48. The antiparasitic combination of any one of paragraphs 42 to 45, wherein the second or more additional antiparasitic active agent(s) comprises, consists essentially of, or consists of, praziquantel.
49. The antiparasitic combination of any one of paragraphs 42 to 45, wherein the second or more additional antiparasitic active agent(s), consists essentially of, or consists of, pyrantel.
50. The antiparasitic combination of any one of paragraphs 42 to 45, wherein the second or more additional antiparasitic active agent(s) comprises, consists essentially of, or consists of, a combination of a macrocyclic lactone and an isoxazoline active agent.
51. The antiparasitic combination of any one of paragraphs 42 to 45, wherein the second or more additional antiparastic active agent(s) comprises, consists essentially of, or consists of, a combination of a macrocyclic lactone, an isoxazoline active agent and praziquantel.
52. The antiparasitic combination of any one of paragraphs 42 to 4, wherein the second or more additional antiparastic active agent(s) comprises, or consists essentially of, consists of, a combination of a macrocyclic lactone, an isoxazoline active agent and pyrantel.
53. The antiparasitic combination of any one of paragraphs 50 to 52, wherein the macrocyclic lactone is selected from the group consisting of moxidectin, milbemycin oxime, ivermectin, eprinomectin and selamectin, or a combination thereof, and/or
wherein the isoxazoline active agent is selected from the group consisting of afoxolaner, esafoxolaner, umifoxolaner, fluralaner, lotilaner and sarolaner, or a combination thereof.
54. The antiparasitic combination of any one of paragraphs 42 to 45, wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; and an isoxazoline active agent selected from the group consisting of afoxolaner, esafoxolaner, umifoxolaner, fluralaner, lotilaner and sarolaner, or a combination thereof.
55. The antiparasitic combination of any one of paragraphs 42 to 45, wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof; and praziquantel.
56. The antiparasitic combination of any one of paragraphs 42 to 45, wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof, and pyrantel.
57. The antiparasitic combination of any one of paragraphs 42 to 45, wherein the second or more additional antiparasitic active agent(s) comprises, consists essentially of, or consists of, a combination of moxidectin or milbemycin oxime; afoxolaner, esafoxolaner, fluralaner or lotilaner; and praziquantel.
58. The antiparasitic combination of any one of paragraphs 42 to 45, wherein the second or more additional antiparasitic active agent(s) comprises, consists essentially of, or consists of, a combination of moxidectin, afoxolaner and praziquantel.
59. The antiparasitic combination of any one of paragraphs 42 to 58, wherein the anthelmintic compound is the compound of Formula (IA), wherein:
L is L1, wherein X is O and R′ is hydrogen;
Q is C—R⁹;
R is iso-propyl, tert-butyl, cyclopropyl, cyclobutyl, —N(CH₃)₂, azetidinyl or morpholinyl, each optionally substituted by one or more fluoro or methyl;
R¹, R⁴ and R⁹ are each hydrogen;
R² is hydrogen, methyl, —CF₃, chloro or fluoro;
R¹⁰ is chloro or fluoro and m is 2 or 3;
W and W¹ are each CH₂;
Z is O; and
Y¹, Y², Y³ and Y⁴ are each CH.
60. The antiparasitic combination of any one of paragraphs 42 to 59, wherein the anthelmintic compound is Compound A472, or a salt thereof:

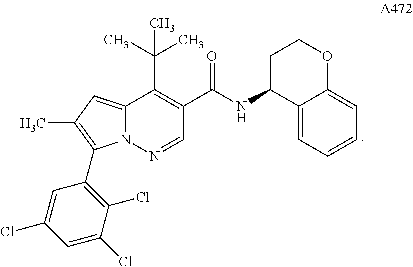 [SMILES: Cc1cc2c(C(C)(C)C)c(C(=O)N[C@H]3CCOc4ccccc43)cnn2c1-c1cc(Cl)cc(Cl)c1Cl]

61. The antiparasitic combination of any one of paragraphs 42 to 58, wherein the anthelmintic compound is the compound of Formula (IC), wherein:
L is L1 wherein X is O and R′ is hydrogen;
Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl;
R¹, R², R⁴ and R⁹ are each hydrogen;
R³ is hydrogen, methyl, —CF₃, chloro or fluoro;
R¹⁰ is CF₃, chloro or fluoro and m is 2 or 3;
W and W¹ are each CH₂;
Z is O; and
Y¹, Y², Y³ and Y⁴ are each CH.
62. The antiparasitic combination of any one of paragraphs 42 to 58 or 61, wherein the anthelmintic compound is Compound IC-1, or a salt thereof.

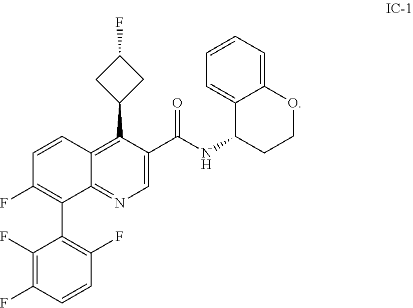 [SMILES: O=C(N[C@H]1CCOc2ccccc21)c1cnc2c(-c3c(F)ccc(F)c3F)c(F)ccc2c1[C@H]1C[C@H](F)C1]

63. The antiparasitic combination of any one of paragraphs 42 to 60, wherein the first antiparasitic active agent is Compound A472, or a salt thereof.

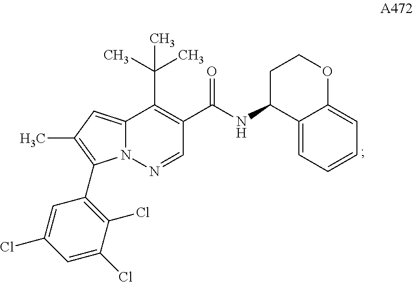 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, a macrocyclic lactone selected from the group consisting of moxidectin, milbemycin oxime, ivermectin, eprinomectin and selamectin, or a combination thereof.
64. The antiparasitic combination of any one of paragraphs 42 to 60, wherein the first antiparasitic active agent is Compound A472, or a salt thereof:

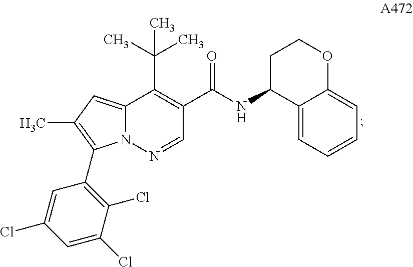 [SMILES: Cc1cc2c(C(C)(C)C)c(C(=O)N[C@H]3CCOc4ccccc43)cnn2c1-c1cc(Cl)cc(Cl)c1Cl]

and wherein the second or more antiparasitic active agent(s) comprises, consists essentially of, or consists of, an isoxazoline active agent selected from the group consisting of afoxolaner, esafoxolaner, umifoxolaner, fluralaner, lotilaner and sarolaner, or a combination thereof.
65. The antiparasitic combination of any one of paragraphs 42 to 60, wherein the first antiparasitic active agent is Compound A472, or a salt thereof:

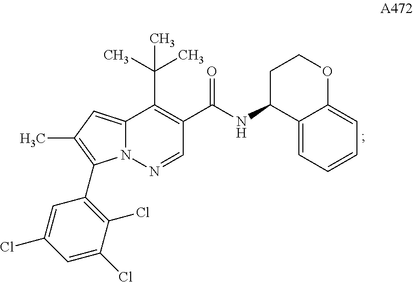 [SMILES: Cc1cc2c(C(C)(C)C)c(C(=O)N[C@H]3CCOc4ccccc43)cnn2c1-c1cc(Cl)cc(Cl)c1Cl]

and wherein the second or more antiparasitic active agent(s) comprises, consists essentially of, or consists of, praziquantel.
66. The antiparasitic combination of any one of paragraphs 42 to 60, wherein the first antiparasitic active agent is Compound A472, or a salt thereof:

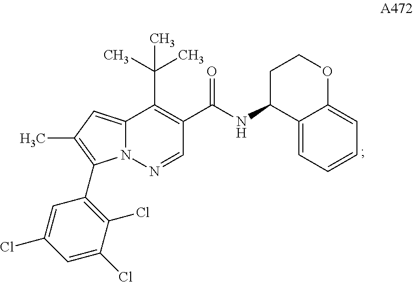 [SMILES: Cc1cc2c(C(C)(C)C)c(C(=O)N[C@H]3CCOc4ccccc43)cnn2c1-c1cc(Cl)cc(Cl)c1Cl]

and wherein the second or more antiparasitic active agent(s) comprises, consists essentially of, or consists of, pyrantel.
67. The antiparasitic combination of any one of paragraphs 42 to 60, wherein the first antiparasitic active agent is Compound A472, or a salt thereof:

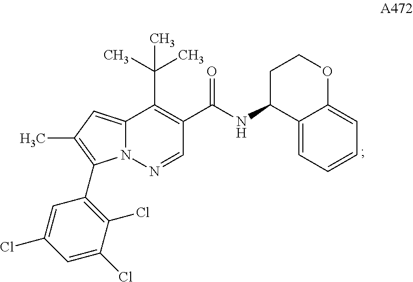 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, a combination of a macrocyclic lactone selected from the group consisting of moxidectin, milbemycine oxime, ivermectin, eprinomectin and selamectin, or a combination thereof; and an isoxazoline active agent selected from the group consisting of afoxolaner, esafoxolaner, umifoxolaner, fluralaner, lotilaner and sarolaner, or a combination thereof.
68. The antiparasitic combination of any one of paragraphs 42 to 60, wherein the first antiparasitic active agent is Compound A472, or a salt thereof:

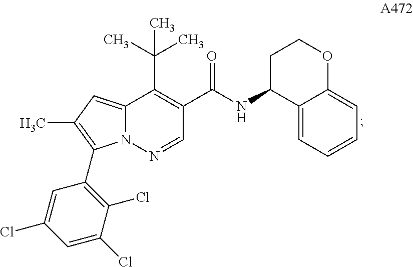 [SMILES: Cc1cc2c(C(C)(C)C)c(C(=O)N[C@H]3CCOc4ccccc43)cnn2c1-c1cc(Cl)cc(Cl)c1Cl]

and wherein the second or more antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof, an isoxazoline active agent selected from the group consisting of afoxolaner, esafoxolaner, umifoxolaner, fluralaner, lotilaner and sarolaner, or a combination thereof; and praziquantel.
69. The antiparasitic combination of any one of paragraphs 42 to 60, wherein the first antiparasitic active agent is Compound A472, or a salt thereof:

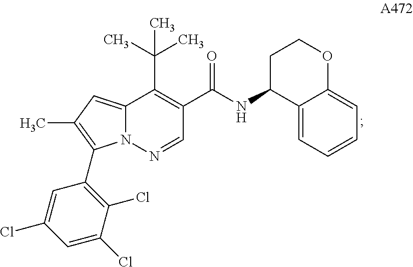 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof, an isoxazoline active agent selected from the group consisting of afoxolaner, esafoxolaner, umifoxolaner, fluralaner, lotilaner and sarolaner, or a combination thereof, and pyrantel.
70. The antiparasitic combination of any one of paragraphs 42 to 60, wherein the first antiparasitic active agent is Compound A472, or a salt thereof:

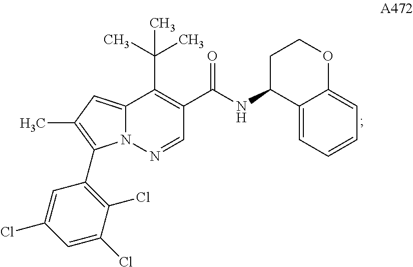 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, afoxolaner.
71. The antiparasitic combination of any one of paragraphs 42 to 60, wherein the first antiparasitic active agent is Compound A472, or a salt thereof:

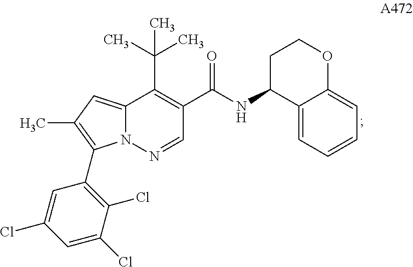 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, esafoxolaner.
72. The antiparasitic combination of any one of paragraphs 42 to 60, wherein the first antiparasitic active agent is Compound A472, or a salt thereof:

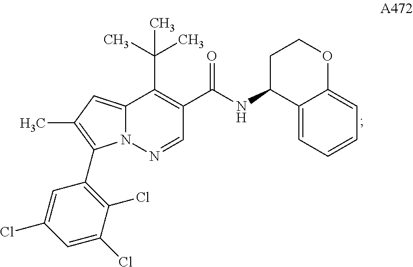 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, umifoxolaner.
73. The antiparasitic combination of any one of paragraphs 42 to 60, wherein the first antiparasitic active agent is Compound A472, or a salt thereof:

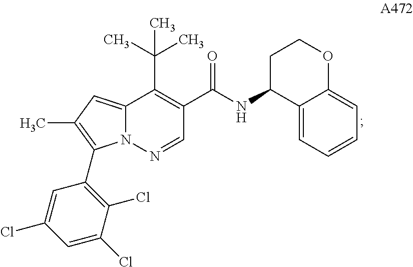 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, fluralaner.
74. The antiparasitic combination of any one of paragraphs 42 to 60, wherein the first antiparasitic active agent is Compound A472, or a salt thereof:

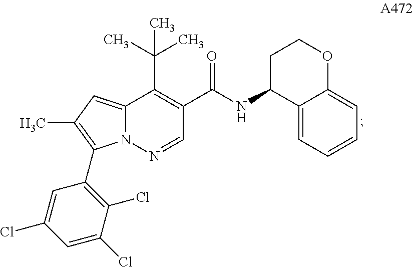 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, sarolaner.
75. The antiparasitic combination of any one of paragraphs 42 to 60, wherein the first antiparasitic active agent is Compound A472, or a salt thereof.

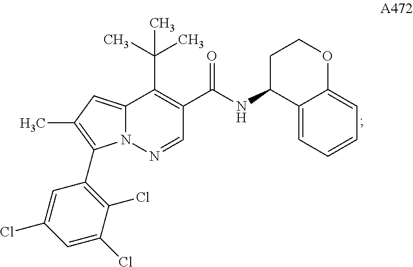 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, lotilaner.
76. The antiparasitic combination of any one of paragraphs 42 to 60, wherein the first antiparasitic active agent is Compound A472, or a salt thereof.

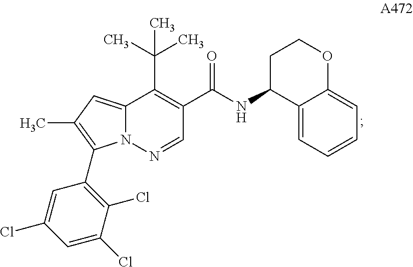 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, moxidectin.
77. The antiparasitic combination of any one of paragraphs 42 to 60, wherein the first antiparasitic active agent is Compound A472, or a salt thereof.

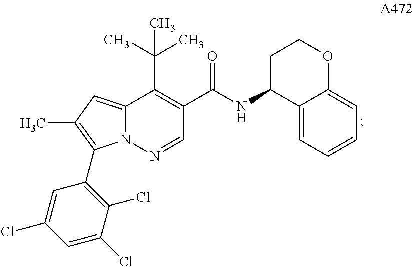 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, milbemycin oxime.
78. The antiparasitic combination of any one of paragraphs 42 to 60, wherein the first antiparasitic active agent is Compound A472, or a salt thereof.

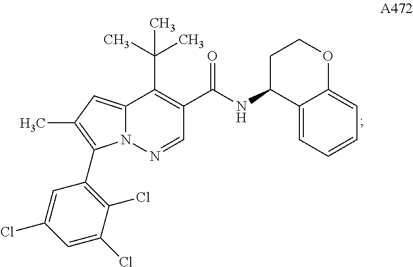 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, eprinomectin.
79. The antiparasitic combination of any one of paragraphs 42 to 60, wherein the first antiparasitic active agent is Compound A472, or a salt thereof.

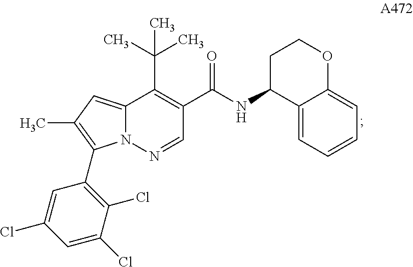 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, ivermectin.
80. The antiparasitic combination of any one of paragraphs 42 to 60, wherein the first antiparasitic active agent is Compound A472, or a salt thereof.

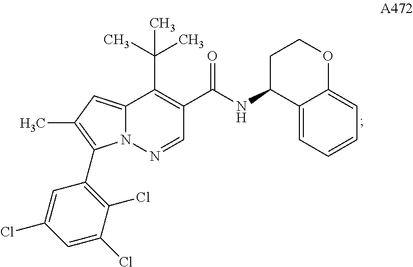 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, selamectin.
81. The antiparasitic combination of any one of paragraphs 42 to 60, wherein the first antiparasitic active agent is Compound A472, or a salt thereof.

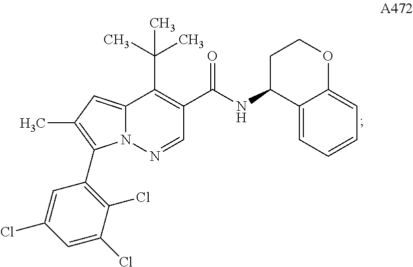 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, afoxolaner and moxidectin.
82. The antiparasitic combination of any one of paragraphs 42 to 60, wherein the first antiparasitic active agent is Compound A472, or a salt thereof.

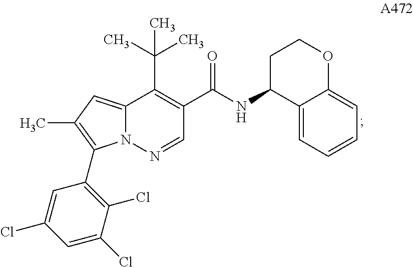 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, esafoxolaner and moxidectin.
83. The antiparasitic combination of any one of paragraphs 42 to 60, wherein the first antiparasitic active agent is Compound A472, or a salt thereof.

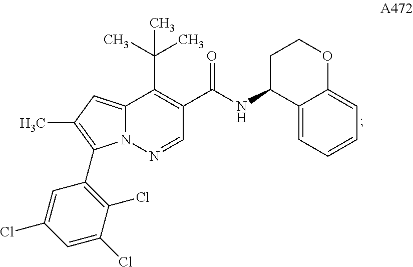 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, umifoxolaner and moxidectin.
84. The antiparasitic combination of any one of paragraphs 42 to 60, wherein the first antiparasitic active agent is Compound A472, or a salt thereof.

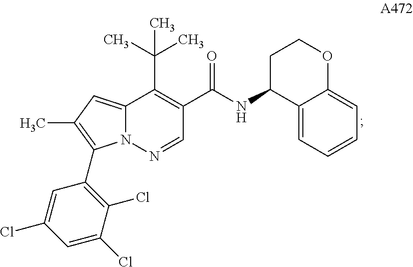 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, fluralaner and moxidectin.
85. The antiparasitic combination of any one of paragraphs 42 to 60, wherein the first antiparasitic active agent is Compound A472, or a salt thereof.

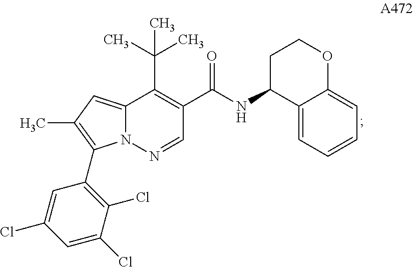 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, sarolaner and moxidectin.
86. The antiparasitic combination of any one of paragraphs 42 to 60, wherein the first antiparasitic active agent is Compound A472, or a salt thereof.

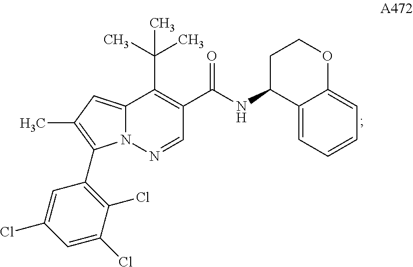 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, lotilaner and moxidectin.
87. The antiparasitic combination of any one of paragraphs 42 to 60, wherein the first antiparasitic active agent is Compound A472, or a salt thereof.

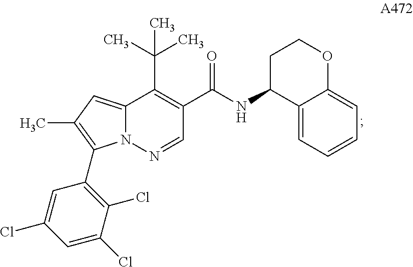 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, afoxolaner, moxidectin and praziquantel.
88. The antiparasitic combination of any one of paragraphs 42 to 60, wherein the first antiparasitic active agent is Compound A472, or a salt thereof.

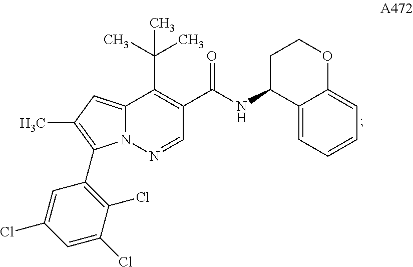 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, esafoxolaner, moxidectin and praziquantel.
89. The antiparasitic combination of any one of paragraphs 42 to 60, wherein the first antiparasitic active agent is Compound A472, or a salt thereof:

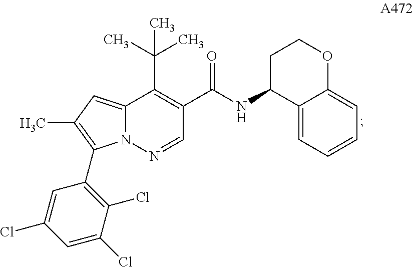 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, umifoxolaner, moxidectin and praziquantel.
90. The antiparasitic combination of any one of paragraphs 42 to 60, wherein the first antiparasitic active agent is Compound A472, or a salt thereof:

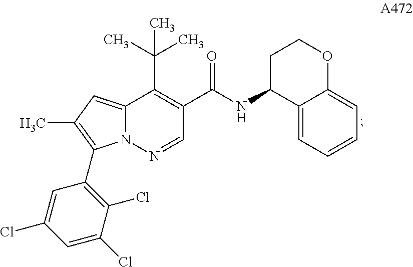 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, fluralaner, moxidectin and praziquantel.
91. The antiparasitic combination of any one of paragraphs 42 to 60, wherein the first antiparasitic active agent is Compound A472, or a salt thereof.

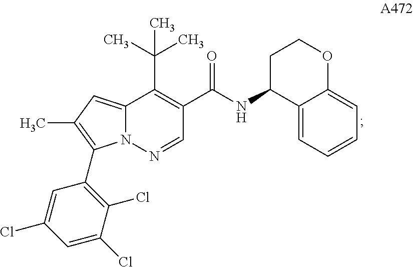 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, sarolaner, moxidectin and praziquantel.
92. The antiparasitic combination of any one of paragraphs 42 to 60, wherein the first antiparasitic active agent is Compound A472, or a salt thereof.

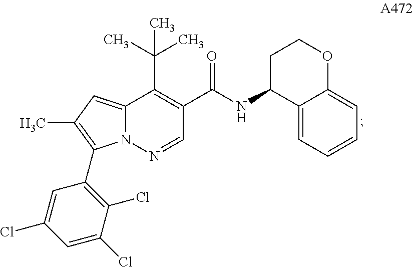 [SMILES: Cc1cc2c(C(C)(C)C)c(C(=O)N[C@H]3CCOc4ccccc43)cnn2c1-c1cc(Cl)cc(Cl)c1Cl]

and wherein the second or more additional antiparasitic active agent(s) comprises, consists essentially of, or consists of, lotilaner, moxidectin and praziquantel.
93. The antiparasitic combination of any one of paragraphs 42 to 58, 61 or 62, wherein the first antiparasitic active agent is Compound IC-1, or a salt thereof:

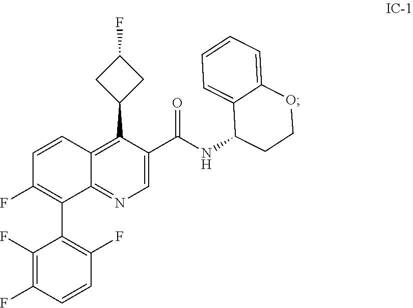 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, a macrocyclic lactone selected from the group consisting of moxidectin, milbemycin oxime, ivermectin, eprinomectin and selamectin, or a combination thereof.
94. The antiparasitic combination of any one of paragraphs 42 to 58, 61 or 62, wherein the first antiparasitic active agent is Compound IC-1, or a salt thereof:

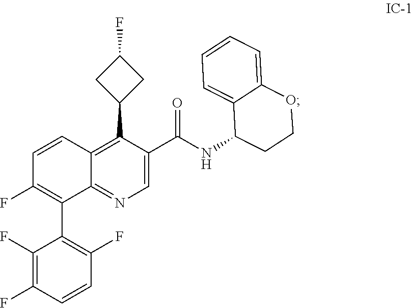 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more antiparasitic active agent(s) comprises, consists essentially of, or consists of, an isoxazoline active agent selected from the group consisting of afoxolaner, esafoxolaner, umifoxolaner, fluralaner, lotilaner and sarolaner, or a combination thereof.
95. The antiparasitic combination of any one of paragraphs 42 to 58, 61 or 62, wherein the first antiparasitic active agent is Compound IC-1, or a salt thereof:

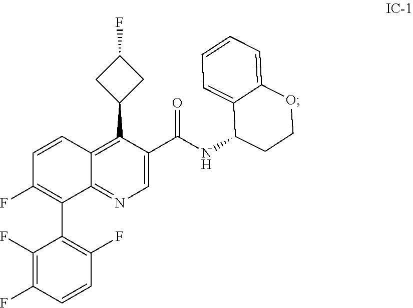 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more antiparasitic active agent(s) comprises, consists essentially of, or consists of, praziquantel.
96. The antiparasitic combination of any one of paragraphs 42 to 58, 61 or 62, wherein the first antiparasitic active agent is Compound IC-1, or a salt thereof:

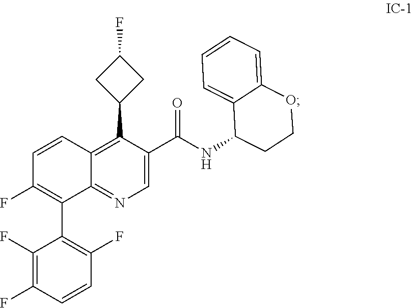 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more antiparasitic active agent(s) comprises, consists essentially of, or consists of, pyrantel.
97. The antiparasitic combination of any one of paragraphs 42 to 58, 61 or 62, wherein the first antiparasitic active agent is Compound IC-1, or a salt thereof:

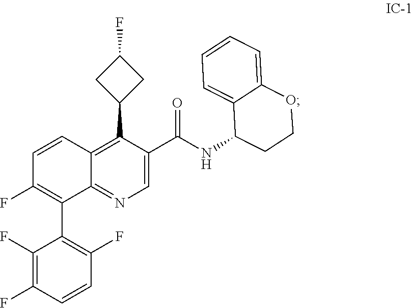 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent comprises, consists essentially of, or consists of, a combination of a macrocyclic lactone selected from the group consisting of moxidectin, milbemycin oxime, ivermectin, eprinomectin and selamectin, or a combination thereof, and an isoxazoline active agent selected from the group consisting of afoxolaner, esafoxolaner, umifoxolaner, fluralaner, lotilaner and sarolaner, or a combination thereof.
98. The antiparasitic combination of any one of paragraphs 42 to 58, 61 or 62, wherein the first antiparasitic active agent is Compound IC-1, or a salt thereof:

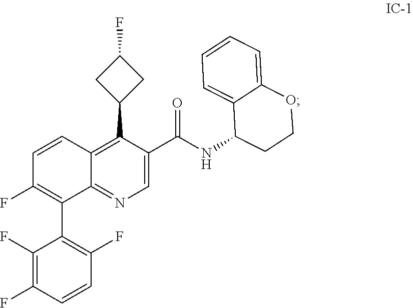 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more antiparasitic active agent(s) comprises, or consists essentially of, consists of, a combination of a macrocyclic lactone selected from the group consisting of moxidectin, milbemycin oxime, ivermectin, eprinomectin and selamectin, or a combination thereof, an isoxazoline active agent selected from the group consisting of afoxolaner, esafoxolaner, umifoxolaner, fluralaner, lotilaner and sarolaner, or a combination thereof; and praziquantel.
99. The antiparasitic combination of any one of paragraphs 42 to 58, 61 or 62, wherein the first antiparasitic active agent is Compound IC-1, or a salt thereof:

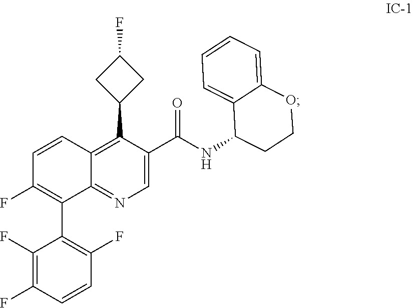 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof, an isoxazoline active agent selected from the group consisting of afoxolaner, esafoxolaner, umifoxolaner, fluralaner, lotilaner and sarolaner, or a combination thereof; and pyrantel.
100. The antiparasitic combination of any one of paragraphs 42 to 58, 61 or 62, wherein the first antiparasitic active agent is Compound IC-1, or a salt thereof:

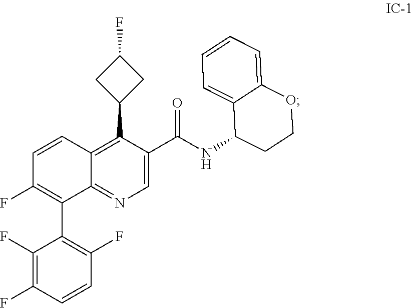 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, afoxolaner.
101. The antiparasitic combination of any one of paragraphs 42 to 58, 61 or 62, wherein the first antiparasitic active agent is Compound IC-1, or a salt thereof:

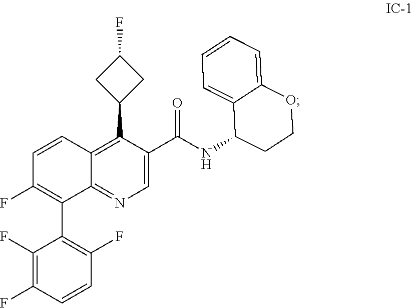 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, esafoxolaner.
102. The antiparasitic combination of any one of paragraphs 42 to 58, 61 or 62, wherein the first antiparasitic active agent is Compound IC-1, or a salt thereof:

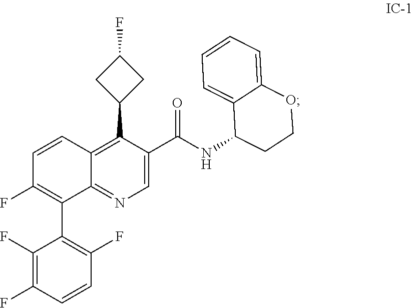 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, umifoxolaner.
103. The antiparasitic combination of any one of paragraphs 42 to 58, 61 or 62, wherein the first antiparasitic active agent is Compound IC-1, or a salt thereof:

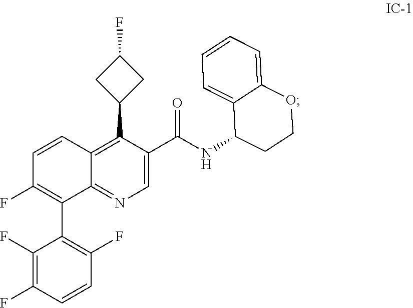 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, fluralaner.
104. The antiparasitic combination of any one of paragraphs 42 to 58, 61 or 62, wherein the first antiparasitic active agent is Compound IC-1, or a salt thereof:

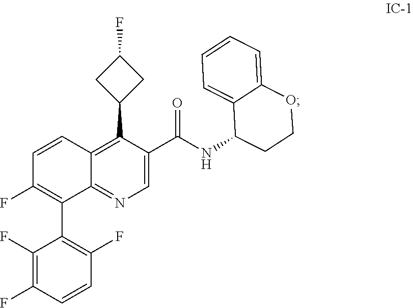 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, sarolaner.
105. The antiparasitic combination of any one of paragraphs 42 to 58, 61 or 62, wherein the first antiparasitic active agent is Compound IC-1, or a salt thereof:

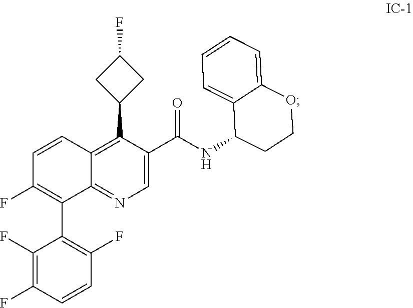 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, lotilaner.
106. The antiparasitic combination of any one of paragraphs 42 to 58, 61 or 62, wherein the first antiparasitic active agent is Compound IC-1, or a salt thereof:

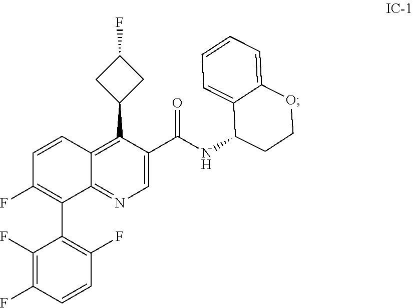 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, moxidectin.
107. The antiparasitic combination of any one of paragraphs 42 to 58, 61 or 62, wherein the first antiparasitic active agent is Compound IC-1, or a salt thereof:

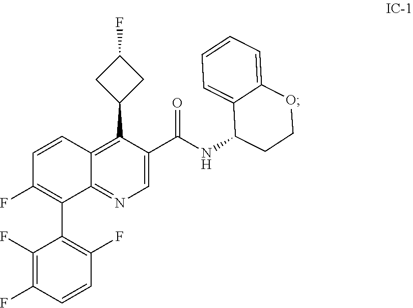 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, milbemycin oxime.
108. The antiparasitic combination of any one of paragraphs 42 to 58, 61 or 62, wherein the first antiparasitic active agent is Compound IC-1, or a salt thereof:

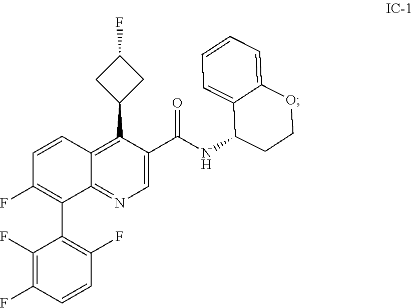 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, eprinomectin.
109. The antiparasitic combination of any one of paragraphs 42 to 58, 61 or 62, wherein the first antiparasitic active agent is Compound IC-1, or a salt thereof:

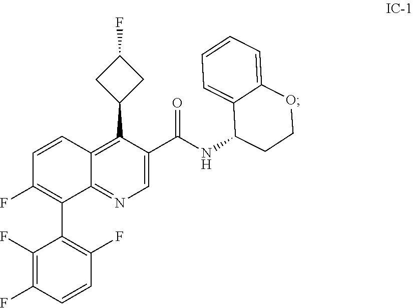 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, ivermectin.
110. The antiparasitic combination of any one of paragraphs 42 to 58, 61 or 62, wherein the first antiparasitic active agent is Compound IC-1, or a salt thereof:

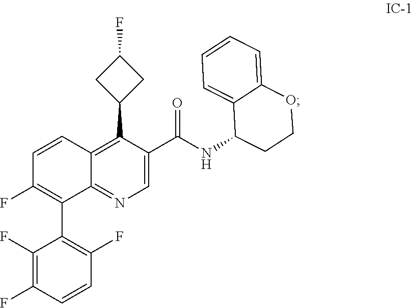 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, selamectin.
111. The antiparasitic combination of any one of paragraphs 42 to 58, 61 or 62, wherein the first antiparasitic active agent is Compound IC-1, or a salt thereof:

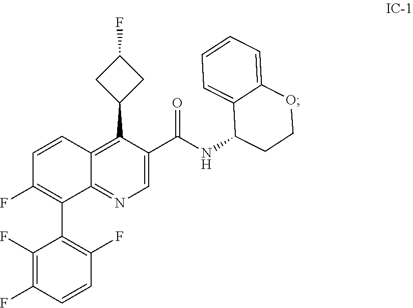 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, afoxolaner and moxidectin.
112. The antiparasitic combination of any one of paragraphs 42 to 58, 61 or 62, wherein the first antiparasitic active agent is Compound IC-1, or a salt thereof:

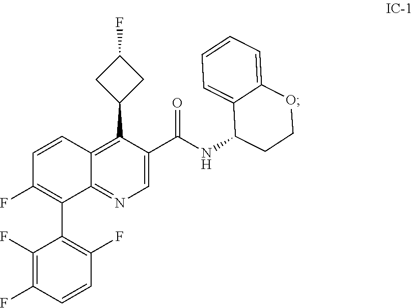 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, esafoxolaner and moxidectin.
113. The antiparasitic combination of any one of paragraphs 42 to 58, 61 or 62, wherein the first antiparasitic active agent is Compound IC-1, or a salt thereof:

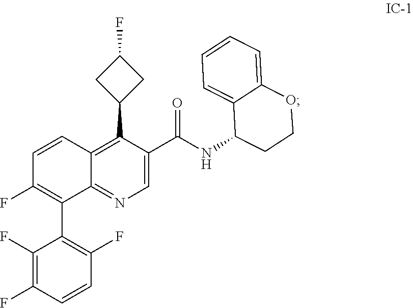 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, umifoxolaner and moxidectin.
114. The antiparasitic combination of any one of paragraphs 42 to 58, 61 or 62, wherein the first antiparasitic active agent is Compound IC-1, or a salt thereof:

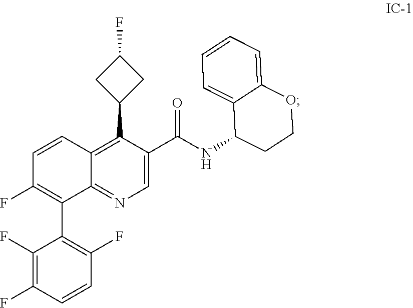 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, fluralaner and moxidectin.
115. The antiparasitic combination of any one of paragraphs 42 to 58, 61 or 62, wherein the first antiparasitic active agent is Compound IC-1, or a salt thereof:

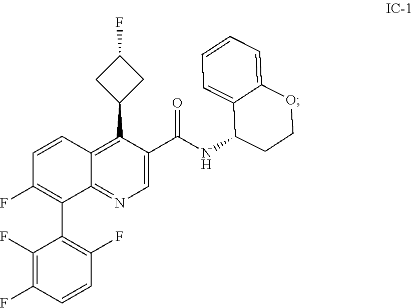 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, sarolaner and moxidectin.
116. The antiparasitic combination of any one of paragraphs 42 to 58, 61 or 62, wherein the first antiparasitic active agent is Compound IC-1, or a salt thereof:

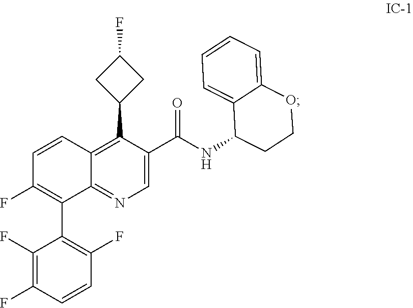 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, lotilaner and moxidectin.
117. The antiparasitic combination of any one of paragraphs 42 to 58, 61 or 62, wherein the first antiparasitic active agent is Compound IC-1, or a salt thereof:

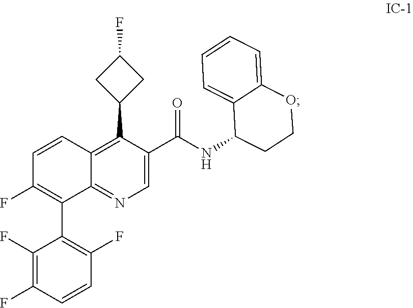 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, afoxolaner, moxidectin and praziquantel.
118. The antiparasitic combination of any one of paragraphs 42 to 58, 61 or 62, wherein the first antiparasitic active agent is Compound IC-1, or a salt thereof:

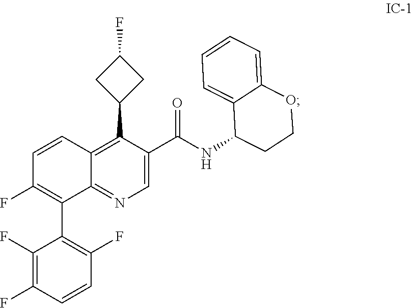 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, esafoxolaner, moxidectin and praziquantel.
119. The antiparasitic combination of any one of paragraphs 42 to 58, 61 or 62, wherein the first antiparasitic active agent is Compound IC-1, or a salt thereof:

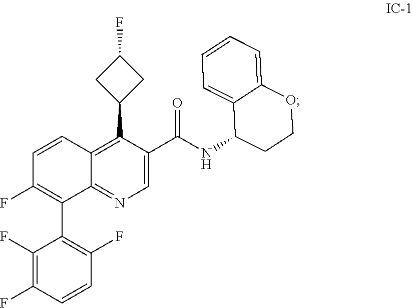 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, umifoxolaner, moxidectin and praziquantel.
120. The antiparasitic combination of any one of paragraphs 42 to 58, 61 or 62, wherein the first antiparasitic active agent is Compound IC-1, or a salt thereof:

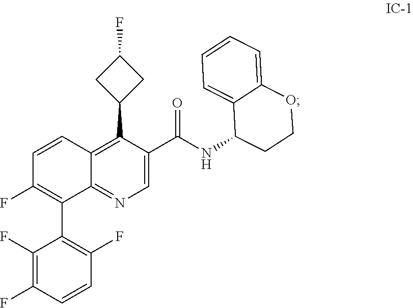 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, fluralaner, moxidectin and praziquantel.
121. The antiparasitic combination of any one of paragraphs 42 to 58, 61 or 62, wherein the first antiparasitic active agent is Compound IC-1, or a salt thereof:

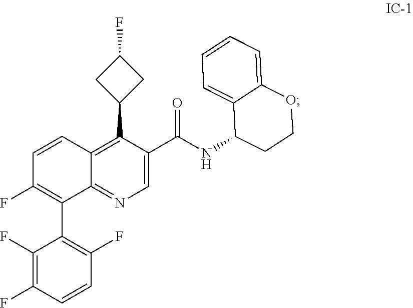 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, sarolaner, moxidectin and praziquantel.
122. The antiparasitic combination of any one of paragraphs 42 to 58, 61 or 62, wherein the first antiparasitic active agent is Compound IC-1, or a salt thereof:

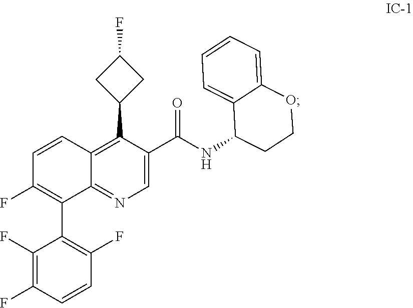 [SMILES: O=C(N[C@H]1CCOc2ccccc21)c1cnc2c(-c3c(F)ccc(F)c3F)c(F)ccc2c1[C@H]1C[C@H](F)C1]

and wherein the second or more additional antiparasitic active agent(s) comprises, consists essentially of, or consists of, lotilaner, moxidectin and praziquantel.
123. A veterinary or pharmaceutical composition comprising the antiparasitic combination of any one of claims 42 to 122 together with a pharmaceutically acceptable carrier.
124. An antiparasitic combination according to any one of claims 42 to 122 for use in treating and/or preventing a parasitic infection and/or infestation in or on an animal.
125. A veterinary or pharmaceutical composition according to claim 123, for use in treating and/or preventing a parasitic infection and/or infestation in or on an animal.
126. A method of treating and/or preventing a parasitic infection and/or infestation in or on an animal, comprising administering an antiparasitic combination according to any one of claims 42 to 122, or the veterinary or pharmaceutical composition according to claim 123, to an animal in need thereof.
127. The antiparasitic combination for use according to claim 124, or the veterinary or pharmaceutical composition for use according to claim 125, or the method according to claim 126, wherein the animal is a human, cat, dog, horse, cow, sheep or fish.
128. The antiparasitic combination for use according to claim 124, or the veterinary or pharmaceutical composition for use according to claim 125, or the method according to claim 126, wherein the parasitic infection and/or infestation is an ectoparasitic and/or endoparasitic infection and/or infestation.
129. The antiparasitic combination for use according to any one of claims 124, 127 or 128, or the veterinary or pharmaceutical composition for use according to any one of claims 125, 127 or 128, or the method according to any one of claims 84, 85 or 86, wherein the parasitic infection and/or infestation is caused by one or more of Dirofilaria immitis, hookworms, roundworms, cestodes, ticks and fleas, or a combination thereof.

Having thus described in detail preferred embodiments of the present invention, it is to be understood that the invention defined by the above paragraphs is not to be limited to particular details set forth in the above description as many apparent variations thereof are

What is claimed is:

1. An antiparasitic combination of two or more antiparasitic active agents, wherein a first antiparasitic active agent in the combination is an anthelmintic compound of Formula (IA), Formula (IB) or Formula (IC), or a combination thereof: 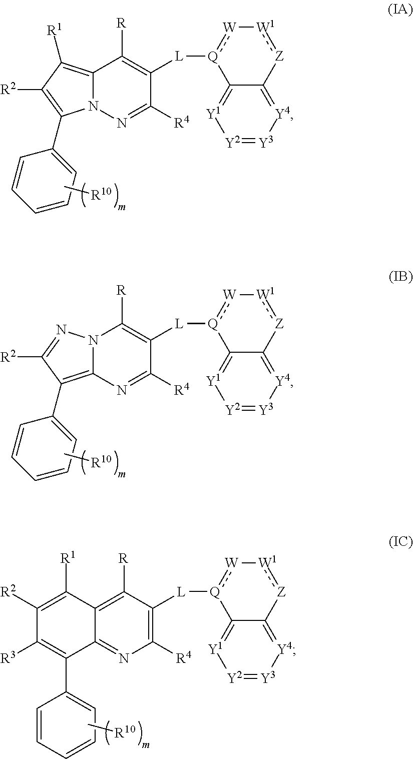
or salts thereof,
wherein:
L is L1 or L2: 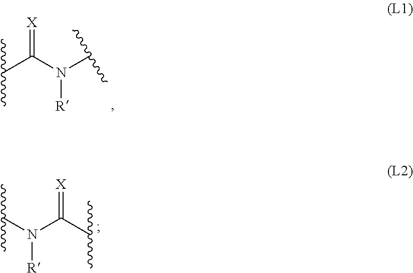
R′ is hydrogen, optionally substituted alkyl or optionally substituted haloalkyl;
R is optionally substituted alkyl, optionally substituted cycloalkyl, optionally substituted cycloalkenyl, optionally substituted alkenyl, optionally substituted alkynyl, optionally substituted heterocyclyl or —NR^{a}R^{b}, wherein R^{a} and R^{b} are independently H or optionally substituted alkyl;
R¹, R², R³ and R⁴ are independently hydrogen, cyano, halogen, optionally substituted alkyl, optionally substituted alkenyl, optionally substituted alkynyl, optionally substituted alkoxy;
R⁵ is independently each occurrence, hydrogen, halogen, cyano, optionally substituted alkyl, optionally substituted alkoxy, optionally substituted alkylcarbonyl, optionally substituted alkoxycarbonyl, optionally substituted aminocarbonyl, optionally substituted alkylaminocarbonyl, optionally substituted dialkylaminocarbonyl, optionally substituted alkylcarbonyloxy, optionally substituted alkylcarbonylamino, —SOₚ(optionally substituted alkyl), —SOₚ(optionally substituted haloalkyl);
R⁶ and R⁷ are independently hydrogen, halogen, optionally substituted alkyl, optionally substituted alkenyl, optionally substituted alkynyl, optionally substituted alkoxy, or R⁶ together with R⁷ for a carbonyl group (C═O);
R⁸ is hydrogen or optionally substituted alkyl;
R⁹ is hydrogen, halogen or optionally substituted alkyl;
R¹⁰ is cyano, halogen, optionally substituted alkyl or optionally substituted alkoxy;
Q is C, C—R⁹ or N;
X is O, S or N—R′;
Y¹, Y², Y³ and Y⁴ are each independently N or —CR⁵—;
W is CR⁶, CR⁶R⁷, O, SOₚ, N or N—R⁸, or W is absent;
W¹ is CR⁶, CR⁶R⁷, O, SOₚ, N or N—R⁸;
Z is CR⁶, CR⁶R⁷, O, SOₚ, N or N—R⁸, and wherein at most three of Y¹, Y², Y³ and Y⁴ are N;
m is 0, 1, 2, 3, 4 or 5;
p is independently in each occurrence is 0, 1, or 2;
the dashed bonds ( [symbol] )signifies a single or double bond; and
wherein “optionally substituted” indicates that the group recited may be optionally substituted with halogen (independently chloro, fluoro, bromo or iodo), hydroxyl, thiol, oxo (C═O), C₁-C₆-alkyl, C₁-C₆-haloalkyl, C₃-C₈-cycloalkyl, C₁-C₆-alkoxy, C₁-C₆-haloalkoxy, —S(O)ₚ(C₁-C₆-alkyl) or —S(O)ₚ(C₁-C₆-haloalkyl) where p is 0, 1 or 2, carboxyl, acetyl, C₁-C₆-alkylcarbonyl, C₁-C₆-haloalkylcarbonyl, C₁-C₆-alkoxycarbonyl, C₁-C₆-haloalkoxycarbonyl, aminocarbonyl, C₁-C₆-alkylaminocarbonyl, di-C₁-C₆-alkylaminocarbonyl, C₁-C₆-haloalkylaminocarbonyl, di-C₁-C₆-haloalkylaminocarbonyl, amino, C₁-C₆-alkylamino, di-C₁-C₆-alkylamino, nitro, cyano, R₃Si— or R₃SiO— where R is C₁-C₆-alkyl, C₁-C₆-haloalkyl or phenyl, SF₅, phenyl, naphthyl, 5- to 10-membered heteroaryl containing from one to three N, O, Si or S heteroatoms; and 3- to 10-membered heterocyclyl containing from one to three N, O, Si or S heteroatoms; and
the second or more additional antiparasitic active agent(s) in the combination is a macrocyclic lactone, an isoxazoline, praziquantel or pyrantel, or a combination thereof.

2. The antiparasitic combination of claim 1, wherein R is optionally substituted alkyl, optionally substituted haloalkyl, optionally substituted cycloalkyl or optionally substituted saturated or unsaturated heterocyclyl.

3. The antiparasitic combination of claim 1 or 2, wherein R¹, R², R³ and R⁴ are each independently hydrogen, halogen, optionally substituted alkyl or optionally substituted haloalkyl.

4. The antiparasitic combination of any one of claims 1 to 3, wherein R¹⁰ is halogen or optionally substituted alkyl.

5. The antiparasitic combination of any one of claims 1 to 4, wherein the second or more additional antiparasitic active agent(s) comprises, consists essentially of, or consists of, a macrocyclic lactone selected from the group consisting of moxidectin, milbemycin oxime, ivermectin, eprinomectin and selamectin, or a combination thereof.

6. The antiparasitic combination of any one of claims 1 to 4, wherein the second or more additional antiparasitic active agent(s) comprises, consists essentially of, or consists of, an isoxazoline active agent selected from the group consisting of afoxolaner, esafoxolaner, umifoxolaner, fluralaner, lotilaner and sarolaner.

7. The antiparasitic combination of any one of claims 1 to 4, wherein the second or more additional antiparasitic active agent(s) comprises, consists essentially of, or consists of, praziquantel.

8. The antiparasitic combination of any one of claims 1 to 4, wherein the second or more additional antiparasitic active agent(s), consists essentially of, or consists of, pyrantel.

9. The antiparasitic combination of any one of claims 1 to 4, wherein the second or more additional antiparasitic active agent(s) comprises, consists essentially of, or consists of, a combination of a macrocyclic lactone and an isoxazoline active agent.

10. The antiparasitic combination of any one of claims 1 to 4, wherein the second or more additional antiparastic active agent(s) comprises, consists essentially of, or consists of, a combination of a macrocyclic lactone, an isoxazoline active agent and praziquantel.

11. The antiparasitic combination of any one of claims 1 to 4, wherein the second or more additional antiparastic active agent(s) comprises, or consists essentially of, consists of, a combination of a macrocyclic lactone, an isoxazoline active agent and pyrantel.

12. The antiparasitic combination of any one of claims 9 to 11, wherein the macrocyclic lactone is selected from the group consisting of moxidectin, milbemycin oxime, ivermectin, eprinomectin and selamectin, or a combination thereof, and/or
wherein the isoxazoline active agent is selected from the group consisting of afoxolaner, esafoxolaner, umifoxolaner, fluralaner, lotilaner and sarolaner, or a combination thereof.

13. The antiparasitic combination of any one of claims 1 to 4, wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; and an isoxazoline active agent selected from the group consisting of afoxolaner, esafoxolaner, umifoxolaner, fluralaner, lotilaner and sarolaner, or a combination thereof.

14. The antiparasitic combination of any one of claims 1 to 4, wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof; and praziquantel.

15. The antiparasitic combination of any one of claims 1 to 4, wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof, an isoxazoline active agent selected from the group consisting of afoxolaner, esafoxolaner, umifoxolaner, fluralaner, lotilaner and sarolaner, or a combination thereof, and pyrantel.

16. The antiparasitic combination of any one of claims 1 to 4, wherein the second or more additional antiparasitic active agent(s) comprises, consists essentially of, or consists of, a combination of moxidectin or milbemycin oxime; afoxolaner, esafoxolaner, fluralaner or lotilaner; and praziquantel.

17. The antiparasitic combination of any one of claims 1 to 4, wherein the second or more additional antiparasitic active agent(s) comprises, consists essentially of, or consists of, a combination of moxidectin, afoxolaner and praziquantel.

18. The antiparasitic combination of any one of claims 1 to 17, wherein the anthelmintic compound is the compound of Formula (IA), wherein:
L is L1, wherein X is O and R′ is hydrogen;
Q is C—R⁹;
R is iso-propyl, tert-butyl, cyclopropyl, cyclobutyl, —N(CH₃)₂, azetidinyl or morpholinyl, each optionally substituted by one or more fluoro or methyl;
R¹, R⁴ and R⁹ are each hydrogen;
R² is hydrogen, methyl, —CF₃, chloro or fluoro;
R¹⁰ is chloro or fluoro and m is 2 or 3;
W and W¹ are each CH₂;
Z is O; and
Y¹, Y², Y³ and Y⁴ are each CH.

19. The antiparasitic combination of any one of claims 1 to 18, wherein the anthelmintic compound is Compound A472, or a salt thereof:

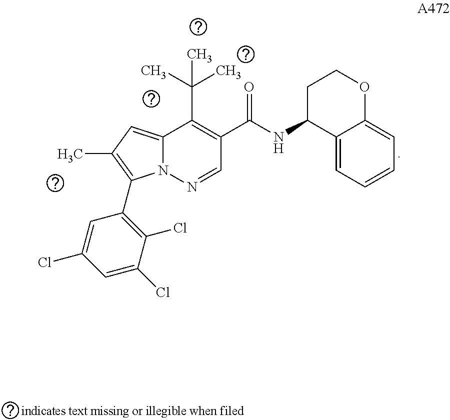 [SMILES: Cc1cc2c(C(C)(C)C)c(C(=O)N[C@H]3CCOc4ccccc43)cnn2c1-c1cc(Cl)cc(Cl)c1Cl]

20. The antiparasitic combination of any one of claims 1 to 17, wherein the anthelmintic compound is the compound of Formula (IC), wherein:
L is L1 wherein X is O and R′ is hydrogen;
Q is C—R⁹; R is ethyl, iso-propyl, cyclobutyl, —N(CH₃)₂, azetidinyl, morpholinyl, oxetanyl or tetrahydropyranyl, each optionally substituted by one or more fluoro or methyl;
R¹, R², R⁴ and R⁹ are each hydrogen;
R³ is hydrogen, methyl, —CF₃, chloro or fluoro;
R¹⁰ is CF₃, chloro or fluoro and m is 2 or 3;
W and W¹ are each CH₂;
Z is O; and
Y¹, Y², Y³ and Y⁴ are each CH.

21. The antiparasitic combination of any one of claims 1 to 17 or 20, wherein the anthelmintic compound is Compound IC-1, or a salt thereof:

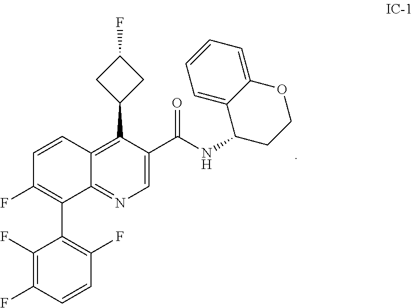 [SMILES: O=C(N[C@H]1CCOc2ccccc21)c1cnc2c(-c3c(F)ccc(F)c3F)c(F)ccc2c1[C@H]1C[C@H](F)C1]

22. The antiparasitic combination of any one of claims 1 to 19, wherein the first antiparasitic active agent is Compound A472, or a salt thereof: 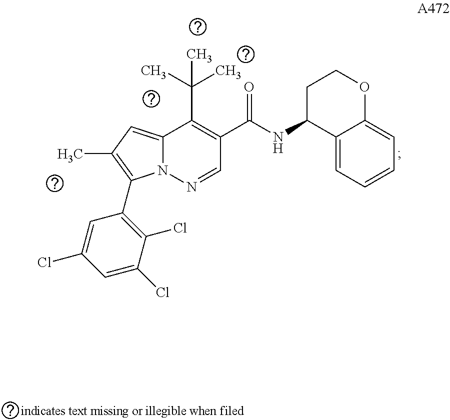 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, a macrocyclic lactone selected from the group consisting of moxidectin, milbemycin oxime, ivermectin, eprinomectin and selamectin, or a combination thereof.

23. The antiparasitic combination of any one of claims 1 to 19, wherein the first antiparasitic active agent is Compound A472, or a salt thereof: 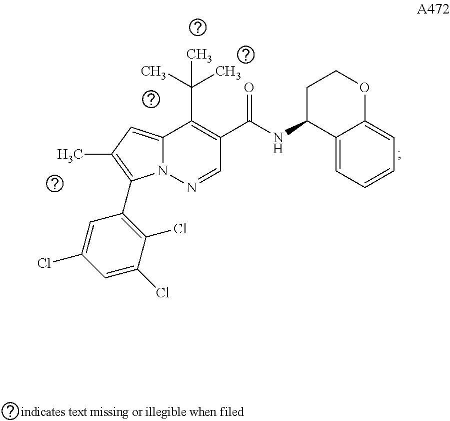 [SMILES: Cc1cc2c(C(C)(C)C)c(C(=O)N[C@H]3CCOc4ccccc43)cnn2c1-c1cc(Cl)cc(Cl)c1Cl]
and wherein the second or more antiparasitic active agent(s) comprises, consists essentially of, or consists of, an isoxazoline active agent selected from the group consisting of afoxolaner, esafoxolaner, umifoxolaner, fluralaner, lotilaner and sarolaner, or a combination thereof.

24. The antiparasitic combination of any one of claims 1 to 19, wherein the first antiparasitic active agent is Compound A472, or a salt thereof: 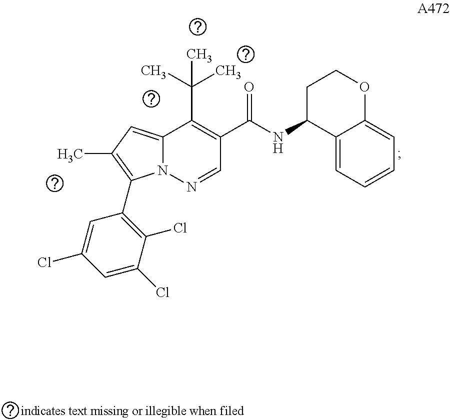 [SMILES: Cc1cc2c(C(C)(C)C)c(C(=O)N[C@H]3CCOc4ccccc43)cnn2c1-c1cc(Cl)cc(Cl)c1Cl]
and wherein the second or more antiparasitic active agent(s) comprises, consists essentially of, or consists of, praziquantel.

25. The antiparasitic combination of any one of claims 1 to 19, wherein the first antiparasitic active agent is Compound A472, or a salt thereof: 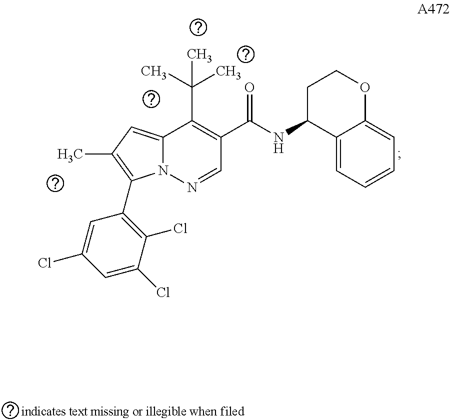 [SMILES: Cc1cc2c(C(C)(C)C)c(C(=O)N[C@H]3CCOc4ccccc43)cnn2c1-c1cc(Cl)cc(Cl)c1Cl]
and wherein the second or more antiparasitic active agent(s) comprises, consists essentially of, or consists of, pyrantel.

26. The antiparasitic combination of any one of claims 1 to 19, wherein the first antiparasitic active agent is Compound A472, or a salt thereof: 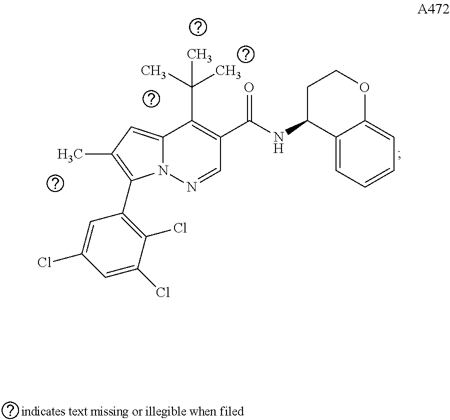 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof, and an isoxazoline active agent selected from the group consisting of afoxolaner, esafoxolaner, umifoxolaner, fluralaner, lotilaner and sarolaner, or a combination thereof.

26. The antiparasitic combination of any one of claims 1 to 19, wherein the first antiparasitic active agent is Compound A472, or a salt thereof: 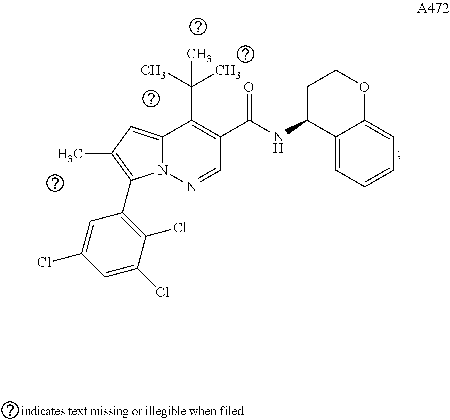 [SMILES: Cc1cc2c(C(C)(C)C)c(C(=O)N[C@H]3CCOc4ccccc43)cnn2c1-c1cc(Cl)cc(Cl)c1Cl]
and wherein the second or more antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof; and praziquantel.

27. The antiparasitic combination of any one of claims 1 to 19, wherein the first antiparasitic active agent is Compound A472, or a salt thereof: 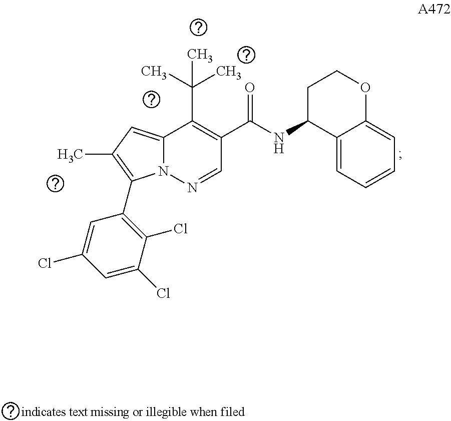 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof; and pyrantel.

28. The antiparasitic combination of any one of claims 1 to 19, wherein the first antiparasitic active agent is Compound A472, or a salt thereof: 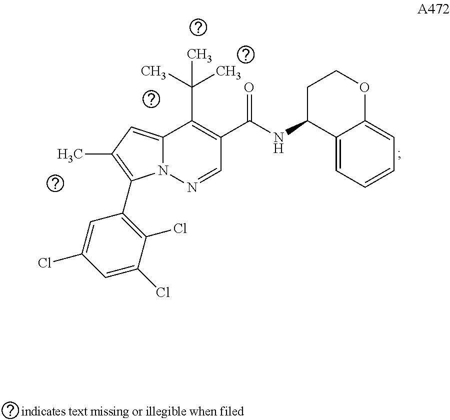 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, afoxolaner.

29. The antiparasitic combination of any one of claims 1 to 19, wherein the first antiparasitic active agent is Compound A472, or a salt thereof: 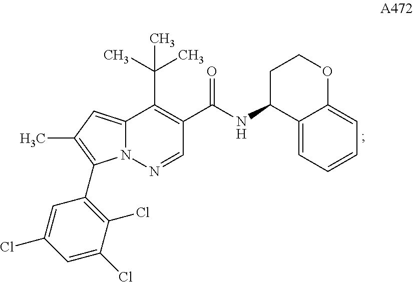 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, esafoxolaner.

30. The antiparasitic combination of any one of claims 1 to 19, wherein the first antiparasitic active agent is Compound A472, or a salt thereof: 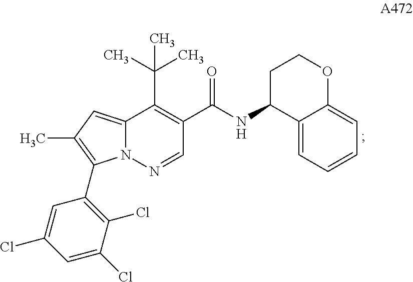 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, umifoxolaner.

31. The antiparasitic combination of any one of claims 1 to 19, wherein the first antiparasitic active agent is Compound A472, or a salt thereof: 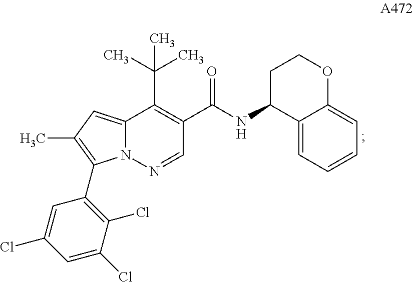 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, fluralaner.

32. The antiparasitic combination of any one of claims 1 to 19, wherein the first antiparasitic active agent is Compound A472, or a salt thereof: 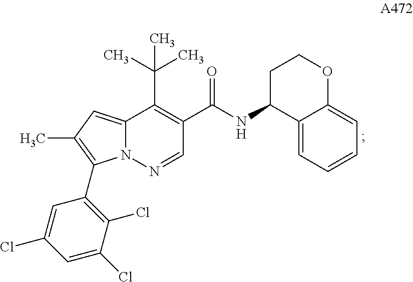 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, sarolaner.

33. The antiparasitic combination of any one of claims 1 to 19, wherein the first antiparasitic active agent is Compound A472, or a salt thereof: 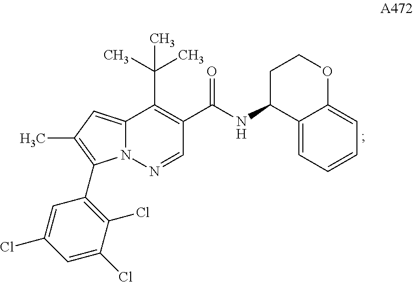 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, lotilaner.

34. The antiparasitic combination of any one of claims 1 to 19, wherein the first antiparasitic active agent is Compound A472, or a salt thereof: 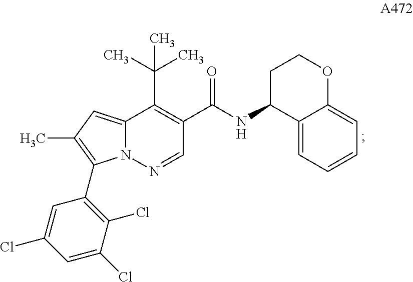 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, moxidectin.

35. The antiparasitic combination of any one of claims 1 to 19, wherein the first antiparasitic active agent is Compound A472, or a salt thereof: 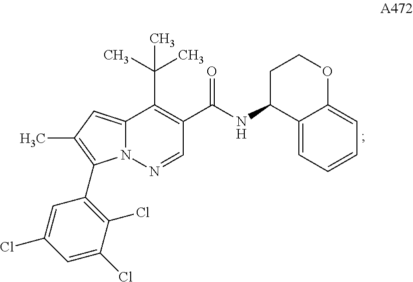 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, milbemycin oxime.

36. The antiparasitic combination of any one of claims 1 to 19, wherein the first antiparasitic active agent is Compound A472, or a salt thereof: 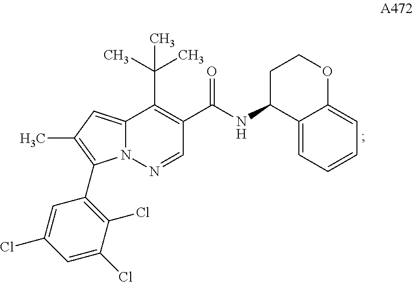 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, eprinomectin.

37. The antiparasitic combination of any one of claims 1 to 19, wherein the first antiparasitic active agent is Compound A472, or a salt thereof: 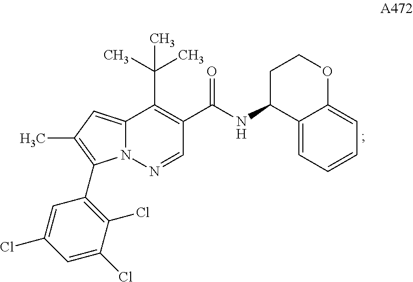 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, ivermectin.

38. The antiparasitic combination of any one of claims 1 to 19, wherein the first antiparasitic active agent is Compound A472, or a salt thereof: 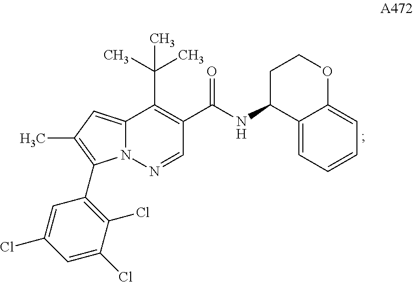 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, selamectin.

39. The antiparasitic combination of any one of claims 1 to 19, wherein the first antiparasitic active agent is Compound A472, or a salt thereof. 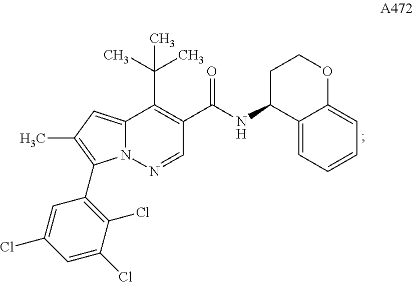 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, afoxolaner and moxidectin.

40. The antiparasitic combination of any one of claims 1 to 19, wherein the first antiparasitic active agent is Compound A472, or a salt thereof. 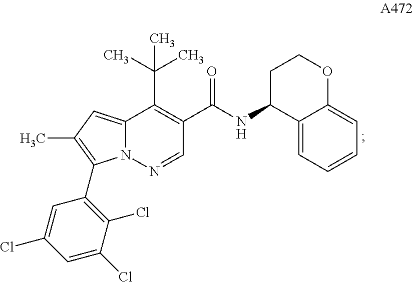 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, esafoxolaner and moxidectin.

41. The antiparasitic combination of any one of claims 1 to 19, wherein the first antiparasitic active agent is Compound A472, or a salt thereof: 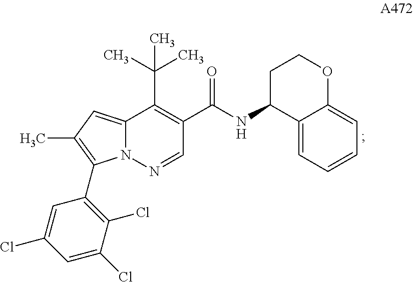 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, umifoxolaner and moxidectin.

42. The antiparasitic combination of any one of claims 1 to 19, wherein the first antiparasitic active agent is Compound A472, or a salt thereof: 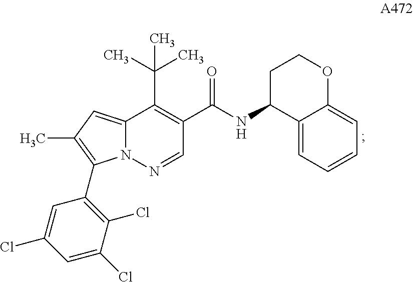 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, fluralaner and moxidectin.

43. The antiparasitic combination of any one of claims 1 to 19, wherein the first antiparasitic active agent is Compound A472, or a salt thereof: 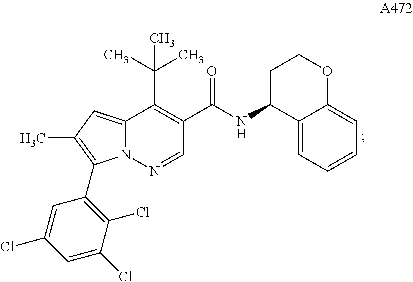 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, sarolaner and moxidectin.

44. The antiparasitic combination of any one of claims 1 to 19, wherein the first antiparasitic active agent is Compound A472, or a salt thereof: 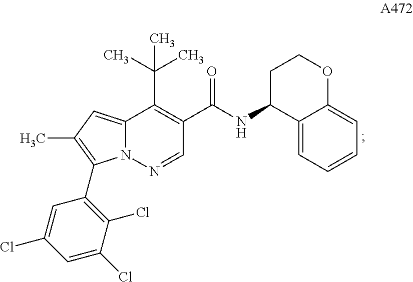 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, lotilaner and moxidectin.

45. The antiparasitic combination of any one of claims 1 to 19, wherein the first antiparasitic active agent is Compound A472, or a salt thereof: 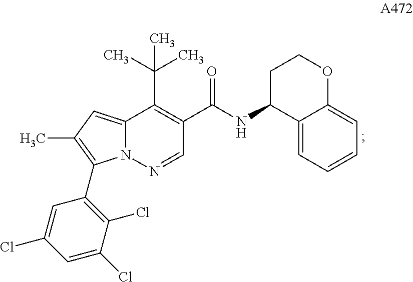 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, afoxolaner, moxidectin and praziquantel.

46. The antiparasitic combination of any one of claims 1 to 19, wherein the first antiparasitic active agent is Compound A472, or a salt thereof: 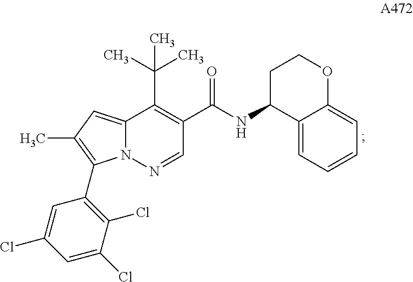 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, esafoxolaner, moxidectin and praziquantel.

47. The antiparasitic combination of any one of claims 1 to 19, wherein the first antiparasitic active agent is Compound A472, or a salt thereof: 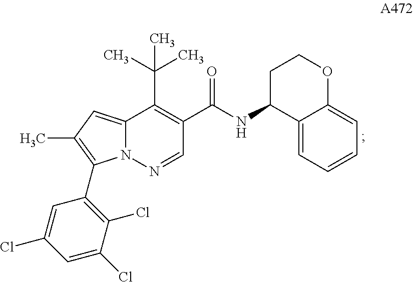 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, umifoxolaner, moxidectin and praziquantel.

48. The antiparasitic combination of any one of claims i to 19, wherein the first antiparasitic active agent is Compound A472, or a salt thereof: 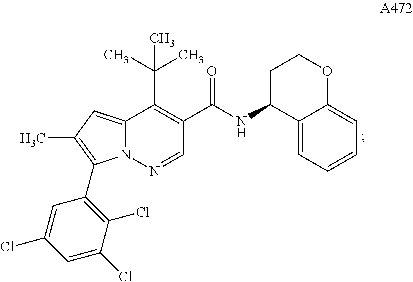 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, fluralaner, moxidectin and praziquantel.

49. The antiparasitic combination of any one of claims 1 to 19, wherein the first antiparasitic active agent is Compound A472, or a salt thereof: 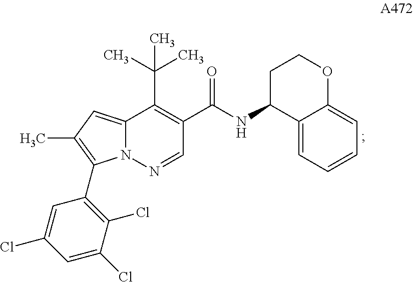 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, sarolaner, moxidectin and praziquantel.

50. The antiparasitic combination of any one of claims 1 to 19, wherein the first antiparasitic active agent is Compound A472, or a salt thereof: 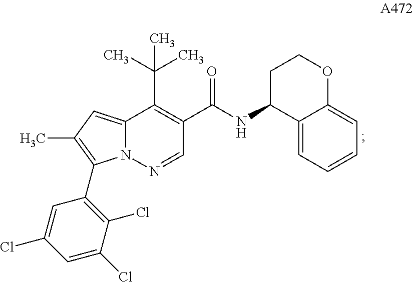 [SMILES: Cc1cc2c(C(C)(C)C)c(C(=O)N[C@H]3CCOc4ccccc43)cnn2c1-c1cc(Cl)cc(Cl)c1Cl]
and wherein the second or more additional antiparasitic active agent(s) comprises, consists essentially of, or consists of, lotilaner, moxidectin and praziquantel.

51. The antiparasitic combination of any one of claims 1 to 17, 20 or 21, wherein the first antiparasitic active agent is Compound IC-1, or a salt thereof: 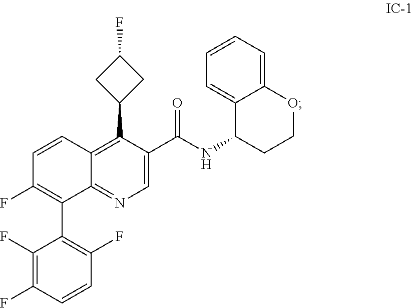 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, a macrocyclic lactone selected from the group consisting of moxidectin, milbemycin oxime, ivermectin, eprinomectin and selamectin, or a combination thereof.

52. The antiparasitic combination of any one of claims 1 to 17, 20 or 21, wherein the first antiparasitic active agent is Compound IC-1, or a salt thereof: 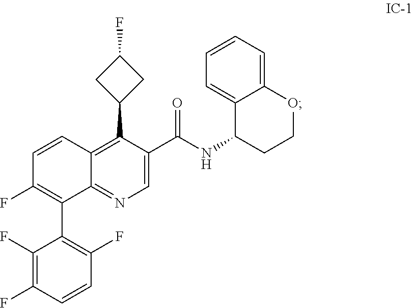 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more antiparasitic active agent(s) comprises, consists essentially of, or consists of, an isoxazoline active agent selected from the group consisting of afoxolaner, esafoxolaner, umifoxolaner, fluralaner, lotilaner and sarolaner, or a combination thereof.

53. The antiparasitic combination of any one of claims 1 to 17, 20 or 21, wherein the first antiparasitic active agent is Compound IC-1, or a salt thereof: 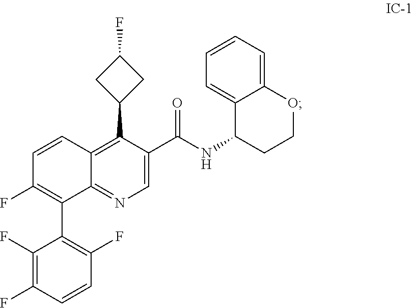 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more antiparasitic active agent(s) comprises, consists essentially of, or consists of, praziquantel.

54. The antiparasitic combination of any one of claims 1 to 17, 20 or 21, wherein the first antiparasitic active agent is Compound IC-1, or a salt thereof: 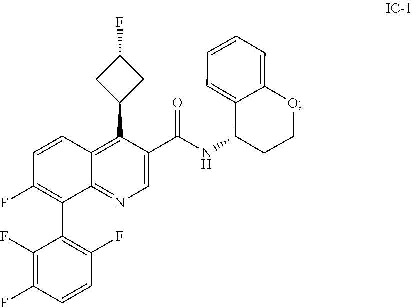 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more antiparasitic active agent(s) comprises, consists essentially of, or consists of, pyrantel.

55. The antiparasitic combination of any one of claims 1 to 17, 20 or 21, wherein the first antiparasitic active agent is Compound IC-1, or a salt thereof: 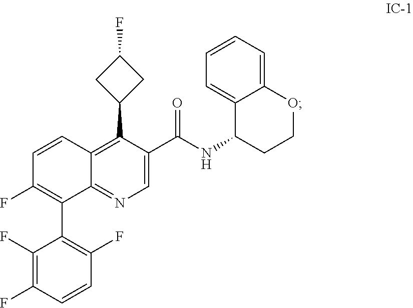 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent comprises, consists essentially of, or consists of, a combination of a macrocyclic lactone selected from the group consisting of moxidectin, milbemycin oxime, ivermectin, eprinomectin and selamectin, or a combination thereof, and an isoxazoline active agent selected from the group consisting of afoxolaner, esafoxolaner, umifoxolaner, fluralaner, lotilaner and sarolaner, or a combination thereof.

56. The antiparasitic combination of any one of claims 1 to 17, 20 or 21, wherein the first antiparasitic active agent is Compound IC-1, or a salt thereof: 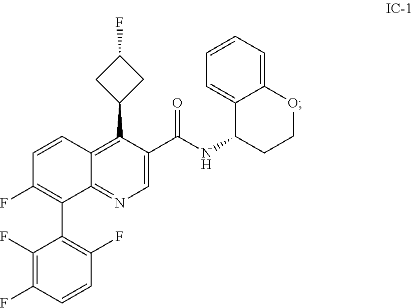 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more antiparasitic active agent(s) comprises, or consists essentially of, consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof, and praziquantel.

57. The antiparasitic combination of any one of claims 1 to 17, 20 or 21, wherein the first antiparasitic active agent is Compound IC-1, or a salt thereof: 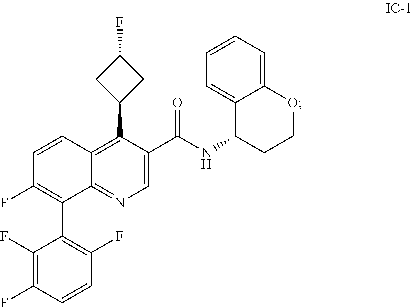 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more antiparasitic active agent(s) comprises, consists essentially of, or consists of, a combination of a macrocyclic lactone selected from the group consisting of moxidectin, milbemycin oxime, ivermectin, eprinomectin and selamectin, or a combination thereof; an isoxazoline active agent selected from the group consisting of afoxolaner, esafoxolaner, umifoxolaner, fluralaner, lotilaner and sarolaner, or a combination thereof, and pyrantel.

58. The antiparasitic combination of any one of claims 1 to 17, 20 or 21, wherein the first antiparasitic active agent is Compound IC-1, or a salt thereof: 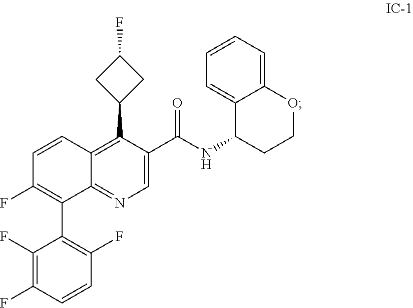 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, afoxolaner.

59. The antiparasitic combination of any one of claims 1 to 17, 20 or 21, wherein the first antiparasitic active agent is Compound IC-1, or a salt thereof: 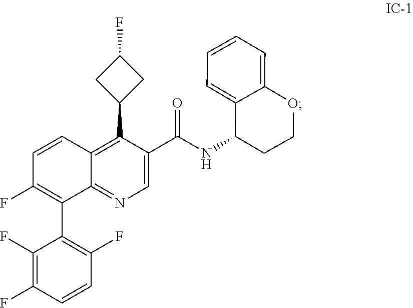 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, esafoxolaner.

60. The antiparasitic combination of any one of claims 1 to 17, 20 or 21, wherein the first antiparasitic active agent is Compound IC-1, or a salt thereof: 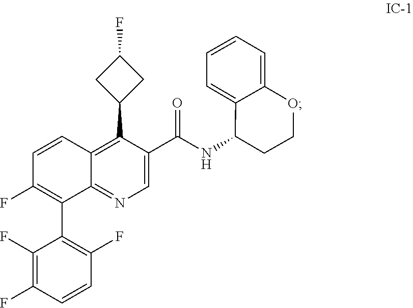 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, umifoxolaner.

61. The antiparasitic combination of any one of claims 1 to 17, 20 or 21, wherein the first antiparasitic active agent is Compound IC-1, or a salt thereof: 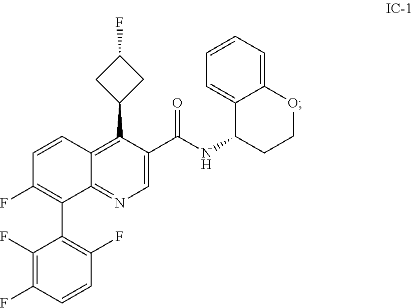 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, fluralaner.

62. The antiparasitic combination of any one of claims 1 to 17, 20 or 21, wherein the first antiparasitic active agent is Compound IC-1, or a salt thereof: 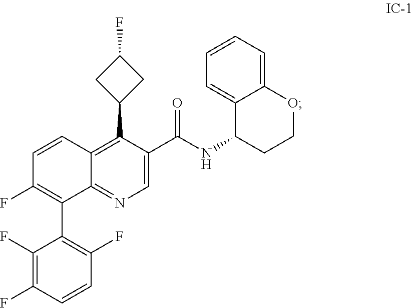 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, sarolaner.

63. The antiparasitic combination of any one of claims 1 to 17, 20 or 21, wherein the first antiparasitic active agent is Compound IC-1, or a salt thereof: 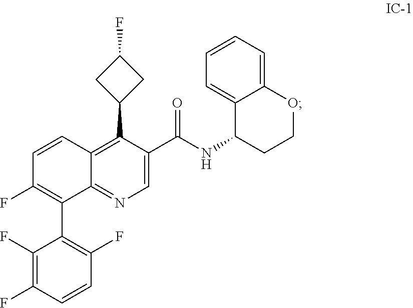 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, lotilaner.

64. The antiparasitic combination of any one of claims 1 to 17, 20 or 21, wherein the first antiparasitic active agent is Compound IC-1, or a salt thereof: 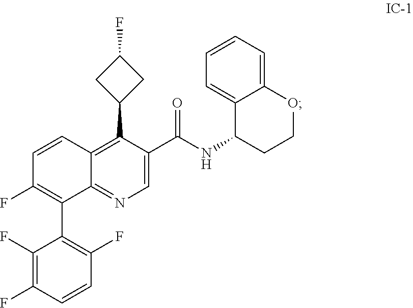 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, moxidectin.

65. The antiparasitic combination of any one of claims 1 to 17, 20 or 21, wherein the first antiparasitic active agent is Compound IC-1, or a salt thereof: 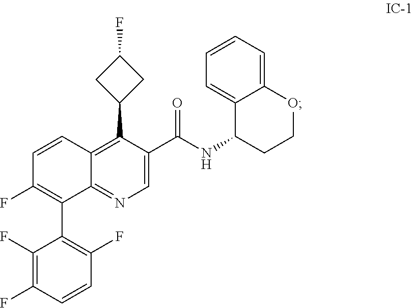 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, milbemycin oxime.

66. The antiparasitic combination of any one of claims 1 to 17, 20 or 21, wherein the first antiparasitic active agent is Compound IC-1, or a salt thereof: 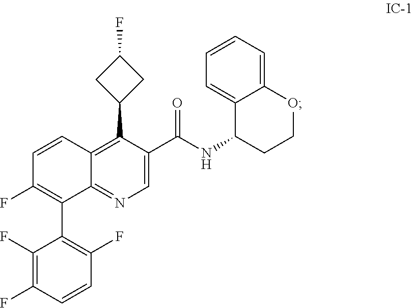 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, eprinomectin.

67. The antiparasitic combination of any one of claims 1 to 17, 20 or 21, wherein the first antiparasitic active agent is Compound IC-1, or a salt thereof: 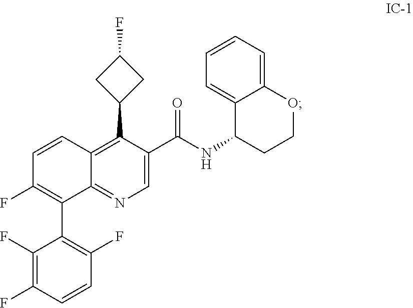 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, ivermectin.

68. The antiparasitic combination of any one of claims 1 to 17, 20 or 21, wherein the first antiparasitic active agent is Compound IC-1, or a salt thereof: 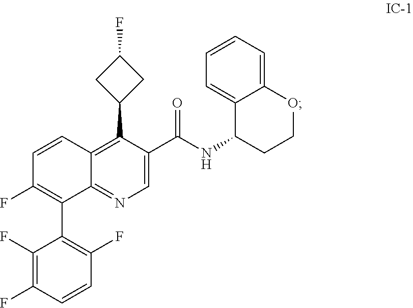 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, selamectin.

69. The antiparasitic combination of any one of claims 1 to 17, 20 or 21, wherein the first antiparasitic active agent is Compound IC-1, or a salt thereof: 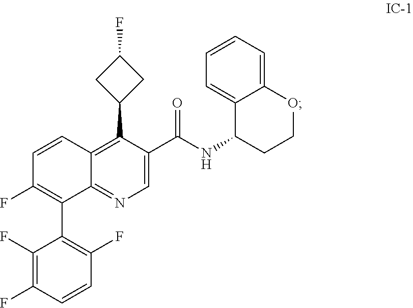 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, afoxolaner and moxidectin.

70. The antiparasitic combination of any one of claims 1 to 17, 20 or 21, wherein the first antiparasitic active agent is Compound IC-1, or a salt thereof: 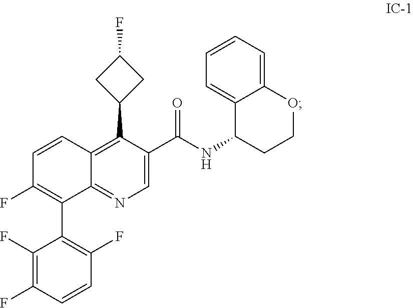 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, esafoxolaner and moxidectin.

71. The antiparasitic combination of any one of claims 1 to 17, 20 or 21, wherein the first antiparasitic active agent is Compound IC-1, or a salt thereof: 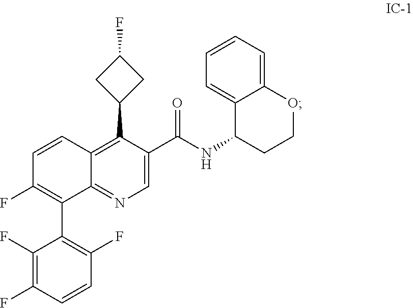 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, umifoxolaner and moxidectin.

72. The antiparasitic combination of any one of claims 1 to 17, 20 or 21, wherein the first antiparasitic active agent is Compound IC-1, or a salt thereof: 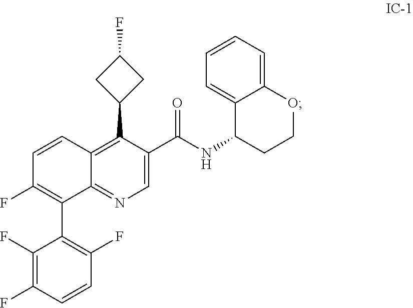 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, fluralaner and moxidectin.

73. The antiparasitic combination of any one of claims 1 to 17, 20 or 21, wherein the first antiparasitic active agent is Compound IC-1, or a salt thereof: 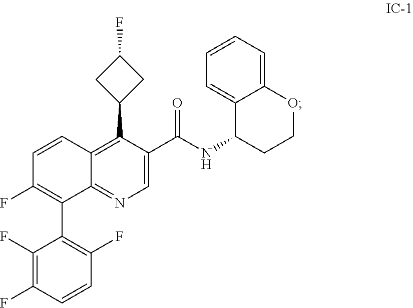 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, sarolaner and moxidectin.

74. The antiparasitic combination of any one of claims 1 to 17, 20 or 21, wherein the first antiparasitic active agent is Compound IC-1, or a salt thereof: 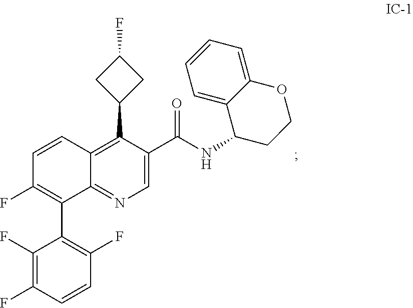 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, lotilaner and moxidectin.

75. The antiparasitic combination of any one of claims 1 to 17, 20 or 21, wherein the first antiparasitic active agent is Compound IC-1, or a salt thereof: 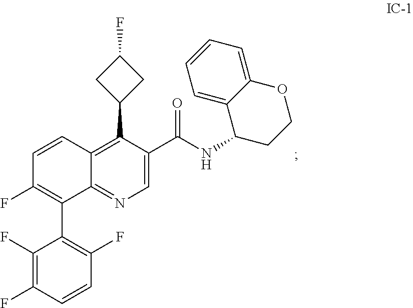 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, afoxolaner, moxidectin and praziquantel.

76. The antiparasitic combination of any one of claims 1 to 17, 20 or 21, wherein the first antiparasitic active agent is Compound IC-1, or a salt thereof: 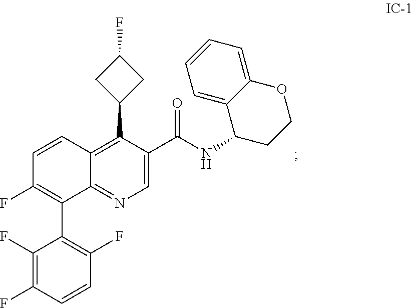 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, esafoxolaner, moxidectin and praziquantel.

77. The antiparasitic combination of any one of claims 1 to 17, 20 or 21, wherein the first antiparasitic active agent is Compound IC-1, or a salt thereof: 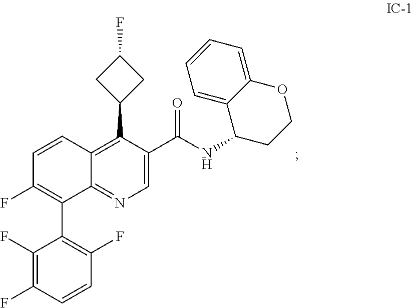 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, umifoxolaner, moxidectin and praziquantel.

78. The antiparasitic combination of any one of claims 1 to 17, 20 or 21, wherein the first antiparasitic active agent is Compound IC-1, or a salt thereof: 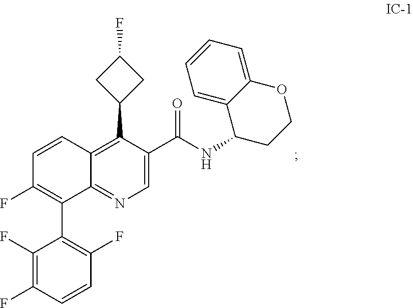 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, fluralaner, moxidectin and praziquantel.

79. The antiparasitic combination of any one of claims 1 to 17, 20 or 21, wherein the first antiparasitic active agent is Compound IC-1, or a salt thereof: 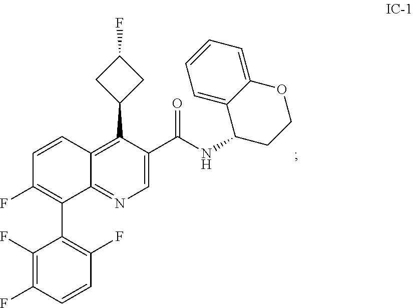 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, sarolaner, moxidectin and praziquantel.

80. The antiparasitic combination of any one of claims 1 to 17, 20 or 21, wherein the first antiparasitic active agent is Compound IC-1, or a salt thereof: 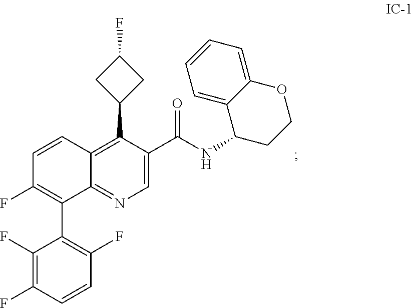 [SMILES: O=C(N[C@H]1CCOc2ccccc21)c1cnc2c(-c3c(F)ccc(F)c3F)c(F)ccc2c1[C@H]1C[C@H](F)C1]
and wherein the second or more additional antiparasitic active agent(s) comprises, consists essentially of, or consists of, lotilaner, moxidectin and praziquantel.

81. A veterinary or pharmaceutical composition comprising the antiparasitic combination of any one of claims 1 to 80 together with a pharmaceutically acceptable carrier.

82. An antiparasitic combination according to any one of claims 1 to 80 for use in treating and/or preventing a parasitic infection and/or infestation in or on an animal.

83. A veterinary or pharmaceutical composition according to claim 81, for use in treating and/or preventing a parasitic infection and/or infestation in or on an animal.

84. A method of treating and/or preventing a parasitic infection and/or infestation in or on an animal, comprising administering an antiparasitic combination according to any one of claims 1 to 80, or the veterinary or pharmaceutical composition according to claim 81, to an animal in need thereof.

85. The antiparasitic combination for use according to claim 82, or the veterinary or pharmaceutical composition for use according to claim 83, or the method according to claim 84, wherein the animal is a human, cat, dog, horse, cow, sheep or fish.

86. The antiparasitic combination for use according to claim 82, or the veterinary or pharmaceutical composition for use according to claim 83, or the method according to claim 84, wherein the parasitic infection and/or infestation is an ectoparasitic and/or endoparasitic infection and/or infestation.

87. The antiparasitic combination for use according to any one of claims 82, 85 or 86, or the veterinary or pharmaceutical composition for use according to any one of claims 83, 85 or 86, or the method according to any one of claims 84, 85 or 86, wherein the parasitic infection and/or infestation is caused by one or more of Dirofilaria immitis, hookworms, roundworms, cestodes, ticks and fleas, or a combination thereof.
